(19) United States
(12) Patent Application Publication
(10) Pub. No.: US 2012/0142668 A1
(43) Pub. Date: Jun. 7, 2012

(54) CATHEPSIN C INHIBITORS

(76) Inventors: Niall Anderson, Stevenage (GB); Ann M. Bullion, King of Prussia, PA (US); Neysa Nevins, Collegeville, PA (US); Michael R. Palovich, King of Prussia, PA (US); Steven L. Sollis, Stevenage (GB); Michael D. Wall, Saffron Walden (GB); Jakob Busch-Petersen, King of Prussia, PA (US); Brian Evans, Buntingford (GB); Huijie Li, King of Prussia, PA (US)

(21) Appl. No.: 13/389,631

(22) PCT Filed: Aug. 11, 2010
(86) PCT No.: PCT/US10/45137

§ 371 Date: Feb. 9, 2012

Related U.S. Application Data
(60) Provisional application No. 61/233,233, filed on Aug. 12, 2009.
(60) Provisional application No. 61/330,944, filed on May 4, 2010.

(51) Int. Cl. A61K 31/397; C07D 211/60; C07C 229/38; C07D 277/46; C07D 209/08; C07D 213/75; C07D 231/40; C07D 401/12; C07D 417/12; A61K 31/165; A61K 31/4453; A61K 31/24; A61K 31/404; A61K 31/4406; A61K 31/415; A61K 31/4468; A61P 11/00; A61K 31/426; A61K 31/454; C07C 237/20

(52) U.S. Cl. 514/210.18; 564/157; 546/226; 560/43; 548/195; 548/510; 546/309; 548/371.7; 546/208; 546/209; 514/616; 514/330; 514/535; 514/415; 514/352; 514/407; 514/329; 514/210.17; 514/371; 514/323; 514/326

(57) ABSTRACT

Disclosed are 4-amino-2-butenamides of Formula (I) having pharmacological activity, pharmaceutical compositions containing them, and methods for the treatment of diseases mediated by the cathepsin C enzyme such as chronic obstructive pulmonary disease.

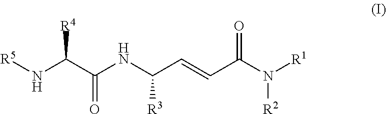

### FIELD OF THE INVENTION

The present invention relates to certain 4-amino-2-butenamides that are cathepsin C inhibitors, pharmaceutical compositions containing these compounds, and their use in the treatment of diseases mediated by the cathepsin C enzyme such as chronic obstructive pulmonary disease.

### BACKGROUND OF THE INVENTION

Cathepsins are a family of enzymes included in the papain superfamily of cysteine proteases. Cathepsins B, C, F, H, K, L, S, V, and X have been described in the scientific literature. Cathepsin C is also known in the literature as Dipeptidyl Peptidase I or “DPPI.”

A number of recently published studies have begun to describe the role cathepsin C plays in certain inflammatory processes. See e.g. Adkison et al., The Journal of Clinical Investigation 109:363-371 (2002); Tran et al., Archives of Biochemistry and Biophysics 403:160-170 (2002); Thiele et al., The Journal of Immunology 158: 5200-5210 (1997); Bidere et al., The Journal of Biological Chemistry 277: 32339-32347 (2002); Mabee et al., The Journal of Immunology 160: 5880-5885; McGuire et al., The Journal of Biological Chemistry, 268: 2458-2467; and Paris et al., FEBS Letters 369: 326-330 (1995). From these studies, it appears that cathepsin C is co-expressed in granules with certain serine proteases and functions to process the pro-forms of these proteases to active forms, which are then released from the granules of inflammatory cells recruited to sites of inflammation. Once activated, these proteases have a number of functions including degradation of various extracellular matrix components, which together can propagate tissue damage and chronic inflammation.

For example, Chronic Obstructive Pulmonary Disease (“COPD”) is a chronic inflammatory disease where cathepsin C appears to play a role. Chronic bronchitis and emphysema usually occur together in COPD patients. Chronic bronchitis is generally characterized by a chronic productive cough, whereas emphysema is generally characterized by permanent enlargement of the airspaces distal to the terminal bronchioles and airway wall destruction.

Cigarette smoking is a significant risk factor for developing COPD. Exposure to cigarette smoke and other noxious particles and gases may result in chronic inflammation of the lung. In response to such exposure, inflammatory cells such as CD8+ T cells, macrophages, and neutrophils are recruited to the area. These recruited inflammatory cells release proteases, which are believed to play a major role in the disease etiology by a number of mechanisms. Proteases believed to be involved in this process include the serine proteases neutrophil elastase (“NE”), cathepsin G, and proteinase 3, all released from neutrophils; granzymes A and B, released from cytotoxic T cells or natural killer cells; and chymases, released from mast cells. Cathepsin C appears to be involved in activating all of these enzymes.

Rheumatoid arthritis (“RA”) is another chronic inflammatory disease where cathepsin C may play a role. Neutrophils are recruited to the site of joint inflammation and release cathepsin G, NE, and proteinase 3, which are believed to be responsible in part for cartilage destruction associated with RA (Hu, Y. and Pham, C. T. (2005) Arthritis Rheum 52: 2553-2558).

Other conditions where cathepsin C may play a role include osteoarthritis, asthma, and Multiple Sclerosis. See e.g. Matsui, K.; Yuyama, N.; Akaiwa, M.; Yoshida, N. L.; Maeda, M.; Sugita, Y.; Izuhara, K., Identification of an alternative splicing variant of cathepsin C/dipeptidyl-peptidase I, Gene. 293(1-2):1-7, 2002 Jun. 26; Wolters, P. J.; Laig-Webster, M.; Caughey, G. H., Dipeptidyl peptidase I cleaves matrix-associated proteins and is expressed mainly by mast cells in normal dog airways, American Journal of Respiratory Cell & Molecular Biology. 22(2):183-90, 2000.

One approach to treating these conditions is to inhibit the activity of the serine proteases involved in the inflammatory process, especially NE activity. See e.g., Ohbayashi, “Neutrophil elastase inhibitors as treatment for COPD”, Expert Opin. Investig. Drugs 11(7): 965-980 (2002); Shapiro, “Neutrophil Elastase: Path Clearer, Pathogen Killer, or Just Pathologic?”, Am. J. Respir. Cell Mol. Biol. 26: 266-268 (2002). In light of the role cathepsin C plays in activating certain serine proteases, especially NE, it is desirable to prepare compounds that inhibit its activity, which thereby inhibit serine protease activity. Thus, there is a need to identify compounds that inhibit cathepsin C, which can be used in the treatment of a variety of conditions mediated by cathepsin C.

There are additional activities of cathepsin C that may also be related to disease etiology. Cathepsin C has been demonstrated to have a role in neutrophil migration in the development of aortic aneurysms by a mechanism which has not been clearly elucidated (Pagano, M. B. et al. (2007) PNAS 104: 2855-2860). Thus, disease processes that involve neutrophil migration, as well as proteolytic enzyme release can be modulated by cathepsin C inhibition. Also, cathepsin C is highly expressed in the lung epithelium where it may play a role in the processing of other enzymes not yet identified. Cathepsin C has also been reported to cleave kallikrein-4, which is believed to play a role in dental enamel maturation (Tye, C. E. et al. (2009) J. Dental Res. 88: 323-327). Finally, cathepsin C is itself released from cells and may play a direct role in the degradation of matrix proteins.

### SUMMARY OF THE INVENTION

The present invention involves novel compounds according to Formula (I) or a pharmaceutically acceptable salt thereof:

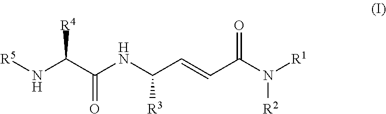

wherein:

R¹ and R² are each independently selected from the group consisting of hydrogen, (C₁-C₈)alkyl, (C₂-C₈)alkenyl, (C₂-C₈)alkynyl, (C₃-C₈)cycloalkyl, (C₅-C₈)cycloalkenyl, (C₆-C₁₀)bicycloalkyl, heterocycloalkyl, (C₃-C₈)cycloalkyl(C₁-C₆)alkyl, (C₅-C₈)cycloalkenyl(C₁-C₆)alkyl, heterocycloalkyl(C₁-C₆)alkyl, aryl, heteroaryl, aryl(C₁-C₆)alkyl, and heteroaryl(C₁-C₆)alkyl;
wherein any (C₁-C₈)alkyl, (C₂-C₈)alkenyl, or (C₂-C₈)alkynyl is optionally substituted one to three times, independently, by —CF₃, cyano, —CO₂(C₁-C₄)alkyl, —CONH(C₁-C₄)alkyl, —CON(C₁-C₄)alkyl(C₁-C₄)alkyl, —SO₂(C₁-C₄)alkyl, —SO₂NH(C₁-C₄)alkyl, —SO₂N(C₁-C₄)alkyl(C₁-C₄)alkyl, amino, (C₁-C₄)alkylamino, ((C₁-C₄)alkyl)((C₁-C₄)alkyl)amino, hydroxyl, or (C₁-C₄)alkoxy;
and wherein any cycloalkyl, cycloalkenyl, bicycloalkyl, or heterocycloalkyl group is optionally substituted one to three times, independently, by (C₁-C₄)alkyl, (C₁-C₄)haloalkyl, cyano, —CO₂(C₁-C₄)alkyl, —CONH(C₁-C₄)alkyl, —CON(C₁-C₄)alkyl(C₁-C₄)alkyl, —SO₂(C₁-C₄)alkyl, —SO₂NH(C₁-C₄)alkyl, —SO₂N(C₁-C₄)alkyl(C₁-C₄)alkyl, amino, (C₁-C₄)alkylamino, ((C₁-C₄)alkyl)((C₁-C₄)alkyl)amino, hydroxyl, (C₁-C₄)alkoxy, aryl, or aryl(C₁-C₄)alkyl, wherein the aryl moiety of said aryl or aryl(C₁-C₄)alkyl is optionally substituted one to three times, independently, by halogen, —CF₃, (C₁-C₄)alkyl, hydroxyl, or (C₁-C₄)alkoxy;
and wherein any aryl or heteroaryl group is optionally substituted one to three times, independently, by halogen, (C₁-C₆)alkyl, (C₃-C₆)cycloalkyl, (C₅-C₆)cycloalkenyl, (C₁-C₆)haloalkyl, cyano, —CO₂(C₁-C₄)alkyl, —CONH(C₁-C₄)alkyl, —CON(C₁-C₄)alkyl(C₁-C₄)alkyl, —SO₂(C₁-C₄)alkyl, —SO₂NH(C₁-C₄)alkyl, —SO₂N(C₁-C₄)alkyl(C₁-C₄)alkyl, amino, (C₁-C₄)alkylamino, ((C₁-C₄)alkyl)((C₁-C₄)alkyl)amino, hydroxyl, (C₁-C₄)alkoxy, (C₁-C₄)alkylthio-, aryl, heteroaryl, aryl(C₁-C₄)alkyl, or heteroaryl(C₁-C₄)alkyl;
   wherein any aryl or heteroaryl moiety of said aryl, heteroaryl, aryl(C₁-C₄)alkyl, or heteroaryl(C₁-C₄)alkyl is optionally substituted one to three times, independently, by halogen, —CF₃, (C₁-C₄)alkyl, hydroxyl, or (C₁-C₄)alkoxy;
   and wherein any (C₃-C₆)cycloalkyl is optionally substituted one to three times, independently, by (C₁-C₄)alkyl, aryl, or heteroaryl;
      wherein said aryl or heteroaryl is optionally substituted one to three times, independently, by halogen, —CF₃, (C₁-C₄)alkyl, hydroxyl, or (C₁-C₄)alkoxy;

or R¹ and R² taken together with the nitrogen to which they are attached represent a 5- to 7-membered saturated or unsaturated ring optionally containing one other heteroatom which is oxygen, nitrogen, or sulfur, wherein said ring is optionally fused to a (C₃-C₈)cycloalkyl, heterocycloalkyl, aryl, or heteroaryl ring;

or R¹ and R² taken together with the nitrogen to which they are attached represent a 6- to 10-membered bridged bicyclic ring system optionally fused to a (C₃-C₈)cycloalkyl, heterocycloalkyl, aryl, or heteroaryl ring;

R³ is hydrogen, (C₁-C₈)alkyl, (C₁-C₈)haloalkyl, (C₂-C₈)alkenyl, (C₂-C₈)alkynyl, (C₃-C₆)cyclo alkyl, (C₅-C₆)cycloalkenyl, (C₃-C₆)cycloalkyl(C₁-C₄)alkyl, (C₅-C₆)cycloalkenyl(C₁-C₄)alkyl, or aryl(C₁-C₄)alkyl, wherein the aryl moiety of the aryl(C₁-C₄)alkyl is optionally substituted one to three times, independently, by halogen, (C₁-C₄)alkyl, or —CF₃;

R⁴ is hydrogen, (C₁-C₄)alkyl, (C₂-C₅)alkenyl, (C₂-C₅)alkynyl, (C₃-C₅)cycloalkyl, (C₃-C₄)cycloalkyl(C₁-C₂)alkyl, cyano(C₁-C₂)alkyl, hydroxy(C₁-C₂)alkyl, methoxy(C₁-C₂)alkyl, aryl(C₁-C₂)alkyl, or heteroaryl(C₁-C₂)alkyl, wherein the heteroaryl moiety of said heteroaryl(C₁-C₂)alkyl is a 5-membered aromatic ring containing one heteroatom which is oxygen or sulfur and optionally containing one or two nitrogen atoms; and

R⁵ is hydrogen or methyl;

or R⁴ and R⁵ taken together with atoms through which they are connected form a 4- to 6-membered saturated ring optionally substituted one or two times, independently, by halogen, —CF₃, cyano, (C₁-C₄)alkyl, amino, (C₁-C₄)alkylamino, ((C₁-C₄)alkyl)((C₁-C₄)alkyl)amino, hydroxyl, (C₁-C₄)alkoxy, or (C₁-C₄)alkylthio-; wherein said ring is optionally fused to a (C₃-C₅)cycloalkyl ring.

The present invention is also directed to the use of a compound of Formula (I) or a pharmaceutically acceptable salt thereof in the prevention, management or treatment of a respiratory or inflammatory disease, such as chronic obstructive pulmonary disease or rhinitis.

In a further aspect, this invention relates to a pharmaceutically acceptable formulation comprising a compound of Formula (I) or a pharmaceutically acceptable salt thereof and a pharmaceutically acceptable excipient.

### DETAILED DESCRIPTION OF THE INVENTION

### Terms and Definitions

As used herein, the term “alkyl” refers to a straight- or branched-chain hydrocarbon radical having the specified number of carbon atoms. As used herein, the terms “(C₁-C₄)alkyl” and “(C₁-C₈)alkyl” refer to an alkyl group having at least 1 and up to 4 or 8 carbon atoms respectively. Examples of such branched or straight-chained alkyl groups useful in the present invention include, but are not limited to, methyl, ethyl, n-propyl, isopropyl, n-butyl, isobutyl, s-butyl, t-butyl, n-pentyl, isopentyl, n-hexyl, n-heptyl, n-octyl, and branched analogs of the latter 3 normal alkanes.

When the term “alkyl” is used in combination with other substituent groups, such as “(C₁-C₄)haloalkyl” or “aryl(C₁-C₄)alkyl”, the term “alkyl” is intended to encompass a divalent straight or branched-chain hydrocarbon radical, wherein the point of attachment is through the alkyl moiety. Examples of “(C₁-C₄)haloalkyl” groups useful in the present invention include, but are not limited to, —CF₃ (trifluoromethyl), —CCl₃ (trichloromethyl), 1,1-difluoroethyl, 2,2,2-trifluoroethyl, and hexafluoroisopropyl. Examples of “aryl(C₁-C₄)alkyl” groups useful in the present invention include, but are not limited to, benzyl (phenylmethyl), 1-methylbenzyl (1-phenylethyl), 1,1-dimethylbenzyl (1-phenylisopropyl), and phenethyl (2-phenylethyl).

As used herein, the term “alkenyl” refers to straight or branched hydrocarbon chains containing the specified number of carbon atoms and at least 1 and up to 3 carbon-carbon double bonds. Examples include ethenyl and propenyl.

As used herein, the term “alkynyl” refers to straight or branched hydrocarbon chains containing the specified number of carbon atoms and at least 1 and up to 3 carbon-carbon triple bonds. Examples include ethynyl and propynyl.

As used herein, the term “cycloalkyl” refers to a non-aromatic, saturated, cyclic hydrocarbon ring containing the specified number of carbon atoms. The term “(C₃-C₈)cycloalkyl” refers to a non-aromatic cyclic hydrocarbon ring having from three to eight ring carbon atoms. Exemplary “(C₃-C₈)cycloalkyl” groups useful in the present invention include cyclopropyl, cyclobutyl, cyclopentyl, cyclohexyl, cycloheptyl, and cyclooctyl.

As used herein, the term “cycloalkenyl” refers to a non-aromatic, cyclic hydrocarbon ring containing the specified number of carbon atoms and at least one carbon-carbon double bond. The term “(C₅-C₈)cycloalkenyl” refers to a non-aromatic cyclic hydrocarbon ring having from five to eight ring carbon atoms. Exemplary “(C₅-C₈)cycloalkenyl” groups useful in the present invention include cyclopentenyl, cyclohexenyl, cycloheptenyl, and cyclooctenyl.

As used herein, the term “bicycloalkyl” refers to a saturated, bridged, bicyclic hydrocarbon ring system containing the specified number of carbon atoms. The term “(C₆-C₁₀)bicycloalkyl” refers to a bicyclic hydrocarbon ring system having from six to ten carbon atoms. Exemplary “(C₆-C₁₀)bicycloalkyl” groups useful in the present invention include bicyclo[2.1.1]hexyl, bicyclo[2.1.1]heptyl, bicyclo[3.2.1]octyl, bicyclo[2.2.2]octyl, bicyclo[3.2.2]nonyl, bicyclo[3.3.1]nonyl, bicyclo[3.3.2]decyl, and bicyclo[4.3.1]decyl.

“Alkoxy” means an alkyl radical containing the specified number of carbon atoms attached through an oxygen linking atom. The term “(C₁-C₄)alkoxy” refers to a straight- or branched-chain hydrocarbon radical having at least 1 and up to 4 carbon atoms attached through an oxygen linking atom. Exemplary “(C₁-C₄)alkoxy” groups useful in the present invention include, but are not limited to, methoxy, ethoxy, n-propoxy, isopropoxy, n-butoxy, s-butoxy, and t-butoxy.

“Alkylthio-” means an alkyl radical containing the specified number of carbon atoms attached through a sulfur linking atom. The term “(C₁-C₄)alkylthio-” refers to a straight- or branched-chain hydrocarbon radical having at least 1 and up to 4 carbon atoms attached through a sulfur linking atom. Exemplary “(C₁-C₄)alkylthio-” groups useful in the present invention include, but are not limited to, methylthio-, ethylthio-, n-propylthio-, isopropylthio-, n-butylthio-, s-butylthio-, and t-butylthio-.

“Heterocycloalkyl” means a non-aromatic heterocyclic ring containing 3-8 or 5-6 ring atoms, being saturated or having one or more degrees of unsaturation and containing one or more heteroatom substitutions selected from O, S, and/or N. Such a ring may be optionally fused to one or more other heterocycloalkyl ring(s) or cycloalkyl ring(s). Examples of “heterocycloalkyl” moieties include, but are not limited to, aziridinyl, thiiranyl, oxiranyl, azetidinyl, oxetanyl, thietanyl, tetrahydrofuranyl, dihydropyranyl, tetrahydropyranyl, 1,4-dioxanyl, 1,3-dioxanyl, piperidinyl, piperazinyl, 2,4-piperazinedionyl, pyrrolidinyl, pyrrolinyl, imidazolidinyl, pyrazolidinyl, pyrazolinyl, morpholinyl, thiomorpholinyl, tetrahydrothiopyranyl, tetrahydrothienyl, and the like.

“Aryl” refers to optionally substituted monocyclic or fused bicyclic groups having 6 to 14 carbon atoms and having at least one aromatic ring that complies with Hückel's Rule. Examples of “aryl” groups are phenyl, naphthyl, indenyl, dihydroindenyl, anthracenyl, phenanthrenyl, and the like. Preferably aryl refers to optionally substituted phenyl.

“Heteroaryl” means an optionally substituted aromatic monocyclic ring or fused bicyclic ring system wherein at least one ring complies with Hückel's Rule, has the specified number of ring atoms, and that ring contains at least one heteroatom selected from N, O, and/or S. Examples of 5-membered “heteroaryl” groups include furanyl, thienyl, pyrrolyl, imidazolyl, pyrazolyl, triazolyl, tetrazolyl, thiazolyl, oxazolyl, isoxazolyl, oxadiazolyl, thiadiazolyl, and isothiazolyl. Examples of 6-membered “heteroaryl” groups include oxo-pyridyl, pyridinyl, pyridazinyl, pyrazinyl, and pyrimidinyl. Examples of 6,6-fused “heteroaryl” groups include quinolinyl, isoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl. Examples of 6,5-fused “heteroaryl” groups include benzofuranyl, benzothienyl, benzimidazolyl, benzthiazolyl, indolizinyl, indolyl, isoindolyl, and indazolyl.

For the avoidance of doubt, all bicyclic ring systems may be attached at any suitable position on either ring.

As used herein, “halogen” or “halo” refers to F, Cl, Br, or I.

“Optionally substituted” indicates that a group, such as alkyl, alkenyl, alkynyl, cycloalkyl, cycloalkenyl, bicycloalkyl, alkoxy, heterocycloalkyl, aryl, or heteroaryl, may be unsubstituted, or the group may be substituted with one or more substituent(s) as defined. In the case where groups may be selected from a number of alternative groups the selected groups may be the same or different.

The term “independently” means that where more than one substituent is selected from a number of possible substituents, those substituents may be the same or different. That is, each substituent is separately selected from the entire group of recited possible substituents (e.g. a group of substituents provided herein for various aryl or heteroaryl is halogen, —CF₃, (C₁-C₄)alkyl, hydroxyl, and (C₁-C₄)alkoxy).

The alternative definitions for the various groups and substituent groups of Formula (I) provided throughout the specification are intended to particularly describe each compound species disclosed herein, individually, as well as groups of one or more compound species. The scope of this invention includes any combination of these group and substituent group definitions. The compounds of the invention are only those which are contemplated to be “chemically stable” as will be appreciated by those skilled in the art.

Suitably, R¹ and R² are each independently selected from the group consisting of hydrogen, (C₁-C₈)alkyl, (C₂-C₈)alkenyl, (C₂-C₈)alkynyl, (C₃-C₈)cycloalkyl, (C₅-C₈)cycloalkenyl, (C₆-C₁₀)bicyclo alkyl, heterocycloalkyl, (C₃-C₈)cycloalkyl(C₁-C₆)alkyl, (C₅-C₈)cycloalkenyl(C₁-C₆)alkyl, heterocycloalkyl(C₁-C₆)alkyl, aryl, heteroaryl, aryl(C₁-C₆)alkyl, and heteroaryl(C₁-C₆)alkyl;
wherein any (C₁-C₈)alkyl, (C₂-C₈)alkenyl, or (C₂-C₈)alkynyl is optionally substituted one to three times, independently, by —CF₃, cyano, —CO₂(C₁-C₄)alkyl, —CONH(C₁-C₄)alkyl, —CON(C₁-C₄)alkyl(C₁-C₄)alkyl, —SO₂(C₁-C₄)alkyl, —SO₂NH(C₁-C₄)alkyl, —SO₂N(C₁-C₄)alkyl(C₁-C₄)alkyl, amino, (C₁-C₄)alkylamino, ((C₁-C₄)alkyl)((C₁-C₄)alkyl)amino, hydroxyl, or (C₁-C₄)alkoxy;
and wherein any cycloalkyl, cycloalkenyl, bicycloalkyl, or heterocycloalkyl group is optionally substituted one to three times, independently, by (C₁-C₄)alkyl, (C₁-C₄)haloalkyl, cyano, —CO₂(C₁-C₄)alkyl, —CONH(C₁-C₄)alkyl, —CON(C₁-C₄)alkyl(C₁-C₄)alkyl, —SO₂(C₁-C₄)alkyl, —SO₂NH(C₁-C₄)alkyl, —SO₂N(C₁-C₄)alkyl(C₁-C₄)alkyl, amino, (C₁-C₄)alkylamino, ((C₁-C₄)alkyl)((C₁-C₄)alkyl)amino, hydroxyl, (C₁-C₄)alkoxy, aryl, or aryl(C₁-C₄)alkyl; wherein the aryl moiety of said aryl or aryl(C₁-C₄)alkyl is optionally substituted one to three times, independently, by halogen, —CF₃, (C₁-C₄)alkyl, hydroxyl, or (C₁-C₄)alkoxy;
and wherein any aryl or heteroaryl group is optionally substituted one to three times, independently, by halogen, (C₁-C₆)alkyl, (C₃-C₆)cycloalkyl, (C₅-C₆)cycloalkenyl, (C₁-C₆)haloalkyl, cyano, —CO₂(C₁-C₄)alkyl, —CONH(C₁-C₄)alkyl, —CON(C₁-C₄)alkyl(C₁-C₄)alkyl, —SO₂(C₁-C₄)alkyl, —SO₂NH(C₁-C₄)alkyl, —SO₂N(C₁-C₄)alkyl(C₁-C₄)alkyl, amino, (C₁-C₄)alkylamino, ((C₁-C₄)alkyl)((C₁-C₄)alkyl)amino, hydroxyl, (C₁-C₄)alkoxy, (C₁-C₄)alkylthio-, aryl, heteroaryl, aryl(C₁-C₄)alkyl, or heteroaryl(C₁-C₄)alkyl;
   wherein any aryl or heteroaryl moiety of said aryl, heteroaryl, aryl(C₁-C₄)alkyl, or heteroaryl(C₁-C₄)alkyl is optionally substituted one to three times, independently, by halogen, —CF₃, (C₁-C₄)alkyl, hydroxyl, or (C₁-C₄)alkoxy;
   and wherein any (C₃-C₆)cycloalkyl is optionally substituted one to three times, independently, by (C₁-C₄)alkyl, aryl, or heteroaryl;
      wherein said aryl or heteroaryl is optionally substituted one to three times, independently, by halogen, —CF₃, (C₁-C₄)alkyl, hydroxyl, or (C₁-C₄)alkoxy.

In another embodiment, R¹ and R² are each independently selected from the group consisting of hydrogen, (C₁-C₆)alkyl, (C₃-C₇)cycloalkyl, (C₇-C₉)bicycloalkyl, heterocycloalkyl, (C₃-C₇)cycloalkyl(C₁-C₄)alkyl, phenyl, heteroaryl, phenyl(C₁-C₄)alkyl, and heteroaryl(C₁-C₄)alkyl;
wherein any (C₁-C₆)alkyl group is optionally substituted one to three times, independently, by (C₃-C₆)cycloalkyl, —CF₃, cyano, —CO₂(C₁-C₄)alkyl, hydroxyl, or (C₁-C₄)alkoxy;
and wherein any cycloalkyl, bicycloalkyl, or heterocycloalkyl group is optionally substituted one to three times, independently, by (C₁-C₄)alkyl, —CF₃, cyano, —CO₂(C₁-C₄)alkyl, hydroxyl, (C₁-C₄)alkoxy, phenyl, or phenyl(C₁-C₂)alkyl;
wherein the phenyl moiety of said phenyl or phenyl(C₁-C₂)alkyl is optionally substituted one to three times, independently, by halogen, —CF₃, (C₁-C₄)alkyl, hydroxyl, or (C₁-C₄)alkoxy;
and wherein any phenyl or heteroaryl group is optionally substituted one to three times, independently, by halogen, (C₁-C₆)alkyl, (C₃-C₆)cycloalkyl, —CF₃, cyano, —CO₂(C₁-C₄)alkyl, —SO₂(C₁-C₄)alkyl, hydroxyl, (C₁-C₄)alkoxy, (C₁-C₄)alkylthio-, phenyl, heteroaryl, phenyl(C₁-C₄)alkyl, or heteroaryl(C₁-C₄)alkyl;
   wherein any phenyl or heteroaryl moiety of said phenyl, heteroaryl, phenyl(C₁-C₄)alkyl, or heteroaryl(C₁-C₄)alkyl is optionally substituted one to three times, independently, by halogen, —CF₃, or (C₁-C₄)alkyl;
   and wherein any (C₃-C₆)cycloalkyl is optionally substituted one to three times, independently, by (C₁-C₄)alkyl, phenyl, or heteroaryl;
      wherein said phenyl or heteroaryl is optionally substituted one to three times, independently, by halogen, —CF₃, or (C₁-C₄)alkyl.

In a further embodiment, R¹ is selected from the group consisting of (C₁-C₆)alkyl, (C₃-C₇)cycloalkyl, (C₇-C₉)bicycloalkyl, heterocycloalkyl, (C₃-C₇)cycloalkyl(C₁-C₂)alkyl, phenyl, heteroaryl, and phenyl(C₁-C₂)alkyl; wherein any cycloalkyl or heterocycloalkyl group is optionally substituted one to two times, independently, by (C₁-C₄)alkyl, —CF₃, hydroxyl, or (C₁-C₄)alkoxy, and wherein any phenyl or heteroaryl group is optionally substituted one to two times, independently, by halogen, (C₁-C₄)alkyl, —CF₃, cyano, —CO₂(C₁-C₄)alkyl, hydroxyl, (C₁-C₄)alkoxy, or (C₁-C₄)alkylthio-. In yet a further embodiment, R¹ is phenyl optionally substituted one to two times, independently, by halogen, (C₁-C₄)alkyl, —CF₃, cyano, —CO₂ (C₁-C₄)alkyl, hydroxyl, (C₁-C₄)alkoxy, or (C₁-C₄)alkylthio-. In yet a further embodiment, R¹ is furanyl, thienyl, pyrrolyl, imidazolyl, pyrazolyl, triazolyl, tetrazolyl, thiazolyl, oxazolyl, isoxazolyl, oxadiazolyl, thiadiazolyl, or isothiazolyl optionally substituted by halogen, (C₁-C₄)alkyl, —CF₃, (C₃-C₆)cycloalkyl, phenyl, halophenyl, phenyl(C₁-C₄)alkyl, halophenyl(C₁-C₄)alkyl, cyano, —CO₂(C₁-C₄)alkyl, (C₁-C₄)alkoxy, or (C₁-C₄)alkylthio-; wherein said (C₃-C₆)cycloalkyl is optionally substituted by (C₁-C₄)alkyl. In yet a further embodiment, R¹ is thiadiazolyl optionally substituted by halogen, (C₁-C₄)alkyl, —CF₃, (C₃-C₆)cycloalkyl, phenyl, halophenyl, phenyl(C₁-C₄)alkyl, cyano, —CO₂(C₁-C₄)alkyl, (C₁-C₄)alkoxy, or (C₁-C₄)alkylthio-; wherein said (C₃-C₆)cycloalkyl is optionally substituted by (C₁-C₄)alkyl. In yet a further embodiment, R¹ is thiadiazolyl optionally substituted by halogen, (C₁-C₄)alkyl, —CF₃, (C₃-C₆)cycloalkyl, phenyl, cyano, —CO₂(C₁-C₄)alkyl, or (C₁-C₄)alkoxy; wherein said (C₃-C₆)cycloalkyl is optionally substituted by (C₁-C₄)alkyl. In selected embodiments, R¹ is methyl, ethyl, n-propyl, isopropyl, s-butyl, t-butyl, cyclopentyl, 3-hydroxycyclopentyl, cyclohexyl, 2-methylcyclohexyl, 4-hydroxycyclohexyl, cycloheptyl, bicyclo[2.2.1]hept-2-yl, tetrahydro-3-furanyl, tetrahydro-2H-pyran-3-yl, tetrahydro-2H-pyran-4-yl, 1-methyl-3-piperidinyl, 1-methyl-4-piperidinyl, phenyl, 3-trifluoromethylphenyl, 4-trifluoromethylphenyl, 3-carboxymethylphenyl, 4-carboxymethylphenyl, 2-methoxyphenyl, 3-methoxyphenyl, 4-methoxyphenyl, 3-pyridinyl, 1H-pyrazol-4-yl, 1,3-thiazol-2-yl, cyclohexylmethyl, benzyl, 5-(1-methylcyclobutyl)-1,3,4-thiadiazol-2-yl, 5-methyl-1,3,4-thiadiazol-2-yl, 5-ethyl-1,3,4-thiadiazol-2-yl, 5-(propan-2-yl)-1,3,4-thiadiazol-2-yl, 5-tert-butyl-1,3,4-thiadiazol-2-yl, 5-(trifluoromethyl)-1,3,4-thiadiazol-2-yl, 5-cyclopropyl-1,3,4-thiadiazol-2-yl, 5-cyclohexyl-1,3,4-thiadiazol-2-yl, 5-phenyl-1,3,4-thiadiazol-2-yl, 5-(4-fluorophenyl)-1,3,4-thiadiazol-2-yl, 5-(4-bromophenyl)-1,3,4-thiadiazol-2-yl, 5-benzyl-1,3,4-thiadiazol-2-yl, 5-(1-methyl-1-phenylethyl)-1,3,4-thiadiazol-2-yl, 5-(2-phenylethyl)-1,3,4-thiadiazol-2-yl, or 5-(methylsulfanyl)-1,3,4-thiadiazol-2-yl.

In another embodiment, R² is hydrogen or (C₁-C₄)alkyl. In selected embodiments, R² is hydrogen or methyl.

In another embodiment, R¹ and R² taken together with the nitrogen to which they are attached represent a 5- to 7-membered saturated or unsaturated ring optionally containing one other heteroatom which is oxygen, nitrogen, or sulfur; wherein said ring is optionally fused to a (C₃-C₈)cycloalkyl, heterocycloalkyl, aryl, or heteroaryl ring. In a further embodiment, R¹ and R² taken together with the nitrogen to which they are attached represent a 5- to 6-membered saturated or unsaturated ring optionally fused to a phenyl moiety. In a selected embodiment, R¹ and R² taken together with the nitrogen to which they are attached represent piperidin-1-yl, 1H-indol-1-yl, 2,3-dihydro-1H-indol-1-yl, or 1,3-dihydro-2H-isoindol-2-yl. In another selected embodiment, R¹ and R² taken together with the nitrogen to which they are attached represent 2,3-dihydro-1H-indol-1-yl.

In another embodiment, R¹ and R² taken together with the nitrogen to which they are attached represent a 6- to 10-membered bridged bicyclic ring system optionally fused to a (C₃-C₈)cycloalkyl, heterocycloalkyl, aryl, or heteroaryl ring. In a further embodiment, R¹ and R² taken together with the nitrogen to which they are attached represent a 7- to 9-membered bridged bicyclic ring system optionally fused to a phenyl moiety. In a selected embodiment, R¹ and R² taken together with the nitrogen to which they are attached represent an 11-azatricyclo[6.2.1.0^{2,7}]undeca-2,4,6-trien-11-yl ring system.

Suitably, R³ is hydrogen, (C₁-C₈)alkyl, (C₁-C₈)haloalkyl, (C₂-C₈)alkenyl, (C₂-C₈)alkynyl, (C₃-C₆)cycloalkyl, (C₅-C₆)cycloalkenyl, (C₃-C₆)cycloalkyl(C₁-C₄)alkyl, (C₅-C₆)cycloalkenyl(C₁-C₄)alkyl, or aryl(C₁-C₄)alkyl; wherein the aryl moiety of the aryl(C₁-C₄)alkyl is optionally substituted one to three times, independently, by halogen, (C₁-C₄)alkyl, or —CF₃.

In another embodiment, R³ is hydrogen, (C₁-C₆)alkyl, (C₁-C₆)haloalkyl, (C₃-C₆)cycloalkyl, (C₃-C₆)cycloalkyl(C₁-C₄)alkyl, or phenyl(C₁-C₄)alkyl; wherein the phenyl moiety of the phenyl(C₁-C₄)alkyl is optionally substituted one to three times, independently, by halogen, (C₁-C₄)alkyl, or —CF₃. In a further embodiment, R³ is (C₁-C₆)alkyl or (C₃-C₆)cycloalkyl(C₁-C₂)alkyl. In selected embodiments, R³ is ethyl, isobutyl, or sec-butyl. In selected embodiments, R³ is cyclopropylmethyl. In a further embodiment, R³ is phenyl(C₁-C₄)alkyl; wherein the phenyl moiety is optionally substituted one to two times, independently, by halogen, (C₁-C₄)alkyl, or —CF₃. In a selected embodiment, R³ is phenethyl.

Suitably, R⁴ is hydrogen, (C₁-C₄)alkyl, (C₂-C₅)alkenyl, (C₂-C₅)alkynyl, (C₃-C₅)cycloalkyl, (C₃-C₄)cycloalkyl(C₁-C₂)alkyl, cyano(C₁-C₂)alkyl, hydroxy(C₁-C₂)alkyl, methoxy(C₁-C₂)alkyl, aryl(C₁-C₂)alkyl, or heteroaryl(C₁-C₂)alkyl; wherein the heteroaryl moiety of said heteroaryl(C₁-C₂)alkyl is a 5-membered aromatic ring containing one heteroatom which is oxygen or sulfur and optionally containing one or two nitrogen atoms.

In another embodiment, R⁴ is hydrogen, (C₁-C₄)alkyl, (C₃-C₅)cycloalkyl, or heteroaryl(C₁-C₂)alkyl; wherein the heteroaryl moiety of said heteroaryl(C₁-C₂)alkyl is a 5-membered aromatic ring containing one heteroatom which is oxygen or sulfur and optionally containing one or two nitrogen atoms. In a further embodiment, R⁴ is (C₁-C₄)alkyl, (C₃-C₅)cycloalkyl, or thienyl(C₁-C₂)alkyl. In selected embodiments, R⁴ is methyl, ethyl, isopropyl, cyclopentyl, or 2-thienylmethyl. In a selected embodiment, R⁴ is methyl. In another selected embodiment, R⁴ is 2-thienylmethyl.

Suitably, R⁵ is hydrogen or methyl. In a selected embodiment, R⁵ is hydrogen.

In another embodiment, R⁴ and R⁵ taken together with atoms through which they are connected form a 4- to 6-membered saturated ring optionally substituted one or two times, independently, by halogen, —CF₃, cyano, (C₁-C₄)alkyl, amino, (C₁-C₄)alkylamino, ((C₁-C₄)alkyl)((C₁-C₄)alkyl)amino, hydroxyl, (C₁-C₄)alkoxy, or (C₁-C₄)alkylthio-; wherein said ring is optionally fused to a (C₃-C₅)cycloalkyl ring. In a further embodiment, R⁴ and R⁵ taken together with atoms through which they are connected form a 4- to 6-membered saturated ring optionally substituted one or two times, independently, by halogen, —CF₃, cyano, methyl, amino, hydroxyl, methoxy, or methylthio-; wherein said ring is optionally fused to a cyclopropyl ring. In a further embodiment, R⁴ and R⁵ taken together with atoms through which they are connected form a 4- to 6-membered saturated ring optionally substituted by F, Cl, —CF₃, cyano, methyl, methoxy, or methylthio-. In a further embodiment, R⁴ and R⁵ taken together with atoms through which they are connected form a 4- to 6-membered saturated ring optionally substituted by F. In a selected embodiment, R⁴ and R⁵ taken together represent —CH₂CH₂—. In another selected embodiment, R⁴ and R⁵ taken together represent —CH₂CH₂CH₂—. In another selected embodiment, R⁴ and R⁵ taken together represent —CH₂CHFCH₂—. In another selected embodiment, R⁴ and R⁵ taken together represent —CH₂CH₂CH₂CH₂—. In another selected embodiment, R⁴ and R⁵ taken together with atoms through which they are connected form a 3-azabicyclo[3.1.0]hexane ring system.

One particular embodiment of the invention is a compound of Formula (I) or a pharmaceutically acceptable salt thereof wherein:

R¹ and R² are each independently selected from the group consisting of hydrogen, (C₁-C₈)alkyl, (C₂-C₈)alkenyl, (C₂-C₈)alkynyl, (C₃-C₈)cycloalkyl, (C₅-C₈)cycloalkenyl, (C₆-C₁₀)bicycloalkyl, heterocycloalkyl, (C₃-C₈)cycloalkyl(C₁-C₆)alkyl, (C₅-C₈)cycloalkenyl(C₁-C₆)alkyl, heterocycloalkyl(C₁-C₆)alkyl, aryl, heteroaryl, aryl(C₁-C₆)alkyl, and heteroaryl(C₁-C₆)alkyl;
wherein any (C₁-C₈)alkyl, (C₂-C₈)alkenyl, or (C₂-C₈)alkynyl is optionally substituted one to three times, independently, by —CF₃, cyano, —CO₂(C₁-C₄)alkyl, —CONH(C₁-C₄)alkyl, —CON(C₁-C₄)alkyl(C₁-C₄)alkyl, —SO₂(C₁-C₄)alkyl, —SO₂NH(C₁-C₄)alkyl, —SO₂N(C₁-C₄)alkyl(C₁-C₄)alkyl, amino, (C₁-C₄)alkylamino, ((C₁-C₄)alkyl)((C₁-C₄)alkyl)amino, hydroxyl, or (C₁-C₄)alkoxy;
and wherein any cycloalkyl, cycloalkenyl, bicycloalkyl, or heterocycloalkyl group is optionally substituted one to three times, independently, by (C₁-C₄)alkyl, —CF₃, cyano, —CO₂(C₁-C₄)alkyl, —CONH(C₁-C₄)alkyl, —CON(C₁-C₄)alkyl(C₁-C₄)alkyl, —SO₂(C₁-C₄)alkyl, —SO₂NH(C₁-C₄)alkyl, —SO₂N(C₁-C₄)alkyl(C₁-C₄)alkyl, amino, (C₁-C₄)alkylamino, ((C₁-C₄)alkyl)((C₁-C₄)alkyl)amino, hydroxyl, (C₁-C₄)alkoxy, aryl, or aryl(C₁-C₄)alkyl, wherein the aryl moiety of said aryl or aryl(C₁-C₄)alkyl is optionally substituted one to three times, independently, by halogen, —CF₃, (C₁-C₄)alkyl, hydroxyl, or (C₁-C₄)alkoxy;
and wherein any aryl or heteroaryl group is optionally substituted one to three times, independently, by halogen, (C₁-C₆)alkyl, (C₃-C₆)cycloalkyl, (C₅-C₆)cycloalkenyl, —CF₃, cyano, —CO₂(C₁-C₄)alkyl, —CONH(C₁-C₄)alkyl, —CON(C₁-C₄)alkyl(C₁-C₄)alkyl, —SO₂(C₁-C₄)alkyl, —SO₂NH(C₁-C₄)alkyl, —SO₂N(C₁-C₄)alkyl(C₁-C₄)alkyl, amino, (C₁-C₄)alkylamino, ((C₁-C₄)alkyl)((C₁-C₄)alkyl)amino, hydroxyl, (C₁-C₄)alkoxy, aryl, heteroaryl, aryl(C₁-C₄)alkyl, or heteroaryl(C₁-C₄)alkyl;
   wherein any aryl or heteroaryl moiety of said aryl, heteroaryl, aryl(C₁-C₄)alkyl, or heteroaryl(C₁-C₄)alkyl is optionally substituted one to three times, independently, by halogen, —CF₃, (C₁-C₄)alkyl, hydroxyl, or (C₁-C₄)alkoxy;
   and wherein any (C₃-C₆)cycloalkyl is optionally substituted one to three times, independently, by (C₁-C₄)alkyl, aryl, or heteroaryl;
      wherein said aryl or heteroaryl is optionally substituted one to three times, independently, by halogen, —CF₃, (C₁-C₄)alkyl, hydroxyl, or (C₁-C₄)alkoxy;

or R¹ and R² taken together with the nitrogen to which they are attached represent a 5- to 7-membered saturated or unsaturated ring optionally containing one other heteroatom which is oxygen, nitrogen, or sulfur, wherein said ring is optionally fused to a (C₃-C₈)cycloalkyl, heterocycloalkyl, aryl, or heteroaryl ring;

or R¹ and R² taken together with the nitrogen to which they are attached represent a 6- to 10-membered bridged bicyclic ring system optionally fused to a (C₃-C₈)cycloalkyl, heterocycloalkyl, aryl, or heteroaryl ring;

R³ is hydrogen, (C₁-C₈)alkyl, (C₂-C₈)alkenyl, (C₂-C₈)alkynyl, (C₃-C₆)cycloalkyl, (C₅-C₆)cycloalkenyl, (C₃-C₆)cycloalkyl(C₁-C₄)alkyl, (C₅-C₆)cycloalkenyl(C₁-C₄)alkyl, or aryl(C₁-C₄)alkyl, wherein the aryl moiety of the aryl(C₁-C₄)alkyl is optionally substituted one to three times, independently, by halogen, (C₁-C₄)alkyl, or —CF₃;

R⁴ is hydrogen, (C₁-C₄)alkyl, (C₂-C₅)alkenyl, (C₂-C₅)alkynyl, (C₃-C₅)cycloalkyl, (C₃-C₄)cycloalkyl(C₁-C₂)alkyl, cyano(C₁-C₂)alkyl, hydroxy(C₁-C₂)alkyl, methoxy(C₁-C₂)alkyl, aryl(C₁-C₂)alkyl, or heteroaryl(C₁-C₂)alkyl, wherein the heteroaryl moiety of said heteroaryl(C₁-C₂)alkyl is a 5-membered monocyclic aromatic ring containing one to three heteroatoms selected independently from oxygen, nitrogen, and sulfur, wherein one of said heteroatoms is oxygen or sulfur; and

R⁵ is hydrogen or methyl;

or R⁴ and R⁵ taken together represent —CH₂CH₂— or —CH₂CH₂CH₂—.

Another particular embodiment of the invention is a compound of Formula (I) or a pharmaceutically acceptable salt thereof wherein:

R¹ and R² are each independently selected from the group consisting of hydrogen, (C₁-C₆)alkyl, (C₃-C₇)cycloalkyl, (C₇-C₉)bicycloalkyl, heterocycloalkyl, (C₃-C₇)cycloalkyl(C₁-C₄)alkyl, phenyl, heteroaryl, phenyl(C₁-C₄)alkyl, and heteroaryl(C₁-C₄)alkyl;
wherein any (C₁-C₆)alkyl group is optionally substituted one to three times, independently, by (C₃-C₆)cycloalkyl, —CF₃, cyano, —CO₂(C₁-C₄)alkyl, hydroxyl, or (C₁-C₄)alkoxy;
and wherein any cycloalkyl, bicycloalkyl, or heterocycloalkyl group is optionally substituted one to three times, independently, by (C₁-C₄)alkyl, —CF₃, cyano, —CO₂(C₁-C₄)alkyl, hydroxyl, (C₁-C₄)alkoxy, phenyl, or phenyl(C₁-C₂)alkyl;
wherein the phenyl moiety of said phenyl or phenyl(C₁-C₂)alkyl is optionally substituted one to three times, independently, by halogen, —CF₃, (C₁-C₄)alkyl, hydroxyl, or (C₁-C₄)alkoxy; and wherein any phenyl or heteroaryl group is optionally substituted one to three times, independently, by halogen, (C₁-C₆)alkyl, (C₃-C₆)cycloalkyl, —CF₃, cyano, —CO₂(C₁-C₄)alkyl, —SO₂(C₁-C₄)alkyl, hydroxyl, (C₁-C₄)alkoxy, phenyl, heteroaryl, phenyl(C₁-C₂)alkyl, or heteroaryl(C₁-C₂)alkyl;
   wherein any phenyl or heteroaryl moiety of said phenyl, heteroaryl, phenyl(C₁-C₂)alkyl, or heteroaryl(C₁-C₂)alkyl is optionally substituted one to three times, independently, by halogen, —CF₃, or (C₁-C₄)alkyl;
   and wherein any (C₃-C₆)cycloalkyl is optionally substituted one to three times, independently, by (C₁-C₄)alkyl, phenyl, or heteroaryl;
      wherein said phenyl or heteroaryl is optionally substituted one to three times, independently, by halogen, —CF₃, or (C₁-C₄)alkyl;

or R¹ and R² taken together with the nitrogen to which they are attached represent a 5- to 6-membered saturated or unsaturated ring optionally fused to a phenyl moiety;

or R¹ and R² taken together with the nitrogen to which they are attached represent a 7- to 9-membered bridged bicyclic ring system optionally fused to a phenyl moiety;

R³ is phenyl(C₁-C₄)alkyl; wherein the phenyl moiety is optionally substituted one to two times, independently, by halogen, (C₁-C₄)alkyl, or —CF₃;

R⁴ is (C₁-C₄)alkyl or thienyl(C₁-C₂)alkyl; and

R⁵ is hydrogen.

Another particular embodiment of the invention is a compound of Formula (I) or a pharmaceutically acceptable salt thereof wherein:

R¹ is selected from the group consisting of (C₁-C₆)alkyl, (C₃-C₇)cycloalkyl, (C₇-C₉)bicycloalkyl, heterocycloalkyl, (C₃-C₇)cycloalkyl(C₁-C₂)alkyl, phenyl, heteroaryl, and phenyl(C₁-C₂)alkyl; wherein any cycloalkyl or heterocycloalkyl group is optionally substituted one to two times, independently, by (C₁-C₄)alkyl, —CF₃, hydroxyl, or (C₁-C₄)alkoxy, and wherein any phenyl or heteroaryl group is optionally substituted one to two times, independently, by halogen, (C₁-C₄)alkyl, —CF₃, cyano, —CO₂(C₁-C₄)alkyl, hydroxyl, or (C₁-C₄)alkoxy;

R² is hydrogen or (C₁-C₄)alkyl;

R³ is phenethyl;

R⁴ is methyl, ethyl, isopropyl, or 2-thienylmethyl; and

R⁵ is hydrogen.

Another particular embodiment of the invention is a compound of Formula (I) or a pharmaceutically acceptable salt thereof wherein:

R¹ and R² taken together with the nitrogen to which they are attached represent a 5- to 6-membered saturated or unsaturated ring optionally fused to a phenyl moiety;

R³ is (C₁-C₆)alkyl; and

R⁴ and R⁵ taken together represent —CH₂CH₂— or —CH₂CH₂CH₂—.

Another particular embodiment of the invention is a compound of Formula (I) or a pharmaceutically acceptable salt thereof wherein:

R¹ and R² taken together with the nitrogen to which they are attached represent 2,3-dihydro-1H-indol-1-yl;

R³ is (C₁-C₆)alkyl or (C₃-C₆)cycloalkyl(C₁-C₂)alkyl; and

R⁴ and R⁵ taken together with atoms through which they are connected form a 4- to 6-membered saturated ring optionally substituted by F, Cl, —CF₃, cyano, methyl, methoxy, or methylthio-.

Another particular embodiment of the invention is a compound of Formula (I) or a pharmaceutically acceptable salt thereof wherein:

R¹ is heteroaryl optionally substituted one to two times, independently, by halogen, (C₁-C₄)alkyl, —CF₃, cyano, —CO₂(C₁-C₄)alkyl, hydroxyl, or (C₁-C₄)alkoxy; wherein said heteroaryl is selected from the group consisting of furanyl, thienyl, pyrrolyl, imidazolyl, pyrazolyl, triazolyl, tetrazolyl, thiazolyl, oxazolyl, isoxazolyl, oxadiazolyl, thiadiazolyl, and isothiazolyl;

R² is hydrogen or methyl;

R³ is (C₁-C₆)alkyl; and

R⁴ and R⁵ taken together represent —CH₂CH₂— or —CH₂CH₂CH₂—.

Another particular embodiment of the invention is a compound of Formula (I) or a pharmaceutically acceptable salt thereof wherein:

R¹ is thiadiazolyl optionally substituted by halogen, (C₁-C₄)alkyl, —CF₃, (C₃-C₆)cycloalkyl, phenyl, cyano, —CO₂(C₁-C₄)alkyl, or (C₁-C₄)alkoxy; wherein said (C₃-C₆)cycloalkyl is optionally substituted by (C₁-C₄)alkyl;

R² is hydrogen or methyl;

R³ is (C₁-C₆)alkyl or (C₃-C₆)cycloalkyl(C₁-C₂)alkyl; and

R⁴ and R⁵ taken together with atoms through which they are connected form a 4- to 6-membered saturated ring optionally substituted by F, Cl, —CF₃, cyano, methyl, methoxy, or methylthio-.

Specific compounds exemplified herein are:
(2E,4S)-4-(L-alanylamino)-6-phenyl-N-(phenylmethyl)-2-hexenamide;
(2E,4S)-4-(L-alanylamino)-N-methyl-6-phenyl-2-hexenamide;
(2E,4S)-4-(L-alanylamino)-N,N-dimethyl-6-phenyl-2-hexenamide;
N¹-[(1S,2E)-4-oxo-1-(2-phenylethyl)-4-(1-piperidinyl)-2-buten-1-yl]-L-alaninamide;
(2E,4S)-4-(L-alanylamino)-N-[4-(methyloxy)phenyl]-6-phenyl-2-hexenamide;
methyl 3-{[(2E,4S)-4-(L-alanylamino)-6-phenyl-2-hexenoyl]amino}benzoate;
(2E,4S)-4-(L-alanylamino)-N-[2-(methyloxy)phenyl]-6-phenyl-2-hexenamide;
(2E,4S)-4-(L-alanylamino)-6-phenyl-N-1,3-thiazol-2-yl-2-hexenamide;
(2E,4S)-4-(L-alanylamino)-6-phenyl-N-[3-(trifluoromethyl)phenyl]-2-hexenamide;
(2E,4S)-4-(L-alanylamino)-N-methyl-N,6-diphenyl-2-hexenamide;
(2E,4S)-4-(L-alanylamino)-6-phenyl-N-[4-(trifluoromethyl)phenyl]-2-hexenamide;
methyl 4-{[(2E,4S)-4-(L-alanylamino)-6-phenyl-2-hexenoyl]amino}benzoate;
(2E,4S)-4-(L-alanylamino)-N-cyclohexyl-N-methyl-6-phenyl-2-hexenamide;
(2E,4S)-4-(L-alanylamino)-N-[(1R,3S)-3-hydroxycyclopentyl]-6-phenyl-2-hexenamide;
(2E,4S)-4-(L-alanylamino)-N-[(1S,4R)-bicyclo[2.2.1]hept-2-yl]-6-phenyl-2-hexenamide;
N¹-[(1S,2E)-4-(1H-indol-1-yl)-4-oxo-1-(2-phenylethyl)-2-buten-1-yl]-L-alaninamide;
(2E,4S)-4-(L-alanylamino)-N-[3-(methyloxy)phenyl]-6-phenyl-2-hexenamide;
(2E,4S)-4-(L-alanylamino)-N-cyclohexyl-6-phenyl-2-hexenamide;
(2E,4S)-4-(L-alanylamino)-6-phenyl-N-3-pyridinyl-2-hexenamide;
(2E,4S)-4-(L-alanylamino)-6-phenyl-N-1H-pyrazol-4-yl-2-hexenamide;
(2E,4S)-4-(L-alanylamino)-6-phenyl-N-propyl-2-hexenamide;
(2E,4S)-4-(L-alanylamino)-N-(1,1-dimethylethyl)-6-phenyl-2-hexenamide;
(2E,4S)-4-(L-alanylamino)-N-cyclopentyl-6-phenyl-2-hexenamide;
(2E,4S)-4-(L-alanylamino)-N-(1-methyl-4-piperidinyl)-6-phenyl-2-hexenamide;
(2E,4S)-4-(L-alanylamino)-6-phenyl-N-(tetrahydro-2H-pyran-4-yl)-2-hexenamide;
(2E,4S)-4-(L-alanylamino)-N-(cyclohexylmethyl)-6-phenyl-2-hexenamide;
(2E,4S)-4-(L-alanylamino)-N-(1-methylethyl)-6-phenyl-2-hexenamide;
(2E,4S)-4-(L-alanylamino)-N-cycloheptyl-6-phenyl-2-hexenamide;
(2E,4S)-4-(L-alanylamino)-N-ethyl-6-phenyl-2-hexenamide;
(2E,4S)-4-(L-alanylamino)-N-[(1R,4S)-bicyclo[2.2.1]hept-2-yl]-6-phenyl-2-hexenamide;
(2E,4S)-4-(L-alanylamino)-N-(1-methylpropyl)-6-phenyl-2-hexenamide;
(2E,4S)-4-(L-alanylamino)-N-(2-methylcyclohexyl)-6-phenyl-2-hexenamide;
(2E,4S)-4-(L-alanylamino)-N-(4-hydroxycyclohexyl)-6-phenyl-2-hexenamide;
(2E,4S)-4-(L-alanylamino)-6-phenyl-N-(tetrahydro-3-furanyl)-2-hexenamide;
(2E,4S)-4-(L-alanylamino)-6-phenyl-N-(tetrahydro-2H-pyran-3-yl)-2-hexenamide;
(2E,4S)-4-(L-alanylamino)-N-(1-methyl-3-piperidinyl)-6-phenyl-2-hexenamide;
(2E,4S)-4-(L-alanylamino)-N-[(1S,3S)-3-hydroxycyclopentyl]-6-phenyl-2-hexenamide;
N¹-[(1S,2E)-4-[(1R,8S)-11-azatricyclo[6.2.1.0^{2,7}]undeca-2,4,6-trien-11-yl]-4-oxo-1-(2-phenylethyl)-2-buten-1-yl]-L-alaninamide;
(2S)-2-amino-N-[(1S,2E)-4-[(1R,8S)-11-azatricyclo[6.2.1.0^{2,7}]undeca-2,4,6-trien-11-yl]-4-oxo-1-(2-phenylethyl)-2-buten-1-yl]butenamide;
N¹-[(1S,2E)-4-(1,3-dihydro-2H-isoindol-2-yl)-1-ethyl-4-oxo-2-buten-1-yl]-L-alaninamide;
N¹-{(1S,2E)-4-[(1R,8S)-11-azatricyclo[6.2.1.0^{2,7}]undeca-2,4,6-trien-11-yl]-1-[(1S)-1-methylpropyl]-4-oxo-2-buten-1-yl}-L-alaninamide;
N¹-{(1S,2E)-4-(1,3-dihydro-2H-isoindol-2-yl)-1-[(15)-1-methylpropyl]-4-oxo-2-buten-1-yl}-L-alaninamide;
(2E,4S)-4-(L-alanylamino)-6-methyl-N-[4-(methyloxy)phenyl]-2-heptenamide;
N¹-[(1S,2E)-4-(1,3-dihydro-2H-isoindol-2-yl)-1-(2-methylpropyl)-4-oxo-2-buten-1-yl]-L-alaninamide;
(2E,4S)—N-[4-(methyloxy)phenyl]-6-phenyl-4-{[3-(2-thienyl)-L-alanyl]amino}-2-hexenamide;
(2E,4S)-4-{[(2S)-2-aminobutanoyl]amino}-N-[4-(methyloxy)phenyl]-6-phenyl-2-hexenamide;
(2E,4S)-6-phenyl-N-propyl-4-{[3-(2-thienyl)-L-alanyl]amino}-2-hexenamide;
(2E,4S)-4-{[(2S)-2-aminobutanoyl]amino}-N-[5-(1-methylcyclobutyl)-1,3,4-thiadiazol-2-yl]-6-phenyl-2-hexenamide;
(2S)—N-[(1S,2E)-4-{[4-(methyloxy)phenyl]amino}-4-oxo-1-(2-phenylethyl)-2-buten-1-yl]-2-azetidinecarboxamide;
(2E,4S)-4-{[(2S)-2-amino-2-cyclopentylacetyl]amino}-N-[4-(methyloxy)phenyl]-6-phenyl-2-hexenamide;
(2E,4S)—N-[4-(methyloxy)phenyl]-6-phenyl-4-(L-valylamino)-2-hexenamide;
(2S)—N-[(1S,2E)-4-(2,3-dihydro-1H-indol-1-yl)-1-ethyl-4-oxo-2-buten-1-yl]-2-azetidinecarboxamide;
(2S)—N-[(1S,2E)-4-(2,3-dihydro-1H-indol-1-yl)-1-(2-methylpropyl)-4-oxo-2-buten-1-yl]-2-azetidinecarboxamide;
(2S)—N-[(1S,2E)-1-(cyclopropylmethyl)-4-(2,3-dihydro-1H-indol-1-yl)-4-oxo-2-buten-1-yl]-2-azetidinecarboxamide;
(4S)—N-[(1S,2E)-4-(2,3-dihydro-1H-indol-1-yl)-1-ethyl-4-oxo-2-buten-1-yl]-4-fluoro-L-prolinamide;
(2S)—N-[(1S,2E)-4-(2,3-dihydro-1H-indol-1-yl)-1-ethyl-4-oxo-2-buten-1-yl]-2-piperidinecarboxamide;
(2S)—N-[(1S,2E)-4-(2,3-dihydro-1H-indol-1-yl)-1-(2-methylpropyl)-4-oxo-2-buten-1-yl]-2-piperidinecarboxamide;
(2S)—N-41S,2E)-1-(2-methylpropyl)-4-oxo-4-{[5-(trifluoromethyl)-1,3,4-thiadiazol-2-yl]amino}-2-buten-1-yl)-2-azetidinecarboxamide;
(2S)—N-((1S,2E)-1-(2-methylpropyl)-4-{methyl[5-(trifluoromethyl)-1,3,4-thiadiazol-2-yl]amino}-4-oxo-2-buten-1-yl)-2-azetidinecarboxamide;
(4S)—N-((1S,2E)-1-ethyl-4-oxo-4-{[5-(trifluoromethyl)-1,3,4-thiadiazol-2-yl]amino}-2-buten-1-yl)-4-fluoro-L-prolinamide;
(2S)—N-((1S,2E)-1-(2-methylpropyl)-4-oxo-4-{[5-(trifluoromethyl)-1,3,4-thiadiazol-2-yl]amino}-2-buten-1-yl)-2-piperidinecarboxamide;
(2S)—N-[(1S,2E)-4-{[5-(1-methyl-1-phenylethyl)-1,3,4-thiadiazol-2-yl]amino}-4-oxo-1-(2-phenylethyl)-2-buten-1-yl]-2-piperidinecarboxamide;
(2S)—N-[(3S,4E)-6-(2,3-dihydro-1H-indol-1-yl)-6-oxo-1-phenylhex-4-en-3-yl]azetidine-2-carboxamide;
(2S)—N-[(3S,4E)-6-(1,3-dihydro-2H-isoindol-2-yl)-6-oxo-1-phenylhex-4-en-3-yl]azetidine-2-carboxamide;
(2E,4S)-4-{[(2S)-2-amino-2-cyclopropylacetyl]amino}-N-(4-methoxyphenyl)-6-phenylhex-2-enamide;
(2S)-2-amino-2-cyclopentyl-N-[(3S,4E)-6-(2,3-dihydro-1H-indol-1-yl)-6-oxo-1-phenylhex-4-en-3-yl]ethanamide;
(2S)-2-amino-2-cyclopentyl-N-[(3S,4E)-6-(1,3-dihydro-2H-isoindol-2-yl)-6-oxo-1-phenylhex-4-en-3-yl]ethanamide;
(2S)-2-amino-N-[(3S,4E)-6-(2,3-dihydro-1H-indol-1-yl)-6-oxo-1-phenylhex-4-en-3-yl]butanamide;
N-[(3S,4E)-6-(2,3-dihydro-1H-indol-1-yl)-6-oxo-1-phenylhex-4-en-3-yl]-3-thiophen-2-yl-L-alaninamide;
N-[(3S,4E)-6-(1,3-dihydro-2H-isoindol-2-yl)-6-oxo-1-phenylhex-4-en-3-yl]-L-isoleucinamide;
N-[(3S,4E)-6-(1,3-dihydro-2H-isoindol-2-yl)-6-oxo-1-phenylhex-4-en-3-yl]-L-alloisoleucinamide;
N-[(3S,4E)-6-(1,3-dihydro-2H-isoindol-2-yl)-6-oxo-1-phenylhex-4-en-3-yl]-3-methyl-L-valinamide;
(2S)—N-[(3S,4E)-6-(1,3-dihydro-2H-isoindol-2-yl)-6-oxo-1-phenylhex-4-en-3-yl]piperidine-2-carboxamide;
N-[(3S,4E)-6-(1,3-dihydro-2H-isoindol-2-yl)-6-oxo-1-phenylhex-4-en-3-yl]-L-prolinamide;
(2S)—N-[(3S,4E)-6-(1,3-dihydro-2H-isoindol-2-yl)-6-oxo-1-phenylhex-4-en-3-yl]-2-(methylamino)butanamide;
(2S)—N-[(3S,4E)-6-oxo-1-phenyl-6-{[5-(trifluoromethyl)-1,3,4-thiadiazol-2-yl]amino}hex-4-en-3-yl]piperidine-2-carboxamide;
(2S)—N-{(3S,4E)-6-[(5-methyl-1,3,4-thiadiazol-2-yl)amino]-6-oxo-1-phenylhex-4-en-3-yl}piperidine-2-carboxamide;
(2S)—N-{(3S,4E)-6-[(5-ethyl-1,3,4-thiadiazol-2-yl)amino]-6-oxo-1-phenylhex-4-en-3-yl}piperidine-2-carboxamide;
(2S)—N-{(3S,4E)-6-[(5-tert-butyl-1,3,4-thiadiazol-2-yl)amino]-6-oxo-1-phenylhex-4-en-3-yl}piperidine-2-carboxamide;
(2S)—N-[(3S,4E)-6-{[5-(methylsulfanyl)-1,3,4-thiadiazol-2-yl]amino}-6-oxo-1-phenylhex-4-en-3-yl]piperidine-2-carboxamide;
(2S)—N-{(3S,4E)-6-oxo-1-phenyl-6-[(5-phenyl-1,3,4-thiadiazol-2-yl)amino]hex-4-en-3-yl}piperidine-2-carboxamide;
(2S)—N-[(3S,4E)-6-{[5-(4-bromophenyl)-1,3,4-thiadiazol-2-yl]amino}-6-oxo-1-phenylhex-4-en-3-yl]piperidine-2-carboxamide;
(2S)—N-[(3S,4E)-6-{[5-(4-fluorophenyl)-1,3,4-thiadiazol-2-yl]amino}-6-oxo-1-phenylhex-4-en-3-yl]piperidine-2-carboxamide;
(2S)—N-{(3S,4E)-6-[(5-cyclopropyl-1,3,4-thiadiazol-2-yl)amino]-6-oxo-1-phenylhex-4-en-3-yl}piperidine-2-carboxamide;
(2S)—N-[(3S,4E)-6-oxo-1-phenyl-6-{[5-(propan-2-yl)-1,3,4-thiadiazol-2-yl]amino}hex-4-en-3-yl]piperidine-2-carboxamide;
(2S)—N-{(3S,4E)-6-[(5-benzyl-1,3,4-thiadiazol-2-yl)amino]-6-oxo-1-phenylhex-4-en-3-yl}piperidine-2-carboxamide;
(2S)—N-[(3S,4E)-6-oxo-1-phenyl-6-{[5-(2-phenylethyl)-1,3,4-thiadiazol-2-yl]amino}hex-4-en-3-yl]piperidine-2-carboxamide;
(2S)—N-{(3S,4E)-6-[(5-cyclohexyl-1,3,4-thiadiazol-2-yl)amino]-6-oxo-1-phenylhex-4-en-3-yl}piperidine-2-carboxamide;
(2S)-2-amino-2-cyclopropyl-N-[(3S,4E)-6-(2,3-dihydro-1H-indol-1-yl)-6-oxo-1-phenylhex-4-en-3-yl]ethanamide;
(2S)-2-amino-2-cyclopropyl-N-[(3S,4E)-6-(1,3-dihydro-2H-isoindol-2-yl)-6-oxo-1-phenylhex-4-en-3-yl]ethanamide;
N-[(3S,4E)-6-(1,3-dihydro-2H-isoindol-2-yl)-6-oxo-1-phenylhex-4-en-3-yl]-L-valinamide;
(1R,2S,5S)—N-[(3S,4E)-6-(2,3-dihydro-1H-indol-1-yl)-6-oxo-1-phenylhex-4-en-3-yl]-3-azabicyclo[3.1.0]hexane-2-carboxamide;
(1S,2R,5R)—N-[(3S,4E)-6-(2,3-dihydro-1H-indol-1-yl)-6-oxo-1-phenylhex-4-en-3-yl]-3-azabicyclo[3.1.0]hexane-2-carboxamide;
N-[(3S,4E)-6-(2,3-dihydro-1H-indol-1-yl)-6-oxo-1-phenylhex-4-en-3-yl]-L-valinamide;
(2E,4S)—N-methyl-6-phenyl-N-[5-(trifluoromethyl)-1,3,4-thiadiazol-2-yl]-4-(L-valylamino)hex-2-enamide;
(2E,4S)-6-phenyl-N-[5-(trifluoromethyl)-1,3,4-thiadiazol-2-yl]-4-(L-valylamino)hex-2-enamide;
(2S)—N-[(3S,4E)-6-oxo-1-phenyl-6-{[5-(trifluoromethyl)-1,3,4-thiadiazol-2-yl]amino}hex-4-en-3-yl]azetidine-2-carboxamide;
(2S)—N-[(3S,4E)-6-{methyl[5-(trifluoromethyl)-1,3,4-thiadiazol-2-yl]amino}-6-oxo-1-phenylhex-4-en-3-yl]azetidine-2-carboxamide;
(2S)—N-[(3S,4E)-6-(2,3-dihydro-1H-indol-1-yl)-6-oxo-1-phenylhex-4-en-3-yl]piperidine-2-carboxamide;
(4S)—N-[(3S,4E)-6-(2,3-dihydro-1H-indol-1-yl)-6-oxo-1-phenylhex-4-en-3-yl]-4-fluoro-L-prolinamide;
N-[(2S,3E)-1-cyclohexyl-5-(1,3-dihydro-2H-isoindol-2-yl)-5-oxopent-3-en-2-yl]-L-alaninamide;
N-[(2S,3E)-1-cyclohexyl-5-(2,3-dihydro-1H-indol-1-yl)-5-oxopent-3-en-2-yl]-1, alaninamide;
(2S)—2-amino-N-[(2S,3E)-1-cyclohexyl-5-(1,3-dihydro-2H-isoindol-2-yl)-5-oxopent-3-en-2-yl]butanamide;
(2S)-2-amino-N-[(2S,3E)-1-cyclohexyl-5-(2,3-dihydro-1H-indol-1-yl)-5-oxopent-3-en-2-yl]butanamide;
N-[(2S,3E)-1-cyclohexyl-5-(1,3-dihydro-2H-isoindol-2-yl)-5-oxopent-3-en-2-yl]-3-thiophen-2-yl-L-alaninamide;
N-[(2S,3E)-1-cyclohexyl-5-(2,3-dihydro-1H-indol-1-yl)-5-oxopent-3-en-2-yl]-3-thiophen-2-yl-L-alaninamide;
(2S)—N-[(2S,3E)-1-cyclohexyl-5-(1,3-dihydro-2H-isoindol-2-yl)-5-oxopent-3-en-2-yl]azetidine-2-carboxamide;
(2S)—N-[(2S,3E)-1-cyclohexyl-5-(2,3-dihydro-1H-indol-1-yl)-5-oxopent-3-en-2-yl]azetidine-2-carboxamide;
(2S)—N-[(2S,3E)-1-cyclohexyl-5-{methyl[5-(trifluoromethyl)-1,3,4-thiadiazol-2-yl]amino}-5-oxopent-3-en-2-yl]azetidine-2-carboxamide;
(2S)—N-[(2S,3E)-1-cyclohexyl-5-oxo-5-{[5-(trifluoromethyl)-1,3,4-thiadiazol-2-yl]amino}pent-3-en-2-yl]azetidine-2-carboxamide;
(2S)—N-{(2S,3E)-5-[(5-tert-butyl-1,3,4-thiadiazol-2-yl)amino]-1-cyclohexyl-5-oxopent-3-en-2-yl}azetidine-2-carboxamide;
(2S)—N-[(2S,3E)-1-cyclobutyl-5-(2,3-dihydro-1H-indol-1-yl)-5-oxopent-3-en-2-yl]azetidine-2-carboxamide;
(2S)—N-[(2S,3E)-1-cyclobutyl-5-(2,3-dihydro-1H-indol-1-yl)-5-oxopent-3-en-2-yl]piperidine-2-carboxamide;
(2S)—N-[(2S,3E)-1-cyclobutyl-5-oxo-5-{[5-(trifluoromethyl)-1,3,4-thiadiazol-2-yl]amino}pent-3-en-2-yl]piperidine-2-carboxamide;
N-[(2E,4S)-1-(1,3-dihydro-2H-isoindol-2-yl)-6,6-dimethyl-1-oxohept-2-en-4-yl]-3-thiophen-2-yl-L-alaninamide;
N-[(2E,4S)-1-(2,3-dihydro-1H-indol-1-yl)-6,6-dimethyl-1-oxohept-2-en-4-yl]-3-thiophen-2-yl-L-alaninamide;
(2E,4S)—N-(4-methoxyphenyl)-6,6-dimethyl-4-{[3-(thiophen-2-yl)-L-alanyl]amino}hept-2-enamide;
(2E,4S)-4-(L-alanylamino)-N-(4-methoxyphenyl)-6,6-dimethylhept-2-enamide;
N-[(2E,4S)-1-(1,3-dihydro-2H-isoindol-2-yl)-6,6-dimethyl-1-oxohept-2-en-4-yl]-L-alaninamide;
N-[(2E,4S)-1-(2,3-dihydro-1H-indol-1-yl)-6,6-dimethyl-1-oxohept-2-en-4-yl]-1, alaninamide;
(2S)-2-amino-N-[(2E,4S)-1-(1,3-dihydro-2H-isoindol-2-yl)-6,6-dimethyl-1-oxohept-2-en-4-yl]butanamide;
(2S)-2-amino-N-[(2E,4S)-1-(2,3-dihydro-1H-indol-1-yl)-6,6-dimethyl-1-oxohept-2-en-4-yl]butanamide;
(2E,4S)-4-{[(2S)-2-aminobutanoyl]amino}-N-(4-methoxyphenyl)-6,6-dimethylhept-2-enamide;
(2E,4S)-4-{[(2S)-2-aminobutanoyl]amino}-N-(4-methoxyphenyl)-6-methylhept-2-enamide;
(2E,4S)—N-(4-methoxyphenyl)-6-methyl-4-{[3-(thiophen-2-yl)-L-alanyl]amino}hept-2-enamide;
N-[(2E,4S)-1-(2,3-dihydro-1H-indol-1-yl)-6-methyl-1-oxohept-2-en-4-yl]-1, alaninamide;
N-[(2E,4S)-1-(2,3-dihydro-1H-indol-1-yl)-6-methyl-1-oxohept-2-en-4-yl]-3-thiophen-2-yl-L-alaninamide;
(2S)-2-amino-N-[(2E,4S)-1-(2,3-dihydro-1H-indol-1-yl)-6-methyl-1-oxohept-2-en-4-yl]butanamide;
N-{(2E,4S)-6-methyl-1-oxo-1-[(1R,4S)-1,2,3,4-tetrahydro-1,4-epiminonaphthalen-9-yl]hept-2-en-4-yl}-3-thiophen-2-yl-L-alaninamide;
(2S)-2-amino-N-{(2E,4S)-6-methyl-1-oxo-1-[(1R,4S)-1,2,3,4-tetrahydro-1,4-epiminonaphthalen-9-yl]hept-2-en-4-yl}butanamide;
(2E,4S)—N-(5-tert-butyl-1,3,4-thiadiazol-2-yl)-6-methyl-4-{[3-(thiophen-2-yl)-L-alanyl]amino}hept-2-enamide;
N-[(2E,4S,5S)-1-(2,3-dihydro-1H-indol-1-yl)-5-methyl-1-oxohept-2-en-4-yl]-L-alaninamide;
(2E,4S)—N-[5-(4-fluorophenyl)-1,3,4-thiadiazol-2-yl]-6-methyl-4-{[3-(thiophen-2-yl)-L-alanyl]amino}hept-2-enamide;
(2S)—N-[(2E,4S)-1-(1,3-dihydro-2H-isoindol-2-yl)-6-methyl-1-oxohept-2-en-4-yl]azetidine-2-carboxamide;
(2S)—N-[(2E,4S)-1-(2,3-dihydro-1H-indol-1-yl)-6-methyl-1-oxohept-2-en-4-yl]azetidine-2-carboxamide;
(2S)—N-{(2E,4S)-1-[(4-methoxyphenyl)amino]-6-methyl-1-oxohept-2-en-4-yl}azetidine-2-carboxamide;
(2S)—N-[(2E,4S)-6-methyl-1-{methyl[5-(trifluoromethyl)-1,3,4-thiadiazol-2-yl]amino}-1-oxohept-2-en-4-yl]piperidine-2-carboxamide;
(2S)—N-{(2S,3E)-5-[(5-tert-butyl-1,3,4-thiadiazol-2-yl)amino]-1-cyclopropyl-5-oxopent-3-en-2-yl}azetidine-2-carboxamide;
(2S)—N-[(2S,3E)-1-cyclopropyl-5-(1,3-dihydro-2H-isoindol-2-yl)-5-oxopent-3-en-2-yl]azetidine-2-carboxamide;
N-[(2E,4S)-1-(1,3-dihydro-2H-isoindol-2-yl)-1-oxohept-2-en-4-yl]-1, alaninamide;
N-[(2E,4S)-1-(2,3-dihydro-1H-indol-1-yl)-1-oxohept-2-en-4-yl]-L-alaninamide;
(2E,4S)-4-(L-alanylamino)-N-(4-methoxyphenyl)hept-2-enamide;
N-[(2E,4S)-1-(1,3-dihydro-2H-isoindol-2-yl)-1-oxohept-2-en-4-yl]-3-thiophen-2-yl-L-alaninamide;
N-[(2E,4S)-1-(2,3-dihydro-1H-indol-1-yl)-1-oxohept-2-en-4-yl]-3-thiophen-2-yl-L-alaninamide;
(2E,4S)—N-(4-methoxyphenyl)-4-{[3-(thiophen-2-yl)-L-alanyl]amino}hept-2-enamide;
(2E,4S)-4-{[(2S)-2-aminobutanoyl]amino}-N-(4-methoxyphenyl)hept-2-enamide;
(2S)-2-amino-N-[(2E,4S)-1-(1,3-dihydro-2H-isoindol-2-yl)-1-oxohept-2-en-4-yl]butanamide;
(2S)-2-amino-N-[(2E,4S)-1-(2,3-dihydro-1H-indol-1-yl)-1-oxohept-2-en-4-yl]butanamide;
(2E,4S)-4-(L-alanylamino)-4-cyclopropyl-N-(4-methoxyphenyl)but-2-enamide;
N-[(1S,2E)-1-cyclopropyl-4-(1,3-dihydro-2H-isoindol-2-yl)-4-oxobut-2-en-1-yl]-L-alaninamide;
N-[(1S,2E)-1-cyclopropyl-4-(2,3-dihydro-1H-indol-1-yl)-4-oxobut-2-en-1-yl]-L-alaninamide;
N-{(1S,2E)-1-cyclopropyl-4-[(4-methoxyphenyl)amino]-4-oxobut-2-en-1-yl}-L-valinamide;
N-[(1S,2E)-1-cyclopropyl-4-(1,3-dihydro-2H-isoindol-2-yl)-4-oxobut-2-en-1-yl]-L-valinamide;
N-[(1S,2E)-1-cyclopropyl-4-(2,3-dihydro-1H-indol-1-yl)-4-oxobut-2-en-1-yl]-L-valinamide;
(2S)-2-amino-2-cyclopentyl-N-[(1S,2E)-1-cyclopropyl-4-(1,3-dihydro-2H-isoindol-2-yl)-4-oxobut-2-en-1-yl]ethanamide;
(2S)-2-amino-2-cyclopentyl-N-[(1S,2E)-1-cyclopropyl-4-(2,3-dihydro-1H-indol-1-yl)-4-oxobut-2-en-1-yl]ethanamide;
(2S)—N-[(1S,2E)-1-cyclopropyl-4-(1,3-dihydro-2H-isoindol-2-yl)-4-oxobut-2-en-1-yl]azetidine-2-carboxamide;
(2S)—N-[(1S,2E)-1-cyclopropyl-4-(2,3-dihydro-1H-indol-1-yl)-4-oxobut-2-en-1-yl]azetidine-2-carboxamide;
(2S)—N-[(1S,2E)-1-cyclopropyl-4-oxo-4-{[5-(trifluoromethyl)-1,3,4-thiadiazol-2-yl]amino}but-2-en-1-yl]azetidine-2-carboxamide;
(2S)—N-{(1S,2E)-4-[(5-tert-butyl-1,3,4-thiadiazol-2-yl)amino]-1-cyclopropyl-4-oxobut-2-en-1-yl}azetidine-2-carboxamide;
(2S)-2-amino-N-[(3S,4E)-6-(1,3-dihydro-2H-isoindol-2-yl)-6-oxohex-4-en-3-yl]butanamide;
N-[(3S,4E)-6-(1,3-dihydro-2H-isoindol-2-yl)-6-oxohex-4-en-3-yl]-3-thiophen-2-yl-L-alaninamide;
(2E,4S)-4-{[(2S)-2-aminobutanoyl]amino}-N-(4-methoxyphenyl)hex-2-enamide;
(2S)-2-amino-N-{(3S,4E)-6-oxo-6-[(1R,4S)-1,2,3,4-tetrahydro-1,4-epiminonaphthalen-9-yl]hex-4-en-3-yl}butanamide;
(2E,4S)—N-(4-methoxyphenyl)-4-{[3-(thiophen-2-yl)-L-alanyl]amino}hex-2-enamide;
N-[(3S,4E)-6-oxo-6-(1,2,3,4-tetrahydro-1,4-epiminonaphthalen-9-yl)hex-4-en-3-yl]-3-thiophen-2-yl-L-alaninamide;
N-[(3S,4E)-6-(2,3-dihydro-1H-indol-1-yl)-6-oxohex-4-en-3-yl]-L-alaninamide;
(2S)-2-amino-N-[(3S,4E)-6-(2,3-dihydro-1H-indol-1-yl)-6-oxohex-4-en-3-yl]butanamide;
N-[(3S,4E)-6-(2,3-dihydro-1H-indol-1-yl)-6-oxohex-4-en-3-yl]-3-thiophen-2-yl-L-alaninamide;
(2S)—N-[(3S,4E)-6-(1,3-dihydro-2H-isoindol-2-yl)-6-oxohex-4-en-3-yl]azetidine-2-carboxamide;
(2S)—N-[(3S,4E)-6-(2,3-dihydro-1H-indol-1-yl)-6-oxohex-4-en-3-yl]azetidine-2-carboxamide;
(2S)—N-{(3S,4E)-6-[(4-methoxyphenyl)amino]-6-oxohex-4-en-3-yl}azetidine-2-carboxamide;
(2S)—N-[(3S,4E)-6-oxo-6-{[5-(trifluoromethyl)-1,3,4-thiadiazol-2-yl]amino}hex-4-en-3-yl]piperidine-2-carboxamide;
(2S)—N-[(3S,4E)-6-{[5-(4-fluorophenyl)-1,3,4-thiadiazol-2-yl]amino}-6-oxohex-4-en-3-yl]piperidine-2-carboxamide;
(2S)—N-[(3S,4E)-6-{methyl[5-(trifluoromethyl)-1,3,4-thiadiazol-2-yl]amino}-6-oxohex-4-en-3-yl]piperidine-2-carboxamide; and
N-[(4E)-6-(2,3-dihydro-1H-indol-1-yl)-1,1,1-trifluoro-6-oxohex-4-en-3-yl]-L-alaninamide.

The invention also includes various isomers of the compounds of Formula (I) and mixtures thereof “Isomer” refers to compounds that have the same composition and molecular weight but differ in physical and/or chemical properties. The structural difference may be in constitution (geometric isomers) or in the ability to rotate the plane of polarized light (stereoisomers). The compounds according to Formula (I) contain two or more asymmetric centers, also referred to as chiral centers, and may, therefore, exist as individual enantiomers, diastereomers, or other stereoisomeric forms, or as mixtures thereof. All such isomeric forms are included within the present invention, including mixtures thereof.

Chiral centers may also be present in a substituent such as an alkyl group. Where the stereochemistry of a chiral center present in Formula (I), or in any chemical structure illustrated herein, is not specified the structure is intended to encompass any stereoisomer and all mixtures thereof. Thus, compounds according to Formula (I) containing two or more chiral centers may be used as racemic mixtures, enantiomerically enriched mixtures, or as enantiomerically pure individual stereoisomers.

Individual stereoisomers of a compound according to Formula (I) which contain two or more asymmetric centers may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

“Enantiomerically enriched” refers to products whose enantiomeric excess is greater than zero. For example, enantiomerically enriched refers to products whose enantiomeric excess is greater than 50% ee, greater than 75% ee, and greater than 90% ee.

“Enantiomeric excess” or “ee” is the excess of one enantiomer over the other expressed as a percentage. As a result, since both enantiomers are present in equal amounts in a racemic mixture, the enantiomeric excess is zero (0% ee). However, if one enantiomer was enriched such that it constitutes 95% of the product, then the enantiomeric excess would be 90% ee (the amount of the enriched enantiomer, 95%, minus the amount of the other enantiomer, 5%).

“Enantiomerically pure” means products whose enantiomeric excess is 99% ee or greater.

The invention also includes various deuterated forms of the compounds of Formula (I). Each available hydrogen atom attached to a carbon atom may be independently replaced with a deuterium atom. A person of ordinary skill in the art will know how to synthesize deuterated forms of the compounds of Formula (I). For example, α-deuterated α-amino acids are commercially available or may be prepared by conventional techniques (see for example: Elemes, Y. and Ragnarsson, U. J. Chem. Soc., Perkin Trans. 1, 1996, 6, 537-40). Employing such compounds according to Scheme 1 or 2 below will allow for the preparation of compounds of Formula (I) in which either or both of the hydrogen atoms at the chiral centers are replaced with a deuterium atom. Similarly, α-amino acids in which deuterium atoms have been incorporated into the sidechains are commercially available or may be prepared by conventional techniques.

Employing such compounds according to Scheme 1 or 2 below will allow for the preparation of compounds of Formula (I) in which deuterium atoms have been incorporated in R³ and/or R⁴. Additionally, replacement of the reagent lithium aluminum hydride with lithium aluminum deuteride according to Scheme 1 below will allow for deuterium substitution at the 13-position of the butenamide of the compounds of Formula (I).

The term “solvate” refers to a complex of variable stoichiometry formed by a solute and a solvent. Such solvents for the purpose of the invention may not interfere with the biological activity of the solute. Examples of suitable solvents include, but are not limited to, water, methanol, ethanol and acetic acid. Preferably, the solvent used is a pharmaceutically acceptable solvent. Examples of suitable pharmaceutically acceptable solvents include, without limitation, water, ethanol and acetic acid. Solvates wherein water is the solvent molecule are typically referred to as “hydrates”. Hydrates include compositions containing stoichiometric amounts of water, as well as compositions containing variable amounts of water. Solvates, particularly hydrates, of the compounds of Formula (I) and salts thereof, are within the scope of the invention.

When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.” It is to be understood that when named or depicted by structure, the disclosed compound, or solvates (particularly, hydrates) thereof, also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

Because of their potential use in medicine, the salts of the compounds of Formula (I) are preferably pharmaceutically acceptable. Suitable pharmaceutically acceptable salts can include acid or base addition salts.

As used herein, the term “pharmaceutically acceptable” means a compound which is suitable for pharmaceutical use. Salts and solvates (e.g. hydrates and hydrates of salts) of the compounds of the invention which are suitable for use in medicine are those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of the invention and their salts and solvates.

Compounds of Formula (I) have one or more nitrogen(s) basic enough to form pharmaceutically acceptable acid addition salts by treatment with a suitable acid. Suitable acids include pharmaceutically acceptable inorganic acids and pharmaceutically acceptable organic acids. Representative pharmaceutically acceptable acid addition salts include acetate, aspartate, benzenesulfonate, benzoate, bicarbonate, bitartrate, bromide, calcium edetate, camsylate, carbonate, chloride, citrate, dihydrochloride, edetate, edisylate, estolate, esylate, formate, fumarate, galacturonate, gluceptate, gluconate, glutamate, glycollylarsanilate, hexanoate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, propionate, salicylate, stearate, subacetate, succinate, sulfate, tannate, tartrate, teoclate, and tosylate salts.

Other iterations of compounds of the invention have an acidic functional group, one acidic enough to form salts. Representative salts include pharmaceutically acceptable metal salts such as sodium, potassium, lithium, calcium, magnesium, aluminum, and zinc salts; carbonates and bicarbonates of a pharmaceutically acceptable metal cation such as sodium, potassium, lithium, calcium, magnesium, aluminum, and zinc; pharmaceutically acceptable organic primary, secondary, and tertiary amines including aliphatic amines, aromatic amines, aliphatic diamines, and hydroxy alkylamines such as methylamine, ethylamine, 2-hydroxyethylamine, diethylamine, triethylamine, ethylenediamine, ethanolamine, diethanolamine, cyclohexylamine, triethanolamine, choline, arginine, lysine, and histidine.

Other non-pharmaceutically acceptable salts, e.g. trifluoroacetate, may be used, for example in the isolation of compounds of the invention, and are included within the scope of this invention.

The invention includes within its scope all possible stoichiometric and non-stoichiometric forms of the salts of the compounds of Formula (I).

It will be appreciated by those skilled in the art that certain protected derivatives of compounds of Formula (I), which may be made prior to a final deprotection stage, may not possess pharmacological activity as such, but may, in certain instances, be administered orally or parenterally and thereafter metabolized in the body to form compounds of the invention which are pharmacologically active. Such derivatives may therefore be described as “prodrugs”. Further, certain compounds of the invention may act as prodrugs of other compounds of the invention. All protected derivatives and prodrugs of compounds of the invention are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Preferred “pro-moieties” for compounds of the invention include: ester, carbonate ester, hemi-ester, phosphate ester, nitro ester, sulfate ester, sulfoxide, amide, carbamate, azo-, phosphamide, glycoside, ether, acetal, and ketal derivatives of the compounds of Formula (I).

The compounds of the invention inhibit the cathepsin C enzyme and can be useful in the treatment of conditions wherein the underlying pathology is (at least in part) attributable to cathepsin C involvement or in conditions wherein cathepsin C inhibition offers some clinical benefit even though the underlying pathology is not (even in part) attributable to cathepsin C involvement. Examples of such conditions include COPD, rheumatoid arthritis, osteoarthritis, asthma, and multiple sclerosis. Accordingly, in another aspect the invention is directed to methods of treating such conditions.

The methods of treatment of the invention comprise administering an effective amount of a compound of the invention to a patient in need thereof.

As used herein, “treatment” in reference to a condition means: (1) the amelioration or prevention of the condition being treated or one or more of the biological manifestations of the condition being treated, (2) the interference with (a) one or more points in the biological cascade that leads to or is responsible for the condition being treated or (b) one or more of the biological manifestations of the condition being treated, or (3) the alleviation of one or more of the symptoms or effects associated with the condition being treated.

As indicated above, “treatment” of a condition includes prevention of the condition. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

An “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, “patient” refers to a human or animal.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, otic, intravaginal, and intranasal administration.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the amount administered and the duration such regimens are administered, for a compound of the invention depend on the condition being treated, the severity of the condition being treated, the age and physical condition of the patient being treated, the medical history of the patient to be treated, the nature of concurrent therapy, the particular route of administration chosen, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change. Typical daily dosages range from 1 mg to 1000 mg.

The invention includes the use of compounds of the invention for the preparation of a composition for treating or ameliorating diseases mediated by the cathepsin C enzyme in a subject in need thereof, wherein the composition comprises a mixture of one or more of the compounds of the invention and an optional pharmaceutically acceptable excipient.

The invention further includes the use of compounds of the invention as an active therapeutic substance, in particular in the treatment of diseases mediated by the cathepsin C enzyme. Specifically, the invention includes the use of compounds of the invention in the treatment of COPD, rheumatoid arthritis, osteoarthritis, asthma, and multiple sclerosis.

In another aspect, the invention includes the use of compounds of the invention in the manufacture of a medicament for use in the treatment of the above disorders.

### Compositions

The compounds of the invention will normally, but not necessarily, be formulated into a pharmaceutical composition prior to administration to a patient. Accordingly, in another aspect the invention is directed to pharmaceutical compositions comprising a compound of the invention and a pharmaceutically acceptable excipient.

The pharmaceutical compositions of the invention may be prepared and packaged in bulk form wherein an effective amount of a compound of the invention can be extracted and then given to the patient such as with powders, syrups, and solutions for injection. Alternatively, the pharmaceutical compositions of the invention may be prepared and packaged in unit dosage form wherein each physically discrete unit contains an effective amount of a compound of the invention. When prepared in unit dosage form, the pharmaceutical compositions of the invention typically contain from 1 mg to 1000 mg.

The pharmaceutical compositions of the invention typically contain one compound of the invention. However, in certain embodiments, the pharmaceutical compositions of the invention contain more than one compound of the invention. For example, in certain embodiments the pharmaceutical compositions of the invention contain two compounds of the invention. In addition, the pharmaceutical compositions of the invention may optionally further comprise one or more additional pharmaceutically active compounds. Conversely, the pharmaceutical compositions of the invention typically contain more than one pharmaceutically acceptable excipient. However, in certain embodiments, the pharmaceutical compositions of the invention contain one pharmaceutically acceptable excipient.

As used herein, “pharmaceutically acceptable excipient” means a material, composition or vehicle involved in giving form or consistency to the composition and which is safe when administered to a patient. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically-acceptable.

The compounds of the invention and the pharmaceutically acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixirs, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting the compound or compounds of the invention once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising an effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

In another aspect, the invention is directed to a dosage form adapted for administration to a patient by inhalation. For example, the compound of the invention may be inhaled into the lungs as a dry powder, an aerosol, a suspension, or a solution.

Dry powder compositions for delivery to the lung by inhalation typically comprise a compound of the invention as a finely divided powder together with one or more pharmaceutically acceptable excipients as finely divided powders. Pharmaceutically acceptable excipients particularly suited for use in dry powders are known to those skilled in the art and include lactose, starch, mannitol, and mono-, di-, and polysaccharides.

The dry powder may be administered to the patient via a reservoir dry powder inhaler (RDPI) having a reservoir suitable for storing multiple (un-metered doses) of medicament in dry powder form. RDPIs typically include a means for metering each medicament dose from the reservoir to a delivery position. For example, the metering means may comprise a metering cup, which is movable from a first position where the cup may be filled with medicament from the reservoir to a second position where the metered medicament dose is made available to the patient for inhalation.

Alternatively, the dry powder may be presented in capsules (e.g. gelatin or plastic), cartridges, or blister packs for use in a multi-dose dry powder inhaler (MDPI). MDPIs are inhalers wherein the medicament is comprised within a multi-dose pack containing (or otherwise carrying) multiple defined doses (or parts thereof) of medicament. When the dry powder is presented as a blister pack, it comprises multiple blisters for containment of the medicament in dry powder form. The blisters are typically arranged in regular fashion for ease of release of the medicament therefrom. For example, the blisters may be arranged in a generally circular fashion on a disc-form blister pack, or the blisters may be elongate in form, for example comprising a strip or a tape. Each capsule, cartridge, or blister may, for example, contain between 20 μg-10 mg of the compound of the invention.

Aerosols may be formed by suspending or dissolving a compound of the invention in a liquified propellant. Suitable propellants include halocarbons, hydrocarbons, and other liquified gases. Representative propellants include: trichlorofluoromethane (propellant 11), dichlorofluoromethane (propellant 12), dichlorotetrafluoroethane (propellant 114), tetrafluoroethane (HFA-134a), 1,1-difluoroethane (HFA-152a), difluoromethane (HFA-32), pentafluoroethane (HFA-12), heptafluoropropane (HFA-227a), perfluoropropane, perfluorobutane, perfluoropentane, butane, isobutane, and pentane. Aerosols comprising a compound of the invention will typically be administered to a patient via a metered dose inhaler (MDI). Such devices are known to those skilled in the art.

The aerosol may contain additional pharmaceutically acceptable excipients typically used with multiple dose inhalers such as surfactants, lubricants, cosolvents and other excipients to improve the physical stability of the formulation, to improve valve performance, to improve solubility, or to improve taste.

Suspensions and solutions comprising a compound of the invention may also be administered to a patient via a nebulizer. The solvent or suspension agent utilized for nebulization may be any pharmaceutically acceptable liquid such as water, aqueous saline, alcohols or glycols, e.g., ethanol, isopropylalcohol, glycerol, propylene glycol, polyethylene glycol, etc. or mixtures thereof. Saline solutions utilize salts which display little or no pharmacological activity after administration. Both organic salts, such as alkali metal or ammonium halogen salts, e.g., sodium chloride, potassium chloride or organic salts, such as potassium, sodium and ammonium salts or organic acids, e.g., ascorbic acid, citric acid, acetic acid, tartaric acid, etc. may be used for this purpose.

Other pharmaceutically acceptable excipients may be added to the suspension or solution. The compound of the invention may be stabilized by the addition of an inorganic acid, e.g., hydrochloric acid, nitric acid, sulfuric acid and/or phosphoric acid; an organic acid, e.g., ascorbic acid, citric acid, acetic acid, and tartaric acid, etc., a complexing agent such as EDTA or citric acid and salts thereof; or an antioxidant such as antioxidant such as vitamin E or ascorbic acid. These may be used alone or together to stabilize the compound of the invention. Preservatives may be added such as benzalkonium chloride or benzoic acid and salts thereof. Surfactant may be added particularly to improve the physical stability of suspensions. These include lecithin, disodium dioctylsulphosuccinate, oleic acid and sorbitan esters.

### Methods of Preparation.

The compounds of Formula (I) may be obtained by using synthetic procedures illustrated in the Schemes below or by drawing on the knowledge of a skilled organic chemist. The synthesis provided in these Schemes are applicable for producing compounds of the invention having a variety of different R¹-R⁴ groups employing appropriate precursors, which are suitably protected if need be, to achieve compatibility with the reactions outlined herein. Subsequent deprotection, where needs be, and then affords compounds of the nature generally disclosed. While the Schemes are shown with compounds only of Formula (I), they are illustrative of processes that may be used to make the compounds of the invention.

Compounds names were generated using the software naming program ACD/Name Pro V6.02 available from Advanced Chemistry Development, Inc., 110 Yonge Street, 14^{th} Floor, Toronto, Ontario, Canada, M5C₁T4 (http://www.acdlabs.com/).

As shown in Scheme 1, the compounds of Formula (I) can be prepared in a multi-step sequence starting from a Boc-protected α-amino acid, such as the commercially available (2S)-2-({[(1,1-dimethylethyl)oxy]carbonyl}amino)-4-phenylbutanoic acid (also known as Boc-L-homophenylalanine), (2S)-2-({[(1,1-dimethylethyl)oxy]carbonyl}amino)butanoic acid, N-{[(1,1-dimethylethyl)oxy]carbonyl}-L-isoleucine, N-(tert-butoxycarbonyl)-L-leucine, 3-cyclopropyl-N-(tert-butoxycarbonyl)-L-alanine, 3-cyclobutyl-N-{[(1,1-dimethylethyl)oxy]carbonyl}-L-alanine N,N-diisopropylamine (1:1), 3-cyclohexyl-N-{[(1,1-dimethylethyl)oxy]carbonyl}-L-alanine, N-{[(1,1-dimethylethyl)oxy]carbonyl}-4-methyl-L-leucine, (2S)-cyclopropyl({[(1,1-dimethylethyl)oxy]carbonyl}amino)ethanoic acid, or N-{[(1,1-dimethylethyl)oxy]carbonyl}-L-norvaline. Formation of an appropriate amide derivative, such as a Weinreb amide, using an appropriate amine or amine salt, such as N,O-dimethylhydroxylamine hydrochloride, with an appropriate coupling reagent, such as the BOP reagent, and an appropriate base, such as DIPEA, in an appropriate solvent, such as CH₂Cl₂, followed by reduction with an appropriate reducing agent, such as LiAlH₄, in an appropriate solvent, such as THF, provides the requisite aldehyde. Enoate formation with an appropriate olefinating reagent, such as methyl (triphenylphosphoranylidene)acetate, in an appropriate solvent, such as Et₂O, is followed by Boc deprotection with an appropriate reagent, such as TFA, in an appropriate solvent, such as CH₂Cl₂. Coupling of the liberated amine with an appropriate Boc-protected α-amino acid, such as N-(tert-butoxycarbonyl)-L-alanine, N-{[(1,1-dimethylethyl)oxy]carbonyl}-3-(2-thienyl)-L-alanine, (2S)-2-({[(1,1-dimethylethyl)oxy]carbonyl}amino)-butanoic acid, (2S)-1-{[(1,1-dimethylethyl)oxy]carbonyl}-2-azetidinecarboxylic acid, (2S)-cyclopentyl({[(1,1-dimethylethyl)oxy]carbonyl}-amino)ethanoic acid, N-(tert-butoxycarbonyl)-L-valine, N-{[(1,1-dimethylethyl)oxy]carbonyl}-3-methyl-L-valine, N-{[(1,1-dimethylethyl)oxy]carbonyl}-L-isoleucine, N-{[(1,1-dimethylethyl)oxy]carbonyl}-L-alloisoleucine, (2S)-cyclopropyl({[(1,1-dimethylethyl)oxy]carbonyl}amino)ethanoic acid, 1-{[(1,1-dimethylethyl)oxy]carbonyl}-L-proline, (4S)-1-{[(1,1-dimethylethyl)oxy]carbonyl}-4-fluoro-L-proline, (2S)-3-{[(1,1-dimethylethyl)oxy]carbonyl}-3-azabicyclo[3.1.0]hexane-2-carboxylic acid, or (2S)-1-{[(1,1-dimethylethyl)oxy]carbonyl}-2-piperidinecarboxylic acid, with an appropriate coupling reagent or reagents, such as EDCI and HOBt, and an appropriate base, such as NMM, in an appropriate solvent, such as DMF, is followed by ester hydrolysis with an appropriate reagent, such as LiOH, in an appropriate solvent, such as THF and/or water. A variety of acyclic or cyclic amines are coupled to the resultant enoic acid with an appropriate coupling reagent or reagents, such as EDCI and HOBt or HATU, and an appropriate base, such as NMM or DIPEA, in an appropriate solvent, such as DMF. Boc deprotection with an appropriate reagent, such as HCl or TFA, results in the formation of the desired compounds of Formula (I), which may be isolated as the corresponding salt form or converted to the free base. The free base form of a compound of Formula (I) may be prepared by any suitable method known to the art, including treatment of the salt with an inorganic or organic base, suitably an inorganic or organic base having a higher pKa than the free base form of the compound.

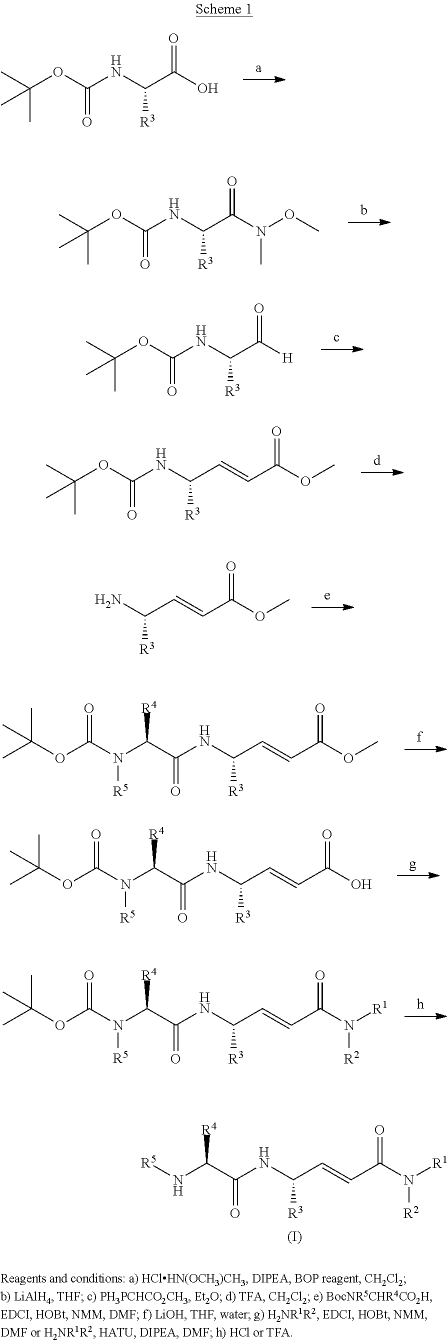

Alternatively, compounds of Formula (I) can be prepared by altering the order of the steps above as depicted in Scheme 2. Thus, ester hydrolysis of the intermediate enoate with an appropriate reagent, such as LiOH, in an appropriate solvent system, such as THF and water, is followed by amide bond formation with an appropriate acyclic or cyclic amine and an appropriate coupling reagent or reagents, such as HATU, and an appropriate base, such as DIPEA, in an appropriate solvent, such as DMF. Boc deprotection with an appropriate reagent, such as HCl, is followed by coupling of the liberated amine with an appropriate Boc-protected α-amino acid, such as N-{[(1,1-dimethylethyl)oxy]carbonyl}-3-(2-thienyl)-L-alanine, (2S)-2-({[(1,1-dimethylethyl)oxy]carbonyl}amino)-butanoic acid, or (2S)-1-{[(1,1-dimethylethyl)oxy]carbonyl}-2-piperidinecarboxylic acid, with an appropriate coupling reagent or reagents, such as HATU or the BOP reagent, and an appropriate base, such as DIPEA, in an appropriate solvent, such as DMF. Boc deprotection with an appropriate reagent, such as HCl, results in the formation of the desired compounds of Formula (I), which may be isolated as the corresponding salt form or converted to the free base using conventional techniques.

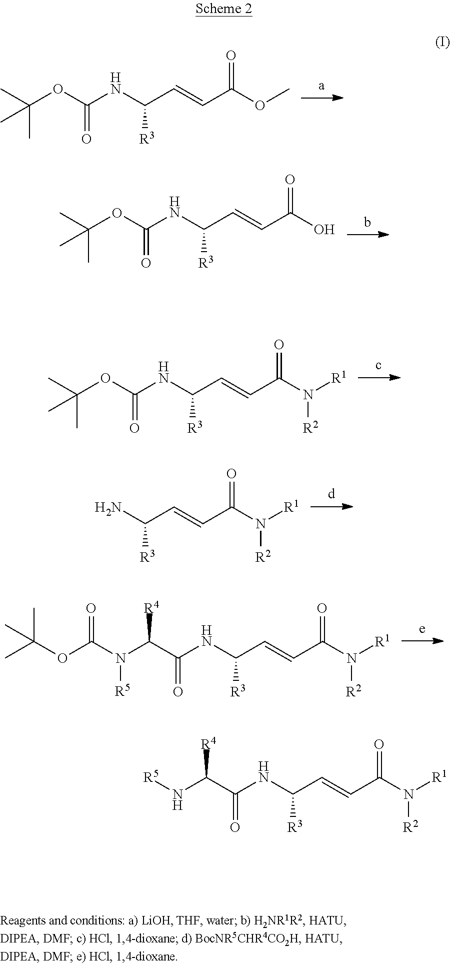

Alternatively, compounds of Formula (I) can be prepared as depicted in Scheme 3. Thus, treatment of an intermediate N-protected α-amino aldehyde with an appropriate amide stabilized Wittig reagent, such as 1-[(triphenyl-λ⁵-phosphanylidene)acetyl]-2,3-dihydro-1H-indole, in an appropriate solvent, such as THF, 2-methyltetrahydrofuran, and/or Et₂O, provides the requisite enamide. Boc deprotection with an appropriate reagent, such as HCl, is followed by coupling of the liberated amine with an appropriate Boc-protected α-amino acid, such as N-{[(1,1-dimethylethyl)oxy]carbonyl}-3-(2-thienyl)-L-alanine, (2S)-2-({[(1,1-dimethylethyl)oxy]carbonyl}amino)-butanoic acid, or (2S)-1-{[(1,1-dimethylethyl)oxy]carbonyl}-2-piperidinecarboxylic acid, with an appropriate coupling reagent or reagents, such as HATU or the BOP reagent, and an appropriate base, such as DIPEA, in an appropriate solvent, such as DMF. Boc deprotection with an appropriate reagent, such as HCl, results in the formation of the desired compounds of Formula (I), which may be isolated as the corresponding salt form or converted to the free base using conventional techniques.

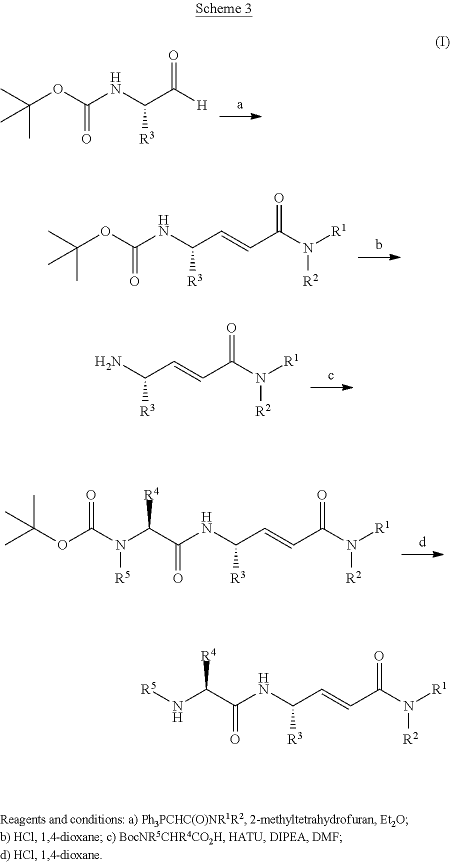

### SYNTHETIC EXAMPLES

The invention will now be described by reference to the following examples which are merely illustrative and are not to be construed as a limitation of the scope of the present invention. All temperatures are given in degrees Celsius, all solvents are highest available purity and all reactions run under anhydrous conditions in an argon (Ar) or nitrogen (N₂) atmosphere where necessary.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel. The CombiFlash® system used for purification in this application was purchased from Isco, Inc. CombiFlash® purification was carried out using prepacked silica gel columns, a detector with UV wavelength at 254 nm and a variety of solvents or solvent combinations. Preparative HPLC was performed using a Gilson Preparative System with variable wavelength UV detection or an Agilent Mass Directed AutoPrep (MDAP) system with both mass and variable wavelength UV detection. A variety of reverse phase columns, e.g., Luna 5u C18(2) 100 A, SunFire C18, XBridge C18 were used in the purification with the choice of column support dependent upon the conditions used in the purification. The compounds are eluted using a gradient of CH₃CN and water. Neutral conditions used an CH₃CN and water gradient with no additional modifier, acidic conditions used an acid modifier, usually 0.1% TFA (added to both the CH₃CN and water) and basic conditions used a basic modifier, usually 0.1% NH₄OH (added to the water). Analytical HPLC was run using an Agilent system with variable wavelength UV detection using reverse phase chromatography with an CH₃CN and water gradient with a 0.05 or 0.1% TFA modifier (added to each solvent). LC-MS was determined using either a PE Sciex Single Quadrupole LC/MS API-150a, or Waters ZQ instruments. The compound is analyzed using a reverse phase column, e.g., Thermo Aquasil/Aquasil C18, Acquity HPLC C18, Thermo Hypersil Gold eluted using an CH₃CN and water gradient with a low percentage of an acid modifier such as 0.02% TFA or 0.1% formic acid.

Nuclear magnetic resonance spectra were recorded at 400 MHz using a Bruker AVANCE 400 or Brucker DPX400 spectrometer. CDCl₃ is deuteriochloroform, DMSO-d₆ is hexadeuteriodimethylsulfoxide, and MeOD is tetradeuteriomethanol. Chemical shifts are reported in parts per million (δ) downfield from the internal standard tetramethylsilane (TMS) or calibrated to the residual proton signal in the NMR solvent (e.g., CHCl₃ in CDCl₃). Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Melting points were determined using an Electrothermal 9100 apparatus (Electrothermal Engineering Ltd.).

Heating of reaction mixtures with microwave irradiations was carried out on a Smith Creator (purchased from Personal Chemistry, Foxboro, Mass., now owned by Biotage), an Emrys Optimizer (purchased from Personal Chemistry) or an Explorer (purchased from CEM, Matthews, N.C.) microwave.

Cartridges or columns containing polymer based functional groups (acid, base, metal chelators, etc) can be used as part of compound workup. The “amine” columns or cartridges are used to neutralize or basify acidic reaction mixtures or products. These include NH2 Aminopropyl SPE-ed SPE Cartridges available from Applied Separations and diethylamino SPE cartridges available from United Chemical Technologies, Inc.

Abbreviations are listed in the table below. All other abbreviations are as described in the ACS Style Guide (American Chemical Society, Washington, D.C., 1986).

Table of Abbreviations

| | |
|---|---|
| BOP reagent: | MDAP: mass directed auto |
| benzotriazole-1-yl-oxy-tris- | preparation (an automated HPLC |
| (dimethylamino)-phosphonium | system which collects samples |
| hexafluorophosphate | based on real time |
| | mass spectral data) |
| HBTU: | HATU: |
| O-benzotriazol-1-yl-N,N,N′,N′- | O-(7-azabenzotriazol-1-yl)- |
| tetramethyluronium | N,N,N′,N′-tetramethyluronium |
| hexafluorophosphate | hexafluorophosphate |
| EDCI: 1-(3-dimethylaminopropyl)-3- | CH₂Cl₂: dichloromethane |
| ethylcarbodiimide hydrochloride | |
| DMAP: 4-(dimethylamino)pyridine | DMSO: dimethyl sulfoxide |
| DIPEA: N,N-diisopropylethylamine | THF: tetrahydrofuran |
| HOBt: 1-hydoxybenzotriazole hydrate | DMF: N,N-dimethylformamide |
| TFA: trifluoroacetic acid | NMM: N-methylmorpholine |
| HCl: hydrochloric acid | EtOAc: ethyl acetate |
| H₂SO₄: sulfuric acid | Et₂O: diethyl ether |
| NaHCO₃: sodium bicarbonate | PE: petroleum ether |
| NaOH: sodium hydroxide | MeOH: methanol |
| Na₂SO₄: sodium sulfate | CH₃CN: acetonitrile |
| MgSO₄: magnesium sulfate | aq.: aqueous |
| NH₄Cl: ammonium chloride | M: molar |
| LiAlH₄: lithium aluminum | mol: mole(s) |
| hydride | |
| L: liter(s) | mmol: millimole(s) |
| mL: milliliter(s) | g: gram(s) |
| min: minute(s) | mg: milligram(s) |
| h: hour(s) | RT: room temperature |

### INTERMEDIATE COMPOUNDS

### Intermediate 1

### 1,1-dimethylethyl (1S)-1-{[methyl(methyloxy)amino]carbonyl}-3-phenylpropyl)carbamate

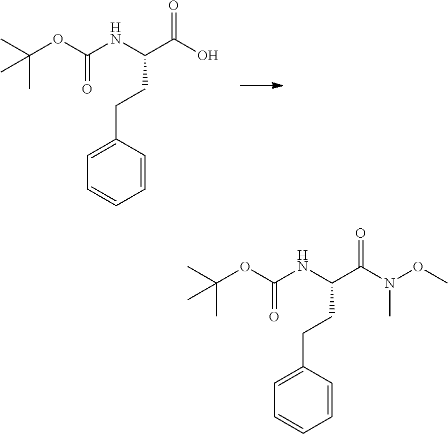

To a mixture of (2S)-2-({[(1,1-dimethylethyl)oxy]carbonyl}amino)-4-phenylbutanoic acid (86.0 g, 0.307 mol), BOP reagent (163.0 g, 0.369 mol) and DIPEA (47.6 g, 0.369 mol) in CH₂Cl₂ (1.05 L) at 0° C. was added dropwise a solution of N,O-dimethylhydroxylamine hydrochloride (36.0 g, 0.369 mol) and DIPEA (47.6 g, 0.369 mol) in CH₂Cl₂ (350 mL). The reaction mixture was stirred at RT overnight. The reaction mixture was washed with 1 M aq. HCl (3×300 mL), saturated aq. NaHCO₃ (2×300 mL), and brine. The organic layer was dried over Na₂SO₄, filtered, and concentrated in vacuo. Purification via flash column chromatography (PE:EtOAc=10:1) afforded the title compound (86.0 g, 87%) as an oil. LC-MS m/z 323 (M-Boc+H)⁺, 1.18 min (ret time).

### Intermediate 2

### 1,1-dimethylethyl[(1S)-1-formyl-3-phenylpropyl]carbamate

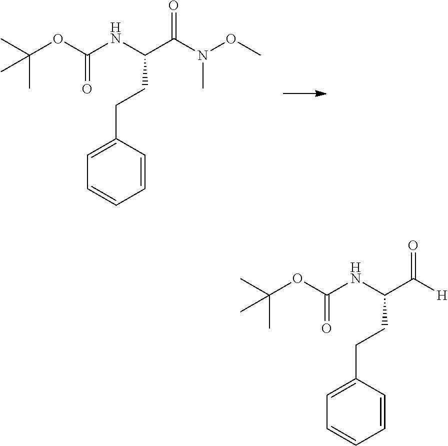

To a solution of 1,1-dimethylethyl((1S)-1-{[methyl(methyloxy)amino]carbonyl}-3-phenylpropyl)carbamate (86.0 g, 0.266 mol) in THF (600 mL) at 0° C. was added LiAlH₄ (13.17 g, 0.347 mol). The reaction mixture was stirred at 0° C. for 1 h, quenched with Na₂SO₄.10H₂O (40.0 g in 600 mL of water), and stirred for an additional 2 h. The reaction mixture was diluted with Et₂O (200 mL) and the layers were separated. The aqueous layer was extracted with Et₂O (3×200 mL). The combined organic layers were washed with 1 M aq. HCl (2×100 mL), saturated aq. NaHCO₃ (2×100 mL), and brine, dried over Na₂SO₄, filtered, and concentrated in vacuo to afford the title compound (60 g, 86%), which was carried to the next step without further purification. ¹H NMR (400 MHz, DMSO-d₆) δ ppm 9.55 (s, 1H), 7.30-7.18 (m, 5H), 5.08 (d, 2H), 4.25 (m, 1H), 2.71 (m, 1H), 2.24 (m, 1H), 1.84 (m, 1H), 1.46 (s, 9H).

### Intermediate 3

### methyl (2E,4S)-4-({[(1,1-dimethylethyl)oxy]carbonyl}amino)-6-phenyl-2-hexenoate

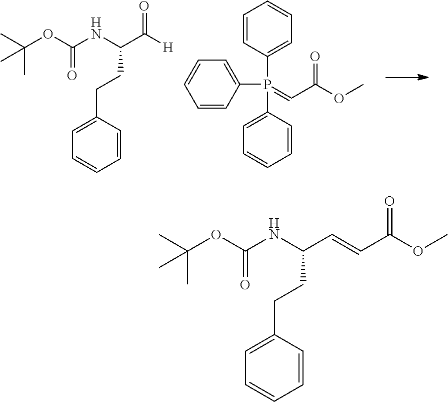

To a stirred solution of methyl(triphenylphosphoranylidene)acetate (40.11 g, 0.12 mol) in Et₂O (300 mL) at RT was added 1,1-dimethylethyl[(1S)-1-formyl-3-phenylpropyl]carbamate (26.0 g, 0.10 mol). The reaction mixture was stirred at RT for 12 h. The solid was removed by filtration and the solution was concentrated in vacuo. Purification via flash column chromatography (PE:EtOAc=20:1) afforded the title compound as an oil (12.0 g, 38%) along with an additional less pure batch (11.0 g, 49%). LC-MS m/z 220 (M-Boc+H)⁺, 1.60 min (ret time).

### Intermediate 4

### methyl (2E,4S)-4-amino-6-phenyl-2-hexenoate trifluoroacetate

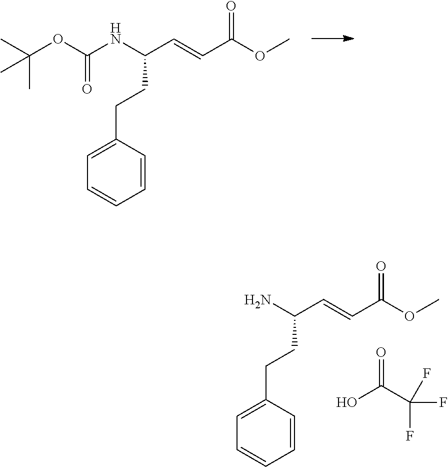

A solution of methyl (2E,4S)-4-({[(1,1-dimethylethyl)oxy]carbonyl}amino)-6-phenyl-2-hexenoate (12.0 g, 37.6 mmol) and TFA (42.89 g, 376 mmol) in CH₂Cl₂ (200 mL) was stirred at RT overnight. Following concentration in vacuo, the residue was diluted with Et₂O (20 mL) and stirred for 2 h. The resultant solid was filtered and dried to give the title compound (10.2 g, 69%) as a white solid. LC-MS m/z 220 (M+H)⁺, 0.92 min (ret time).

### Intermediate 5

### methyl (2E,4S)-4-[(N-{[(1,1-dimethylethyl)oxy]carbonyl}-L-alanyl)amino]-6-phenyl-2-hexenoate

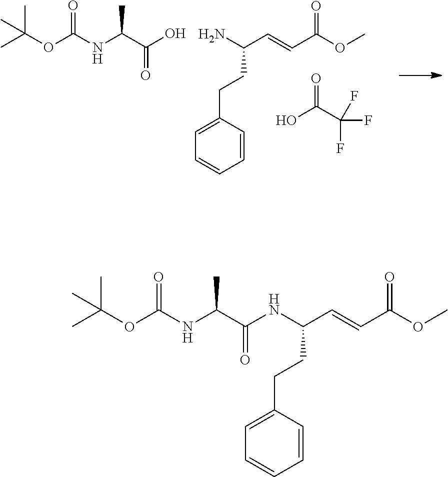

A mixture of methyl (2E,4S)-4-amino-6-phenyl-2-hexenoate trifluoroacetate (10.2 g, 32.2 mmol), N-(tert-butoxycarbonyl)-L-alanine (6.41 g, 33.9 mmol), EDCI (12.31 g, 64.4 mmol), HOBt (8.70 g, 64.4 mmol), and NMM (9.77 g, 96.6 mmol) in DMF (80.0 mL) was stirred at RT overnight. The reaction mixture was poured into water and extracted with CH₂Cl₂. The organic layer was washed with water and brine, dried over Na₂SO₄, filtered, and concentrated in vacuo. Purification via flash column chromatography (PE:EtOAc=2:1) afforded the title compound (11.0 g, 88%) as an oil. LC-MS m/z 391 (M+H)⁺, 1.47 min (ret time).

### Intermediate 6

### (2E,4S)-4-[(N-{[(1,1-dimethylethyl)oxy]carbonyl}-L-alanyl)amino]-6-phenyl-2-hexenoic acid

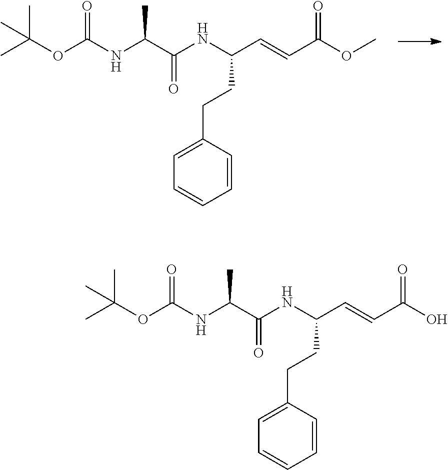

To a solution of methyl (2E,4S)-4-[(N-{[(1,1-dimethylethyl)oxy]carbonyl}-L-alanyl)amino]-6-phenyl-2-hexenoate (11.0 g, 28.0 mmol) in THF (250 mL) and water (250 mL) was added LiOH (5.9 g, 140 mmol). After stirring overnight at RT, the reaction mixture was acidified with 1 M aq. HCl to pH ˜2-3 and then extracted with EtOAc (3×200 mL). The combined organic layers were washed with water and brine, dried over Na₂SO₄, filtered, and concentrated in vacuo to afford the title compound (8.9 g, 85%) as a yellow solid. LC-MS m/z 321 (M-55+H)⁺, 1.34 min (ret time).

### Intermediate 7

### 1,1-dimethylethyl[(1S)-1-methyl-2-oxo-2-({(1S,2E)-4-oxo-1-(2-phenylethyl)-4-[(phenylmethyl)amino]-2-buten-1-yl}amino)ethyl]carbamate

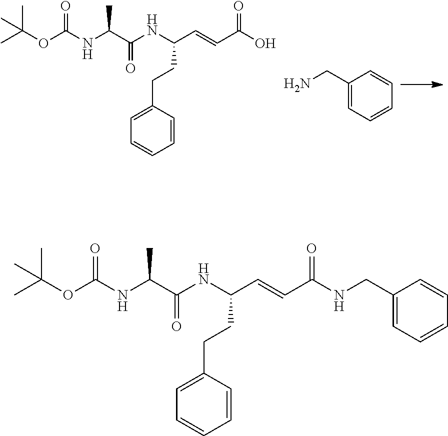

A mixture of (2E,4S)-4-[(N-{[(1,1-dimethylethyl)oxy]carbonyl}-L-alanyl)amino]-6-phenyl-2-hexenoic acid (2.00 g, 5.31 mmol), (phenylmethyl)amine (0.625 g, 5.84 mmol), EDCI (2.03 g, 10.62 mmol), HOBt (1.43 g, 10.62 mmol), and NMM (1.61 g, 15.93 mmol) in DMF (30.0 mL) was stirred at RT overnight. Saturated NH₄Cl (20.0 mL) was added to quench the reaction. A solid that precipitated out from the solution was collected by filtration, washed with water (20.0 mL), and dried in vacuo to afford the title compound (2.00 g, 81%). LC-MS m/z 466 (M+H)⁺, 1.64 min (ret time).

### Intermediate 8

### 1,1-dimethylethyl((1S)-1-methyl-2-{[(1S,2E)-4-(methylamino)-4-oxo-1-(2-phenylethyl)-2-buten-1-yl]amino}-2-oxoethyl)carbamate

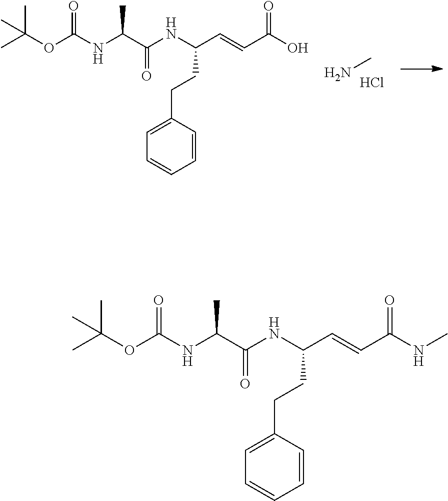

A mixture of (2E,4S)-4-[(N-{[(1,1-dimethylethyl)oxy]carbonyl}-L-alanyl)amino]-6-phenyl-2-hexenoic acid (2.00 g, 5.31 mmol), methylamine hydrochloride (0.391 g, 5.84 mmol), EDCI (2.03 g, 10.62 mmol), HOBt (1.43 g, 10.62 mmol), and NMM (1.61 g, 15.93 mmol) in DMF (30.0 mL) was stirred at RT overnight. Saturated NH₄Cl (20.0 mL) was added to quench the reaction. A solid that precipitated out from the solution was collected by filtration, washed with water (20.0 mL), and dried in vacuo to afford the title compound (1.7 g, 81%). LC-MS m/z 390 (M+H)⁺, 1.30 min (ret time).

### Intermediate 9

### 1,1-dimethylethyl((1S)-2-{[(1S,2E)-4-(dimethylamino)-4-oxo-1-(2-phenylethyl)-2-buten-1-yl]amino}-1-methyl-2-oxoethyl)carbamate

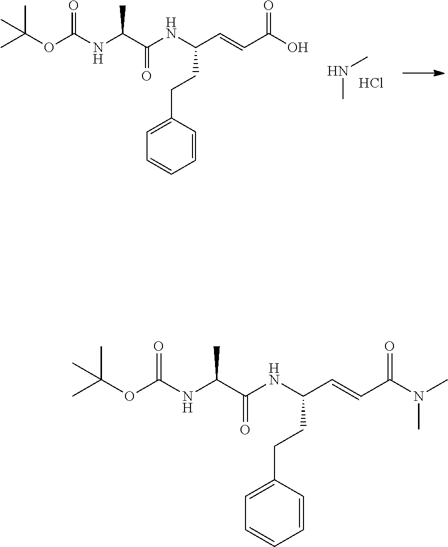

A mixture of (2E,4S)-4-[(N-{[(1,1-dimethylethyl)oxy]carbonyl}-L-alanyl)amino]-6-phenyl-2-hexenoic acid (2.00 g, 5.38 mmol), dimethylamine hydrochloride (0.473 g, 5.84 mmol), EDCI (2.03 g, 10.62 mmol), HOBt (1.43 g, 10.62 mmol), and NMM (1.61 g, 15.93 mmol) in DMF (30.0 mL) was stirred at RT overnight. Saturated NH₄Cl (20.0 mL) was added to quench the reaction. A solid that precipitated out from the solution was collected by filtration, washed with water (20.0 mL), and dried in vacuo to afford the title compound (2.00 g, 92%). LC-MS m/z 403 (M+H)⁺, 1.34 min (ret time).

### Intermediate 10

### 1,1-dimethylethyl((1S)-1-methyl-2-oxo-2-{[(1S,2E)-4-oxo-1-(2-phenylethyl)-4-(1-piperidinyl)-2-buten-1-yl]amino}ethyl)carbamate

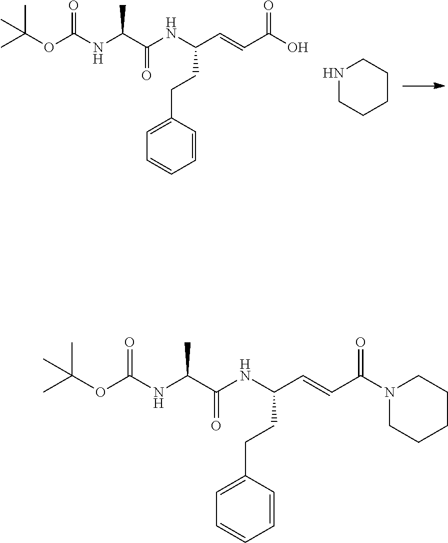

A mixture of (2E,4S)-4-[(N-{[(1,1-dimethylethyl)oxy]carbonyl}-L-alanyl)amino]-6-phenyl-2-hexenoic acid (2.00 g, 5.31 mmol), piperidine (0.496 g, 5.84 mmol), EDCI (2.03 g, 10.62 mmol), HOBt (1.43 g, 10.62 mmol), and NMM (1.61 g, 15.93 mmol) in DMF (30.0 mL) was stirred at RT overnight. Saturated NH₄Cl (20.0 mL) was added to quench the reaction. A solid that precipitated out from the solution was collected by filtration, washed with water (20.0 mL), and dried in vacuo to afford the title compound (1.00 g, 43%) as a white solid. LC-MS m/z 444 (M+H)⁺, 1.46 min (ret time).

### Intermediate 11

### 1,1-dimethylethyl((1S)-1-methyl-2-{[(1S,2E)-4-{[4-(methyloxy)phenyl]amino}-4-oxo-1-(2-phenylethyl)-2-buten-1-yl]amino}-2-oxoethyl)carbamate

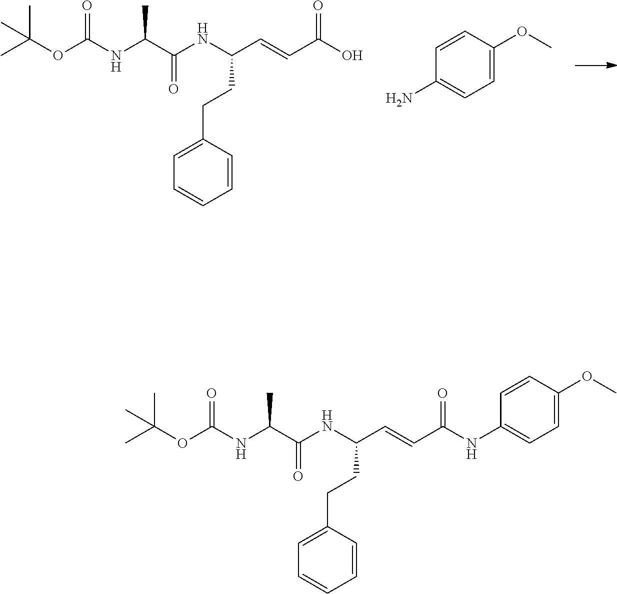

A mixture of (2E,4S)-4-[(N-{[(1,1-dimethylethyl)oxy]carbonyl}-L-alanyl)amino]-6-phenyl-2-hexenoic acid (2.00 g, 5.31 mmol), 4-(methyloxy)aniline (0.72 g, 5.84 mmol), EDCI (2.03 g, 10.62 mmol), HOBt (1.43 g, 10.62 mmol), and NMM (1.61 g, 15.93 mmol) in DMF (30.0 mL) was stirred at RT overnight. Saturated NH₄Cl (20.0 mL) was added to quench the reaction. A solid that precipitated out from the solution was collected by filtration, washed with water (20.0 mL), and dried in vacuo to afford the title compound (1.20 g, 48%) as a white solid. LC-MS m/z 482 (M+H)⁺, 1.48 min (ret time).

### Intermediate 12

### 1,1-dimethylethyl((1S)-2-{[(1S,2E)-4-(1H-indol-1-yl)-4-oxo-1-(2-phenylethyl)-2-buten-1-yl]amino}-1-methyl-2-oxoethyl)carbamate

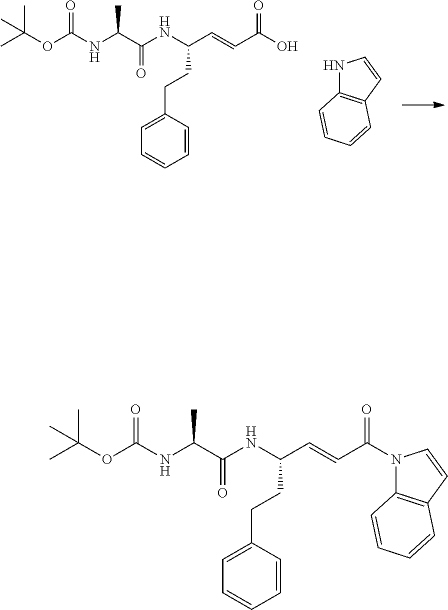

A solution of (2E,4S)-4-[(N-{[(1,1-dimethylethyl)oxy]carbonyl}-L-alanyl)amino]-6-phenyl-2-hexenoic acid (0.97 g, 2.58 mmol), HATU (1.061 g, 2.71 mmol) and DIPEA (2.0 mL, 11.42 mmol) in DMF (4.0 mL) was stirred at RT for 45 min in a vial. In a separate vial, to a solution of 1H-indole (0.357 g, 3.05 mmol) in DMF (4.0 mL) was added sodium hydride (0.117 g, 2.93 mmol), and the reaction mixture was stirred at RT for 40 min. The contents of the two reaction vessels were combined with additional DMF (50.0 mL) and the reaction mixture was stirred at RT for 50 min. The reaction mixture was partitioned between EtOAc (80 mL) and water (40 mL). The aqueous layer was separated and extracted with EtOAc (40 mL). The combined organic layers were washed water (2×40 mL) and brine (40 mL), and then concentrated in vacuo. Purification via flash column chromatography (10-30% EtOAc/hexanes) afforded the title compound (0.19 g, 15%) as a clear, colorless oil. LC-MS m/z 476 (M+H)⁺, 1.07 min (ret time).

### Intermediate 13

### 1,1-dimethylethyl((1S)-2-{[(1S,2E)-4-[(1R,8S)-11-azatricyclo[6.2.1.0^{2,7}]undeca-2,4,6-trien-11-yl]-4-oxo-1-(2-phenylethyl)-2-buten-1-yl]amino}-1-methyl-2-oxoethyl)carbamate

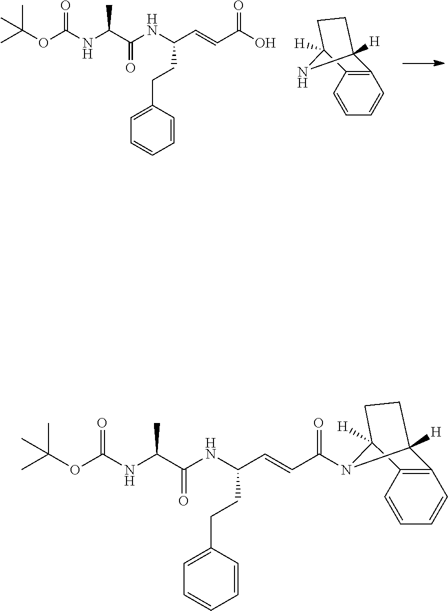

A solution of (2E,4S)-4-[(N-{[(1,1-dimethylethyl)oxy]carbonyl}-L-alanyl)amino]-6-phenyl-2-hexenoic acid (100 mg, 0.266 mmol), HATU (105 mg, 0.267 mmol), and DIPEA (0.186 mL, 1.063 mmol) in CH₂Cl₂ (5.0 mL) was stirred at RT for 30 min. (1R,8S)-11-azatricyclo[6.2.1.0^{2,7}]undeca-2,4,6-triene was added and stirring continued for 10 min. The reaction mixture was diluted with water (10 mL) and EtOAc (20 mL). Following separation of the layers, the organic layer was washed three times with water (10 mL) and twice with brine (10 mL) and then concentrated in vacuo to afford the title compound (140 mg). LC-MS m/z 504 (M+H)⁺, 1.25 min (ret time).

### Intermediate 14

### methyl (2E,4S)-4-{[(2S)-2-({[(1,1-dimethylethyl)oxy]carbonyl}amino)butanoyl]amino}-6-phenyl-2-hexenoate

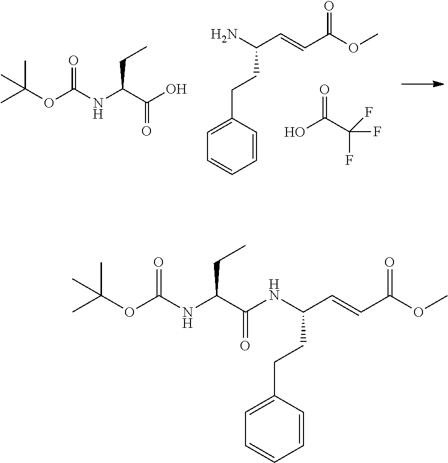

A mixture of methyl (2E,4S)-4-amino-6-phenyl-2-hexenoate trifluoroacetate (2.90 g, 8.70 mmol), (2S)-2-({[(1,1-dimethylethyl)oxy]carbonyl}amino)butanoic acid (1.86 g, 9.14 mmol), EDCI (3.34 g, 17.4 mmol), HOBt (2.66 g, 17.4 mmol), and NMM (2.87 mL, 26.1 mmol) in DMF (20.0 mL) was stirred at RT for 3 h. Water was added to the reaction mixture which was stirred an additional 10-15 min. An off white solid was collected by filtration, dissolved in CH₂Cl₂, dried over Na₂SO₄, filtered, and concentrated in vacuo to afford the title compound (3.29 g, 93%) as a white solid. LC-MS m/z 405 (M+H)⁺, 1.13 min (ret time).

### Intermediate 15

### (2E,4S)-4-{[(2S)-2-({[(1,1-dimethylethyl)oxy]carbonyl}amino)butanoyl]amino}-6-phenyl-2-hexenoic acid

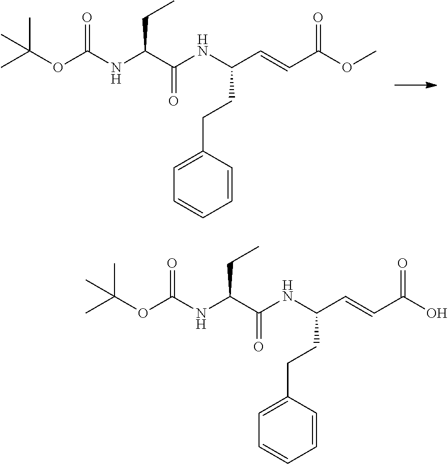

To a solution of methyl (2E,4S)-4-{[(2S)-2-({[(1,1-dimethylethyl)oxy]carbonyl}-amino)butanoyl]amino}-6-phenyl-2-hexenoate (3.29 g, 8.13 mmol) in THF (50 mL) and water (50 mL) was added LiOH (0.974 g, 40.7 mmol). After stirring at RT for 15 h, the reaction mixture was acidified with 1 M aq. HCl to pH ˜3 and then extracted with EtOAc (50 mL). The organic layer was washed with water (50 mL) and brine (50 mL), dried over Na₂SO₄, filtered, and concentrated in vacuo. The resultant oil was diluted with Et₂O followed by hexanes. Concentration in vacuo produced a white foam which was collected by scraping to afford the title compound (2.84 g, 89%) as a white solid. LC-MS m/z 391 (M−55+H)⁺, 1.03 min (ret time).

### Intermediate 16

### 1,1-dimethylethyl((1S)-1-{[methyl(methyloxy)amino]carbonyl}propyl)carbamate

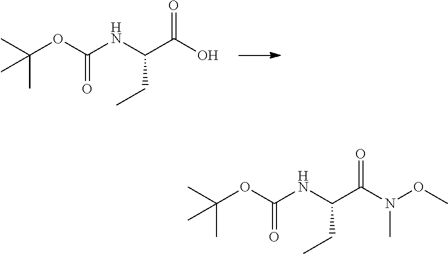

To a solution of (2S)-2-({[(1,1-dimethylethyl)oxy]carbonyl}amino)butanoic acid (2.50 g, 12.3 mmol) in THF (15.0 mL) was added 1,1′-carbonyldiimidazole (2.39 g, 14.8 mmol) portionwise over about 10 min. After stirring 30 min at RT, a solution of N,O-dimethylhydroxylamine hydrochloride (1.32 g, 13.5 mmol) and DIPEA (2.36 mL, 13.5 mmol) in DMF (4.0 mL) was added. The reaction mixture was stirred for 2 h at RT, followed by concentration in vacuo. The residue was diluted with EtOAc (50 mL) and washed with 1 M aq. HCl (2×20 mL), saturated aq. NaHCO₃ (2×20 mL), and brine (20 mL). The organic layer was dried over Na₂SO₄, filtered, and concentrated in vacuo to afford the title compound (2.60 g, 88%) as a clear, colorless oil. LC-MS m/z 247 (M+H)⁺, 0.94 min (ret time).

### Intermediate 17

### 1,1-dimethylethyl[(1S)-1-formylpropyl]carbamate

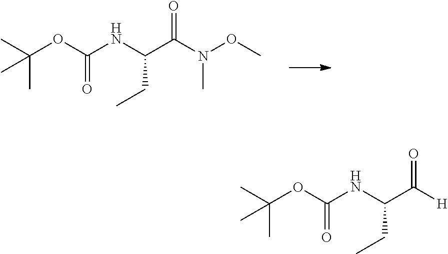

To a solution of LiAlH₄ (0.453 g, 11.9 mmol) in Et₂O (20 mL) at 0° C. was added dropwise a solution of 1,1-dimethylethyl 415)-1-{[methyl(methyloxy)amino]carbonyl}-propyl)carbamate (2.67 g, 10.8 mmol) in Et₂O (15 mL). The reaction mixture was stirred for 30 min at 0° C. and quenched with EtOAc (6.5 mL) followed by 5% aq. potassium bisulfate (6.5 mL). The reaction mixture was washed with 1 M aq. HCl (3×10 mL), saturated aq. NaHCO₃ (3×10 mL), and brine (10 mL). The organic layer was dried over Na₂SO₄, filtered, and concentrated in vacuo to afford the title compound as a clear, colorless oil, which was carried to the next step without further purification.

### Intermediate 18

### methyl (2E,4S)-4-({[(1,1-dimethylethyl)oxy]carbonyl}amino)-2-hexenoate

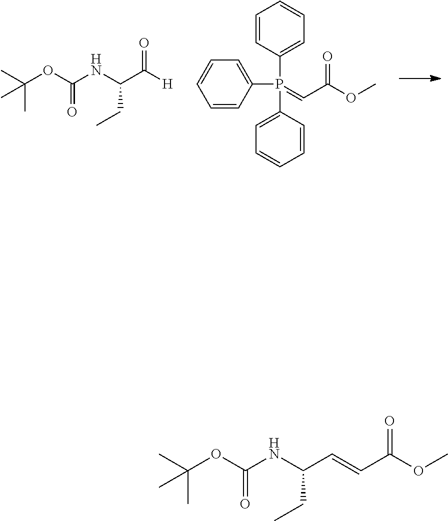

To a stirred solution of methyl(triphenylphosphoranylidene)acetate (4.35 g, 13.0 mmol) in Et₂O (25 mL) at RT was added a solution of Intermediate 17 in Et₂O (15 mL). The reaction mixture was stirred at RT overnight. The solid was removed by filtration and the solution was concentrated in vacuo. Purification via flash column chromatography (0-50% EtOAc/hexanes) afforded the title compound (1.44 g, 55% over two steps) as a clear, colorless oil. LC-MS m/z 244 (M+H)⁺, 0.98 min (ret time).

### Intermediate 19

### methyl (2E,4S)-4-amino-2-hexenoate trifluoroacetate

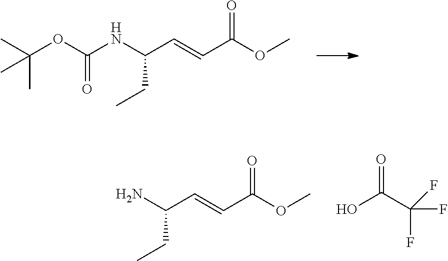

To a solution of methyl (2E,4S)-4-({[(1,1-dimethylethyl)oxy]carbonyl}amino)-2-hexenoate (1.44 g, 5.92 mmol) in CH₂Cl₂ (30 mL) was added TFA (4.56 mL, 59.2 mmol). The reaction mixture was stirred at RT for 2.5 h and then concentrated in vacuo. The resultant oil was diluted with Et₂O (5 mL), hexanes was added with stirring until the mixture became cloudy, and the mixture was concentrated in vacuo to give an off white solid. The solid was triturated and washed with Et₂O to afford the title compound (1.19 g, 78%) as a white solid. LC-MS m/z 144 (M+H)⁺, 0.46 min (ret time).

### Intermediate 20

### methyl (2E,4S)-4-[(N-{[(1,1-dimethylethyl)oxy]carbonyl}-L-alanyl)amino]-2-hexenoate

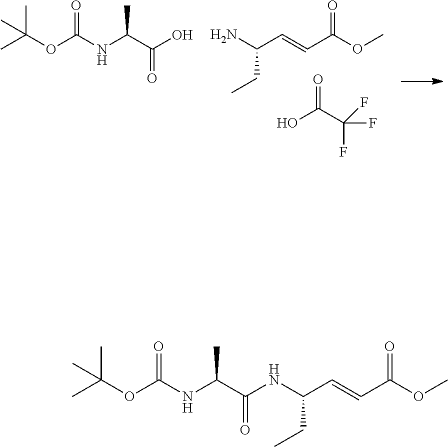

A mixture of methyl (2E,4S)-4-amino-2-hexenoate trifluoroacetate (1.19 g, 4.63 mmol), N-(tert-butoxycarbonyl)-L-alanine (0.919 g, 4.86 mmol), EDCI (1.77 g, 9.25 mmol), HOBt (1.42 g, 9.25 mmol), and NMM (1.53 mL, 13.9 mmol) in DMF (10.0 mL) was stirred for 1 h at RT. Water (100 mL) was added with stirring, followed by extraction with EtOAc (100 mL). The organic layer was washed with water (5×100 mL) and brine (100 mL), dried over Na₂SO₄, filtered, and concentrated in vacuo to afford the title compound (1.42 g, 98%) as a colorless glass. LC-MS m/z 315 (M+H)⁺, 1.02 min (ret time).

### Intermediate 21

### (2E,4S)-4-[(N-{[(1,1-dimethylethyl)oxy]carbonyl}-L-alanyl)amino]-2-hexenoic acid

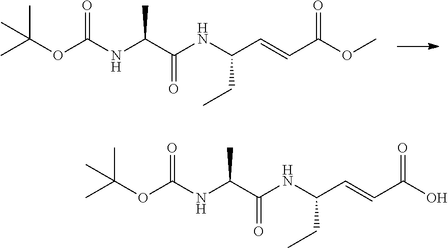

To a solution of methyl (2E,4S)-4-[(N-{[(1,1-dimethylethyl)oxy]carbonyl}-L-alanyl)amino]-2-hexenoate (1.42 g, 4.52 mmol) in THF (25 mL) and water (25 mL) was added LiOH (0.541 g, 22.6 mmol). After stirring for 15 h at RT, the reaction mixture was acidified with 1 M aq. HCl to pH=3 and then extracted with EtOAc (100 mL). The combined organic layers were washed with water (100 mL) and brine (100 mL), dried over Na₂SO₄, filtered, and concentrated in vacuo. The resultant oil was diluted with Et₂O and hexanes and concentrated in vacuo to afford the title compound (1.1 g, 81%) as a white solid. LC-MS m/z 301 (M+H)⁺, 0.91 min (ret time).

### Intermediate 22

### 1,1-dimethylethyl((1S)-2-{[(1S,2E)-4-(1,3-dihydro-2H-isoindol-2-yl)-1-ethyl-4-oxo-2-buten-1-yl]amino}-1-methyl-2-oxoethyl)carbamate

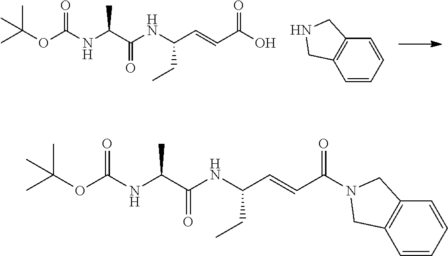

A solution of (2E,4S)-4-[(N-{[(1,1-dimethylethyl)oxy]carbonyl}-L-alanyl)amino]-2-hexenoic acid (100 mg, 0.333 mmol), HATU (127 mg, 0.333 mmol), and DIPEA (0.223 mL, 1.332 mmol) in CH₂Cl₂ (4.0 mL) was stirred at RT for 30 min. 2,3-dihydro-1H-isoindole (0.038 mL, 0.333 mmol) was added and stirring continued overnight. The reaction mixture was partitioned between water (10 mL) and EtOAc (20 mL). The organic layer was washed with water (3×10 mL) and brine (2×10 mL), and concentrated in vacuo to afford the title compound (120 mg, 90%). LC-MS m/z 402 (M+H)⁺, 1.39 min (ret time).

### Intermediate 23

### N²-{[(1,1-dimethylethyl)oxy]carbonyl}-N¹-methyl-N¹-(methyloxy)-L-isoleucinamide

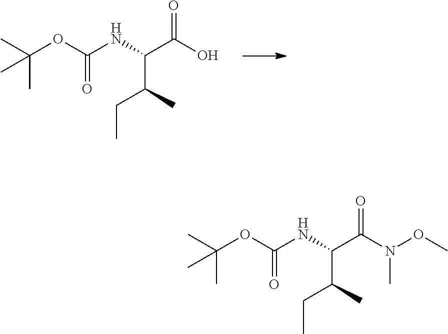

To a solution of N-{[(1,1-dimethylethyl)oxy]carbonyl}-L-isoleucine (3.00 g, 13.0 mmol) in THF (15.0 mL) was added 1,1′-carbonyldiimidazole (2.52 g, 15.6 mmol) portionwise over about 10 min. After stirring 30 min at RT, a solution of N,O-dimethylhydroxylamine hydrochloride (1.39 g, 14.3 mmol) and DIPEA (2.49 mL, 14.3 mmol) in DMF (4.0 mL) was added. The reaction mixture was stirred for 2 h at RT, followed by concentration in vacuo. The residue was diluted with EtOAc (50 mL) and washed with 1 M aq. HCl (2×20 mL), saturated aq. NaHCO₃ (2×20 mL), and brine (20 mL). The organic layer was dried over Na₂SO₄, filtered, and concentrated in vacuo. Purification via flash column chromatography (30-100% EtOAc/hexanes) afforded the title compound (2.62 g, 74%) as a clear, colorless oil. LC-MS m/z 275 (M+H)⁺, 1.07 min (ret time).

### Intermediate 24

### 1,1-dimethylethyl[(1S,2S)-1-formyl-2-methylbutyl]carbamate

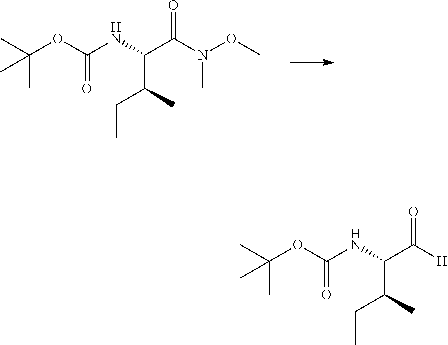

To a solution of LiAlH₄ (0.399 g, 10.5 mmol) in Et₂O (20 mL) at 0° C. was added dropwise a solution of N²-{[(1,1-dimethylethyl)oxy]carbonyl}-N¹-methyl-N¹-(methyloxy)-L-isoleucinamide (2.62 g, 9.55 mmol) in Et₂O (15 mL). The reaction mixture was stirred for 30 min at 0° C. and quenched with EtOAc (6.5 mL) followed by 5% aq. potassium bisulfate (6.5 mL). The reaction mixture was washed with 1 M aq. HCl (3×10 mL), saturated aq. NaHCO₃ (3×10 mL), and brine (10 mL). The organic layer was dried over Na₂SO₄, filtered, and concentrated in vacuo to afford the title compound as a clear, colorless oil, which was carried to the next step without further purification.

### Intermediate 25

### methyl (2E,4S,5S)-4-({[(1,1-dimethylethyl)oxy]carbonyl}amino)-5-methyl-2-heptenoate

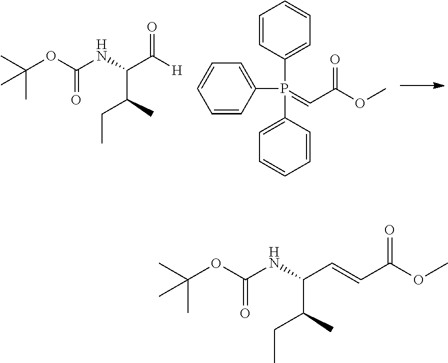

To a stirred solution of methyl(triphenylphosphoranylidene)acetate (3.83 g, 11.5 mmol) in Et₂O (25 mL) at RT was added a solution of Intermediate 24 in Et₂O (15 mL). The reaction mixture was stirred for 15 h at RT. The solid was removed by filtration and the solution was concentrated in vacuo. Purification via flash column chromatography (0-50% EtOAc/hexanes) afforded the title compound (1.90 g, 73% over two steps) as a clear, colorless oil. LC-MS m/z 272 (M+H)⁺, 1.20 min (ret time).

### Intermediate 26

### methyl (2E,4S,5S)-4-amino-5-methyl-2-heptenoate trifluoroacetate

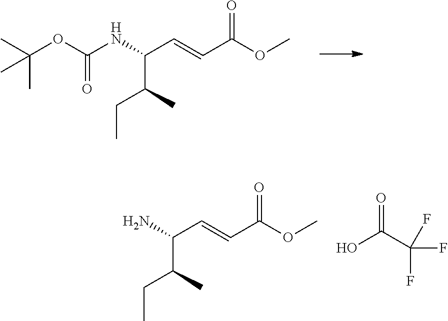

To a solution of methyl (2E,4S,5S)-4-({[(1,1-dimethylethyl)oxy]carbonyl}amino)-5-methyl-2-heptenoate (1.90 g, 7.00 mmol) in CH₂Cl₂ (30 mL) was added TFA (5.39 mL, 70.0 mmol). The reaction mixture was stirred at RT for 2.5 h and then concentrated in vacuo. The resultant oil was diluted with Et₂O (5 mL), hexanes was added with stirring until the mixture became cloudy, and the mixture was concentrated in vacuo to give an off white solid. The solid was triturated and washed with Et₂O to afford the title compound (1.77 g, 89%) as a white solid. LC-MS m/z 172 (M+H)⁺, 0.87 min (ret time).

### Intermediate 27

### methyl (2E,4S,5S)-4-[(N-{[(1,1-dimethylethyl)oxy]carbonyl}-L-alanyl)amino]-5-methyl-2-heptenoate

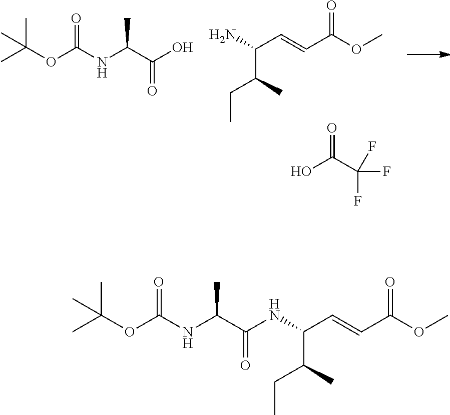

A mixture of methyl (2E,4S,5S)-4-amino-5-methyl-2-heptenoate trifluoroacetate (1.77 g, 6.20 mmol), N-(tert-butoxycarbonyl)-L-alanine (1.23 g, 6.52 mmol), EDCI (2.38 g, 12.4 mmol), HOBt (1.90 g, 12.4 mmol), and NMM (2.05 mL, 18.6 mmol) in DMF (15.0 mL) was stirred for 1 h at RT. Water (100 mL) was added with stirring, followed by extraction with EtOAc (100 mL). The organic layer was washed with water (5×100 mL) and brine (100 mL), dried over Na₂SO₄, filtered, and concentrated in vacuo to afford the title compound (1.79 g, 84%) as a colorless glass. LC-MS m/z 343 (M+H)⁺, 1.14 min (ret time).

### Intermediate 28

### (2E,4S,5S)-4-[(N-{[(1,1-dimethylethyl)oxy]carbonyl}-L-alanyl)amino]-5-methyl-2-heptenoic acid

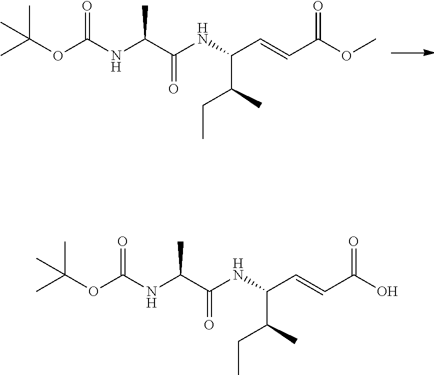

To a solution of methyl (2E,4S,5S)-4-[(N-{[(1,1-dimethylethyl)oxy]carbonyl}-L-alanyl)amino]-5-methyl-2-heptenoate (1.79 g, 5.23 mmol) in THF (25 mL) and water (25 mL) was added LiOH (0.626 g, 26.1 mmol). After stirring for 15 h at RT, the reaction mixture was acidified with 1 M aq. HCl to pH=3 and then extracted with EtOAc (100 mL). The combined organic layers were washed with water (100 mL) and brine (100 mL), dried over Na₂SO₄, filtered, and concentrated in vacuo. The resultant oil was diluted with Et₂O and hexanes and concentrated in vacuo to afford the title compound (1.42 g, 83%) as a fluffy white solid. LC-MS m/z 329 (M+H)⁺, 1.02 min (ret time).

### Intermediate 29

### 1,1-dimethylethyl[(1S)-2-({(1S,2E)-4-[(1R,8S)-11-azatricyclo[6.2.1.0^{2,7}]undeca-2,4,6-trien-11-yl]-1-[(1S)-1-methylpropyl]-4-oxo-2-buten-1-yl}amino)-1-methyl-2-oxoethyl]carbamate

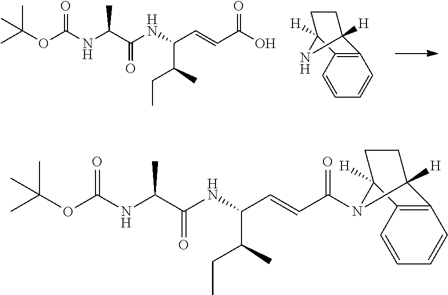

A solution of (2E,4S,5S)-4-[(N-{[(1,1-dimethylethyl)oxy]carbonyl}-L-alanyl)amino]-5-methyl-2-heptenoic acid (100 mg, 0.305 mmol), HATU (116 mg, 0.305 mmol), and DIPEA (0.204 mL, 1.218 mmol) in CH₂Cl₂ (4.0 mL) was stirred at RT for 30 min. (1R,8S)-11-azatricyclo[6.2.1.0^{2,7}]undeca-2,4,6-triene (44.2 mg, 0.305 mmol) was added and stirring continued overnight. The reaction mixture was partitioned between water (10 mL) and EtOAc (20 mL). The organic layer was washed with water (3×10 mL) and brine (2×10 mL), and concentrated in vacuo to afford the title compound (140 mg, 100%). LC-MS m/z 456 (M+H)⁺, 1.22 min (ret time).

### Intermediate 30

### 1,1-dimethylethyl[(1S)-2-({(1S,2E)-4-(1,3-dihydro-2H-isoindol-2-yl)-1-[(1S)-1-methylpropyl]-4-oxo-2-buten-1-yl}amino)-1-methyl-2-oxoethyl]carbamate

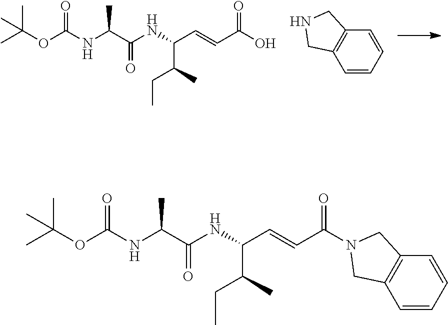

A solution of (2E,4S,5S)-4-[(N-{[(1,1-dimethylethyl)oxy]carbonyl}-L-alanyl)amino]-5-methyl-2-heptenoic acid (100 mg, 0.305 mmol), HATU (116 mg, 0.305 mmol), and DIPEA (0.204 mL, 1.218 mmol) in CH₂Cl₂ (4.0 mL) was stirred at RT for 30 min. 2,3-dihydro-1H-isoindole (0.035 mL, 0.305 mmol) was added and stirring continued overnight. The reaction mixture was partitioned between water (10 mL) and EtOAc (20 mL). The organic layer was washed with water (3×10 mL) and brine (2×10 mL), and concentrated in vacuo to afford the title compound (120 mg, 92%). LC-MS m/z 430 (M+H)⁺, 1.63 min (ret time).

### Intermediate 31

### N²-{[(1,1-dimethylethyl)oxy]carbonyl}-N¹-methyl-N¹-(methyloxy)-L-leucinamide

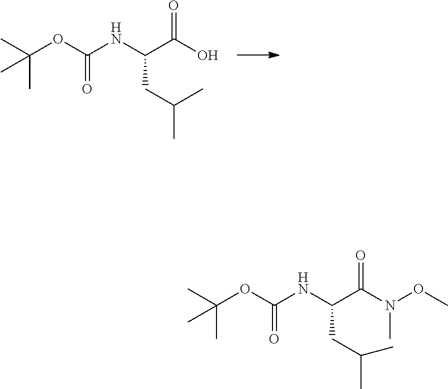

To a solution of N-(tert-butoxycarbonyl)-L-leucine (3.00 g, 13.0 mmol) in THF (25.0 mL) was added 1,1′-carbonyldiimidazole (2.52 g, 15.6 mmol) portionwise over about 10 min. After stirring 1 h at RT, a solution of N,O-dimethylhydroxylamine hydrochloride (1.39 g, 14.3 mmol) and DIPEA (2.49 mL, 14.3 mmol) in DMF (6.0 mL) was added. The reaction mixture was stirred for 2.5 h at RT, followed by concentration in vacuo. The residue was diluted with EtOAc (50 mL) and washed with 1 M aq. HCl (2×20 mL), saturated aq. NaHCO₃ (2×20 mL), and brine (20 mL). The organic layer was dried over Na₂SO₄, filtered, and concentrated in vacuo to afford the title compound (2.34 g, 66%) as a clear, colorless oil. LC-MS m/z 275 (M+H)⁺, 1.17 min (ret time).

### Intermediate 32

### 1,1-dimethylethyl[(1S)-1-formyl-3-methylbutyl]carbamate

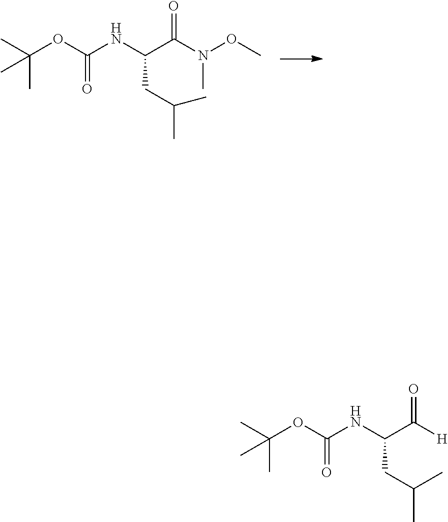

To a solution of LiAlH₄ (0.356 g, 9.38 mmol) in Et₂O (20 mL) at 0° C. was added dropwise a solution of N²-{[(1,1-dimethylethyl)oxy]carbonyl}-N¹-methyl-N¹-(methyloxy)-L-leucinamide (2.34 g, 8.53 mmol) in Et₂O (15 mL). The reaction mixture was stirred for 30 min at 0° C. and quenched with EtOAc (6 mL) followed by 5% aq. potassium bisulfate (6 mL). The reaction mixture was washed with 1 M aq. HCl (2×10 mL), saturated aq. NaHCO₃ (2×10 mL), and brine (10 mL). The organic layer was dried over Na₂SO₄, filtered, and concentrated in vacuo to afford the title compound as a clear, colorless oil, which was carried to the next step without further purification.

### Intermediate 33

### methyl (2E,4S)-4-({[(1,1-dimethylethyl)oxy]carbonyl}amino)-6-methyl-2-heptenoate

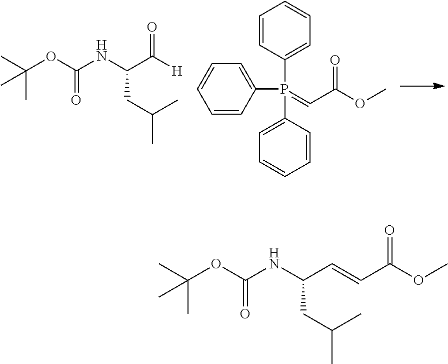

To a stirred solution of methyl(triphenylphosphoranylidene)acetate (3.42 g, 10.2 mmol) in Et₂O (25 mL) at RT was added a solution of Intermediate 32 in Et₂O (15 mL). The reaction mixture was stirred for 15 h at RT. The solid was removed by filtration and the solution was concentrated in vacuo. Purification via flash column chromatography (0-50% EtOAc/hexanes) afforded the title compound (1.74 g, 75% over two steps) as a clear, colorless oil. LC-MS m/z 272 (M+H)⁺, 1.22 min (ret time).

### Intermediate 34

### methyl (2E,4S)-4-amino-6-methyl-2-heptenoate trifluoroacetate

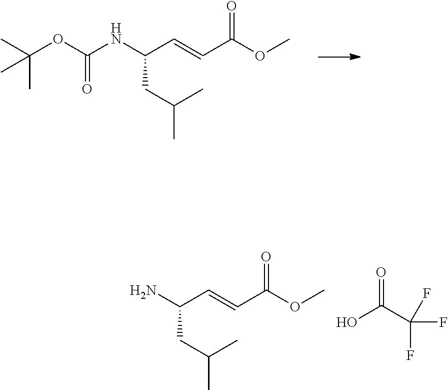

To a solution of methyl (2E,4S)-4-({[(1,1-dimethylethyl)oxy]carbonyl}amino)-6-methyl-2-heptenoate (1.74 g, 6.41 mmol) in CH₂Cl₂ (30 mL) was added TFA (4.94 mL, 64.1 mmol). The reaction mixture was stirred at RT for 2.5 h and then concentrated in vacuo. The resultant yellow oil was diluted with Et₂O (10 mL), hexanes was added with stirring until the mixture became cloudy, and the mixture was concentrated in vacuo to give a yellow solid. The solid was triturated and washed with Et₂O to afford the title compound (1.35 g, 74%) as an off white solid. LC-MS m/z 172 (M+H)⁺, 0.83 min (ret time).

### Intermediate 35

### methyl (2E,4S)-4-[(N-{[(1,1-dimethylethyl)oxy]carbonyl}-L-alanyl)amino]-6-methyl-2-heptenoate

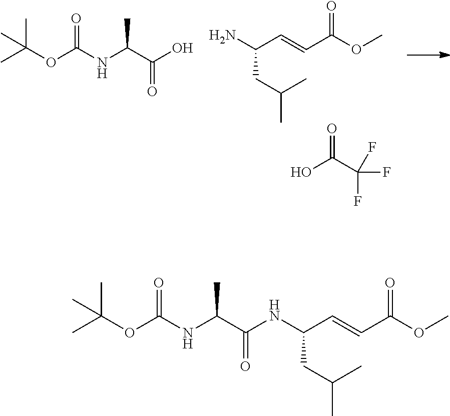

A mixture of methyl (2E,4S)-4-amino-6-methyl-2-heptenoate trifluoroacetate (1.35 g, 4.73 mmol), N-(tert-butoxycarbonyl)-L-alanine (0.94 g, 4.97 mmol), EDCI (1.81 g, 9.47 mmol), HOBt (1.45 g, 9.47 mmol), and NMM (1.56 mL, 14.2 mmol) in DMF (10.0 mL) was stirred for 1 h at RT. Water (100 mL) was added with stirring, followed by extraction with EtOAc (100 mL). The organic layer was washed with water (5×100 mL) and brine (100 mL), dried over Na₂SO₄, filtered, and concentrated in vacuo to afford the title compound (1.67 g, >100%, contained some residual solvent). LC-MS m/z 343 (M+H)⁺, 1.15 min (ret time).

### Intermediate 36

### (2E,4S)-4-[(N-{[(1,1-dimethylethyl)oxy]carbonyl}-L-alanyl)amino]-6-methyl-2-heptenoic

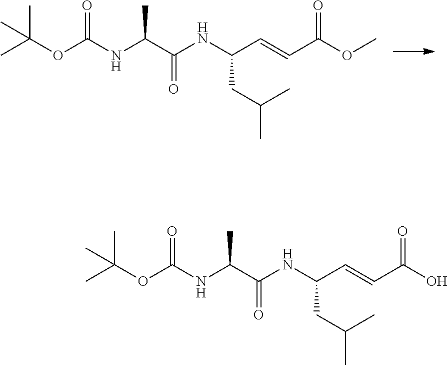

To a solution of methyl (2E,4S)-4-[(N-{[(1,1-dimethylethyl)oxy]carbonyl}-L-alanyl)amino]-6-methyl-2-heptenoate (1.62 g, 4.73 mmol) in THF (25 mL) and water (25 mL) was added LiOH (0.566 g, 23.7 mmol). After stirring for 15 h at RT, the reaction mixture was acidified with 1 M aq. HCl to pH=3 and then extracted with EtOAc (100 mL). The combined organic layers were washed with water (100 mL) and brine (100 mL), dried over Na₂SO₄, filtered, and concentrated in vacuo. The resultant oil was diluted with Et₂O and hexanes and concentrated in vacuo to afford the title compound (1.17 g, 75%) as a white solid. LC-MS m/z 329 (M+H)⁺, 1.03 min (ret time).

### Intermediate 37

### 1,1-dimethylethyl((1S)-1-methyl-2-{[(1S,2E)-4-{[4-(methyloxy)phenyl]amino}-1-(2-methylpropyl)-4-oxo-2-buten-1-yl]amino}-2-oxoethyl)carbamate

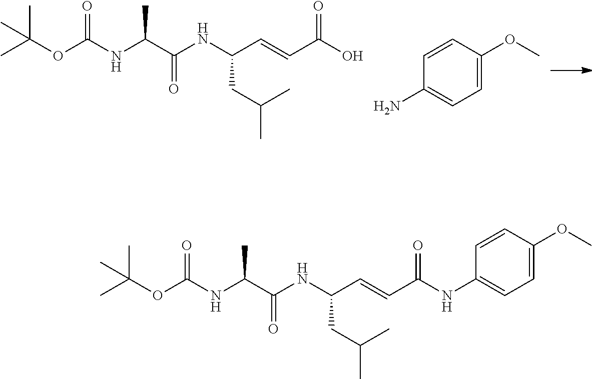

A solution of (2E,4S)-4-[(N-{[(1,1-dimethylethyl)oxy]carbonyl}-L-alanyl)amino]-6-methyl-2-heptenoic acid (100 mg, 0.305 mmol), HATU (116 mg, 0.305 mmol), and DIPEA (0.204 mL, 1.218 mmol) in CH₂Cl₂ (4.0 mL) was stirred at RT for 30 min. 4-(methyloxy)-aniline (37.5 mg, 0.305 mmol) was added and stirring continued overnight. The reaction mixture was partitioned between water (10 mL) and EtOAc (20 mL). The organic layer was washed with water (3×10 mL) and brine (2×10 mL), and concentrated in vacuo to afford the title compound (130 mg, 98%). LC-MS m/z 434 (M+H)⁺, 1.30 min (ret time).

### Intermediate 38

### 1,1-dimethylethyl((1S)-2-{[(1S,2E)-4-(1,3-dihydro-2H-isoindol-2-yl)-1-(2-methylpropyl)-4-oxo-2-buten-1-yl]amino}-1-methyl-2-oxoethyl)carbamate

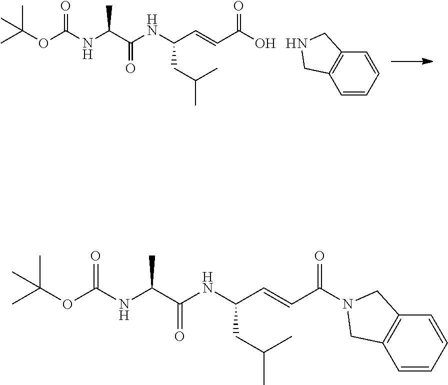

A solution of (2E,4S)-4-[(N-{[(1,1-dimethylethyl)oxy]carbonyl}-L-alanyl)amino]-6-methyl-2-heptenoic acid (100 mg, 0.305 mmol), HATU (116 mg, 0.305 mmol), and DIPEA (0.204 mL, 1.218 mmol) in CH₂Cl₂ (4.0 mL) was stirred at RT for 30 min. 2,3-dihydro-1H-isoindole (0.035 mL, 0.305 mmol) was added and stirring continued overnight. The reaction mixture was partitioned between water (10 mL) and EtOAc (20 mL). The organic layer was washed with water (3×10 mL) and brine (2×10 mL), and concentrated in vacuo to afford the title compound (120 mg, 92%). LC-MS m/z 430 (M+H)⁺, 1.62 min (ret time).

### Intermediate 39

### (2E,4S)-4-({[(1,1-dimethylethyl)oxy]carbonyl}amino)-6-phenyl-2-hexenoic acid

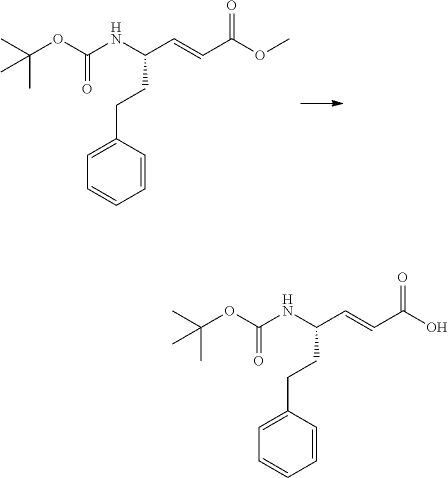

To a solution of methyl (2E,4S)-4-({[(1,1-dimethylethyl)oxy]carbonyl}amino)-6-phenyl-2-hexenoate (1.00 g, 3.13 mmol) in THF (30 mL) and water (3 mL) was added 4 M aq. LiOH (2.35 mL, 9.39 mmol). After stirring for 15 h at RT, additional LiOH (75 mg, 3.1 mmol) in water (1.0 mL) was added. Following an additional 15 h of stirring, the reaction mixture was acidified with 2 M aq. HCl to pH ˜5-6 and then partitioned between water and EtOAc. Following extraction of the aqueous layer with EtOAc, the combined organic layers were washed with water and brine, dried over Na₂SO₄, filtered, and concentrated in vacuo to afford the title compound (1.23 g, >100%, contained some residual solvent) as a yellow oil which partly solidified upon standing. LC-MS m/z 306 (M+H)⁺, 1.03 min (ret time).

### Intermediate 40

### 1,1-dimethylethyl[(1S,2E)-4-{[4-(methyloxy)phenyl]amino}-4-oxo-1-(2-phenylethyl)-2-buten-1-yl]carbamate

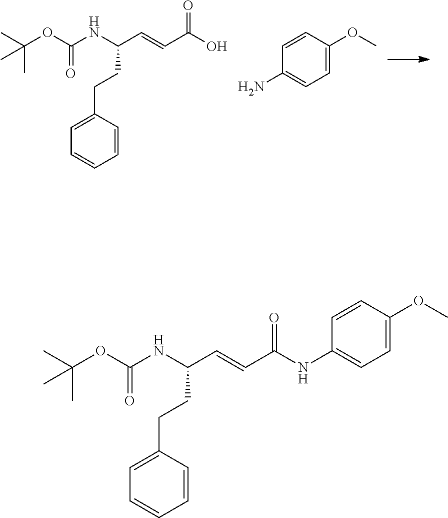

A mixture of (2E,4S)-4-({[(1,1-dimethylethyl)oxy]carbonyl}amino)-6-phenyl-2-hexenoic acid (0.956 g, 3.13 mmol), 4-(methyloxy)aniline (0.386 g, 3.13 mmol), HATU (1.19 g, 3.13 mmol), and DIPEA (1.64 mL, 9.39 mmol) in DMF (20.0 mL) was stirred at RT for 20 min. The reaction mixture was diluted with water (50 mL). A solid that precipitated out from the solution was collected by filtration and washed with water. The solid was dissolved in EtOAc (˜100 mL), dried over Na₂SO₄, filtered, and concentrated in vacuo. The resultant light tan solid was triturated with Et₂O to afford the title compound (0.877 g, 69%). LC-MS m/z 411 (M+H)⁺, 1.15 min (ret time).

### Intermediate 41

### (2E,4S)-4-amino-N-[4-(methyloxy)phenyl]-6-phenyl-2-hexenamide hydrochloride

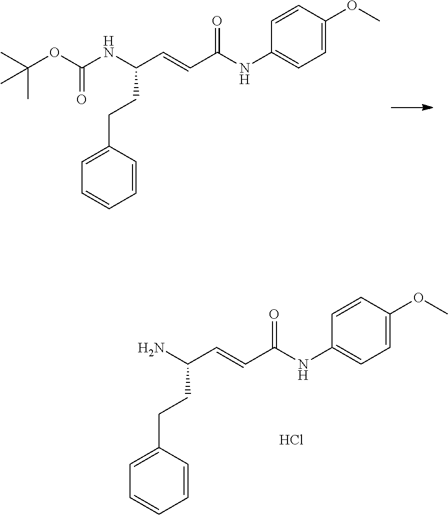

A solution of 1,1-dimethylethyl[(1S,2E)-4-{[4-(methyloxy)phenyl]amino}-4-oxo-1-(2-phenylethyl)-2-buten-1-yl]carbamate (0.877 g, 2.14 mmol) in HCl (4 M solution in 1,4-dioxane, 3.0 mL, 12.0 mmol) was stirred at RT for 10 min. The reaction mixture was concentrated in vacuo. The resultant purple gum was triturated with hexanes (˜5 mL) and Et₂O (˜1-2 mL), and the resultant off white solid was further washed with hexanes to afford the title compound (0.618 g, 83%). LC-MS m/z 311 (M+H)⁺, 0.74 min (ret time).

### Intermediate 42

### 1,1-dimethylethyl[(1S)-2-{[(1S,2E)-4-{[4-(methyloxy)phenyl]amino}-4-oxo-1-(2-phenylethyl)-2-buten-1-yl]amino}-2-oxo-1-(2-thienylmethyl)ethyl]carbamate

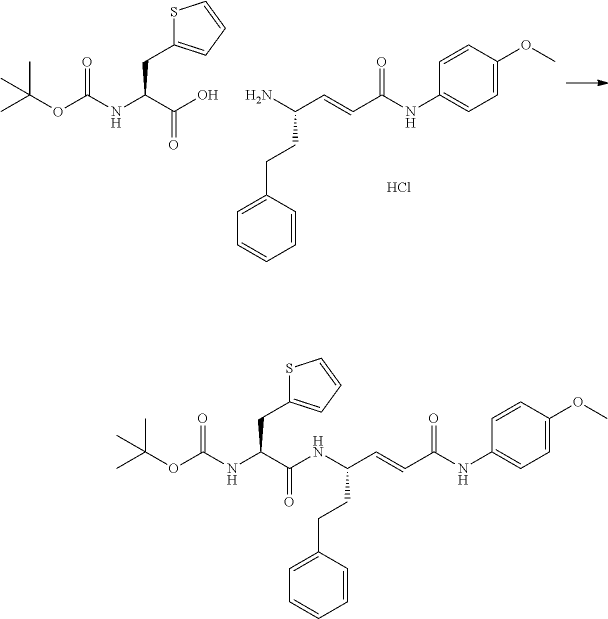

A mixture of (2E,4S)-4-amino-N-[4-(methyloxy)phenyl]-6-phenyl-2-hexenamide hydrochloride (0.309 g, 0.891 mmol), N-{[(1,1-dimethylethyl)oxy]carbonyl}-3-(2-thienyl)-L-alanine (0.242 g, 0.891 mmol), HATU (0.339 g, 0.891 mmol), and DIPEA (0.47 mL, 2.70 mmol) in DMF (8.0 mL) was stirred at RT for 20 min. The reaction mixture was diluted with water (10 mL). A solid that precipitated out from the solution was collected by filtration and washed with water followed by Et₂O. The solid was dissolved in EtOAc (˜100 mL), washed with brine (2×50 mL), dried over Na₂SO₄, filtered, and concentrated in vacuo to afford the title compound (0.411 g, 82%) as an off white solid. LC-MS m/z 564 (M+H)⁺, 1.21 min (ret time).

### Intermediate 43

### 1,1-dimethylethyl[(1S)-1-({[(1S,2E)-4-{[4-(methyloxy)phenyl]amino}-4-oxo-1-(2-phenylethyl)-2-buten-1-yl]amino}carbonyl)propyl]carbamate

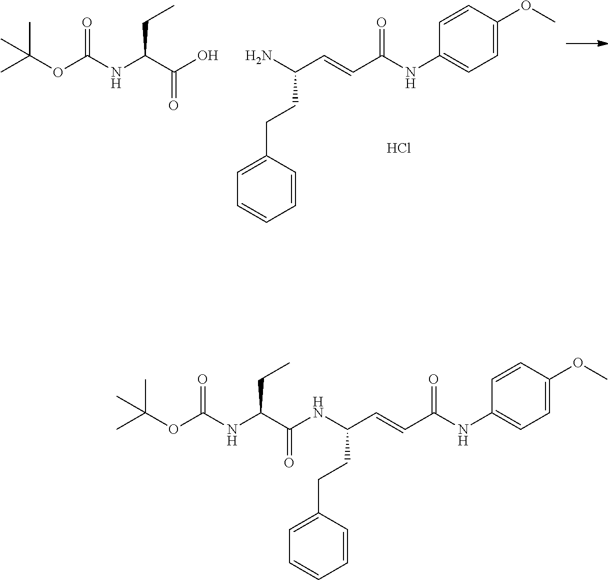

A mixture of (2E,4S)-4-amino-N-[4-(methyloxy)phenyl]-6-phenyl-2-hexenamide hydrochloride (0.309 g, 0.891 mmol), (2S)-2-({[(1,1-dimethylethyl)oxy]carbonyl}amino)-butanoic acid (0.181 g, 0.891 mmol), HATU (0.339 g, 0.891 mmol), and DIPEA (0.47 mL, 2.70 mmol) in DMF (8.0 mL) was stirred at RT for 20 min. The reaction mixture was diluted with water (10 mL). A solid that precipitated out from the solution was collected by filtration and washed with water followed by Et₂O. The solid was dissolved in EtOAc (100 mL), washed with brine (50 mL), dried over Na₂SO₄, filtered, and concentrated in vacuo to afford the title compound (0.426 g, 96%) as an off white solid. LC-MS m/z 496 (M+H)⁺, 1.10 min (ret time).

### Intermediate 44

### methyl (2E,4S)-4-{[N-{[(1,1-dimethylethyl)oxy]carbonyl}-3-(2-thienyl)-L-alanyl]amino}-6-phenyl-2-hexenoate

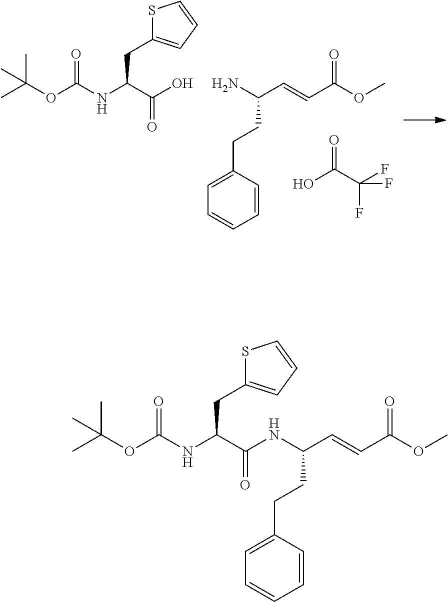

A mixture of methyl (2E,4S)-4-amino-6-phenyl-2-hexenoate trifluoroacetate (2.90 g, 8.70 mmol), N-{[(1,1-dimethylethyl)oxy]carbonyl}-3-(2-thienyl)-L-alanine (2.48 g, 9.14 mmol), EDCI (3.34 g, 17.4 mmol), HOBt (2.66 g, 17.4 mmol), and NMM (2.90 mL, 26 mmol) in DMF (20.0 mL) was stirred at RT for 3 h. The reaction mixture was diluted with water (˜30 mL) and stirred at RT for about 5 min. A solid that precipitated out from the solution was collected by filtration and washed with water. The solid was dissolved in CH₂Cl₂, water was removed by pipette, and the remaining organic solution was dried over Na₂SO₄, filtered, and concentrated in vacuo. Purification via flash column chromatography (0-40% EtOAc/CH₂Cl₂) afforded the title compound (3.81 g, 93%) as a white solid. LC-MS m/z 473 (M+H)⁺, 1.23 min (ret time).

### Intermediate 45

### (2E,4S)-4-{[N-{[(1,1-dimethylethyl)oxy]carbonyl}-3-(2-thienyl)-L-alanyl]amino}-6-phenyl-2-hexenoic acid

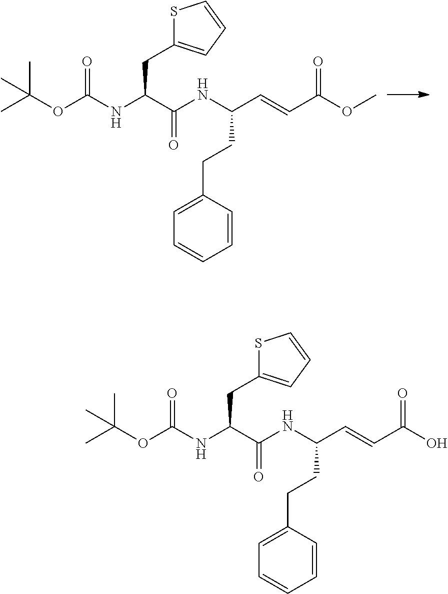

To a solution of methyl (2E,4S)-4-{[N-{[(1,1-dimethylethyl)oxy]carbonyl}-3-(2-thienyl)-L-alanyl]amino}-6-phenyl-2-hexenoate (3.81 g, 8.06 mmol) in THF (50 mL) and water (50 mL) was added LiOH (0.965 g, 40.3 mmol). After stirring for 15 h at RT, the reaction mixture was acidified with 1 M aq. HCl to pH=3 and then extracted with EtOAc (50 mL). The combined organic layers were washed with water (50 mL) and brine (50 mL), dried over Na₂SO₄, filtered, and concentrated in vacuo to afford the title compound (3.41 g, 92%) as an off white solid. LC-MS m/z 459 (M+H)⁺, 1.12 min (ret time).

### Intermediate 46

### 5-(1-methylcyclobutyl)-1,3,4-thiadiazol-2-amine

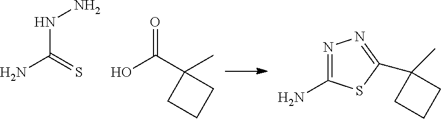

To a solution of 1-methylcyclobutanecarboxylic acid (prepared by the method of Cowling, S. J. and Goodby, J. W. Chem. Commun., 2006, 4107-4109) (6.87 g, 60.2 mmol) in phosphorus oxychloride (2.0 mL, 21.5 mmol) was added thiosemicarbazide (5.49 g, 60.2 mmol). The reaction mixture was heated to 100° C. for 2 h and then allowed to cool to RT. Purification via flash column chromatography (CH₂Cl₂) afforded the title compound (9.4 g, 92%). LC-MS m/z 170 (M+H)⁺, 0.61 min (ret time).

### Intermediate 47

### 1,1-dimethylethyl[(1S)-1-({[(1S,2E)-4-{[5-(1-methylcyclobutyl)-1,3,4-thiadiazol-2-yl]amino}-4-oxo-1-(2-phenylethyl)-2-buten-1-yl]amino}carbonyl)propyl]carbamate

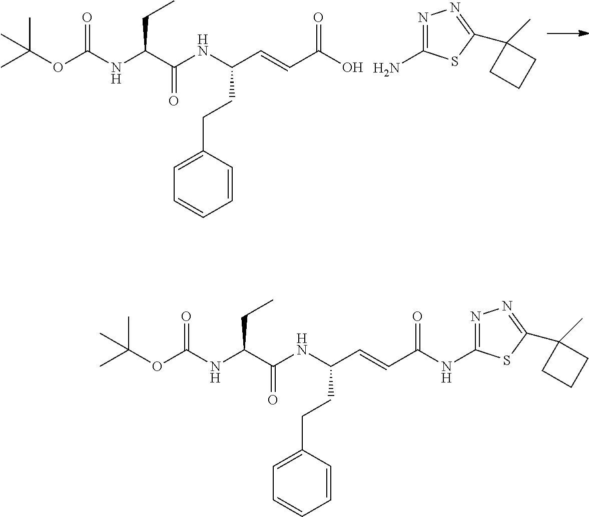

A solution of (2E,4S)-4-{[(2S)-2-({[(1,1-dimethylethyl)oxy]carbonyl}amino)butan-oyl]amino}-6-phenyl-2-hexenoic acid (100 mg, 0.256 mmol), HATU (97 mg, 0.256 mmol), and DIPEA (0.179 mL, 1.024 mmol) in CH₂Cl₂ (5.0 mL) was stirred at RT for 30 min. 541-methylcyclobutyl)-1,3,4-thiadiazol-2-amine (43.3 mg, 0.256 mmol) was added and stirring continued for 10 min. The reaction mixture was partitioned between water (10 mL) and EtOAc (20 mL). The organic layer was washed with water (3×10 mL) and brine (2×10 mL), and concentrated in vacuo to afford the title compound (140 mg, 100%). LC-MS m/z 542 (M+H)⁺, 1.32 min (ret time).

### Intermediate 48

### 1,1-dimethylethyl((1S)-1-cyclopentyl-2-{[(1S,2E)-4-{[4-(methyloxy)phenyl]amino}-4-oxo-1-(2-phenylethyl)-2-buten-1-yl]amino}-2-oxoethyl)carbamate

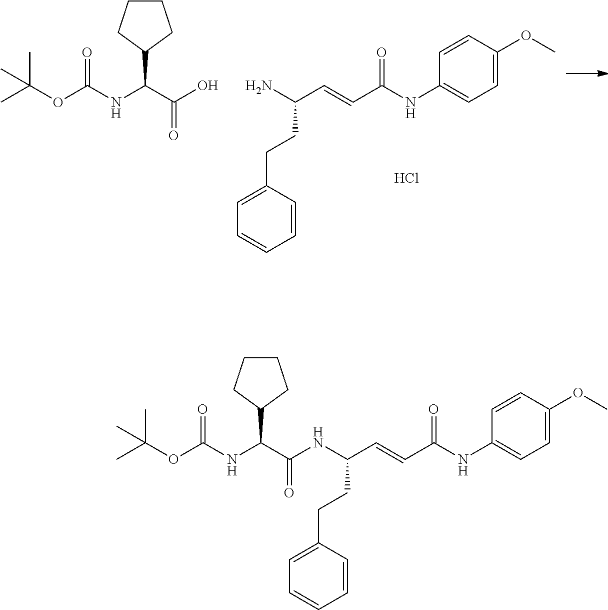

A mixture of (2E,4S)-4-amino-N-[4-(methyloxy)phenyl]-6-phenyl-2-hexenamide hydrochloride (130 mg, 0.306 mmol), (2S)-cyclopentyl({[(1,1-dimethylethyl)oxy]carbonyl}-amino)ethanoic acid (130 mg, 0.306 mmol), BOP reagent (135 mg, 0.306 mmol), and DIPEA (0.160 mL, 0.919 mmol) in DMF (2.0 mL) was stirred at RT for 1 h. The reaction mixture was diluted with water. A solid that precipitated out from the solution was collected by filtration, washed with water, and dried in vacuo to afford the title compound (70 mg, 43%) as a white solid. LC-MS m/z 536 (M+H)⁺, 1.26 min (ret time).

### Intermediate 49

### 1,1-dimethylethyl[(1S)-2-methyl-1-({[(1S,2E)-4-{[4-(methyloxy)phenyl]amino}-4-oxo-1-(2-phenylethyl)-2-buten-1-yl]amino}carbonyl)propyl]carbamate

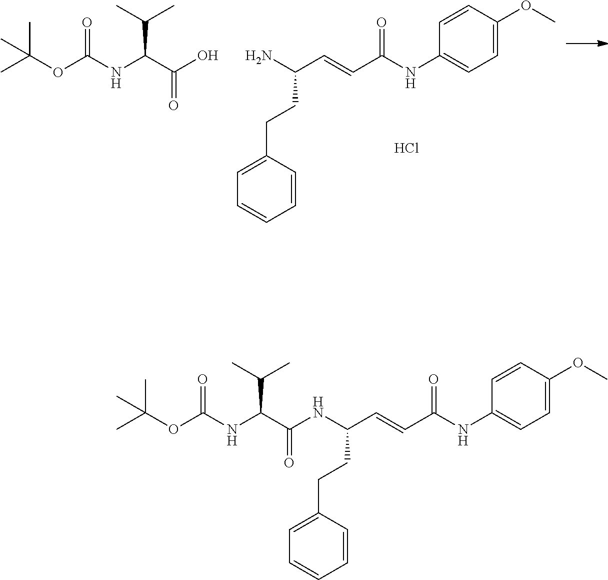

A mixture of (2E,4S)-4-amino-N-[4-(methyloxy)phenyl]-6-phenyl-2-hexenamide hydrochloride (148 mg, 0.349 mmol), N-(tert-butoxycarbonyl)-L-valine (76 mg, 0.349 mmol), BOP reagent (154 mg, 0.349 mmol), and DIPEA (0.183 mL, 1.046 mmol) in DMF (2.0 mL) was stirred at RT for 3 h. The reaction mixture was diluted with water. A solid that precipitated out from the solution was collected by filtration, washed with water, and dried in vacuo to afford the title compound (85 mg, 48%) as an off white solid. LC-MS m/z 510 (M+H)⁺, 1.13 min (ret time).

### Intermediate 50

### 1,1-dimethylethyl (2S)-2-({[(1S,2E)-1-ethyl-4-(methyloxy)-4-oxo-2-buten-1-yl]amino}carbonyl)-1-azetidinecarboxylate

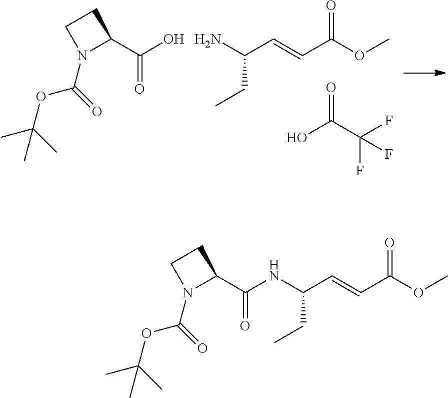

A solution of (2S)-1-{[(1,1-dimethylethyl)oxy]carbonyl}-2-azetidinecarboxylic acid (3.00 g, 14.91 mmol), HATU (5.67 g, 14.91 mmol), and DIPEA (7.81 mL, 44.7 mmol) in CH₂Cl₂ (40.0 mL) and DMF (4.0 mL) was stirred at RT for 30 min. Methyl (2E,4S)-4-amino-2-hexenoate trifluoroacetate (3.84 g, 14.91 mmol) was added and stirring continued overnight. Water was added and the reaction mixture was extracted with EtOAc. The organic layer was washed twice with water and once with brine, dried over MgSO₄, filtered, and concentrated in vacuo to afford the title compound (4.83 g, 99%). LC-MS m/z 327 (M+H)⁺, 0.83 min (ret time).

### Intermediate 51

### (2E,4S)-4-{[((2S)-1-{[(1,1-dimethylethyl)oxy]carbonyl}-2-azetidinyl)carbonyl]amino}-2-hexenoic acid

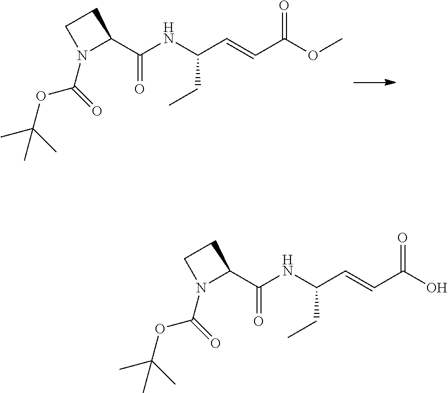

To a solution of 1,1-dimethylethyl (2S)-2-({[(1S,2E)-1-ethyl-4-(methyloxy)-4-oxo-2-buten-1-yl]amino}carbonyl)-1-azetidinecarboxylate (4.83 g, 14.80 mmol) in THF (75 mL) and water (75 mL) was added LiOH (4.83 g, 14.80 mmol). After stirring overnight at RT, the reaction mixture was concentrated in vacuo. The reaction mixture was acidified with 1 M aq. HCl to pH=3 and then extracted with EtOAc. The organic layer was washed twice with water and once with brine, dried over MgSO₄, filtered, and concentrated in vacuo to afford the title compound (4.50 g, 97%). LC-MS m/z 313 (M+H)⁺, 0.78 min (ret time).

### Intermediate 52

### 1,1-dimethylethyl (2S)-2-({[(1S,2E)-4-(2,3-dihydro-1H-indol-1-yl)-1-ethyl-4-oxo-2-buten-1-yl]amino}carbonyl)-1-azetidinecarboxylate

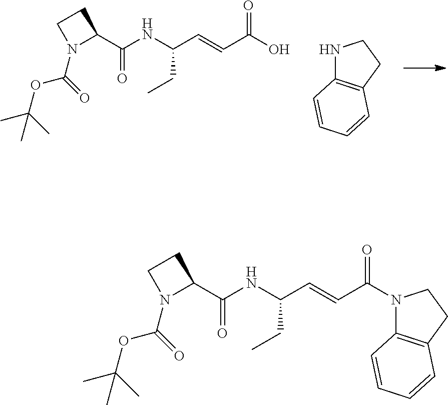

A solution of (2E,4S)-4-{[((2S)-1-{[(1,1-dimethylethyl)oxy]carbonyl}-2-azetidinyl)carbonyl]amino}-2-hexenoic acid (1.47 g, 4.71 mmol), HATU (1.789 g, 4.71 mmol), and DIPEA (2.466 mL, 14.12 mmol) in CH₂Cl₂ (16.0 mL) and DMF (2.0 mL) was stirred at RT for 30 min. 2,3-Dihydro-1H-indole (0.529 mL, 4.71 mmol) was added and stirring continued overnight. The reaction mixture was concentrated in vacuo and purified by flash column chromatography (0-90% EtOAc/hexanes) to afford the title compound (0.900 g, 46%). LC-MS m/z 414 (M+H)⁺, 1.07 min (ret time).

### Intermediate 53

### 1,1-dimethylethyl (2S)-2-({[(1S,2E)-4-(methyloxy)-1-(2-methylpropyl)-4-oxo-2-buten-1-yl]amino}carbonyl)-1-azetidinecarboxylate

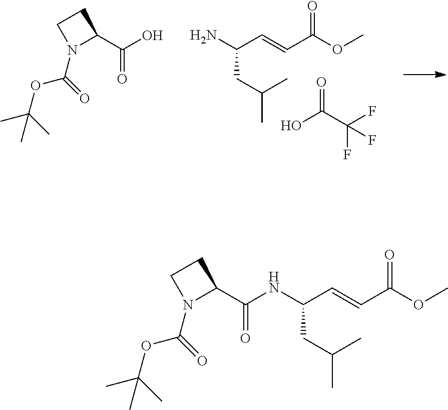

A solution of (2S)-1-{[(1,1-dimethylethyl)oxy]carbonyl}-2-azetidinecarboxylic acid (403 mg, 2.005 mmol), HATU (762 mg, 2.005 mmol), and DIPEA (1.226 mL, 7.02 mmol) in CH₂Cl₂ (20.0 mL) and DMF (5.0 mL) was stirred at RT for 30 min. A solution of methyl (2E,4S)-4-amino-6-methyl-2-heptenoate trifluoroacetate (572 mg, 2.005 mmol) in DMF (5.0 mL) was added and stirring continued for 1 h. Water (100 mL) was added and the reaction mixture was extracted with EtOAc (100 mL). The organic layer was washed with water (5×100 mL) and brine (100 mL), dried over Na₂SO₄, filtered, and concentrated in vacuo to afford the title compound (609 mg, 86%) as a yellow solid. LC-MS m/z 355 (M+H)⁺, 1.06 min (ret time).

### Intermediate 54

### (2E,4S)-4-{[((2S)-1-{[(1,1-dimethylethyl)oxy]carbonyl}-2-azetidinyl)carbonyl]amino}-6-methyl-2-heptenoic acid

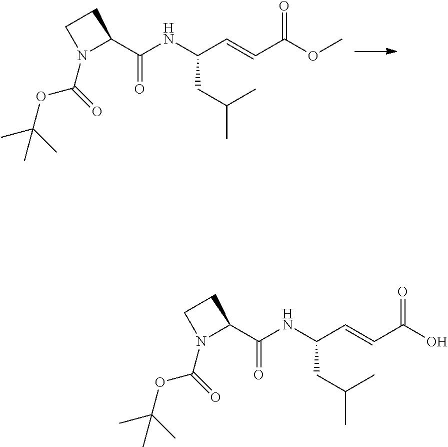

To a solution of 1,1-dimethylethyl (2S)-2-({[(1S,2E)-4-(methyloxy)-1-(2-methylpropyl)-4-oxo-2-buten-1-yl]amino}carbonyl)-1-azetidinecarboxylate (609 mg, 1.718 mmol) in THF (25 mL), water (25 mL), and MeOH (5.0 mL) was added LiOH (206 mg, 8.59 mmol). After stirring for 15 h at RT, the reaction mixture was concentrated in vacuo. Water (10 mL) was added, the reaction mixture was acidified with 1 M aq. HCl to pH=3, and then extracted with EtOAc (100 mL). The organic layer was washed with water (100 mL) and brine (100 mL), dried over MgSO₄, filtered, and concentrated in vacuo. The resultant yellow oil was diluted with Et₂O and hexanes and concentrated in vacuo to afford the title compound (485 mg, 83%) as a white solid. LC-MS m/z 341 (M+H)⁺, 0.92 min (ret time).

### Intermediate 55

### 1,1-dimethylethyl (2S)-2-({[(1S,2E)-4-(2,3-dihydro-1H-indol-1-yl)-1-(2-methylpropyl)-4-oxo-2-buten-1-yl]amino}carbonyl)-1-azetidinecarboxylate

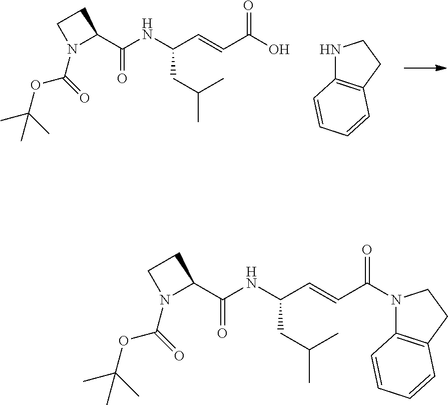

A solution of (2E,4S)-4-{[((2S)-1-{[(1,1-dimethylethyl)oxy]carbonyl}-2-azetidinyl)carbonyl]amino}-6-methyl-2-heptenoic acid (100 mg, 0.294 mmol), HATU (112 mg, 0.294 mmol), and DIPEA (0.154 mL, 0.881 mmol) in CH₂Cl₂ (4.0 mL) and DMF (1.0 mL) was stirred at RT for 30 min. 2,3-Dihydro-1H-indole (0.033 mL, 0.294 mmol) was added and stirring continued overnight. The reaction mixture was partitioned between water (10 mL) and EtOAc (20 mL). The organic layer was washed with water (3×10 mL) and brine (2×10 mL), and concentrated in vacuo to afford the title compound (174 mg, >100%). LC-MS m/z 442 (M+H)⁺, 1.16 min (ret time).

### Intermediate 56

### 3-cyclopropyl-N²-{[(1,1-dimethylethyl)oxy]carbonyl}-N¹-methyl-N¹-(methyloxy)-L-alaninamide

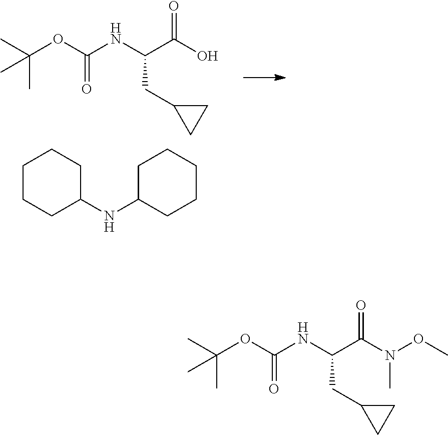

To a solution of 3-cyclopropyl-N-(tert-butoxycarbonyl)-L-alanine N,N-dicyclohexylamine (5.00 g, 12.18 mmol) in THF (17.0 mL) and DMF (3.0 mL) was added 1,1′-carbonyldiimidazole (2.369 g, 14.61 mmol) portionwise over about 10 min. After stirring 30 min at RT, a solution of N,O-dimethylhydroxylamine hydrochloride (1.307 g, 13.40 mmol) and DIPEA (2.340 mL, 13.40 mmol) in DMF (4.0 mL) was added. The reaction mixture was stirred for 3 h at RT, diluted with EtOAc, and washed twice with 1 M aq. HCl and twice with saturated aq. NaHCO₃. The organic layer was dried over Na₂SO₄, filtered, and concentrated in vacuo to afford the title compound (2.65 g, 80%). LC-MS m/z 273 (M+H)⁺, 0.98 min (ret time).

### Intermediate 57

### 1,1-dimethylethyl[(1S)-2-cyclopropyl-1-formylethyl]carbamate

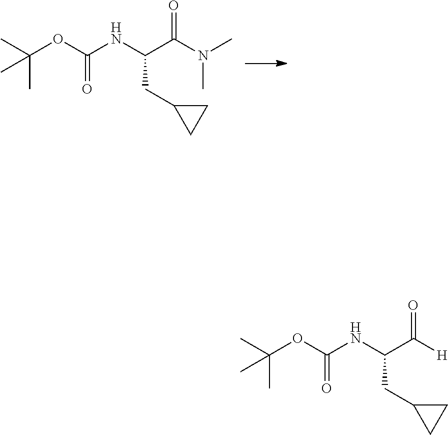

To a solution of LiAlH₄ (0.406 g, 10.70 mmol) in Et₂O (20 mL) at 0° C. was added dropwise a solution of 3-cyclopropyl-N²-{[(1,1-dimethylethyl)oxy]carbonyl}-N¹-methyl-N¹-(methyloxy)-L-alaninamide (2.65 g, 9.73 mmol) in Et₂O (15 mL). The reaction mixture was stirred for 30 min at 0° C. and quenched with EtOAc (5 mL) followed by 5% aq. potassium bisulfate (6 mL). The reaction mixture was washed with 1 M aq. HCl (2×40 mL), saturated aq. NaHCO₃ (2×40 mL), and brine (40 mL). The organic layer was dried over Na₂SO₄, filtered, and concentrated in vacuo to afford the title compound as a clear, colorless oil, which was carried to the next step without further purification.

### Intermediate 58

### methyl (2E,4S)-5-cyclopropyl-4-({[(1,1-dimethylethyl)oxy]carbonyl}amino)-2-pentenoate

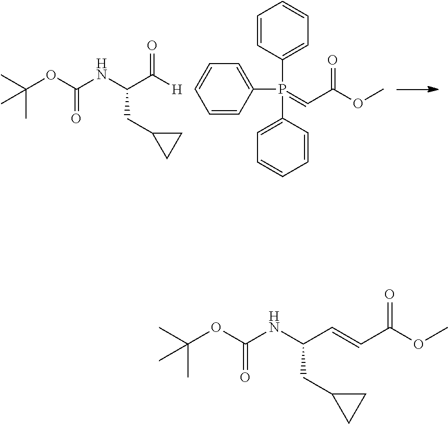

To a solution of methyl(triphenylphosphoranylidene)acetate (3.90 g, 11.68 mmol) in Et₂O (30 mL) at RT was added a solution of Intermediate 57 in Et₂O (20 mL). The reaction mixture was stirred for 15 h at RT. The solid was removed by filtration and the solution was concentrated in vacuo. Purification via flash column chromatography (0-50% EtOAc/hexanes) afforded the title compound (1.59 g, 61% over two steps) as a clear, colorless oil. LC-MS m/z 270 (M+H)⁺, 1.10 min (ret time).

### Intermediate 59

### methyl (2E,4S)-4-amino-5-cyclopropyl-2-pentenoate trifluoroacetate

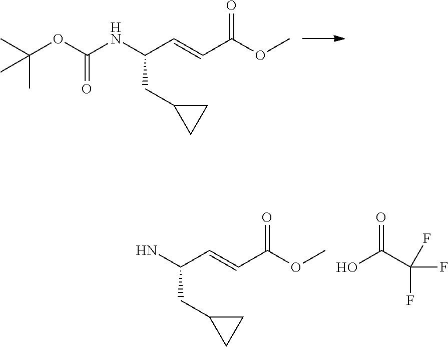

To a solution of methyl (2E,4S)-5-cyclopropyl-4-({[(1,1-dimethylethyl)oxy]-carbonyl}amino)-2-pentenoate (0.90 g, 3.34 mmol) in CH₂Cl₂ (15 mL) was added TFA (4.12 mL, 53.5 mmol). The reaction mixture was stirred at RT for 2.5 h and then concentrated in vacuo. The resultant yellow oil was diluted with Et₂O, concentrated in vacuo, washed with Et₂O and filtered to afford the title compound (635 mg, 67%) as an off white solid. LC-MS m/z 170 (M+H)⁺, 0.50 min (ret time).

### Intermediate 60

### 1,1-dimethylethyl (2S)-2-({[(1S,2E)-1-(cyclopropylmethyl)-4-(methyloxy)-4-oxo-2-buten-1-yl]amino}carbonyl)-1-azetidinecarboxylate

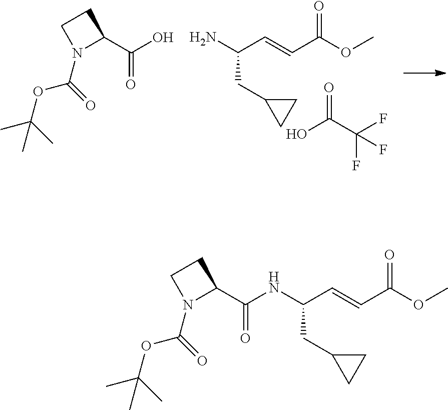

A solution of (2S)-1-{[(1,1-dimethylethyl)oxy]carbonyl}-2-azetidinecarboxylic acid (496 mg, 2.466 mmol), HATU (852 mg, 2.242 mmol), and DIPEA (1.370 mL, 7.85 mmol) in CH₂Cl₂ (20.0 mL) and DMF (5.0 mL) was stirred at RT for 30 min. A solution of methyl (2E,4S)-4-amino-5-cyclopropyl-2-pentenoate trifluoroacetate (635 mg, 2.242 mmol) in DMF (5.0 mL) was added and stirring continued for 1 h. Water (100 mL) was added and the reaction mixture was extracted with EtOAc (100 mL). The organic layer was washed with water (5×100 mL) and brine (100 mL), dried over Na₂SO₄, filtered, and concentrated in vacuo to afford the title compound (450 mg, 57%) as a yellow solid. LC-MS m/z 353 (M+H)⁺, 0.99 min (ret time).

### Intermediate 61

### (2E,4S)-5-cyclopropyl-4-{[((2S)-1-{[(1,1-dimethylethyl)oxy]carbonyl}-2-azetidinyl)carbonyl]amino}-2-pentenoic acid

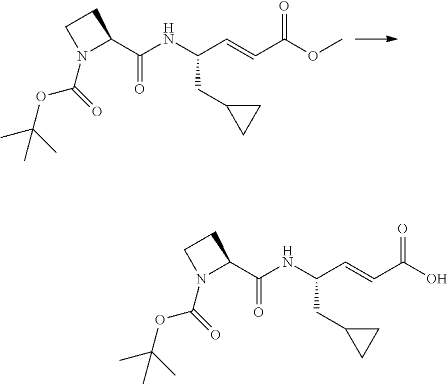

To a solution of 1,1-dimethylethyl (2S)-2-({[(1S,2E)-1-(cyclopropylmethyl)-4-(methyloxy)-4-oxo-2-buten-1-yl]amino}carbonyl)-1-azetidinecarboxylate (450 mg, 1.277 mmol) in THF (25 mL), water (25 mL), and MeOH (5.0 mL) was added LiOH (153 mg, 6.38 mmol). After stirring for 15 h at RT, the reaction mixture was concentrated in vacuo. Water (10 mL) was added, the reaction mixture was acidified with 1 M aq. HCl to pH=3, and then extracted with EtOAc (100 mL). The organic layer was washed with water (100 mL) and brine (100 mL), dried over MgSO₄, filtered, and concentrated in vacuo to afford the title compound (341 mg, 79%) as a white solid. LC-MS m/z 339 (M+H)⁺, 0.83 min (ret time).

### Intermediate 62

### 1,1-dimethylethyl (2S)-2-({[(1S,2E)-1-(cyclopropylmethyl)-4-(2,3-dihydro-1H-indol-1-yl)-4-oxo-2-buten-1-yl]amino}carbonyl)-1-azetidinecarboxylate

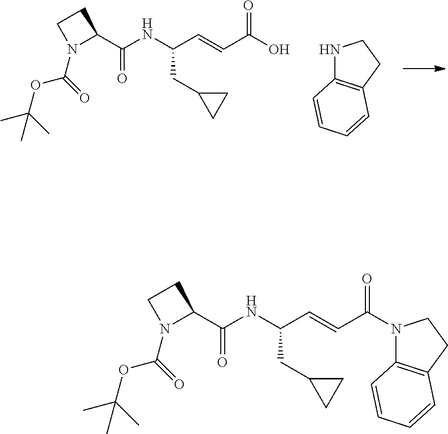

A solution of (2E,4S)-5-cyclopropyl-4-{[((2S)-1-{[(1,1-dimethylethyl)oxy]carbonyl}-2-azetidinyl)carbonyl]amino}-2-pentenoic acid (110 mg, 0.325 mmol), HATU (124 mg, 0.325 mmol), and DIPEA (0.170 mL, 0.975 mmol) in CH₂Cl₂ (4.0 mL) and DMF (1.0 mL) was stirred at RT for 30 min. 2,3-Dihydro-1H-indole (0.037 mL, 0.325 mmol) was added and stirring continued overnight. The reaction mixture was partitioned between water (10 mL) and EtOAc (20 mL). The organic layer was washed with water (3×10 mL) and brine (2×10 mL), and concentrated in vacuo to afford the title compound (181 mg, >100%). LC-MS m/z 440 (M+H)⁺, 1.14 min (ret time).

### Intermediate 63

### 1,1-dimethylethyl (2S,4S)-2-({[(1S,2E)-1-ethyl-4-(methyloxy)-4-oxo-2-buten-1-yl]amino}carbonyl)-4-fluoro-1-pyrrolidinecarboxylate

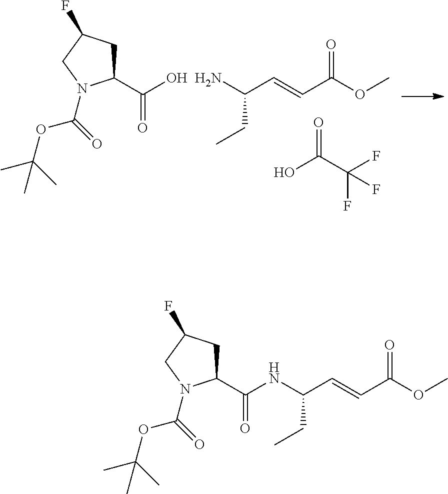

A solution of (4S)-1-{[(1,1-dimethylethyl)oxy]carbonyl}-4-fluoro-L-proline (499 mg, 2.140 mmol), HATU (814 mg, 2.140 mmol), and DIPEA (1.121 mL, 6.42 mmol) in CH₂Cl₂ (8.0 mL) and DMF (2.0 mL) was stirred at RT for 30 min. Methyl (2E,4S)-4-amino-2-hexenoate trifluoroacetate (550 mg, 2.14 mmol) was added and stirring continued for 2 h. Water was added and the reaction mixture was extracted with EtOAc. The organic layer was washed twice with water and once with brine, dried over MgSO₄, filtered, and concentrated in vacuo to afford the title compound (750 mg, 98%). LC-MS m/z 359 (M+H)⁺, 0.83 min (ret time).

### Intermediate 64

### (2E,4S)-4-[((4S)-1-{[(1,1-dimethylethyl)oxy]carbonyl}-4-fluoro-L-prolyl)amino]-2-hexenoic acid

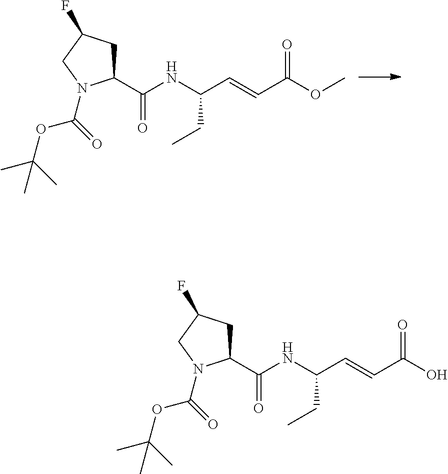

To a solution of 1,1-dimethylethyl (2S,4S)-2-({[(1S,2E)-1-ethyl-4-(methyloxy)-4-oxo-2-buten-1-yl]amino}carbonyl)-4-fluoro-1-pyrrolidinecarboxylate (750 mg, 2.093 mmol) in THF (25 mL) and water (25 mL) was added LiOH (251 mg, 10.46 mmol). After stirring overnight at RT, the reaction mixture was concentrated in vacuo. The reaction mixture was acidified with 1 M aq. HCl to pH=3 and then extracted with EtOAc. The organic layer was washed twice with water and once with brine, dried over MgSO₄, filtered, and concentrated in vacuo to afford the title compound (710 mg, 99%). LC-MS m/z 345 (M+H)⁺, 0.70 min (ret time).

### Intermediate 65

### 1,1-dimethylethyl (2S,4S)-2-({[(1S,2E)-4-(2,3-dihydro-1H-indol-1-yl)-1-ethyl-4-oxo-2-buten-1-yl]amino}carbonyl)-4-fluoro-1-pyrrolidinecarboxylate

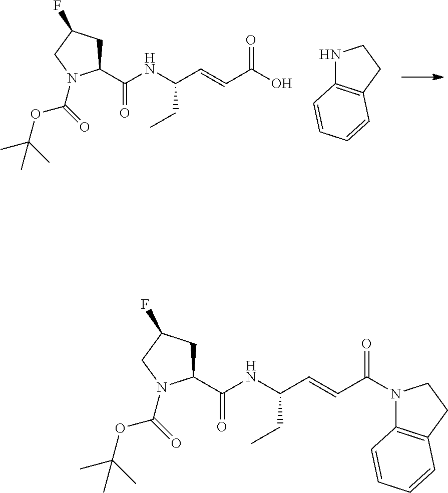

A solution of (2E,4S)-4-[((4S)-1-{[(1,1-dimethylethyl)oxy]carbonyl}-4-fluoro-L-prolyl)amino]-2-hexenoic acid (200 mg, 0.581 mmol), HATU (221 mg, 0.581 mmol), and DIPEA (0.304 mL, 1.742 mmol) in CH₂Cl₂ (8.0 mL) and DMF (2.0 mL) was stirred at RT for 30 min. 2,3-Dihydro-1H-indole (0.065 mL, 0.581 mmol) was added and stirring continued overnight. The reaction mixture was concentrated in vacuo and purified by reverse phase HPLC (YMC C18 S-85 μm/12 nm 75×30 mm preparatory column), eluting with a linear gradient running from 10% CH₃CN/H₂O (0.1% TFA) to 80% CH₃CN/H₂O (0.1% TFA) over 15 min. to afford the title compound (60 mg, 23%). LC-MS m/z 446 (M+H)⁺, 1.01 min (ret time).

### Intermediate 66

### 1,1-dimethylethyl (2S)-2-({[(1S,2E)-1-ethyl-4-(methyloxy)-4-oxo-2-buten-1-yl]amino}carbonyl)-1-piperidinecarboxylate

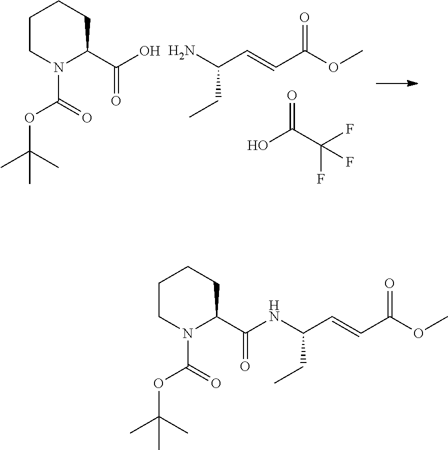

A solution of (2S)-1-{[(1,1-dimethylethyl)oxy]carbonyl}-2-piperidinecarboxylic acid (1.845 g, 8.05 mmol), HATU (3.06 g, 8.05 mmol), and DIPEA (4.22 mL, 24.14 mmol) in CH₂Cl₂ (8.0 mL) and DMF (2.0 mL) was stirred at RT for 30 min. Methyl (2E,4S)-4-amino-2-hexenoate trifluoroacetate (2.07 g, 8.05 mmol) was added and stirring continued overnight. The reaction mixture was diluted with CH₂Cl₂ and washed twice with water and once with brine. The organic layer was concentrated in vacuo, diluted with EtOAc, washed twice with water and once with brine, dried over MgSO₄, filtered, and concentrated in vacuo to afford the title compound (3.05 g, >100%). LC-MS m/z 355 (M+H)⁺, 1.06 min (ret time).

### Intermediate 67

### (2E,4S)-4-{[((2S)-1-{[(1,1-dimethylethyl)oxy]carbonyl}-2-piperidinyl)carbonyl]amino}-2-hexenoic acid

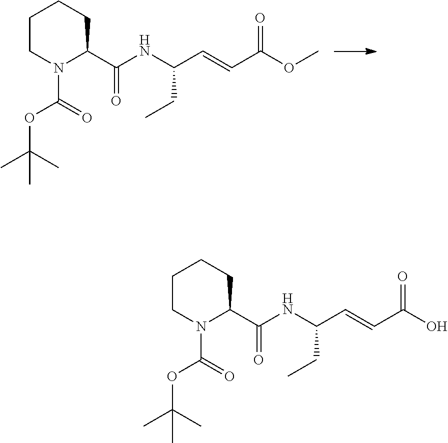

To a solution of 1,1-dimethylethyl (2S)-2-({[(1S,2E)-1-ethyl-4-(methyloxy)-4-oxo-2-buten-1-yl]amino}carbonyl)-1-piperidinecarboxylate (3.05 g, 8.61 mmol) in THF (25 mL), MeOH (5.0 mL), and water (25 mL) was added LiOH (1.031 g, 43.1 mmol). After stirring overnight at RT, the reaction mixture was concentrated in vacuo. The reaction mixture was acidified with 1 M aq. HCl to pH=3 and then extracted with EtOAc. The organic layer was washed twice with water and once with brine, dried over MgSO₄, filtered, and concentrated in vacuo to afford the title compound (3.00 g, >100%). LC-MS m/z 341 (M+H)⁺, 0.90 min (ret time).

### Intermediate 68

### 1,1-dimethylethyl (2S)-2-({[(1S,2E)-4-(2,3-dihydro-1H-indol-1-yl)-1-ethyl-4-oxo-2-buten-1-yl]amino}carbonyl)-1-piperidinecarboxylate

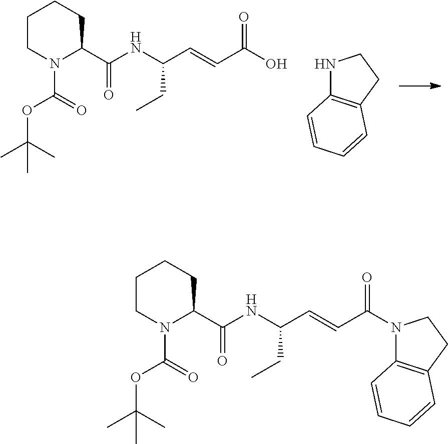

A solution of (2E,4S)-4-{[((2S)-1-{[(1,1-dimethylethyl)oxy]carbonyl}-2-piperidinyl)carbonyl]amino}-2-hexenoic acid (300 mg, 0.881 mmol), HATU (335 mg, 0.881 mmol), and DIPEA (0.462 mL, 2.64 mmol) in CH₂Cl₂ (8.0 mL) and DMF (2.0 mL) was stirred at RT for 30 min. 2,3-Dihydro-1H-indole (0.099 mL, 0.881 mmol) was added and stirring continued overnight. The reaction mixture was diluted with CH₂Cl₂ and washed twice with water and once with brine. The organic layer was concentrated in vacuo, diluted with EtOAc, washed twice with water and once with brine, dried over MgSO₄, filtered, concentrated in vacuo, and purified by reverse phase HPLC(YMC C18 S-15 μm/12 nm 75×30 mm preparatory column), eluting with a linear gradient running from 10% CH₃CN/H₂O (0.1% TFA) to 80% CH₃CN/H₂O (0.1% TFA) over 15 min. to afford the title compound (200 mg, 51%). LC-MS m/z 442 (M+H)⁺, 1.15 min (ret time).

### Intermediate 69

### 1,1-dimethylethyl (2S)-2-({[(1S,2E)-4-(methyloxy)-1-(2-methylpropyl)-4-oxo-2-buten-1-yl]amino}carbonyl)-1-piperidinecarboxylate

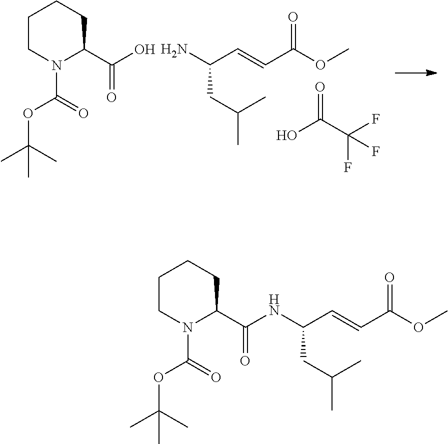

A solution of (2S)-1-{[(1,1-dimethylethyl)oxy]carbonyl}-2-piperidinecarboxylic acid (1.268 g, 5.53 mmol), HATU (2.103 g, 5.53 mmol), and DIPEA (2.90 mL, 16.59 mmol) in CH₂Cl₂ (10.0 mL) and DMF (2.0 mL) was stirred at RT for 30 min. A solution of methyl (2E,4S)-4-amino-6-methyl-2-heptenoate trifluoroacetate (0.947 g, 5.53 mmol) in CH₂Cl₂ (6.0 mL) was added and stirring continued overnight. The reaction mixture was diluted with EtOAc and washed twice with water and once with brine. The organic layer was dried over MgSO₄, filtered, and concentrated in vacuo to afford the title compound (2.11 g, 100%). LC-MS m/z 383 (M+H)⁺, 1.13 min (ret time).

### Intermediate 70

### (2E,4S)-4-{[((2S)-1-{[(1,1-dimethylethyl)oxy]carbonyl}-2-piperidinyl)carbonyl]amino}-6-methyl-2-heptenoic acid

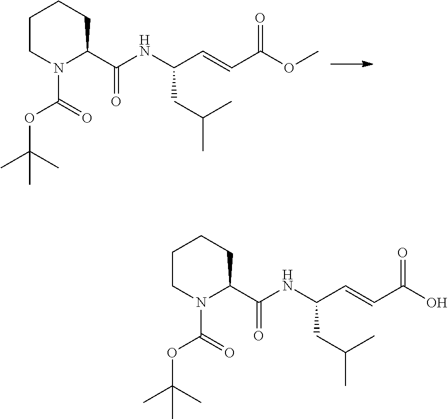

To a solution of 1,1-dimethylethyl (2S)-2-({[(1S,2E)-4-(methyloxy)-1-(2-methylpropyl)-4-oxo-2-buten-1-yl]amino}carbonyl)-1-piperidinecarboxylate (2.11 g, 5.52 mmol) in THF (25 mL), MeOH (5.0 mL), and water (25 mL) was added LiOH (0.661 g, 27.6 mmol). After stirring overnight at RT, the reaction mixture was concentrated in vacuo. The reaction mixture was acidified with 1 M aq. HCl to pH=3 and then extracted with EtOAc. The organic layer was washed twice with water and once with brine, dried over MgSO₄, filtered, and concentrated in vacuo to afford the title compound (2.01 g, 99%). LC-MS m/z 369 (M+H)⁺, 1.05 min (ret time).

### Intermediate 71

### 1,1-dimethylethyl (2S)-2-({[(1S,2E)-4-(2,3-dihydro-1H-indol-1-yl)-1-(2-methylpropyl)-4-oxo-2-buten-1-yl]amino}carbonyl)-1-piperidinecarboxylate

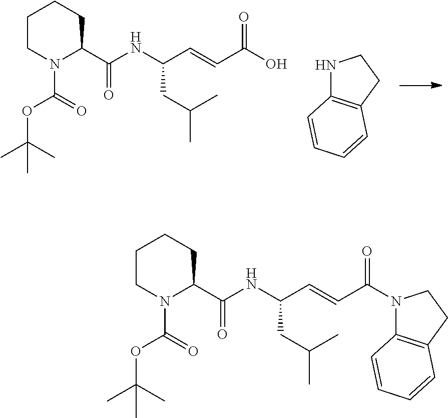

A solution of (2E,4S)-4-{[((2S)-1-{[(1,1-dimethylethyl)oxy]carbonyl}-2-piperidinyl)carbonyl]amino}-6-methyl-2-heptenoic acid (200 mg, 0.543 mmol), HATU (206 mg, 0.543 mmol), and DIPEA (0.284 mL, 1.628 mmol) in CH₂Cl₂ (8.0 mL) and DMF (2.0 mL) was stirred at RT for 30 min. 2,3-Dihydro-1H-indole (0.061 mL, 0.543 mmol) was added and stirring continued overnight. The reaction mixture was diluted with EtOAc and washed twice with water and once with brine. The organic layer was dried over MgSO₄, filtered, concentrated in vacuo, and purified by reverse phase HPLC(YMC C18 S-15 μm/12 nm 75×30 mm preparatory column), eluting with a linear gradient running from 10% CH₃CN/H₂O (0.1% TFA) to 80% CH₃CN/H₂O (0.1% TFA) over 15 min. to afford the title compound (143 mg, 56%). LC-MS m/z 470 (M+H)⁺, 1.28 min (ret time).

### Intermediate 72

### (2E,4S)-4-({[(1,1-dimethylethyl)oxy]carbonyl}amino)-6-methyl-2-heptenoic acid

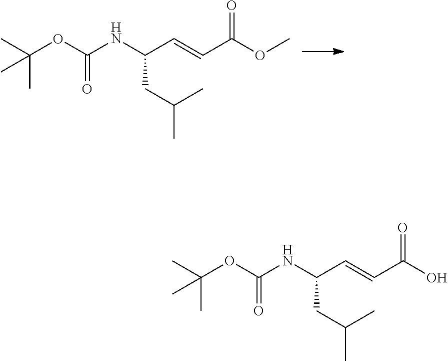

To a solution of methyl (2E,4S)-4-({[(1,1-dimethylethyl)oxy]carbonyl}amino)-6-methyl-2-heptenoate (5.00 g, 18.43 mmol) in THF (15 mL), MeOH (15.0 mL), and water (15 mL) was added LiOH (2.206 g, 92.00 mmol). After stirring for 2 h at RT, the reaction mixture was concentrated in vacuo. The reaction mixture was acidified with 6 M aq. HCl to pH=5 and then extracted with EtOAc. The organic layer was washed with water, dried over Na₂SO₄, filtered, and concentrated in vacuo to afford the title compound (4.7 g, 99%) as a white semi-solid. LC-MS m/z 158 (M+H-Boc)', 0.94 min (ret time).

### Intermediate 73

### 1,1-dimethylethyl[(1S,2E)-4-(2,3-dihydro-1H-indol-1-yl)-1-(2-methylpropyl)-4-oxo-2-buten-1-yl]carbamate

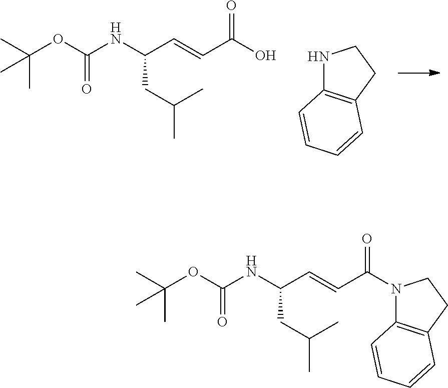

To a solution of (2E,4S)-4-({[(1,1-dimethylethyl)oxy]carbonyl}amino)-6-methyl-2-heptenoic acid (4.70 g, 18.26 mmol) in DMF (30.0 mL) were added BOP reagent (8.08 g, 18.26 mmol) and DIPEA (6.38 mL, 36.5 mmol). After stirring at RT for 5 min, 2,3-dihydro-1H-indole (2.053 mL, 18.26 mmol) was added and stirring continued overnight. The reaction mixture was diluted with water and extracted with EtOAc. The organic layer was washed with brine, dried over Na₂SO₄, filtered, concentrated in vacuo and purified by flash column chromatography (0-20% EtOAc/hexanes) to afford the title compound (4.83 g, 74%) as a white solid. LC-MS m/z 359 (M+H)⁺, 1.18 min (ret time).

### Intermediate 74

### [(1S,2E)-4-(2,3-dihydro-1H-indol-1-yl)-1-(2-methylpropyl)-4-oxo-2-buten-1-yl]amine

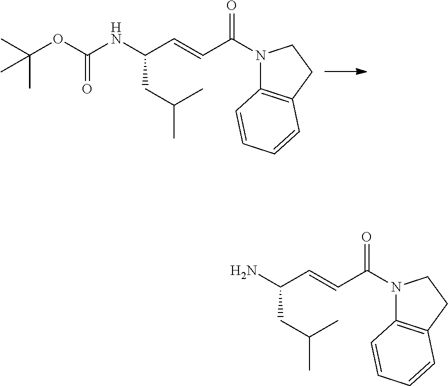

To a solution of 1,1-dimethylethyl[(1S,2E)-4-(2,3-dihydro-1H-indol-1-yl)-1-(2-methylpropyl)-4-oxo-2-buten-1-yl]carbamate (4.82 g, 13.45 mmol) in CH₂Cl₂ (30.0 mL) was added TFA (10.36 mL, 134.5 mmol). The reaction mixture was stirred for 2 h at RT and then basified with 6 M aq. NaOH. Following separation of the layers, the organic layer was washed with water, dried over Na₂SO₄, filtered, and concentrated in vacuo to afford the title compound (3.30 g, 95%). LC-MS m/z 259 (M+H)⁺, 0.77 min (ret time).

### Intermediate 75

### 1,1-dimethylethyl (2S)-2-({[(1S,2E)-4-(2,3-dihydro-1H-indol-1-yl)-1-(2-methylpropyl)-4-oxo-2-buten-1-yl]amino}carbonyl)-1-piperidinecarboxylate

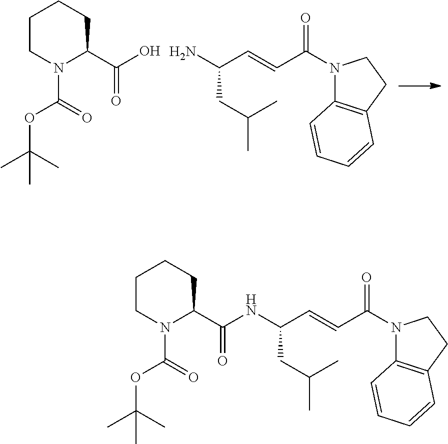

To a solution of (2S)-1-{[(1,1-dimethylethyl)oxy]carbonyl}-2-piperidinecarboxylic acid (1.775 g, 7.74 mmol) in DMF (20.0 mL) were added BOP reagent (3.42 g, 7.74 mmol) and DIPEA (2.70 mL, 15.48 mmol). After stirring at RT for 5 min, [(1S,2E)-4-(2,3-dihydro-1H-indol-1-yl)-1-(2-methylpropyl)-4-oxo-2-buten-1-yl]amine (2.00 g, 7.74 mmol) was added and stirring continued for 1 h. The reaction mixture was diluted with water and extracted with EtOAc. The organic layer was washed with brine, dried over Na₂SO₄, filtered, concentrated in vacuo and purified by flash column chromatography (0-30% EtOAc/hexanes) to afford the title compound (3.08 g, 85%) as a white solid. LC-MS m/z 470 (M+H)⁺, 1.26 min (ret time).

Alternatively, the title compound could be prepared by the following procedure:

To a solution of (2S)-1-{[(1,1-dimethylethyl)oxy]carbonyl}-2-piperidinecarboxylic acid (3.28 g, 14.32 mmol) and HATU (5.45 g, 14.32 mmol) in DMF (15 mL) was added NMM (3.15 mL, 28.6 mmol). After stirring at RT under nitrogen for 30 min, a solution of [(1S,2E)-4-(2,3-dihydro-1H-indol-1-yl)-1-(2-methylpropyl)-4-oxo-2-buten-1-yl]amine (3.70 g, 14.32 mmol) in CH₂Cl₂ (15.0 mL) was added and the reaction mixture was left to stand at RT under nitrogen for 18 h. The reaction mixture was diluted with CH₂Cl₂ (100 mL) and washed with water (2×100 mL) and brine (100 mL). The organic phase was passed through a hydrophobic frit and concentrated in vacuo to ˜30 mL. Purification via flash column chromatography (0-50% EtOAc/cyclohexane) afforded the title compound (5.68 g, 84%) as a white foam. LC-MS m/z 470 (M+H)⁺, 1.26 min (ret time). ¹H NMR (400 MHz, DMSO-d₆) δ ppm 8.12 (br. s, 1H), 8.02-7.87 (m, 1H), 7.23 (d, J=7.3 Hz, 1H), 7.15 (t, J=7.5 Hz, 1H), 7.00 (t, J=7.5 Hz, 1H), 6.78 (dd, J=5.3, 15 Hz, 1H), 6.35 (d, J=15 Hz, 1H), 4.66-4.49 (m, 2H), 4.13 (m, 2H), 3.79 (d, J=12.8 Hz, 1H), 3.21-3.05 (m, 3H), 2.07 (d, J=13 Hz, 1H), 1.72-1.53 (m, 4H), 1.52-1.13 (m, 13H), 0.93-0.87 (m, 6H).

### Intermediate 76

### 1,1-dimethylethyl (2S)-2-{[((1S,2E)-1-(2-methylpropyl)-4-oxo-4-{[5-(trifluoromethyl)-1,3,4-thiadiazol-2-yl]amino}-2-buten-1-yl)amino]carbonyl}-1-azetidinecarboxylate

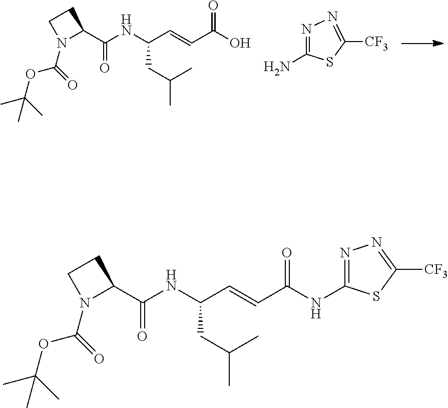

A solution of (2E,4S)-4-{[((2S)-1-{[(1,1-dimethylethyl)oxy]carbonyl}-2-azetidinyl)carbonyl]amino}-6-methyl-2-heptenoic acid (400 mg, 1.175 mmol), HATU (447 mg, 1.175 mmol), and DIPEA (0.616 mL, 3.53 mmol) in CH₂Cl₂ (4.0 mL) and DMF (1.0 mL) was stirred at RT for 30 min. 5-(Trifluoromethyl)-1,3,4-thiadiazol-2-amine (219 mg, 1.293 mmol) was added and stirring continued for 1 h. The reaction mixture was partitioned between water (10 mL) and EtOAc (20 mL). The organic layer was washed with water (3×10 mL) and brine (2×10 mL), and concentrated in vacuo to afford the title compound (656 mg, >100%). LC-MS m/z 492 (M+H)⁺, 1.17 min (ret time).

### Intermediate 77

### 1,1-dimethylethyl (2S)-2-{[((1S,2E)-1-(2-methylpropyl)-4-{methyl[5-(trifluoromethyl)-1,3,4-thiadiazol-2-yl]amino}-4-oxo-2-buten-1-yl)amino]carbonyl}-1-azetidinecarboxylate

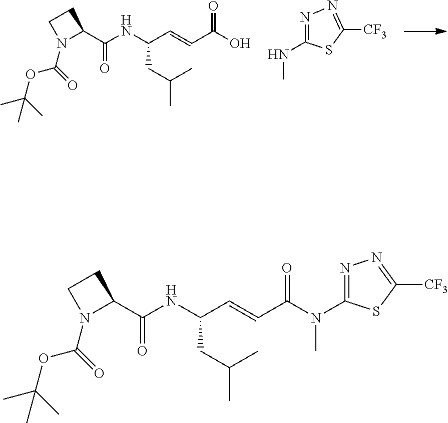

A solution of (2E,4S)-4-{[((2S)-1-{[(1,1-dimethylethyl)oxy]carbonyl}-2-azetidinyl)carbonyl]amino}-6-methyl-2-heptenoic acid (600 mg, 1.763 mmol), HATU (737 mg, 1.939 mmol), and DIPEA (1.539 mL, 8.81 mmol) in CH₂Cl₂ (4.0 mL) and DMF (1.0 mL) was stirred at RT for 30 min. N-methyl-5-(trifluoromethyl)-1,3,4-thiadiazol-2-amine (323 mg, 1.763 mmol) was added and stirring continued overnight. The reaction mixture was partitioned between water (10 mL) and EtOAc (20 mL). The organic layer was washed with water (3×10 mL) and brine (2×10 mL), and concentrated in vacuo to afford the title compound (516 mg, 58%). LC-MS m/z 506 (M+H)⁺, 1.22 min (ret time).

### Intermediate 78

### 1,1-dimethylethyl (2S,4S)-2-{[((1S,2E)-1-ethyl-4-oxo-4-{[5-(trifluoromethyl)-1,3,4-thiadiazol-2-yl]amino}-2-buten-1-yl)amino]carbonyl}-4-fluoro-1-pyrrolidinecarboxylate

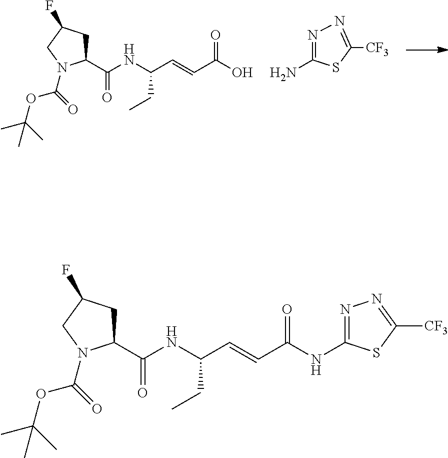

A solution of (2E,4S)-4-[((4S)-1-{[(1,1-dimethylethyl)oxy]carbonyl}-4-fluoro-L-prolyl)amino]-2-hexenoic acid (513 mg, 1.490 mmol), HATU (566 mg, 1.490 mmol), and DIPEA (0.781 mL, 4.47 mmol) in CH₂Cl₂ (4.0 mL) and DMF (1.0 mL) was stirred at RT for 30 min. 5-(Trifluoromethyl)-1,3,4-thiadiazol-2-amine (252 mg, 1.490 mmol) was added and stirring continued overnight. The reaction mixture was concentrated in vacuo and purified by reverse phase HPLC(YMC C18 S-15 μm/12 nm 75×30 mm preparatory column), eluting with a linear gradient running from 10% CH₃CN/H₂O (0.1% TFA) to 80% CH₃CN/H₂O (0.1% TFA) over 15 min. to afford the title compound (460 mg, 62%). LC-MS m/z 496 (M+H)⁺, 1.08 min (ret time).

### Intermediate 79

### 1,1-dimethylethyl (2S)-2-{[((1S,2E)-1-(2-methylpropyl)-4-oxo-4-{[5-(trifluoromethyl)-1,3,4-thiadiazol-2-yl]amino}-2-buten-1-yl)amino]carbonyl}-1-piperidinecarboxylate

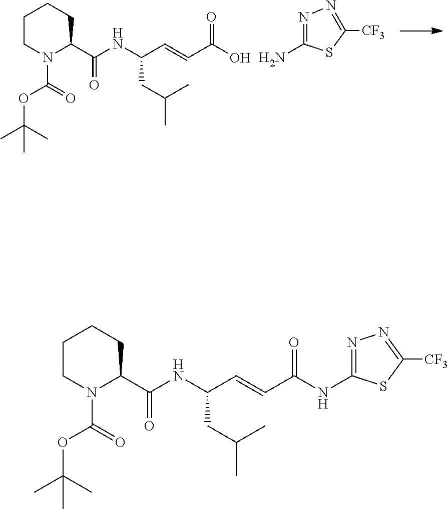

A solution of (2E,4S)-4-{[((2S)-1-{[(1,1-dimethylethyl)oxy]carbonyl}-2-piperidinyl)carbonyl]amino}-6-methyl-2-heptenoic acid (200 mg, 0.543 mmol), HATU (206 mg, 0.543 mmol), and DIPEA (0.284 mL, 1.628 mmol) in CH₂Cl₂ (8.0 mL) and DMF (2.0 mL) was stirred at RT for 30 min. 5-(Trifluoromethyl)-1,3,4-thiadiazol-2-amine (92 mg, 0.543 mmol) was added and stirring continued overnight. The reaction mixture was diluted with EtOAc, washed twice with water and once with brine, dried over MgSO₄, filtered, concentrated in vacuo, and purified by reverse phase HPLC(YMC C18 S-15 μm/12 nm 75×30 mm preparatory column), eluting with a linear gradient running from 10% CH₃CN/H₂O (0.1% TFA) to 80% CH₃CN/H₂O (0.1% TFA) over 15 min. to afford the title compound (60 mg, 21%). LC-MS m/z 520 (M+H)⁺, 1.31 min (ret time).

### Intermediate 80

### 1-(chloroacetyl)-2,3-dihydro-1H-indole

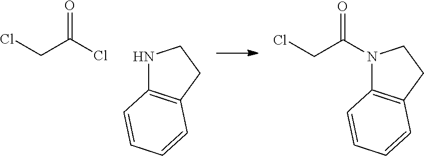

To a solution of 2,3-dihydro-1H-indole (37.6 mL, 336 mmol) in acetone (300 mL) at 0° C. was added chloroacetyl chloride (40.3 mL, 504 mmol) dropwise over 30 min. The reaction mixture was stirred at RT for 1 h and then re-cooled to 0° C. Water (300 mL) was added and the reaction mixture was extracted with EtOAc (2×500 mL). The combined organic phases were washed with saturated aq. NaHCO₃ (400 mL) and brine (200 mL), dried over MgSO₄, and concentrated in vacuo to afford the title compound (50.12 g, 76%) as a brown solid. LC-MS m/z 196/198 (M+H)⁺, 0.84 min (ret time). ¹H NMR (400 MHz, CDCl₃) δ ppm 8.23 (d, J=8 Hz, 1H), 7.27-7.20 (m, 2H), 7.08 (t, J=7.5 Hz, 1H), 4.18 (t, J=8.3 Hz, 2H), 4.17 (s, 2H), 3.26 (t, J=8.3 Hz, 2H).

### Intermediate 81

### [2-(2,3-dihydro-1H-indol-1-yl)-2-oxoethyl](triphenyl)phosphonium chloride

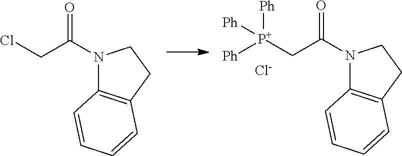

To a solution of 1-(chloroacetyl)-2,3-dihydro-1H-indole (50.12 g, 256 mmol) in toluene (500 mL) was added triphenylphosphine (67.2 g, 256 mmol). The reaction mixture was heated at reflux under nitrogen with rapid stirring for 24 hours. The reaction mixture was allowed to cool to RT. The solid was collected by filtration and washed with toluene (200 ml). The solid was dried in vacuo to afford the title compound (110 g, 94%) as a tan solid. LC-MS m/z 422 (M)⁺, 0.92 min (ret time). ¹H NMR (400 MHz, CDCl₃) δ ppm 8.03-7.95 (m, 6H), 7.88 (d, J=8 Hz, 1H), 7.77-7.71 (m, 3H), 7.68-7.62 (m, 6H), 7.18 (d, J=7.3 Hz, 1H), 7.09 (t, J=7.3 Hz, 1H), 7.02 (t, J=7.3 Hz, 1H), 5.96 (d, J=12.8 Hz, 2H), 4.78 (t, J=8.3 Hz, 2H), 3.27 (t, J=8.3 Hz, 2H).

### Intermediate 82

### 1-[(triphenyl-λ⁵-phosphanylidene)acetyl]-2,3-dihydro-1H-indole

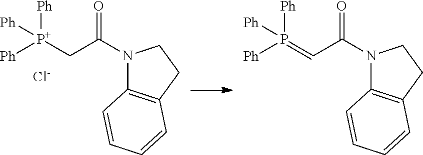

A suspension of [2-(2,3-dihydro-1H-indol-1-yl)-2-oxoethyl](triphenyl)phosphonium chloride (110 g, 240 mmol) in toluene (500 mL) was treated with 2.0 M aq. NaOH (500 mL, 1000 mmol) and the reaction mixture was stirred at RT for 30 min. CH₂Cl₂ (200 mL) was added and the reaction mixture was stirred at RT for 3 h. The two phases were separated and the aqueous phase was extracted with CH₂Cl₂ (100 mL). The combined organic phases were concentrated in vacuo to provide a tan foam which was treated with Et₂O (300 mL). The resultant solid was collected by filtration, washed with Et₂O, and dried to afford the title compound (102.7 g, 101%) as an off-white solid. LC-MS m/z 422 (M+H)⁺, 0.91 min (ret time). ¹H NMR (400 MHz, CDCl₃) δ ppm 8.03 (d, J=8 Hz, 1H), 7.77-7.70 (m, 6H), 7.57-7.52 (m, 3H), 7.49-7.43 (m, 6H), 7.08 (d, J=7.3 Hz, 1H), 7.05 (t, J=7.5 Hz, 1H), 6.76 (t, J=8 Hz, 1H), 4.06 (t, J=8.5 Hz, 2H), 3.12 (t, J=8.5 Hz, 2H), 2.94 (br. d, J=15.8 Hz, 1H).

### Intermediate 83

### 1,1-dimethylethyl[(1S)-3-methyl-1-(4-morpholinylcarbonyl)butyl]carbamate

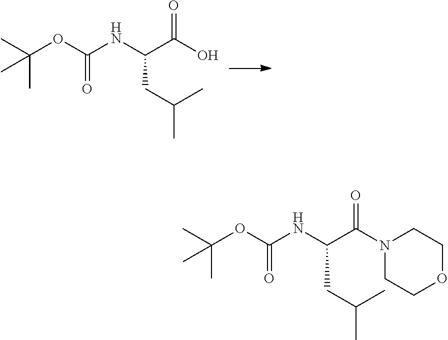

To a solution of N-(tert-butoxycarbonyl)-L-leucine (25 g, 108 mmol) in CH₂Cl₂ (200 mL) at 0° C. were added morpholine (10.36 mL, 119 mmol) and NMM (13.07 mL, 119 mmol). 1,1′-Carbonyldiimidazole (22.79 g, 119 mmol) was added to the reaction mixture portionwise over 15 min. After stirring 20 h at RT, the reaction mixture was washed successively with 1 M aq. HCl (2×250 mL), water (250 mL), saturated aq. NaHCO₃ (2×250 mL), and brine (250 mL). The organic phase was dried over MgSO₄ and concentrated in vacuo to afford the title compound (23.39 g, 72%) as a pale yellow gum. ¹H NMR (400 MHz, CDCl₃) δ ppm 5.24 (d, J=8.8 Hz, 1H), 4.64 (m, 1H), 3.76-3.45 (m, 8H), 1.80-1.65 (m, 1H), 1.54-1.35 (m, 11H), 0.98 (d, J=6.5 Hz, 3H), 0.93 (d, J=6.8 Hz, 3H).

### Intermediate 84

### 1,1-dimethylethyl[(1S,2E)-4-(2,3-dihydro-1H-indol-1-yl)-1-(2-methylpropyl)-4-oxo-2-buten-1-yl]carbamate

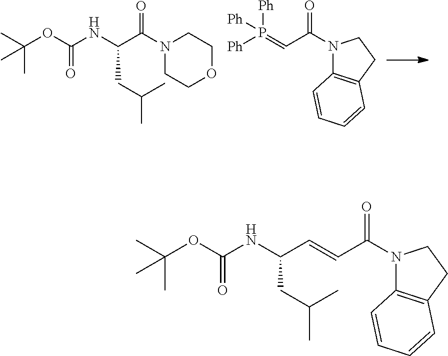

To a solution of 1,1-dimethylethyl[(1S)-3-methyl-1-(4-morpholinylcarbonyl)butyl]carbamate (9.36 g, 31.2 mmol) in 2-methyltetrahydrofuran (100 mL) at 0° C. was added dropwise LiAlH₄ (1 M solution in Et₂O, 37.4 mL, 37.4 mmol), maintaining the temperature below 5° C. The reaction mixture was stirred at 0° C. for 50 min. The reaction was quenched with 5% aq. potassium hydrogen sulphate (50 mL) maintaining the temperature below 10° C. The phases were separated and the aqueous phase was extracted with 2-methyltetrahydrofuran (2×100 mL). The combined organic phases were washed with brine, dried over MgSO₄, and filtered. To this solution was added 1-[(triphenyl-λ⁵-phosphanylidene)acetyl]-2,3-dihydro-1H-indole (13.13 g, 31.2 mmol) and the reaction mixture was stirred for 20 h at RT. The solvent was removed in vacuo and the residue was dissolved in CH₂Cl₂ (50 mL). Purification via flash column chromatography (0-40% EtOAc/cyclohexane) afforded the title compound (5.16 g, 46%) as a white solid. LC-MS m/z 359 (M+H)⁺, 1.23 min (ret time). ¹H NMR (400 MHz, DMSO-d₆) δ ppm 8.12 (br. s, 1H), 7.24 (d, J=7 Hz, 1H), 7.15 (t, J=7.8 Hz, 1H), 7.06 (d, J=8.3 Hz, 1H), 7.00 (t, J=7.3 Hz, 1H), 6.70 (dd, J=6.3, 15 Hz, 1H), 6.39 (d, J=15 Hz, 1H), 4.26-4.09 (m, 3H), 3.15 (t, J=7.8 Hz, 2H), 1.61 (m, 1H), 1.46-1.27 (m, 11H), 0.90 (d, J=3 Hz, 3H), 0.88 (d, J=2.8 Hz, 3H).

### Intermediate 85

### 1,1-dimethylethyl[(1S,2E)-4-(2,3-dihydro-1H-indol-1-yl)-1-(2-methylpropyl)-4-oxo-2-buten-1-yl]carbamate

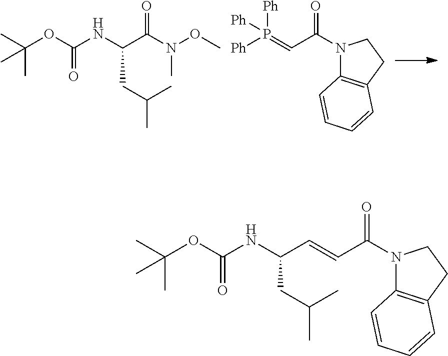

To a solution of N²-{[(1,1-dimethylethyl)oxy]carbonyl}-N¹-methyl-N¹-(methyloxy)-L-leucinamide (7.92 g, 28.9 mmol) in 2-methyltetrahydrofuran (150 mL) at −5° C. was added dropwise LiAlH₄ (1 M solution in Et₂O, 36.1 mL, 36.1 mmol), maintaining the temperature below 0° C. The reaction mixture was stirred at −5° C. for 20 min. The reaction was quenched with a solution of potassium hydrogen sulphate (6.88 g, 50.5 mmol) in water (150 mL) maintaining the temperature below 10° C. The phases were separated and the aqueous phase was extracted with 2-methyltetrahydrofuran (150 mL). The combined organic phases were washed successively with 2 M aq. HCl (2×100 mL), saturated aq. NaHCO₃ (2×100 mL), and brine (100 mL), dried over MgSO₄, and filtered. To this solution was added 1-[(triphenyl-λ⁵-phosphanylidene)acetyl]-2,3-dihydro-1H-indole (12.17 g, 28.9 mmol) and the reaction mixture was stirred under nitrogen for 20 h at RT. The solvent was removed in vacuo and the residue was dissolved in CH₂Cl₂ (20 mL). Purification via flash column chromatography (0-50% EtOAc/cyclohexane) afforded the title compound (8.45 g, 82%) as a white solid. LC-MS m/z 359 (M+H)⁺, 1.23 min (ret time). ¹H NMR (400 MHz, DMSO-d₆) δ ppm 8.12 (br. s, 1H), 7.24 (d, J=7 Hz, 1H), 7.15 (t, J=7.8 Hz, 1H), 7.06 (d, J=8.3 Hz, 1H), 7.00 (t, J=7.3 Hz, 1H), 6.70 (dd, J=6.3, 15 Hz, 1H), 6.39 (d, J=15 Hz, 1H), 4.26-4.09 (m, 3H), 3.15 (t, J=7.8 Hz, 2H), 1.61 (m, 1H), 1.46-1.27 (m, 11H), 0.90 (d, J=3 Hz, 3H), 0.88 (d, J=2.8 Hz, 3H).

### Intermediate 86

### 1,1-dimethylethyl {(1S)-1-(cyclobutylmethyl)-2-[methyl(methyloxy)amino]-2-oxoethyl}carbamate

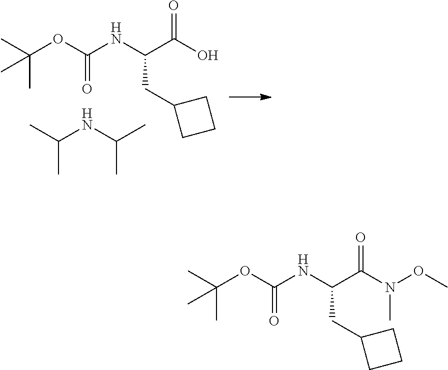

To a solution of 3-cyclobutyl-N-{[(1,1-dimethylethyl)oxy]carbonyl}-L-alanine N,N-diisopropylamine (1:1) (5.00 g, 12.18 mmol) in THF (20 mL) was added 1,1′-carbonyldiimidazole (2.82 g, 17.4 mmol) portionwise over about 10 min. After stirring 30 min at RT, a solution of N,O-dimethylhydroxylamine hydrochloride (1.56 g, 16.0 mmol) and DIPEA (2.79 mL, 16.0 mmol) in DMF (4.0 mL) was added. The reaction mixture was stirred for 20 h at RT, then additional DMF (6.0 mL) and DIPEA (5.0 mL) were added. After stirring at RT for a further 7 h, additional N,O-dimethylhydroxylamine hydrochloride (0.5 g, 5.1 mmol) was added and the mixture was stirred at RT for another 15 h. The reaction mixture was then diluted with EtOAc (100 mL) and washed with 1 M aq. HCl (2×50 mL) followed by saturated aq. NaHCO₃ (2×50 mL). The organic layer was dried over Na₂SO₄, filtered, and concentrated in vacuo to afford the title compound (3.51 g, 84%) as a clear, colorless oil. LC-MS m/z 287 (M+H)⁺, 1.05 min (ret time).

### Intermediate 87

### 1,1-dimethylethyl[(1S)-2-cyclobutyl-1-formylethyl]carbamate

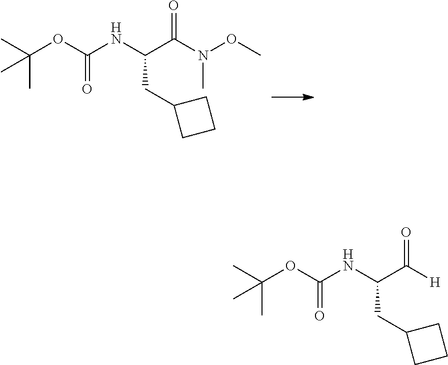

To a solution of LiAlH₄ (0.512 g, 13.48 mmol) in Et₂O (30 mL) at 0° C. was added dropwise a solution of 1,1-dimethylethyl {(15)-1-(cyclobutylmethyl)-2-[methyl(methyloxy)amino]-2-oxoethyl}carbamate (3.51 g, 12.26 mmol) in Et₂O (20 mL) so that the internal temperature did not exceed 5° C. The reaction mixture was stirred for 30 min at 0° C. and quenched by the dropwise addition of EtOAc (10 mL) followed by 5% aq. potassium bisulfate (10 mL), maintaining the internal temperature≦5° C. The reaction mixture was washed with 1 M aq. HCl (2×40 mL), saturated aq. NaHCO₃ (2×40 mL), and brine (40 mL). The organic layer was dried over Na₂SO₄, filtered, and concentrated in vacuo to afford the title compound as a clear, colorless oil, which was carried to the next step without further purification. LC-MS m/z 228 (M+H)⁺, 0.97 min (ret time).

### Intermediate 88

### methyl (2E,4S)-5-cyclobutyl-4-({[(1,1-dimethylethyl)oxy]carbonyl}amino)-2-pentenoate

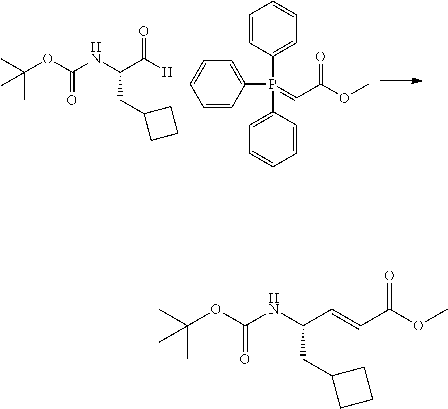

To a stirred solution of methyl(triphenylphosphoranylidene)acetate (4.92 g, 14.7 mmol) in Et₂O (40 mL) at RT was added a solution of Intermediate 87 in Et₂O (20 mL). The mixture was stirred for 15 h at RT. The solid was removed by filtration and the solution was concentrated in vacuo. Purification via flash column chromatography (0-50% EtOAc/hexanes) afforded the title compound (2.5 g, 72% over two steps) as a clear, colorless oil. LC-MS m/z 284 (M+H)⁺, 1.15 min (ret time).

### Intermediate 89

### 2-({[(1,1-dimethylethyl)oxy]carbonyl}amino)-4,4,4-trifluorobutanoic acid

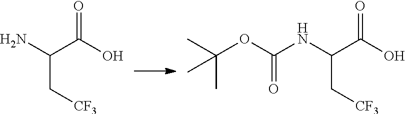

To a solution of 2-amino-4,4,4-trifluorobutanoic acid (3.0 g, 19.1 mmol) in 1 M aq. NaOH (28.6 mL, 28.6 mmol), t-butanol (35 mL), and water (40 ml) was added di-tert-butyl dicarbonate (6.65 mL, 28.6 mmol) in one portion. After stirring at RT for 15 h, the mixture was diluted with water (20 mL) and hexanes (120 mL). The aq. layer was separated, cooled to 0° C., and acidified to pH 2-3 with 1 M aq. potassium bisulfate with vigorous stirring. The mixture was extracted with EtOAc, dried over Na₂SO₄, filtered, and concentrated in vacuo to afford the title compound (5.92 g, 121%, wet with residual solvent) as a yellow oil which partly solidified to form an off white solid upon standing. LC-MS m/z 258 (M+H)⁺, 0.85 min (ret time).

### Intermediate 90

### 1,1-dimethylethyl (3,3,3-trifluoro-1-{[methyl(methyloxy)amino]carbonyl}propyl)carbamate

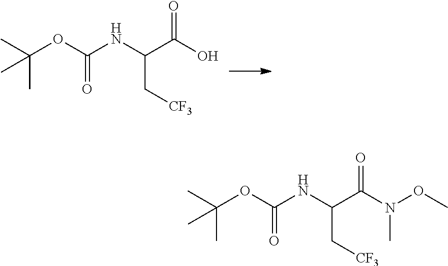

To a solution of 2-({[(1,1-dimethylethyl)oxy]carbonyl}amino)-4,4,4-trifluorobutanoic acid (5.92 g, 23.0 mmol) in THF (35 mL) was added 1,1′-carbonyldiimidazole (3.67 g, 22.7 mmol) portionwise over about 10 min. After stirring for 1 h at RT, a solution of N,O-dimethylhydroxylamine hydrochloride (2.03 g, 20.8 mmol) and DIPEA (3.63 mL, 20.8 mmol) in DMF (8.0 mL) was added. The reaction mixture was stirred at RT for 15 h, followed by concentration in vacuo. The residue was diluted with EtOAc (100 mL) and washed with 1 M aq. HCl (2×50 mL), saturated aq. NaHCO₃ (2×50 mL), and brine (50 mL). The organic layer was dried over Na₂SO₄, filtered, and concentrated in vacuo to give the title compound (5.33 g, 93% over two steps) as an off-white solid. LC-MS m/z 301 (M+H)⁺, 0.97 min (ret time).

### Intermediate 91

### 1,1-dimethylethyl (3,3,3-trifluoro-1-formylpropyl)carbamate

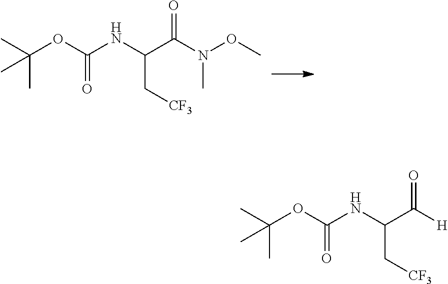

To a solution of LiAlH₄ (0.741 g, 19.5 mmol) in Et₂O (40 mL) at 0° C. was added dropwise a solution of 1,1-dimethylethyl (3,3,3-trifluoro-1-{[methyl(methyloxy)amino]carbonyl}propyl)carbamate (5.33 g, 17.8 mmol) in Et₂O (30 mL) so that the internal temperature did not exceed 5° C. The reaction mixture was stirred for 30 min at 0° C. and quenched by the dropwise addition of EtOAc (10 mL) followed by 5% aq. potassium bisulfate (10 mL), maintaining the internal temperature ≦5° C. The reaction mixture was then washed with 1 M aq. HCl (2×40 mL), saturated aq. NaHCO₃ (2×40 mL), and brine (40 mL). The organic layer was dried over Na₂SO₄, filtered, and concentrated in vacuo to afford the title compound as a clear, colorless oil, which was carried to the next step without further purification. LC-MS m/z 242 (M+H)⁺, 0.74 min (ret time).

### Intermediate 92

### methyl (2E)-4-({[(1,1-dimethylethyl)oxy]carbonyl}amino)-6,6,6-trifluoro-2-hexenoate

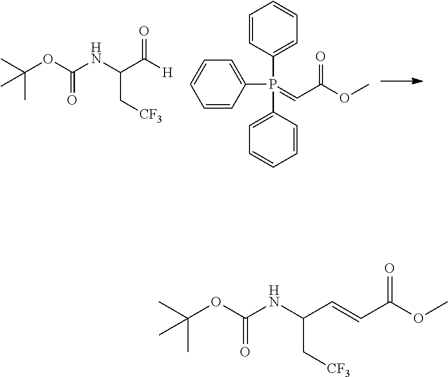

To a stirred solution of methyl(triphenylphosphoranylidene)acetate (7.12 g, 21.3 mmol) in Et₂O (40 mL) at RT was added a solution of Intermediate 91 in Et₂O (30 mL). The reaction mixture was stirred at RT for 15 h. The solid was removed by filtration and the solution was concentrated in vacuo. Purification via flash column chromatography (0-70% EtOAc/hexanes) afforded the title compound (4.14 g, 78% over two steps) as a white solid. LC-MS m/z 298 (M+H)⁺, 1.03 min (ret time).

### Intermediates 93-96

Intermediates 93-96 (Table I) were prepared from commercially available Boc-protected α-amino acids according to the procedures of Intermediates 31-33.

TABLE I

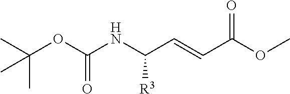

| | | | LC-MS | |
|---|---|---|---|---|
| | | | m/z | ret time |
| Intermediate | R³ | Name | (M + H)⁺ | (min) |
| 93 | 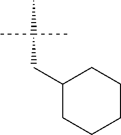 | methyl (2E,4S)-5-cyclohexyl-4- ({[(1,1-dimethylethyl)oxy]- carbonyl}amino)-2-pentenoate | 312 | 1.31 |
| 94 | 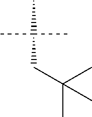 | methyl (2E,4S)-4-({[(1,1- dimethylethyl)oxy]carbonyl} amino)-6,6-dimethyl-2-heptenoate | 286 | 1.19 |
| 95 | 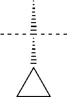 | methyl (2E,4S)-4-cyclopropyl-4- ({[(1,1-dimethylethyl)oxy]- carbonyl}amino)-2-butenoate | 256 | 1.03 |
| 96 | 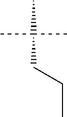 | methyl (2E,4S)-4-({[(1,1- dimethylethyl)oxy]carbonyl}- amino)-2-heptenoate | 258 | 1.10 |

### Intermediate 97

### 1,1-dimethylethyl (2S,4S)-2-({[(1S,2E)-4-(2,3-dihydro-1H-indol-1-yl)-1-(2-methylpropyl)-4-oxo-2-buten-1-yl]amino}carbonyl)-4-fluoro-1-pyrrolidinecarboxylate

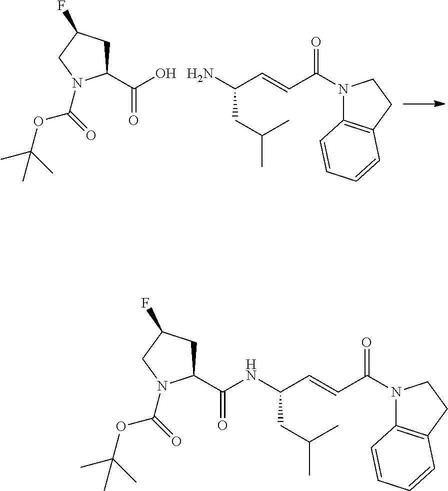

A mixture of [(1S,2E)-4-(2,3-dihydro-1H-indol-1-yl)-1-(2-methylpropyl)-4-oxo-2-buten-1-yl]amine (150 mg, 0.581 mmol), (4S)-1-{[(1,1-dimethylethyl)oxy]carbonyl}-4-fluoro-L-proline (135 mg, 0.581 mmol), HATU (221 mg, 0.581 mmol), and DIPEA (0.203 mL, 1.161 mmol) in DMF (2.0 mL) was stirred at RT for 1 h. The crude reaction mixture was purified by reverse phase HPLC, eluting with a linear gradient running from 40% CH₃CN/H₂O (0.1% formic acid) to 90% CH₃CN/H₂O (0.1% formic acid), to afford the title compound (52 mg, 19%). LC-MS m/z 474 (M+H)⁺, 1.12 min (ret time).

### COMPOUNDS OF FORMULA (I)

### Example 1

### (2E,4S)-4-(L-alanylamino)-6-phenyl-N-(phenylmethyl)-2-hexenamide hydrochloride

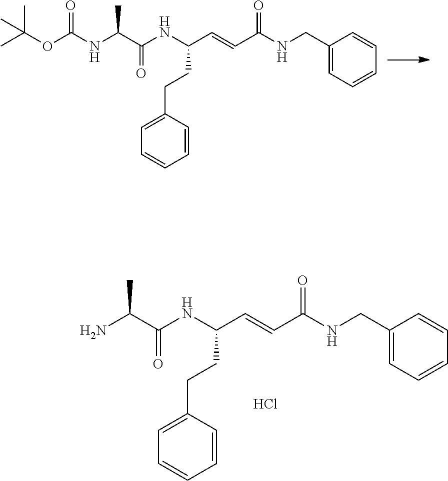

A solution of 1,1-dimethylethyl[(1S)-1-methyl-2-oxo-2-({(1S,2E)-4-oxo-1-(2-phenylethyl)-4-[(phenylmethyl)amino]-2-buten-1-yl}amino)ethyl]carbamate (2.00 g, 4.3 mmol) in concentrated HCl (2.0 mL) was stirred at RT for 1 h. The reaction mixture was basified with saturated aq. NaHCO₃ to pH 8 or 9 and then extracted with EtOAc (4×100 mL). The combined organic layers were washed with water (2×50 mL), dried over Na₂SO₄, filtered, and concentrated in vacuo to afford the free base of the title compound (0.60 g). The free base was stirred in 1 M HCl in Et₂O (20 mL) for 2 h. The resultant solid was collected by filtration and washed with Et₂O (10 mL) to afford the title compound (0.30 g, 18%) as a white solid. LC-MS m/z 366 (M+H)⁺, 1.59 min (ret time).

### Example 2

### (2E,4S)-4-(L-alanylamino)-N-methyl-6-phenyl-2-hexenamide hydrochloride

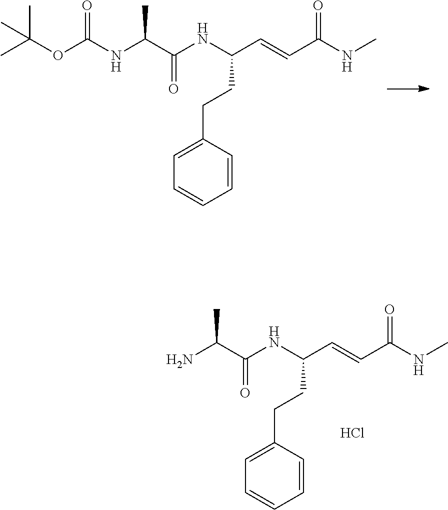

To a solution of 1,1-dimethylethyl((15)-1-methyl-2-{[(1S,2E)-4-(methylamino)-4-oxo-1-(2-phenylethyl)-2-buten-1-yl]amino}-2-oxoethyl)carbamate (1.7 g, 4.3 mol) in CH₂Cl₂ (30 mL) was added TFA (10 mL). The reaction mixture was stirred at RT for 2 h. Solvent was removed in vacuo and Et₂O (30 mL) was added. A solid was filtered and washed with saturated aq. NaHCO₃ (20 mL) and then water (10 mL) to afford the free base of the title compound (400 mg). The free base was stirred in 1 M HCl in Et₂O (20 mL) for 2 h. The resultant solid was collected by filtration and washed with Et₂O (20 mL) to afford the title compound (0.28 g, 20%) as a white solid. LC-MS m/z 290 (M+H)⁺, 1.34 min (ret time).

### Example 3

### (2E,4S)-4-(L-alanylamino)-N,N-dimethyl-6-phenyl-2-hexenamide hydrochloride

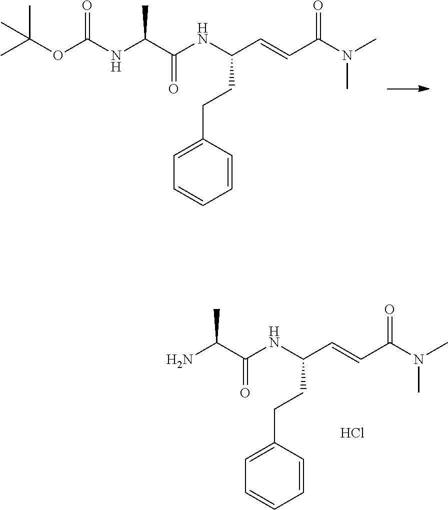

A solution of 1,1-dimethylethyl((1S)-2-{[(1S,2E)-4-(dimethylamino)-4-oxo-1-(2-phenylethyl)-2-buten-1-yl]amino}-1-methyl-2-oxoethyl)carbamate (2.00 g, 4.96 mmol) in concentrated HCl (2.0 mL) was stirred at RT for 1 h. The reaction mixture was basified with saturated aq. NaHCO₃ to pH 8 or 9 and then extracted with EtOAc (4×100 mL). The combined organic layers were washed with water (2×50 mL), dried over Na₂SO₄, filtered, and concentrated in vacuo to afford the free base of the title compound. The free base was stirred in 1 M HCl in Et₂O (20 mL) for 2 h. The resultant solid was collected by filtration and washed with Et₂O (10 mL) to afford the title compound (0.30 g, 20%) as a white solid. LC-MS m/z 304 (M+H)⁺, 1.39 min (ret time).

### Example 4

### N¹-[(1S,2E)-4-oxo-1-(2-phenylethyl)-4-(1-piperidinyl)-2-buten-1-yl]-L-alaninamide hydrochloride

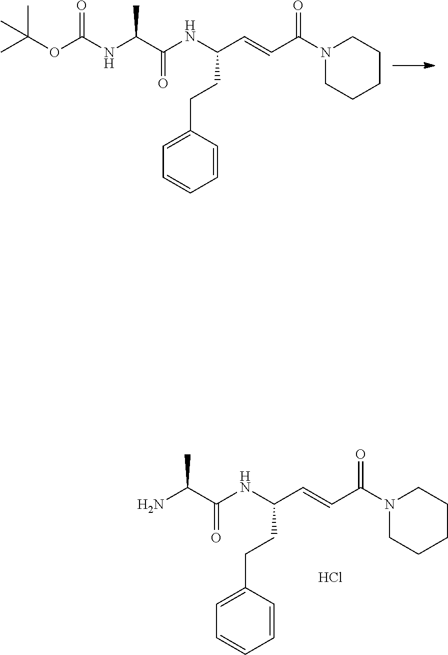

A solution of 1,1-dimethylethyl((15)-1-methyl-2-oxo-2-{[(1S,2E)-4-oxo-1-(2-phenylethyl)-4-(1-piperidinyl)-2-buten-1-yl]amino}ethyl)carbamate (1.00 g, 2.25 mmol) in concentrated HCl (2.0 mL) was stirred at RT for 1 h. The reaction mixture was basified with saturated aq. NaHCO₃ to pH 8 or 9 and then extracted with EtOAc (4×100 mL). The combined organic layers were washed with water (2×50 mL), dried over Na₂SO₄, filtered, and concentrated in vacuo to afford the free base of the title compound. The free base was stirred in 1 M HCl in Et₂O (20 mL) for 2 h. The resultant solid was collected by filtration and washed with Et₂O (10 mL) to afford the title compound (0.30 g, 36%) as a white solid. LC-MS m/z 344 (M+H)⁺, 1.57 min (ret time).

### Example 5

### (2E,4S)-4-(L-alanylamino)-N-[4-(methyloxy)phenyl]-6-phenyl-2-hexenamide hydrochloride

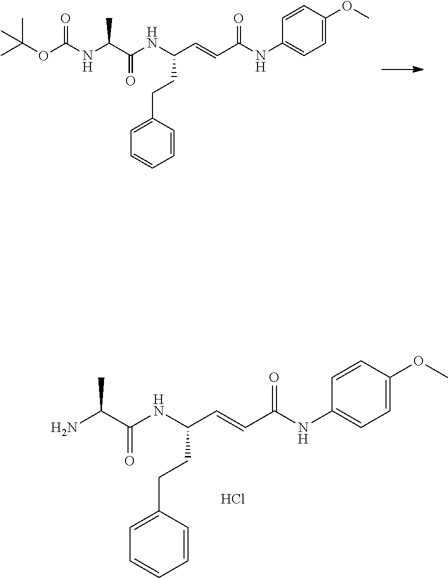

To a solution of 1,1-dimethylethyl((15)-1-methyl-2-{[(1S,2E)-4-{[4-(methyloxy)phenyl]amino}-4-oxo-1-(2-phenylethyl)-2-buten-1-yl]amino}-2-oxoethyl)carbamate (1.2 g, 2.49 mmol) in methanol (20 mL) was added concentrated HCl (10 mL). The reaction mixture was stirred at RT for 1 h. The reaction mixture was basified with saturated aq. NaHCO₃ to pH 8 or 9 and then extracted with EtOAc (4×100 mL). The combined organic layers were washed with water (2×50 mL), dried over Na₂SO₄, filtered, and concentrated in vacuo to afford the free base of the title compound. The free base was stirred in 1 M HCl in Et₂O (20 mL) for 2 h. The resultant solid was collected by filtration and washed with Et₂O (10 mL) to afford the title compound (0.60 g, 58%) as a white solid. LC-MS m/z 382 (M+H)⁺, 1.61 min (ret time). ¹HNMR (400 MHz, DMSO-d₆) δ ppm 10.02 (1H, s), 8.67 (1H, d, J=8.2 Hz), 8.03 (3H, br. s.), 7.55 (2H, m, J=9.3 Hz), 7.32-7.18 (5H, m), 6.90 (2H, m, J=9.3 Hz), 6.69 (1H, dd, J=15.4, 6.7 Hz), 6.18 (1H, d, J=15.4 Hz), 4.48-4.39 (1H, m), 3.91 (1H, q, J=7.1 Hz), 3.71 (3H, s), 2.71-2.58 (2H, m), 1.97-1.79 (2H, m), 1.44 (3H, d, J=7.1 Hz).

### Example 6

### methyl 3-{[(2E,4S)-4-(L-alanylamino)-6-phenyl-2-hexenoyl]amino}benzoate trifluoroacetate

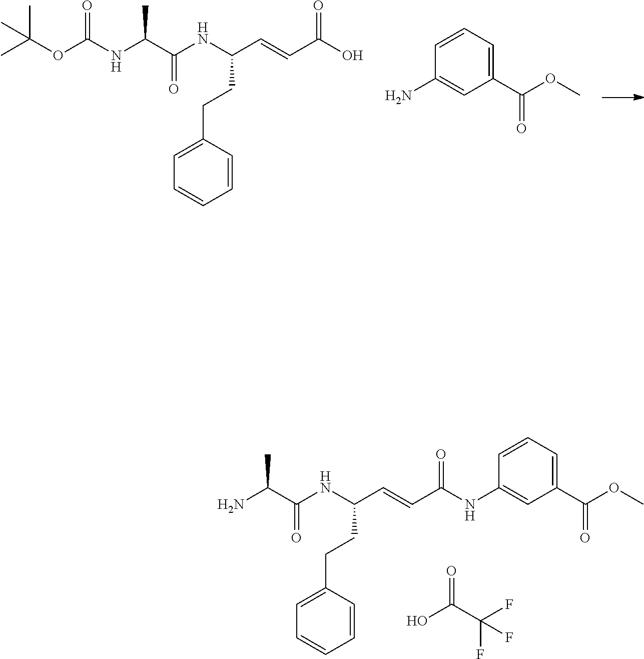

To a solution of (2E,4S)-4-[(N-{[(1,1-dimethylethyl)oxy]carbonyl}-L-alanyl)amino]-6-phenyl-2-hexenoic acid (37.6 mg, 0.100 mmol) and HATU (38.0 mg, 0.100 mmol) in DMF (0.2 mL) was added DIPEA (0.05 mL, 0.286 mmol). The reaction mixture was shaken for 2 min and then dispensed to methyl 3-aminobenzoate (15.0 mg, 0.099 mmol). The reaction vial was capped and shaken to aid dispersion. The reaction mixture then stood at RT for 18 h. Solvent was removed under a stream of nitrogen in a Radleys blowdown apparatus to ⅓ original volume. TFA (0.20 mL) was added, and the vial was capped and shaken for 2 min. The reaction mixture stood at RT for 2 h. The crude product was purified by reverse phase HPLC using an MDAP equipped with a SunFire C18 column with a gradient of CH₃CN in water containing a TFA modifier. The solvent was evaporated in vacuo to afford the title compound (21.5 mg, 37%). LC-MS m/z 410 (M+H)⁺, 1.7 min (ret time).

### Example 7

### (2E,4S)-4-(L-alanylamino)-N-[2-(methyloxy)phenyl]-6-phenyl-2-hexenamide trifluoroacetate

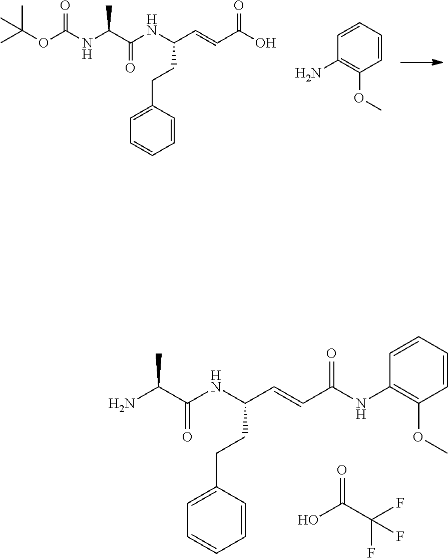

To a solution of (2E,4S)-4-[(N-{[(1,1-dimethylethyl)oxy]carbonyl}-L-alanyl)amino]-6-phenyl-2-hexenoic acid (37.6 mg, 0.100 mmol) and HATU (38.0 mg, 0.100 mmol) in DMF (0.2 mL) was added DIPEA (0.05 mL, 0.286 mmol). The reaction mixture was shaken for 2 min and then dispensed to 2-(methyloxy)aniline (9.6 mg, 0.078 mmol). The reaction vial was capped and shaken to aid dispersion. The reaction mixture then stood at RT for 18 h. Solvent was removed under a stream of nitrogen in a Radleys blowdown apparatus to ⅓ original volume. TFA (0.20 mL) was added, and the vial was capped and shaken for 2 min. The reaction mixture stood at RT for 2 h. The crude product was purified by reverse phase HPLC using an MDAP equipped with a SunFire C18 column with a gradient of CH₃CN in water containing a TFA modifier. The solvent was evaporated in vacuo to afford the title compound (18 mg, 33%). LC-MS m/z 382 (M+H)⁺, 1.67 min (ret time).

### Example 8

### (2E,4S)-4-(L-alanylamino)-6-phenyl-N-1,3-thiazol-2-yl-2-hexenamide trifluoroacetate

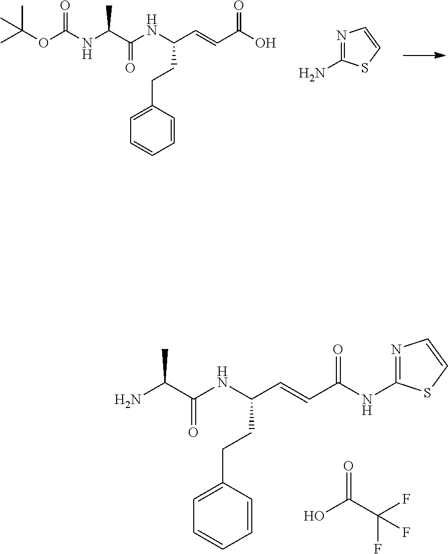

To a solution of (2E,4S)-4-[(N-{[(1,1-dimethylethyl)oxy]carbonyl}-L-alanyl)amino]-6-phenyl-2-hexenoic acid (37.6 mg, 0.100 mmol) and HATU (38.0 mg, 0.100 mmol) in DMF (0.2 mL) was added DIPEA (0.05 mL, 0.286 mmol). The reaction mixture was shaken for 2 min and then dispensed to 1,3-thiazol-2-amine (9.1 mg, 0.091 mmol). The reaction vial was capped and shaken to aid dispersion. The reaction mixture then stood at RT for 18 h. Solvent was removed under a stream of nitrogen in a Radleys blowdown apparatus to ⅓ original volume. TFA (0.20 mL) was added, and the vial was capped and shaken for 2 min. The reaction mixture stood at RT for 2 h. The crude product was purified by reverse phase HPLC using an MDAP equipped with a SunFire C18 column with a gradient of CH₃CN in water containing a TFA modifier. The solvent was evaporated in vacuo to afford the title compound (15 mg, 29%). LC-MS m/z 359 (M+H)⁺, 1.51 min (ret time).

### Example 9

### (2E,4S)-4-(L-alanylamino)-6-phenyl-N-[3-(trifluoromethyl)phenyl]-2-hexenamide trifluoroacetate

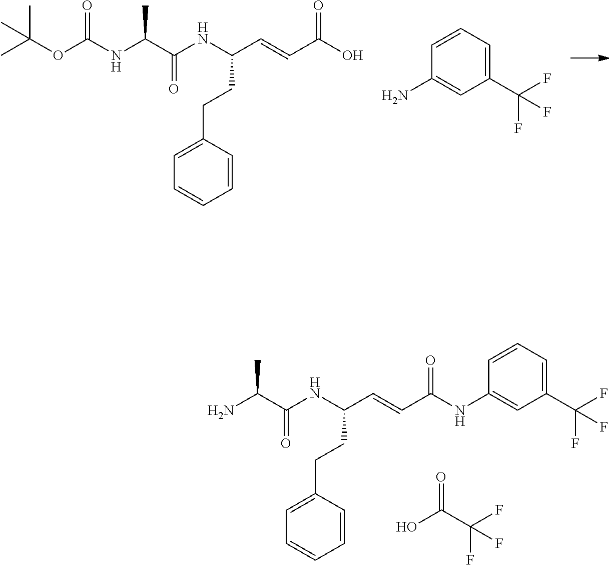

To a solution of (2E,4S)-4-[(N-{[(1,1-dimethylethyl)oxy]carbonyl}-L-alanyl)amino]-6-phenyl-2-hexenoic acid (37.6 mg, 0.100 mmol) and HATU (38.0 mg, 0.100 mmol) in DMF (0.2 mL) was added DIPEA (0.05 mL, 0.286 mmol). The reaction mixture was shaken for 2 min and then dispensed to 3-(trifluoromethyl) aniline (16.1 mg, 0.100 mmol). The reaction vial was capped and shaken to aid dispersion. The reaction mixture then stood at RT for 18 h. Solvent was removed under a stream of nitrogen in a Radleys blowdown apparatus to ⅓ original volume. HCl (2 M solution in 1,4-dioxane, 0.20 mL) was added, and the vial was capped and shaken for 2 min. The reaction mixture stood at RT for 2 h. The crude product was purified by reverse phase HPLC using an MDAP equipped with a SunFire C18 column with a gradient of CH₃CN in water containing a TFA modifier. The solvent was evaporated in vacuo to afford the title compound (3.1 mg, 5%). LC-MS m/z 420 (M+H)⁺, 0.85 min (ret time).

### Example 10

### (2E,4S)-4-(L-alanylamino)-N-methyl-N,6-diphenyl-2-hexenamide trifluoroacetate

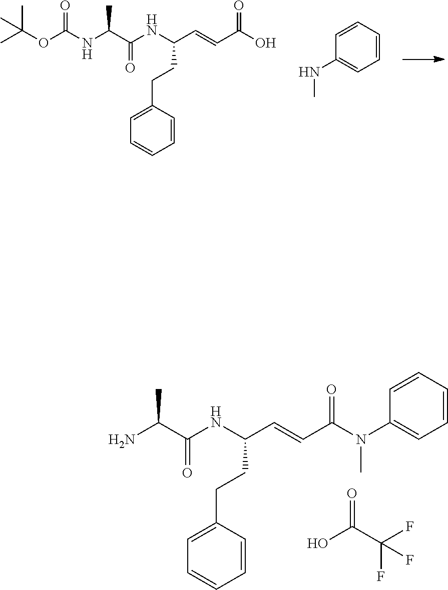

To a solution of (2E,4S)-4-[(N-{[(1,1-dimethylethyl)oxy]carbonyl}-L-alanyl)amino]-6-phenyl-2-hexenoic acid (37.6 mg, 0.100 mmol) and HATU (38.0 mg, 0.100 mmol) in DMF (0.2 mL) was added DIPEA (0.05 mL, 0.286 mmol). The reaction mixture was shaken for 2 min and then dispensed to N-methylaniline (10.7 mg, 0.100 mmol). The reaction vial was capped and shaken to aid dispersion. The reaction mixture then stood at RT for 18 h. Solvent was removed under a stream of nitrogen in a Radleys blowdown apparatus to ⅓ original volume. HCl (2 M solution in 1,4-dioxane, 0.20 mL) was added, and the vial was capped and shaken for 2 min. The reaction mixture stood at RT for 2 h. The crude product was purified by reverse phase HPLC using an MDAP equipped with a SunFire C18 column with a gradient of CH₃CN in water containing a TFA modifier. The solvent was evaporated in vacuo to afford the title compound (18 mg, 37%). LC-MS m/z 366 (M+H)⁺, 0.75 min (ret time).

### Example 11

### (2E,4S)-4-(L-alanylamino)-6-phenyl-N-[4-(trifluoromethyl)phenyl]-2-hexenamide formate

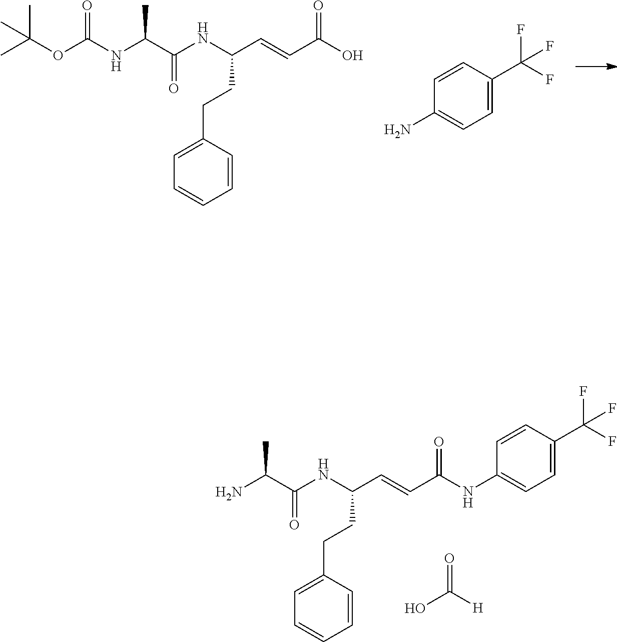

To a solution of (2E,4S)-4-[(N-{[(1,1-dimethylethyl)oxy]carbonyl}-L-alanyl)amino]-6-phenyl-2-hexenoic acid (37.6 mg, 0.100 mmol) and HATU (38.0 mg, 0.100 mmol) in DMF (0.2 mL) was added DIPEA (0.05 mL, 0.286 mmol). The reaction mixture was shaken for 2 min and then dispensed to 4-(trifluoromethyl)aniline (16.1 mg, 0.100 mmol). The reaction vial was capped and shaken to aid dispersion. The reaction mixture then stood at RT for 72 h. Solvent was removed under a stream of nitrogen in a Radleys blowdown apparatus to ⅓ original volume. HCl (2 M solution in 1,4-dioxane, 0.20 mL) was added, and the vial was capped and shaken for 2 min. The reaction mixture stood at RT for 2 h. Additional HCl (2 M solution in 1,4-dioxane, 0.20 mL) was added and the reaction mixture stood at RT for a further 18 h. The crude product was purified by reverse phase HPLC using an MDAP equipped with a SunFire C18 column with a gradient of CH₃CN in water containing a TFA modifier. The solvent was evaporated in vacuo to afford an impure fraction of the TFA salt of the title compound. The TFA salt was dissolved in 1:1 MeOH:DMSO (0.6 mL) and purified by reverse phase HPLC using an MDAP equipped with an Atlantis column with a gradient of CH₃CN in water containing a formic acid modifier. The solvent was removed under a stream of nitrogen in the Radleys blowdown apparatus to afford the title compound (4.0 mg, 8%). LC-MS m/z 420 (M+H)⁺, 1.96 min (ret time).

### Example 12

### methyl 4-{[(2E,4S)-4-(L-alanylamino)-6-phenyl-2-hexenoyl]amino}benzoate formate

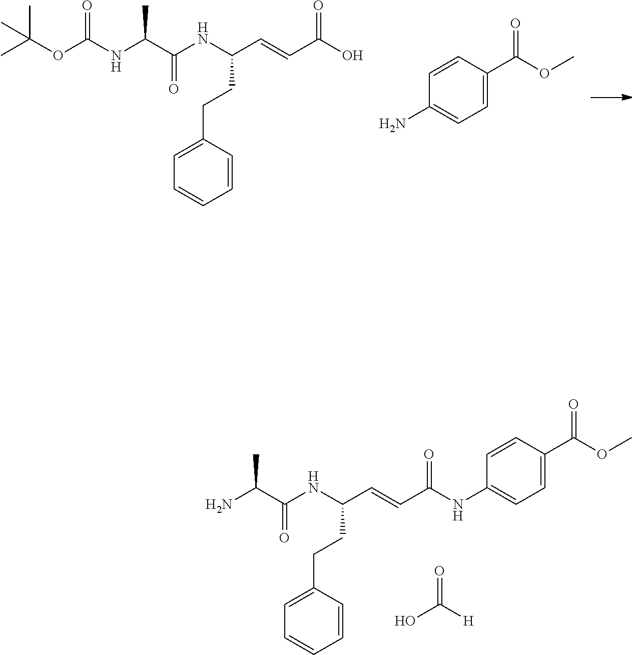

To a solution of (2E,4S)-4-[(N-{[(1,1-dimethylethyl)oxy]carbonyl}-L-alanyl)amino]-6-phenyl-2-hexenoic acid (37.6 mg, 0.100 mmol) and HATU (38.0 mg, 0.100 mmol) in DMF (0.2 mL) was added DIPEA (0.05 mL, 0.286 mmol). The reaction mixture was shaken for 2 min and then dispensed to methyl 4-aminobenzoate (15.1 mg, 0.100 mmol). The reaction vial was capped and shaken to aid dispersion. The reaction mixture then stood at RT for 72 h. Solvent was removed under a stream of nitrogen in a Radleys blowdown apparatus to ⅓ original volume. HCl (2 M solution in 1,4-dioxane, 0.20 mL) was added, and the vial was capped and shaken for 2 min. The reaction mixture stood at RT for 2 h. Additional HCl (2 M solution in 1,4-dioxane, 0.20 mL) was added and the reaction mixture stood at RT for a further 18 h. The crude product was purified by reverse phase HPLC using an MDAP equipped with a SunFire C18 column with a gradient of CH₃CN in water containing a TFA modifier. The solvent was evaporated in vacuo to afford an impure fraction of the TFA salt of the title compound. The TFA salt was dissolved in 1:1 MeOH:DMSO (0.6 mL) and purified by reverse phase HPLC using an MDAP equipped with an Atlantis column with a gradient of CH₃CN in water containing a formic acid modifier. The solvent was removed under a stream of nitrogen in the Radleys blowdown apparatus to afford the title compound (3.2 mg, 7%). LC-MS m/z 410 (M+H)⁺, 1.75 min (ret time).

### Example 13

### (2E,4S)-4-(L-alanylamino)-N-cyclohexyl-N-methyl-6-phenyl-2-hexenamide trifluoroacetate

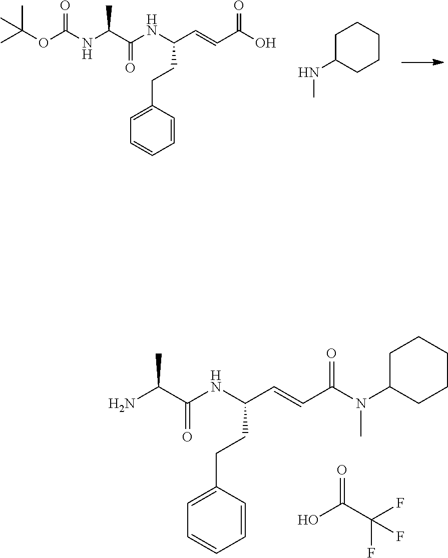

To a solution of (2E,4S)-4-[(N-{[(1,1-dimethylethyl)oxy]carbonyl}-L-alanyl)amino]-6-phenyl-2-hexenoic acid (37.6 mg, 0.100 mmol) and HATU (38.0 mg, 0.100 mmol) in DMF (0.2 mL) was added DIPEA (0.05 mL, 0.286 mmol). The reaction mixture was shaken for 2 min and then dispensed to cyclohexyl(methyl)amine (11.3 mg, 0.100 mmol). The reaction vial was capped and shaken to aid dispersion. The reaction mixture then stood at RT for 72 h. Solvent was removed under a stream of nitrogen in a Radleys blowdown apparatus to ⅓ original volume. HCl (2 M solution in 1,4-dioxane, 0.20 mL) was added, and the vial was capped and shaken for 2 min. The reaction mixture stood at RT for 2 h. Additional HCl (2 M solution in 1,4-dioxane, 0.20 mL) was added and the reaction mixture stood at RT for a further 18 h. The crude product was purified by reverse phase HPLC using an MDAP equipped with a SunFire C18 column with a gradient of CH₃CN in water containing a TFA modifier. The solvent was evaporated in vacuo to afford the title compound (25 mg, 47%). LC-MS m/z 372 (M+H)⁺, 1.75 min (ret time).

### Example 14

### (2E,4S)-4-(L-alanylamino)-N-[(1R,3S)-3-hydroxycyclopentyl]-6-phenyl-2-hexenamide formate

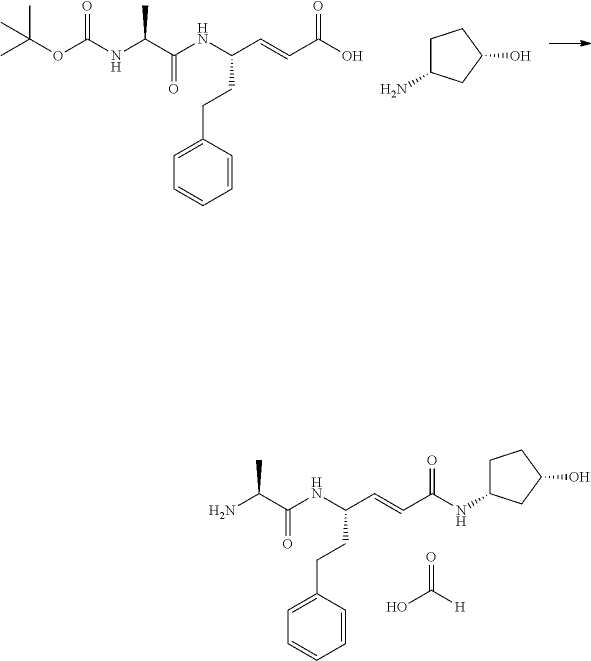

To a solution of (2E,4S)-4-[(N-{[(1,1-dimethylethyl)oxy]carbonyl}-L-alanyl)amino]-6-phenyl-2-hexenoic acid (37.6 mg, 0.100 mmol) and HATU (38.0 mg, 0.100 mmol) in DMF (0.2 mL) was added DIPEA (0.05 mL, 0.286 mmol). The reaction mixture was shaken for 2 min and then (1S,3R)-3-aminocyclopentanol (10.1 mg, 0.100 mmol) in DMF (0.10 mL) was added. The reaction vial was capped and shaken to aid dispersion. The reaction mixture then stood at RT overnight. Solvent was removed under a stream of nitrogen in a Radleys blowdown apparatus to ⅓ original volume. HCl (4 M solution in 1,4-dioxane, 0.20 mL) was added, and the vial was capped and shaken for 2 min. The reaction mixture stood at RT for 2 h. 1:1 DMSO:MeOH (0.30 mL) was added and the reaction was filtered and purified by reverse phase HPLC using an MDAP equipped with a SunFire C18 column with a gradient of CH₃CN in water containing a TFA modifier. The solvent was removed under a stream of nitrogen in a Radleys blowdown apparatus to afford an impure fraction of the TFA salt of the title compound. The TFA salt was dissolved in DMSO (0.5 mL) and purified by reverse phase HPLC using an MDAP equipped with an Atlantis column with a gradient of CH₃CN in water containing a formic acid modifier. The solvent was removed under a stream of nitrogen in the Radleys blowdown apparatus to afford the title compound (4.0 mg, 9%). LC-MS m/z 360 (M+H)⁺, 1.33 min (ret time).

### Example 15

### (2E,4S)-4-(L-alanylamino)-N-[(1S,4R)-bicyclo[2.2.1]hept-2-yl]-6-phenyl-2-hexenamide trifluoroacetate

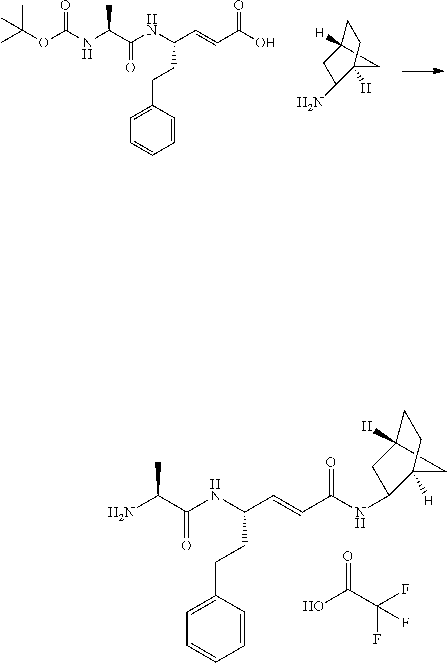

To a solution of (2E,4S)-4-[(N-{[(1,1-dimethylethyl)oxy]carbonyl}-L-alanyl)amino]-6-phenyl-2-hexenoic acid (37.6 mg, 0.100 mmol) and HATU (38.0 mg, 0.100 mmol) in DMF (0.2 mL) was added DIPEA (0.05 mL, 0.286 mmol). The reaction mixture was shaken for 2 min and then (1S,4R)-bicyclo[2.2.1]heptan-2-amine (11.1 mg, 0.100 mmol) in DMF (0.10 mL) was added. The reaction vial was capped and shaken to aid dispersion. The reaction mixture then stood at RT overnight. Solvent was removed under a stream of nitrogen in a Radleys blowdown apparatus to ⅓ original volume. HCl (4 M solution in 1,4-dioxane, 0.20 mL) was added, and the vial was capped and shaken for 2 min. The reaction mixture stood at RT for 2 h. 1:1 DMSO:MeOH (0.30 mL) was added and the reaction was filtered and purified by reverse phase HPLC using an MDAP equipped with a SunFire C18 column with a gradient of CH₃CN in water containing a TFA modifier. The solvent was removed under a stream of nitrogen in a Radleys blowdown apparatus to afford the title compound (7.0 mg, 14%). LC-MS m/z 370 (M+H)⁺, 1.58 min (ret time).

### Example 16

N¹-[(1S,2E)-4-(1H-indol-1-yl)-4-oxo-1-(2-phenylethyl)-2-buten-1-yl]-L-alaninamide trifluoroacetate

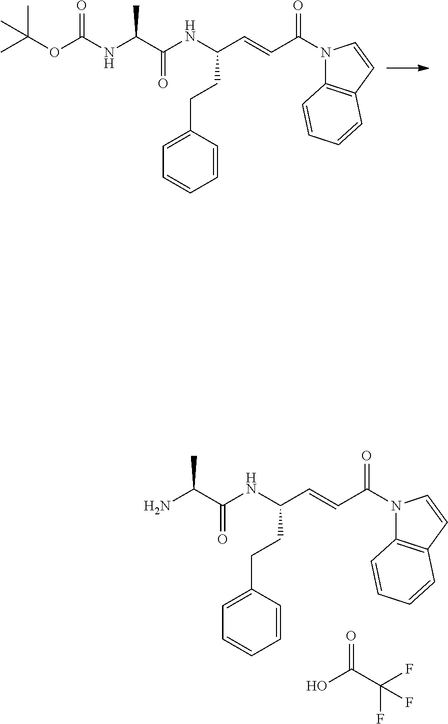

To a solution of 1,1-dimethylethyl((1S)-2-{[(1S,2E)-4-(1H-indol-1-yl)-4-oxo-1-(2-phenylethyl)-2-buten-1-yl]amino}-1-methyl-2-oxoethyl)carbamate (190 mg, 0.40 mmol) in CH₂Cl₂ (5.0 mL) was added TFA (1.1 mL, 14.28 mmol). The reaction mixture was stirred at RT for 40 min and then concentrated under a stream of nitrogen at 50° C. The crude product was dissolved in DMSO (4.0 mL), filtered through a 0.45 μm Acrodisc® filter, and purified by reverse phase HPLC(YMC C18 S-5 μm/12 nm 50×20 mm preparatory column), eluting at 20 mL/min with a linear gradient running from 10% CH₃CN/H₂O (0.1% TFA) to 80% CH₃CN/H₂O (0.1% TFA) over 15 min. The desired fractions were concentrated under a stream of nitrogen at 50° C., dissolved in water (2.0 mL), and lyophilized on a Genevac HT-4× to afford the title compound (110 mg, 56%). LC-MS m/z 376 (M+H)⁺, 0.98 min (ret time). ¹H NMR (400 MHz, MeOD) δ ppm 8.43 (d, J=8.0 Hz, 1H), 7.75 (d, J=3.8 Hz, 1H), 7.60 (d, J=7.3 Hz, 1H), 7.33 (s, 1H), 7.32-7.26 (m, 5H), 7.19 (t, J=7.0 Hz, 1H), 7.09 (dd, J=15.3, 6.4 Hz, 1H), 6.95 (d, J=15.3 Hz, 1H), 6.73 (d, J=3.8 Hz, 1H), 4.74-4.66 (m, 1H), 4.04 (q, J=7.0 Hz, 1H), 2.81-2.75 (m, 2H), 2.10-2.03 (m, 2H), 1.60 (d, J=7.0 Hz, 3H).

### Examples 17-20

General Experimental for Array Table 1

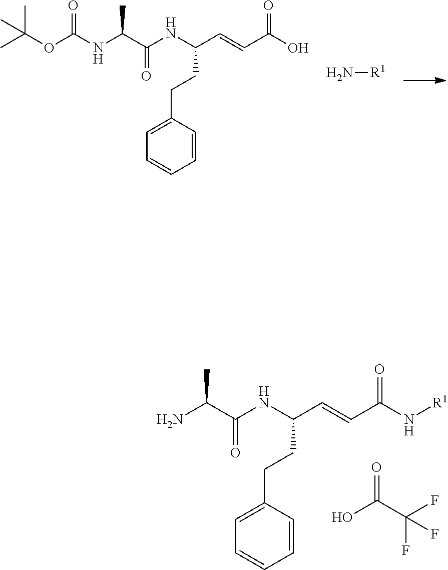

Using array chemistry, following the procedure as described here in the preparation of Examples 17-20. A mixture of (2E,4S)-4-[(N-{[(1,1-dimethylethyl)oxy]carbonyl}-L-alanyl)amino]-6-phenyl-2-hexenoic acid (37.6 mg, 0.1 mmol) and HATU (38 mg, 0.1 mmol) in DMF (0.2 mL) was added to empty micronic vials. DIPEA (0.035 mL, 0.2 mmol) was added to each. Each vial was capped and shaken for 1 min to aid dispersion. The pre-weighed appropriate amines (0.1 mmol) were added and the vials were shaken at RT for 18 h. Solvent was removed under a stream of nitrogen in a Radleys blow down apparatus to ⅓ of the original volume. TFA (0.20 mL) was added to each reaction and the vial was capped, shaken for 2 min and left to stand at RT for 1 h. Each crude product was purified by reverse phase HPLC using an MDAP equipped with a SunFire C18 column with a gradient of CH₃CN in water containing a TFA modifier. The solvent was removed under a stream of nitrogen in the Radleys blowdown apparatus to afford the corresponding products as TFA salts. Examples 17-20 listed in Table 1.

TABLE 1
Examples 17-20.

| | | | LC-MS | |
|---|---|---|---|---|
| | | | m/z | ret time |
| Example | R¹ | Name | (M + H)⁺ | (min) |
| 17 | 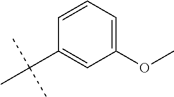 | (2E,4S)-4-(L-alanylamino)-N-[3- (methyloxy)phenyl]-6-phenyl-2- hexenamide trifluoroacetate | 382 | 1.63 |
| 18 | 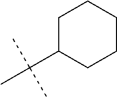 | (2E,4S)-4-(L-alanylamino)-N- cyclohexyl-6-phenyl-2- hexenamide trifluoroacetate | 358 | 1.98 |
| 19 | 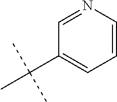 | (2E,4S)-4-(L-alanylamino)-6- phenyl-N-3-pyridinyl-2- hexenamide trifluoroacetate | 353 | 1.06 |
| 20 | 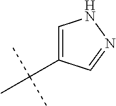 | (2E,4S)-4-(L-alanylamino)-6- phenyl-N-1H-pyrazol-4-yl-2- hexenamide trifluoroacetate | 342 | 1.19 |

### Examples 21-37

### General Experimental for Array Table 2

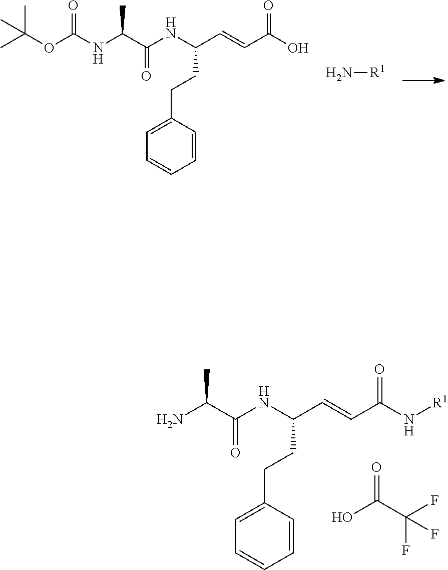

Using array chemistry, following the procedure as described here in the preparation of Examples 21-37. (2E,4S)-4-[(N-{[(1,1-dimethylethyl)oxy]carbonyl}-L-alanyl)amino]-6-phenyl-2-hexenoic acid (715 mg, 1.90 mmol) was added to HATU (722 mg, 1.90 mmol) and then dissolved in DMF (3.8 mL). The solution was dispensed out into micronic vials (0.2 mL, 0.100 mmol) per vial. DIPEA (0.05 mL, 0.286 mmol) was added to each. Each vial was capped and shaken to aid dispersion. A solution of each pre-weighed appropriate amine (0.100 mmol) in DMF (0.10 mL) was added and the vials were shaken for 5 min and then left standing at RT overnight. Solvent was removed under a stream of nitrogen in a Radleys blow down apparatus to ⅓ of the original volume. TFA (0.20 mL) was added to each reaction and the vial was capped, shaken for 2 min and left to stand at RT for 2 h. 1:1 DMSO:MeOH (0.30 mL) was added to each reaction, which was then filtered and purified by reverse phase HPLC using an MDAP equipped with a SunFire C18 column with a gradient of CH₃CN in water containing a TFA modifier. The solvent was removed under a stream of nitrogen in the Radleys blowdown apparatus to afford the corresponding products as TFA salts. Examples 21-37 listed in Table 2.

TABLE 2
Examples 21-37.

| | | | LC-MS | |
|---|---|---|---|---|
| | | | m/z | ret time |
| Example | R¹ | Name | (M + H)⁺ | (min) |
| 21 | 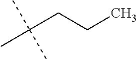 | (2E,4S)-4-(L-alanylamino)-6- phenyl-N-propyl-2-hexenamide trifluoroacetate | 318 | 0.65 |
| 22 | 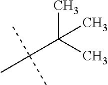 | (2E,4S)-4-(L-alanylamino)-N- (1,1-dimethylethyl)-6-phenyl-2- hexenamide trifluoroacetate | 332 | 0.71 |
| 23 | 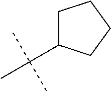 | (2E,4S)-4-(L-alanylamino)-N- cyclopentyl-6-phenyl-2- hexenamide trifluoroacetate | 344 | 0.72 |
| 24 | 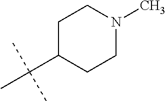 | (2E,4S)-4-(L-alanylamino)-N-(1- methyl-4-piperidinyl)-6-phenyl- 2-hexenamide trifluoroacetate | 373 | 0.43 |
| 25 | 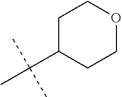 | (2E,4S)-4-(L-alanylamino)-6- phenyl-N-(tetrahydro-2H-pyran- 4-yl)-2-hexenamide trifluoroacetate | 360 | 0.59 |
| 26 | 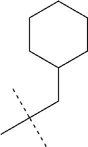 | (2E,4S)-4-(L-alanylamino)-N- (cyclohexylmethyl)-6-phenyl-2- hexenamide trifluoroacetate | 372 | 0.82 |
| 27 | 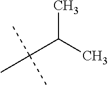 | (2E,4S)-4-(L-alanylamino)-N-(1- methylethyl)-6-phenyl-2- hexenamide trifluoroacetate | 318 | 0.64 |
| 28 | 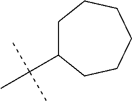 | (2E,4S)-4-(L-alanylamino)-N- cycloheptyl-6-phenyl-2- hexenamide trifluoroacetate | 372 | 0.81 |
| 29 | 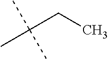 | (2E,4S)-4-(L-alanylamino)-N- ethyl-6-phenyl-2-hexenamide trifluoroacetate | 304 | 0.59 |
| 30 | 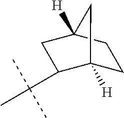 | (2E,4S)-4-(L-alanylamino)-N- [(1R,4S)-bicyclo[2.2.1]hept-2- yl]-6-phenyl-2-hexenamide trifluoroacetate | 370 | 0.78 |
| 31 | 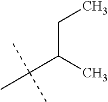 | (2E,4S)-4-(L-alanylamino)-N-(1- methylpropyl)-6-phenyl-2- hexenamide trifluoroacetate | 332 | 0.69 |
| 32 | 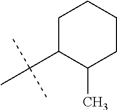 | (2E,4S)-4-(L-alanylamino)-N-(2- methylcyclohexyl)-6-phenyl-2- hexenamide trifluoroacetate | 372 | 0.81 |
| 33 | 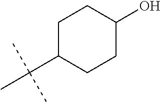 | (2E,4S)-4-(L-alanylamino)-N-(4- hydroxycyclohexyl)-6-phenyl-2- hexenamide trifluoroacetate | 374 | 0.57 |
| 34 | 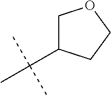 | (2E,4S)-4-(L-alanylamino)-6- phenyl-N-(tetrahydro-3-furanyl)- 2-hexenamide trifluoroacetate | 346 | 0.59 |
| 35 | 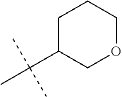 | (2E,4S)-4-(L-alanylamino)-6- phenyl-N-(tetrahydro-2H-pyran- 3-yl)-2-hexenamide trifluoroacetate | 360 | 0.63 |
| 36 | 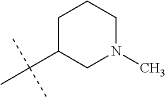 | (2E,4S)-4-(L-alanylamino)-N-(1- methyl-3-piperidinyl)-6-phenyl- 2-hexenamide trifluoroacetate | 373 | 0.45 |
| 37 | 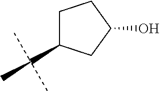 | (2E,4S)-4-(L-alanylamino)-N- [(1S,3S)-3-hydroxycyclopentyl]- 6-phenyl-2-hexenamide trifluoroacetate | 360 | 1.24 |

### Example 38

### N¹-[(1S,2E)-4-[(1R,8S)-11-azatricyclo[6.2.1.0^{2,7}]undeca-2,4,6-trien-11-yl]-4-oxo-1-(2-phenylethyl)-2-buten-1-yl]-L-alaninamide trifluoroacetate

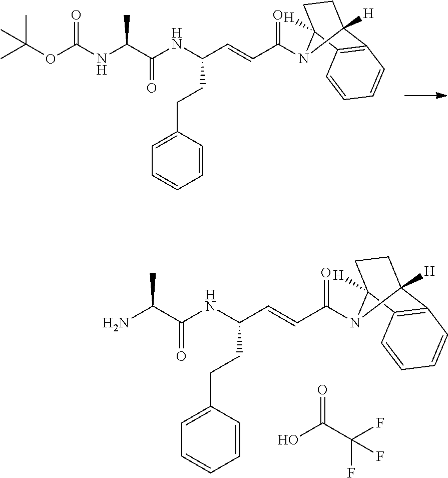

To a solution of 1,1-dimethylethyl((1S)-2-{[(1S,2E)-4-[(1R,8S)-11-azatricyclo[6.2.1.0^{2,7}]undeca-2,4,6-trien-11-yl]-4-oxo-1-(2-phenylethyl)-2-buten-1-yl]amino}-1-methyl-2-oxoethyl)carbamate (140 mg, 0.278 mmol) in CH₂Cl₂ (4.0 mL) was added TFA (0.043 mL, 0.556 mmol). After stirring overnight at RT, the reaction mixture was concentrated in vacuo and purified by reverse phase HPLC(YMC C18 S-15 μm/12 nm 75×30 mm preparatory column) eluting at 35 mL/min with a linear gradient running from 10% CH₃CN/H₂O (0.1% TFA) to 80% CH₃CN/H₂O (0.1% TFA) over 15 min. The solvent was evaporated in vacuo to afford the title compound (56 mg, 39%). LC-MS m/z 404 (M+H)⁺, 0.95 min (ret time). ¹H NMR (400 MHz, MeOD) δ ppm 1.29-1.49 (m, 2H) 1.55 (m, 3H) 1.83-2.04 (m, 2H) 2.04-2.28 (m, 2H) 2.58-2.79 (m, 2H) 3.97 (t, J=6.8 Hz, 1H) 4.49-4.60 (m, 1H) 5.49 (d, J=3.3 Hz, 1H) 5.56 (d, J=3.5 Hz, 1H) 6.40 (d, J=15.3 Hz, 1H) 6.67 (dd, J=15.3, 6.8 Hz, 1H) 7.13-7.43 (m, 9H).

### Example 39

### (2S)-2-amino-N-[(1S,2E)-4-[(1R,8S)-11-azatricyclo[6.2.1.0^{2,7}]undeca-2,4,6-trien-11-yl]-4-oxo-1-(2-phenylethyl)-2-buten-1-yl]butanamide trifluoroacetate

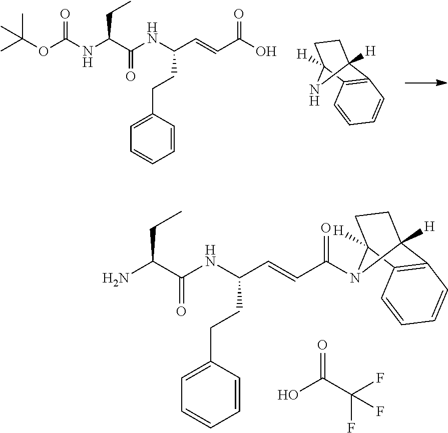

A solution of (2E,4S)-4-{[(2S)-2-({[(1,1-dimethylethyl)oxy]carbonyl}amino)-butanoyl]amino}-6-phenyl-2-hexenoic acid (120 mg, 0.308 mmol), HATU (237 mg, 0.603 mmol), and DIPEA (0.25 mL, 1.428 mmol) in DMF (1.0 mL) was stirred for 27 min in a vial. (1R,8S)-11-azatricyclo[6.2.1.0^{2,7}]undeca-2,4,6-triene (52 mg, 0.355 mmol) was added and stirring continued for 20 min. The reaction mixture was concentrated and treated with TFA (1.2 mL, 15.6 mmol) in CH₂Cl₂ (1.0 mL). The reaction mixture was stirred for 1 h 50 min at RT. The reaction mixture was then concentrated under a stream of nitrogen at 50° C. The crude product was dissolved in DMSO (3.0 mL), filtered through a 0.45 μm Acrodisc® filter, and purified by reverse phase HPLC(YMC C18 S-5 μm/12 nm 50×20 mm preparatory column), eluting at 20 mL/min with a linear gradient running from 10% CH₃CN/H₂O (0.1% TFA) to 80% CH₃CN/H₂O (0.1% TFA) over 15 min. The desired fractions were concentrated under a stream of nitrogen at 50° C., dissolved in water (2.0 mL), and lyophilized on a Genevac HT-4× to afford the title compound (47 mg, 26%). LC-MS m/z 418 (M+H)⁺, 0.92 min (ret time). ¹H NMR (400 MHz, MeOD) δ ppm 7.11-7.00 (m, 9H), 6.50 (dd, J=7.0, 15.3 Hz, 1H), 6.24 (d, J=15.3, 1H), 5.36 (s, 1H), 5.29 (s, 1H), 4.39 (d, J=6.3 Hz, 1H), 3.70 (m, 1H), 2.50 (m, 1H), 1.92 (m, 1H), 1.76 (m, 4H), 1.19 (m, 2H), 0.87 (m, 3H).

### Example 40

### N¹-[(1S,2E)-4-(1,3-dihydro-2H-isoindol-2-yl)-1-ethyl-4-oxo-2-buten-1-yl]-L-alaninamide trifluoroacetate

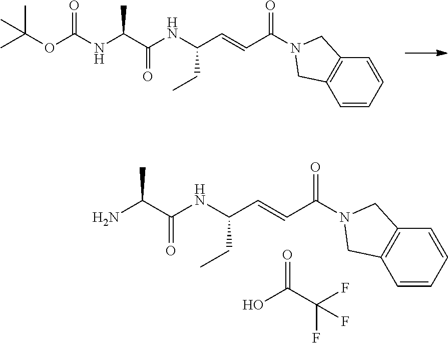

To a solution of 1,1-dimethylethyl((1S)-2-{[(1S,2E)-4-(1,3-dihydro-2H-isoindol-2-yl)-1-ethyl-4-oxo-2-buten-1-yl]amino}-1-methyl-2-oxoethyl)carbamate (120 mg, 0.299 mmol) in CH₂Cl₂ (4.0 mL) was added TFA (0.184 mL, 2.391 mmol). The reaction mixture was stirred at RT overnight. The solvent was removed and the residue was purified via reverse phase HPLC(YMC C18 S-5 μm/12 nm 75×30 mm preparatory column), eluting at 35 mL/min with a linear gradient running from 10% CH₃CN/H₂O (0.1% TFA) to 80% CH₃CN/H₂O (0.1% TFA) over 15 min to afford the title compound (100 mg, 81%). LC-MS m/z 302 (M+H)⁺, 0.75 min (ret time). ¹H NMR (400 MHz, MeOD) δ ppm 1.03 (3H, t, J=7.4 Hz) 1.58 (3H, d, J=7.0 Hz) 1.65-1.79 (2H, m) 4.00 (1H, q, J=7.0 Hz) 4.52 (1H, q, J=6.6 Hz) 4.82 (2H, s) 4.99 (2H, s) 6.47 (1H, dd, J=15.3, 1.2 Hz) 6.80 (1H, dd, J=15.2, 6.4 Hz) 7.31-7.38 (4H, m).

### Example 41

N¹-{(1S,2E)-4-[(1R,8S)-11-azatricyclo[6.2.1.0^{2,7}]undeca-2,4,6-trien-11-yl]-1-[(1S)-1-methylpropyl]-4-oxo-2-buten-1-yl}-L-alaninamide trifluoroacetate

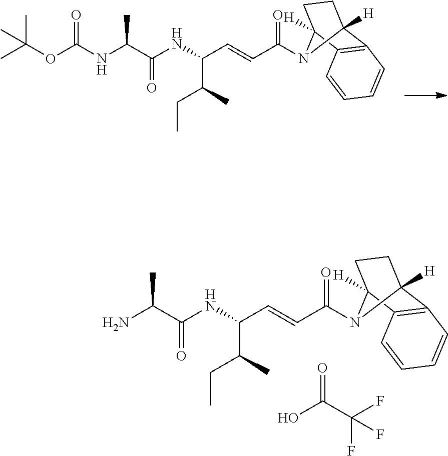

To a solution of 1,1-dimethylethyl[(1S)-2-({(1S,2E)-4-[(1R,8S)-11-azatricyclo[6.2.1.0^{2,7}]undeca-2,4,6-trien-11-yl]-1-[(1S)-1-methylpropyl]-4-oxo-2-buten-1-yl}amino)-1-methyl-2-oxoethyl]carbamate (140 mg, 0.307 mmol) in CH₂Cl₂ (4.0 mL) was added TFA (0.095 mL, 1.229 mmol). The reaction mixture was stirred at RT overnight. The solvent was removed and the residue was purified via reverse phase HPLC(YMC C18 S-5 μm/12 nm 75×30 mm preparatory column), eluting at 35 mL/min with a linear gradient running from 10% CH₃CN/H₂O (0.1% TFA) to 80% CH₃CN/H₂O (0.1% TFA) over 15 min to afford the title compound (55 mg, 38%). LC-MS m/z 356 (M+H)⁺, 0.92 min (ret time). ¹H NMR (400 MHz, MeOD) δ ppm 0.90 (1H, s) 0.97 (1H, br. s.) 0.94 (4H, dd, J=11.2, 4.1 Hz) 1.20 (1H, dd, J=12.8, 5.3 Hz) 1.37 (1H, d, J=7.8 Hz) 1.43 (1H, d, J=8.0 Hz) 1.52 (4H, dd, J=17.6, 7.3 Hz) 1.66 (1H, br. s.) 1.68 (1H, ddd, J=4.4, 2.1, 2.0 Hz) 2.13 (1H, t, J=8.3 Hz) 3.97 (1H, dd, J=11.5, 7.0 Hz) 4.44 (1H, t, J=7.3 Hz) 5.50 (1H, br. s.) 5.56 (1H, d, J=3.3 Hz) 6.42 (1H, d, J=15.8 Hz) 6.66 (1H, ddd, J=15.2, 7.6, 2.3 Hz) 7.20 (2H, dd, J=5.3, 3.0 Hz) 7.32 (2H, td, J=8.3, 3.3 Hz).

### Example 42

### N¹-{(1S,2E)-4-(1,3-dihydro-2H-isoindol-2-yl)-1-[(1S)-1-methylpropyl]-4-oxo-2-buten-1-yl}-L-alaninamide trifluoroacetate

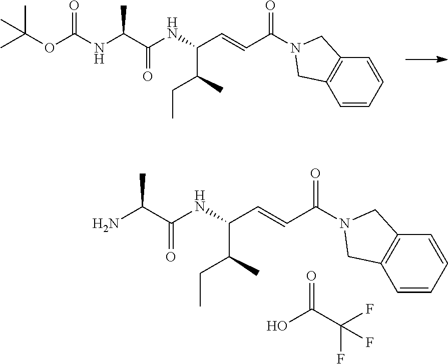

To a solution of 1,1-dimethylethyl[(1S)-2-({(1S,2E)-4-(1,3-dihydro-2H-isoindol-2-yl)-1-[(1S)-1-methylpropyl]-4-oxo-2-buten-1-yl}amino)-1-methyl-2-oxoethyl]carbamate (120 mg, 0.279 mmol) in CH₂Cl₂ (4.0 mL) was added TFA (0.172 mL, 2.235 mmol). The reaction mixture was stirred at RT overnight. The solvent was removed and the residue was purified via reverse phase HPLC(YMC C18 S-5 μm/12 nm 75×30 mm preparatory column), eluting at 35 mL/min with a linear gradient running from 10% CH₃CN/H₂O (0.1% TFA) to 80% CH₃CN/H₂O (0.1% TFA) over 15 min to afford the title compound (90 mg, 73%). LC-MS m/z 330 (M+H)⁺, 0.69 min (ret time). ¹H NMR (400 MHz, MeOD) δ ppm 0.96-1.04 (6H, m) 1.25 (1H, dd, J=8.0, 5.8 Hz) 1.55-1.62 (1H, m) 1.57 (3H, d, J=7.0 Hz) 1.70-1.77 (1 H, m) 4.02 (1H, q, J=7.0 Hz) 4.52 (1H, t, J=6.4 Hz) 4.83 (2H, s) 4.98 (2H, d, J=4.3 Hz) 6.48 (1H, dd, J=15.2, 1.1 Hz) 6.82 (1H, dd, J=15.2, 7.1 Hz) 7.31-7.38 (4H, m).

### Example 43

### (2E,4S)-4-(L-alanylamino)-6-methyl-N-[4-(methyloxy)phenyl]-2-heptenamide trifluoroacetate

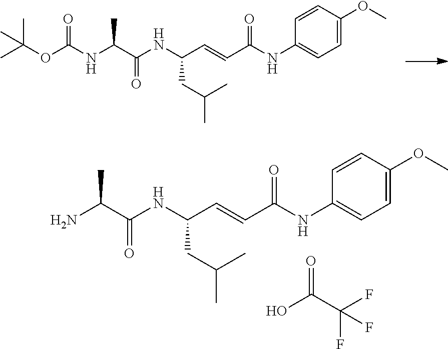

To a solution of 1,1-dimethylethyl((1S)-1-methyl-2-{[(1S,2E)-4-{[4-(methyloxy)phenyl]amino}-1-(2-methylpropyl)-4-oxo-2-buten-1-yl]amino}-2-oxoethyl)carbamate (130 mg, 0.300 mmol) in CH₂Cl₂ (4.0 mL) was added TFA (0.092 mL, 1.199 mmol). The reaction mixture was stirred at RT overnight. The solvent was removed and the residue was purified via reverse phase HPLC(YMC C18 S-5 μm/12 nm 75×30 mm preparatory column), eluting at 35 mL/min with a linear gradient running from 10% CH₃CN/H₂O (0.1% TFA) to 80% CH₃CN/H₂O (0.1% TFA) over 15 min to afford the title compound (90 mg, 67%). LC-MS m/z 334 (M+H)⁺, 0.92 min (ret time). ¹H NMR (400 MHz, MeOD) δ ppm 0.99 (6H, t, J=6.0 Hz) 1.52 (1H, d, J=2.3 Hz) 1.57 (3H, d, J=7.0 Hz) 1.53-1.59 (1H, m) 1.72 (1H, dd, J=7.9, 6.6 Hz) 3.79 (3H, s) 3.96 (1H, q, J=7.0 Hz) 4.68 (1H, t, J=5.5 Hz) 6.19 (1H, dd, J=15.3, 1.2 Hz) 6.76 (1H, dd, J=15.3, 6.3 Hz) 6.90 (1H, q, J=5.5 Hz) 6.90 (1H, d, J=9.3 Hz) 7.52 (1H, q, J=5.5 Hz) 7.52 (1H, d, J=9.0 Hz).

### Example 44

### N¹-[(1S,2E)-4-(1,3-dihydro-2H-isoindol-2-yl)-1-(2-methylpropyl)-4-oxo-2-buten-1-yl]-L-alaninamide trifluoroacetate

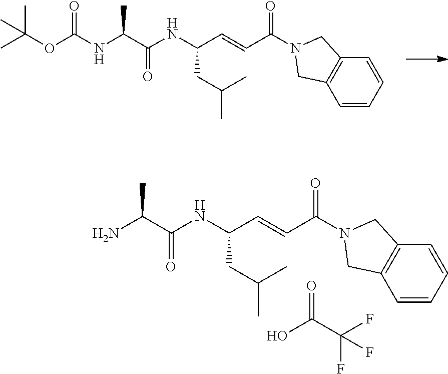

To a solution of 1,1-dimethylethyl((1S)-2-{[(1S,2E)-4-(1,3-dihydro-2H-isoindol-2-yl)-1-(2-methylpropyl)-4-oxo-2-buten-1-yl]amino}-1-methyl-2-oxoethyl)carbamate (120 mg, 0.279 mmol) in CH₂Cl₂ (4.0 mL) was added TFA (0.172 mL, 2.235 mmol). The reaction mixture was stirred at RT overnight. The solvent was removed and the residue was purified via reverse phase HPLC(YMC C18 S-5 μm/12 nm 75×30 mm preparatory column), eluting at 35 mL/min with a linear gradient running from 10% CH₃CN/H₂O (0.1% TFA) to 80% CH₃CN/H₂O (0.1% TFA) over 15 min to afford the title compound (100 mg, 81%). LC-MS m/z 330 (M+H)⁺, 0.89 min (ret time). ¹H NMR (400 MHz, MeOD) δ ppm 1.00 (6H, t, J=6.5 Hz) 1.50-1.60 (5H, m) 1.65-1.80 (1H, m) 3.97 (1H, q, J=7.0 Hz) 4.66-4.73 (1H, m) 4.75 (1H, s) 4.83 (2H, s) 4.99 (2H, d, J=1.2 Hz) 6.46 (1H, dd, J=15.2, 1.1 Hz) 6.79 (1H, dd, J=15.1, 6.5 Hz) 7.31-7.38 (5H, m).

### Example 45

### (2E,4S)—N-[4-(methyloxy)phenyl]-6-phenyl-4-{[3-(2-thienyl)-L-alanyl]amino}-2-hexenamide hydrochloride

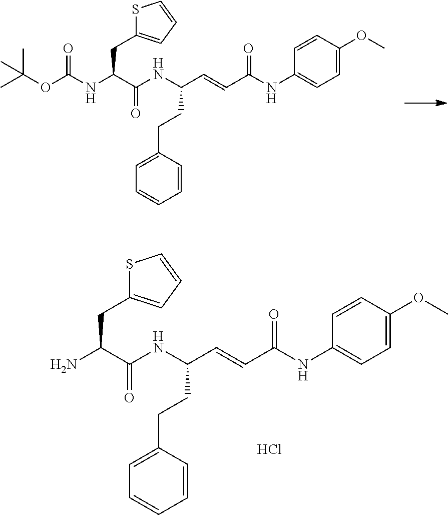

A solution of 1,1-dimethylethyl[(1S)-2-{[(1S,2E)-4-{[4-(methyloxy)phenyl]amino}-4-oxo-1-(2-phenylethyl)-2-buten-1-yl]amino}-2-oxo-1-(2-thienylmethyl)ethyl]carbamate (411 mg, 0.73 mmol) in HCl (4 M solution in 1,4-dioxane, 3.0 mL, 12.0 mmol) was stirred at RT for 20 min. The solvent was removed and the residue was purified via reverse phase HPLC using an MDAP equipped with a SunFire C18 column with a linear gradient running from 20% CH₃CN/H₂O (0.1% TFA) to 50% CH₃CN/H₂O (0.1% TFA) to afford the title compound (56 mg, 15%) as a white solid. LC-MS m/z 464 (M+H)⁺, 1.11 min (ret time). ¹H NMR (400 MHz, DMSO-d₆) δ ppm 10.02 (1H, s), 8.88 (1H, d, J=7.78 Hz), 8.32 (2H, br. s.), 7.58 (2H, m, J=9.03 Hz), 7.43 (1H, m, J=3.26 Hz), 7.30 (2H, m), 7.20-7.27 (3H, m), 6.99 (2H, d, J=3.26 Hz), 6.90 (2H, m, J=9.29 Hz), 6.64 (1H, dd, J=15.31, 6.27 Hz), 6.18 (1 H, d, J=15.31 Hz), 4.42 (1H, m), 4.05 (1H, t, J=6.78 Hz), 3.73 (3H, s), 3.43 (1H, m), 3.31 (1H, m), 2.65 (2H, m), 1.87 (2H, m).

### Example 46

### (2E,4S)-4-{[(2S)-2-aminobutanoyl]amino}-N-[4-(methyloxy)phenyl]-6-phenyl-2-hexenamide hydrochloride

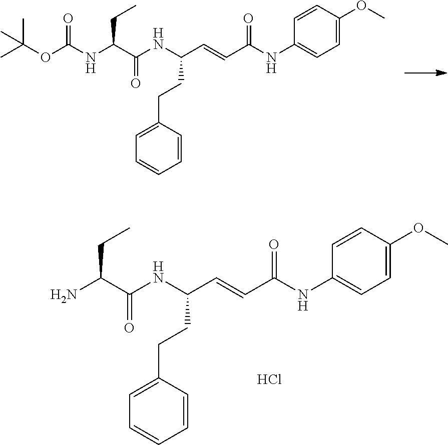

A solution of 1,1-dimethylethyl[(1S)-1-({[(1S,2E)-4-{[4-(methyloxy)phenyl]amino}-4-oxo-1-(2-phenylethyl)-2-buten-1-yl]amino}carbonyl)propyl]carbamate (426 mg, 0.87 mmol) in HCl (4 M solution in 1,4-dioxane, 3.0 mL, 12.0 mmol) was stirred at RT for 20 min. The solvent was removed and the residue was purified via reverse phase HPLC using an MDAP equipped with a SunFire C18 column with a linear gradient running from 10% CH₃CN/H₂O (0.1% TFA) to 35% CH₃CN/H₂O (0.1% TFA) to afford the title compound (95 mg, 25%) as a white solid. LC-MS m/z 396 (M+H)⁺, 1.00 min (ret time). ¹H NMR (400 MHz, DMSO-d₆) δ ppm 9.97 (1H, s), 8.71 (1H, d, J=8.03 Hz), 8.14 (2H, br. s.), 7.49 (2H, m, J=9.03 Hz), 7.21-7.13 (5H, m), 6.81 (2H, m, J=9.03 Hz), 6.60 (1H, dd, J=15.31, 6.27 Hz), 6.11 (1H, d, J=15.31 Hz), 4.37 (1H, m), 3.71 (1H, t, J=6.15 Hz), 3.64 (3H, s), 2.58 (2 H, m), 1.78 (4H, m), 0.85 (3H, t, J=7.40 Hz).

### Example 47

### (2E,4S)-6-phenyl-N-propyl-4-{[3-(2-thienyl)-L-alanyl]amino}-2-hexenamide trifluoroacetate

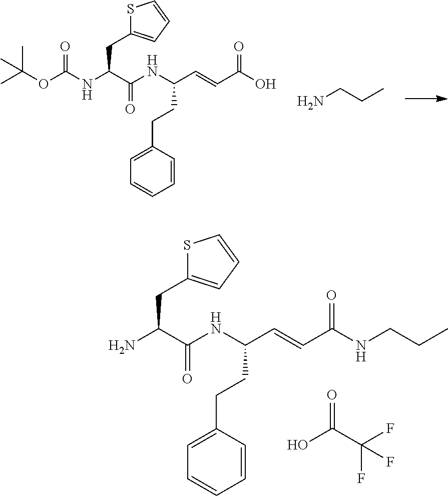

A solution of (2E,4S)-4-{[N-{[(1,1-dimethylethyl)oxy]carbonyl}-3-(2-thienyl)-L-alanyl]amino}-6-phenyl-2-hexenoic acid (78.6 mg, 0.171 mmol), HATU (87 mg, 0.222 mmol), and DIPEA (0.15 mL, 0.857 mmol) in DMF (1.0 mL) was stirred for 25 min in a vial. Propylamine (0.02 mL, 0.242 mmol) was added and stirring continued for 45 min. The reaction mixture was concentrated and treated with TFA (0.3 mL, 3.89 mmol) in CH₂Cl₂ (2.0 mL). The reaction mixture was stirred for 6 h at RT. The reaction mixture was then concentrated, dissolved in DMSO (2.0 mL), filtered through a 0.45 μm Acrodisc® filter, and purified by reverse phase HPLC(YMC C18 S-5 μm/12 nm 50×20 mm preparatory column), eluting at 20 mL/min with a linear gradient running from 10% CH₃CN/H₂O (0.1% TFA) to 80% CH₃CN/H₂O (0.1% TFA) over 15 min to afford the title compound (62.4 mg, 71%). LC-MS m/z 400 (M+H)⁺, 0.81 min (ret time). ¹H NMR (400 MHz, MeOD) δ ppm 0.96 (t, J=7.4 Hz, 3H) 1.54-1.61 (m, 2H) 1.92 (q, J=7.5 Hz, 2H) 2.65-2.75 (m, 2H) 3.23 (t, J=7.0 Hz, 2H) 3.32-3.39 (m, 1H) 3.47 (q, J=6.3 Hz, 1H) 4.12 (t, J=7.0 Hz, 1H) 4.52 (q, J=6.8 Hz, 1H) 5.98 (d, J=15.3 Hz, 1H) 6.59 (dd, J=15.6, 6.8 Hz, 1H) 6.98-7.06 (m, 2H) 7.15-7.23 (m, 3H) 7.28 (t, J=7.4 Hz, 2H) 7.35 (dd, J=4.5, 1.5 Hz, 1H).

### Example 48

### (2E,4S)-4-{[(2S)-2-aminobutanoyl]amino}-N-[5-(1-methylcyclobutyl)-1,3,4-thiadiazol-2-yl]-6-phenyl-2-hexenamide trifluoroacetate

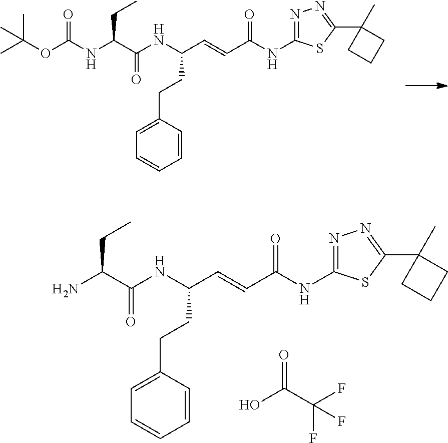

To a solution of 1,1-dimethylethyl[(15)-1-({[(1S,2E)-4-{[5-(1-methylcyclobutyl)-1,3,4-thiadiazol-2-yl]amino}-4-oxo-1-(2-phenylethyl)-2-buten-1-yl]amino}carbonyl)propyl]-carbamate (140 mg, 0.258 mmol) in CH₂Cl₂ (4.0 mL) was added TFA (0.080 mL, 1.034 mmol). The reaction mixture was stirred at RT overnight. The solvent was removed and the residue was purified via reverse phase HPLC(YMC C18 S-5 μm/12 nm 75×30 mm preparatory column), eluting at 35 mL/min with a linear gradient running from 10% CH₃CN/H₂O (0.1% TFA) to 80% CH₃CN/H₂O (0.1% TFA) over 15 min to afford the title compound (70 mg, 49%). LC-MS m/z 442 (M+H)⁺, 1.00 min (ret time). ¹H NMR (400 MHz, MeOD) δ ppm 1.09 (3H, q, J=7.3 Hz) 1.66 (3H, s) 1.90-2.10 (5H, m) 2.15-2.25 (3 H, m) 2.57-2.65 (2H, m) 2.70-2.77 (2H, m) 3.87 (1H, t, J=6.4 Hz) 4.64 (1H, q, J=6.5 Hz) 6.31 (1H, dd, J=15.4, 1.1 Hz) 7.02 (1H, dd, J=15.4, 6.4 Hz) 7.19-7.34 (5H, m).

### Example 49

### (2S)—N-[(1S,2E)-4-{[4-(methyloxy)phenyl]amino}-4-oxo-1-(2-phenylethyl)-2-buten-1-yl]-2-azetidinecarboxamide trifluoroacetate

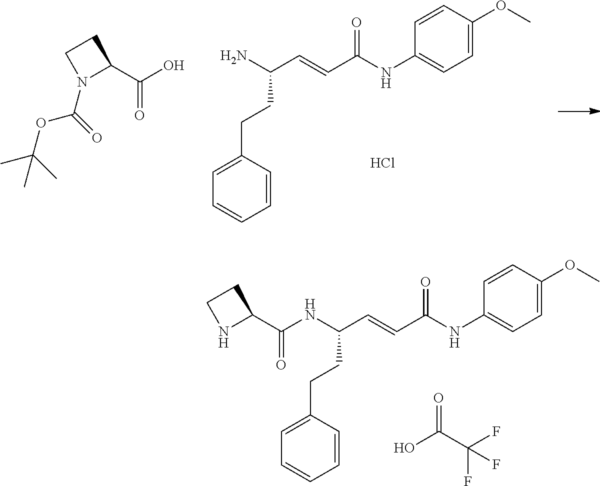

A mixture of (2E,4S)-4-amino-N-[4-(methyloxy)phenyl]-6-phenyl-2-hexenamide hydrochloride (120 mg, 0.283 mmol), (2S)-1-{[(1,1-dimethylethyl)oxy]carbonyl}-2-azetidinecarboxylic acid (56.9 mg, 0.283 mmol), BOP reagent (125 mg, 0.283 mmol), and DIPEA (0.148 mL, 0.848 mmol) in DMF (2.0 mL) was stirred at RT for 1 h. The reaction mixture was diluted with water and extracted with EtOAc. The organic layer was washed with brine and water, dried over Na₂SO₄, filtered, and concentrated in vacuo. The residue was dissolved in CH₂Cl₂ (3.0 mL) and treated with TFA (0.4 mL, 5.19 mmol). The reaction mixture was stirred for 1 h at RT. The reaction mixture was then concentrated in vacuo, dissolved in MeOH, and purified by reverse phase HPLC (SunFire C18 preparatory column), eluting with a linear gradient running from 10% CH₃CN/H₂O (0.1% TFA) to 60% CH₃CN/H₂O (0.1% TFA) to afford the title compound (93 mg, 64%) as a white solid. LC-MS m/z 394 (M+H)⁺, 0.74 min (ret time). ¹H NMR (400 MHz, MeOD) δ ppm 7.47-7.58 (m, 2H), 7.16-7.35 (m, 5H), 6.86-6.96 (m, 2H), 6.79 (dd, J=15.4, 6.4 Hz, 1H), 6.17-6.28 (m, 1H), 5.02 (dd, J=9.3, 7.8 Hz, 1H), 4.60 (q, J=6.5 Hz, 1H), 4.16 (q, J=9.4 Hz, 1H), 4.00 (td, J=9.9, 6.3 Hz, 1H), 3.79 (s, 3H), 2.83-2.98 (m, J=12.2, 9.5, 9.5, 6.3 Hz, 1H), 2.73 (td, J=7.7, 3.3 Hz, 2H), 2.55-2.69 (m, J=12.1, 9.7, 7.8, 7.8 Hz, 1H), 1.90-2.09 (m, 2H).

### Example 50

### (2E,4S)-4-{[(2S)-2-amino-2-cyclopentylacetyl]amino}-N-[4-(methyloxy)phenyl]-6-phenyl-2-hexenamide trifluoroacetate

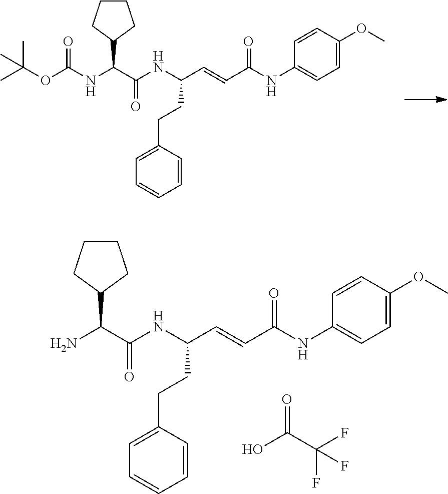

To a solution of 1,1-dimethylethyl((1S)-1-cyclopentyl-2-{[(1S,2E)-4-{[4-(methyloxy)phenyl]amino}-4-oxo-1-(2-phenylethyl)-2-buten-1-yl]amino}-2-oxoethyl)-carbamate (70 mg, 0.131 mmol) in CH₂Cl₂ (2.0 mL) was added TFA (0.2 mL, 2.60 mmol). The reaction mixture was stirred for 2 h at RT. The reaction mixture was then concentrated in vacuo, dissolved in MeOH, and purified by reverse phase HPLC (SunFire C18 preparatory column), eluting with a linear gradient running from 10% CH₃CN/H₂O (0.1% TFA) to 60% CH₃CN/H₂O (0.1% TFA) to afford the title compound (48 mg, 66%) as a white solid. LC-MS m/z 436 (M+H)⁺, 0.84 min (ret time). ¹H NMR (400 MHz, MeOD) δ ppm 7.48-7.57 (m, 2H), 7.15-7.37 (m, 5H), 6.85-6.96 (m, 2H), 6.75 (dd, J=15.3, 7.8 Hz, 1H), 6.25 (d, J=15.3 Hz, 1H), 4.59 (q, J=7.1 Hz, 1H), 3.80 (s, 3H), 3.67 (d, J=8.3 Hz, 1H), 2.73 (td, J=7.8, 4.0 Hz, 2H), 2.20-2.36 (m, 1H), 1.96-2.08 (m, 2H), 1.85-1.94 (m, 1H), 1.71-1.83 (m, 3H), 1.57-1.71 (m, 2H), 1.31-1.53 (m, 2H).

### Example 51

### (2E,4S)—N-[4-(methyloxy)phenyl]-6-phenyl-4-(L-valylamino)-2-hexenamide trifluoroacetate

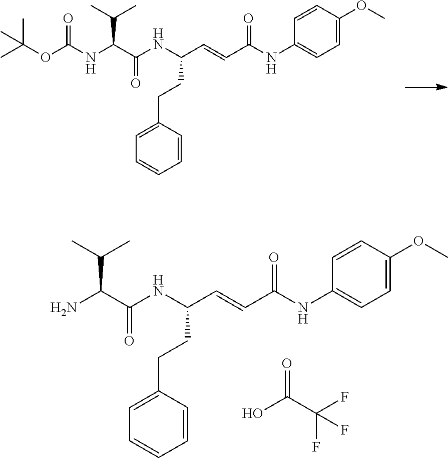

To a solution of 1,1-dimethylethyl[(15)-2-methyl-1-({[(1S,2E)-4-{[4-(methyloxy)-phenyl]amino}-4-oxo-1-(2-phenylethyl)-2-buten-1-yl]amino}carbonyl)propyl]carbamate (85 mg, 0.167 mmol) in CH₂Cl₂ (2.0 mL) was added TFA (0.3 mL, 3.89 mmol). The reaction mixture was stirred for 1 h at RT. The reaction mixture was then concentrated in vacuo, dissolved in MeOH, and purified by reverse phase HPLC (SunFire C18 preparatory column), eluting with a linear gradient running from 10% CH₃CN/H₂O (0.1% TFA) to 60% CH₃CN/H₂O (0.1% TFA) to afford the title compound (61 mg, 69%) as a colorless solid. LC-MS m/z 410 (M+H)⁺, 0.80 min (ret time). ¹H NMR (400 MHz, MeOD) δ ppm 7.48-7.58 (m, 2H), 7.15-7.35 (m, 5H), 6.86-6.95 (m, 2H), 6.78 (dd, J=15.3, 7.3 Hz, 1H), 6.19-6.29 (m, 1H), 4.61 (q, J=7.3 Hz, 1H), 3.79 (s, 3H), 3.70 (d, J=5.8 Hz, 1H), 2.73 (td, J=7.8, 4.5 Hz, 2H), 2.25 (dd, J=12.7, 6.9 Hz, 1H), 1.91-2.06 (m, 2H), 1.11 (d, J=6.8 Hz, 3H), 1.06 (d, J=6.8 Hz, 3H).

### Example 52

### (2S)—N-[(1S,2E)-4-(2,3-dihydro-1H-indol-1-yl)-1-ethyl-4-oxo-2-buten-1-yl]-2-azetidinecarboxamide trifluoroacetate

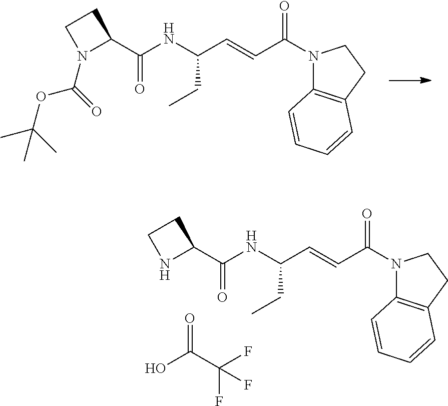

To a solution of 1,1-dimethylethyl (2S)-2-({[(1S,2E)-4-(2,3-dihydro-1H-indol-1-yl)-1-ethyl-4-oxo-2-buten-1-yl]amino}carbonyl)-1-azetidinecarboxylate (900 mg, 2.176 mmol) in CH₂Cl₂ (30.0 mL) was added TFA (1.34 mL, 17.41 mmol). The reaction mixture was stirred overnight at RT. The reaction mixture was then concentrated in vacuo and purified by reverse phase HPLC(YMC C18 S-15 μm/12 nm 75×30 mm preparatory column), eluting with a linear gradient running from 10% CH₃CN/H₂O (0.1% TFA) to 80% CH₃CN/H₂O (0.1% TFA) over 15 min. to afford the title compound (470 mg, 51%). LC-MS m/z 314 (M+H)⁺, 0.61 min (ret time). ¹H NMR (400 MHz, MeOD) δ ppm 8.56 (d, J=7.5 Hz, 1H), 8.17 (d, J=7.8 Hz, 1H), 7.25 (d, J=7.0 Hz, 1H), 7.19 (t, J=7.7 Hz, 1H), 7.07 (t, J=7.7 Hz, 1H), 6.82 (dd, J=15.1, 6.5 Hz, 1H), 6.52 (d, J=15.3 Hz, 1H), 5.07 (t, J=9.0 Hz, 1H), 4.51-4.60 (m, 1H), 4.23 (t, J=7.9 Hz, 2H), 4.15 (q, J=9.3 Hz, 1H), 3.99 (td, J=10.0, 6.1 Hz, 1H), 3.23 (t, J=8.0 Hz, 2H), 2.80-2.97 (m, 1H), 2.46-2.63 (m, 1H), 1.63-1.85 (m, 2H), 1.02 (t, J=7.4 Hz, 3H).

### Example 53

### (2S)—N-[(1S,2E)-4-(2,3-dihydro-1H-indol-1-yl)-1-(2-methylpropyl)-4-oxo-2-buten-1-yl]-2-azetidinecarboxamide trifluoroacetate

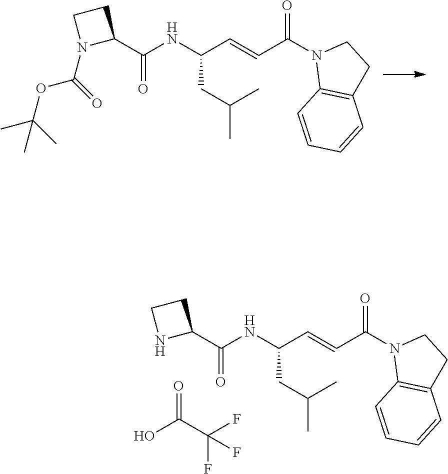

To a solution of 1,1-dimethylethyl (2S)-2-({[(1S,2E)-4-(2,3-dihydro-1H-indol-1-yl)-1-(2-methylpropyl)-4-oxo-2-buten-1-yl]amino}carbonyl)-1-azetidinecarboxylate (174 mg, 0.394 mmol) in CH₂Cl₂ (4.0 mL) was added TFA (0.486 mL, 6.30 mmol). The reaction mixture was stirred overnight at RT. The reaction mixture was then concentrated in vacuo and purified by reverse phase HPLC(YMC C18 S-15 μm/12 nm 75×30 mm preparatory column), eluting with a linear gradient running from 10% CH₃CN/H₂O (0.1% TFA) to 80% CH₃CN/H₂O (0.1% TFA) over 15 min. to afford the title compound (57 mg, 32%). LC-MS m/z 342 (M+H)⁺, 0.81 min (ret time). ¹H NMR (400 MHz, MeOD) δ ppm 8.17 (d, J=8.0 Hz, 1H), 7.24 (d, J=7.0 Hz, 1H), 7.19 (t, J=7.5 Hz, 1H), 7.10-7.02 (m, 1H), 6.82 (dd, J=6.5, 15.1 Hz, 1H), 6.50 (d, J=15.1 Hz, 1H), 5.07 (dd, J=7.8, 9.3 Hz, 1H), 4.73 (d, J=8.0 Hz, 1H), 4.24-4.17 (m, 3H), 4.15 (s, 1H), 4.00 (br. s., 1H), 3.99 (d, J=6.0 Hz, 1H), 3.21 (t, J=8.3 Hz, 2H), 2.88 (d, J=2.5 Hz, 1H), 2.60-2.53 (m, 1H), 1.74 (br. s., 1H), 1.72 (d, J=6.5 Hz, 1H), 1.59-1.51 (m, 2H), 0.99 (t, J=6.3 Hz, 6H).

### Example 54

### (2S)—N-[(1S,2E)-1-(cyclopropylmethyl)-4-(2,3-dihydro-1H-indol-1-yl)-4-oxo-2-buten-1-yl]-2-azetidinecarboxamide trifluoroacetate

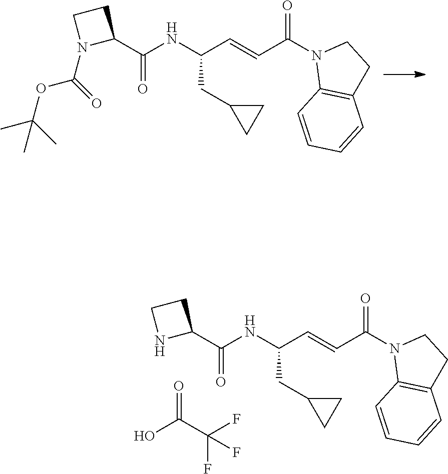

To a solution of 1,1-dimethylethyl (2S)-2-({[(1S,2E)-1-(cyclopropylmethyl)-4-(2,3-dihydro-1H-indol-1-yl)-4-oxo-2-buten-1-yl]amino}carbonyl)-1-azetidinecarboxylate (181 mg, 0.412 mmol) in CH₂Cl₂ (4.0 mL) was added TFA (0.508 mL, 6.59 mmol). The reaction mixture was stirred overnight at RT. The reaction mixture was then concentrated in vacuo and purified by reverse phase HPLC(YMC C18 S-15 μm/12 nm 75×30 mm preparatory column), eluting with a linear gradient running from 10% CH₃CN/H₂O (0.1% TFA) to 80% CH₃CN/H₂O (0.1% TFA) over 15 min. to afford the title compound (97 mg, 52%). LC-MS m/z 340 (M+H)⁺, 0.74 min (ret time). ¹H NMR (400 MHz, MeOD) δ ppm 8.00 (d, J=7.8 Hz, 1H), 7.08 (d, J=7.0 Hz, 1H), 7.02 (t, J=7.7 Hz, 1H), 6.85-6.93 (m, 1H), 6.72 (dd, J=15.2, 6.4 Hz, 1H), 6.35 (d, J=15.1 Hz, 1H), 4.90 (dd, J=9.3, 7.8 Hz, 1H), 4.57 (q, J=6.4 Hz, 1H), 3.94-4.11 (m, 3H), 3.82 (td, J=10.0, 6.3 Hz, 1H), 3.05 (t, J=8.2 Hz, 2H), 2.69-2.75 (m, 1H), 2.38-2.45 (m, 1H), 1.43 (td, J=7.0, 3.8 Hz, 2H), 0.57-0.71 (m, 1H), 0.31-0.38 (m, 2H), 0.00 (dt, J=8.7, 4.5 Hz, 2H).

### Example 55

### (4S)—N-[(1S,2E)-4-(2,3-dihydro-1H-indol-1-yl)-1-ethyl-4-oxo-2-buten-1-yl]-4-fluoro-L-prolinamide trifluoroacetate

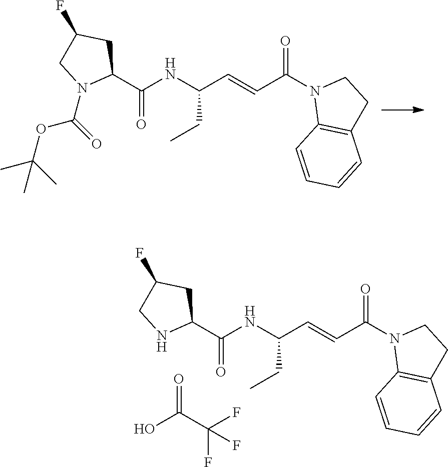

To a solution of 1,1-dimethylethyl (2S,4S)-2-({[(1S,2E)-4-(2,3-dihydro-1H-indol-1-yl)-1-ethyl-4-oxo-2-buten-1-yl]amino}carbonyl)-4-fluoro-1-pyrrolidinecarboxylate (60 mg, 0.135 mmol) in CH₂Cl₂ (6.0 mL) was added TFA (0.083 mL, 1.077 mmol). The reaction mixture was stirred overnight at RT. The reaction mixture was then concentrated in vacuo and purified by reverse phase HPLC(YMC C18 S-15 μm/12 nm 75×30 mm preparatory column), eluting with a linear gradient running from 10% CH₃CN/H₂O (0.1% TFA) to 80% CH₃CN/H₂O (0.1% TFA) over 15 min. to afford the title compound (30 mg, 58%). LC-MS m/z 346 (M+H)⁺, 0.70 min (ret time). ¹H NMR (400 MHz, MeOD) δ ppm 8.61 (d, J=7.3 Hz, 1H), 8.17 (d, J=7.8 Hz, 1H), 7.26 (d, J=7.3 Hz, 1H), 7.19 (t, J=7.7 Hz, 1H), 7.07 (td, J=7.4, 1.0 Hz, 1H), 6.85 (dd, J=15.1, 5.8 Hz, 1H), 6.47 (d, J=15.1 Hz, 1H), 5.49 (br. d., J=51.9 Hz, 1H), 4.52-4.60 (m, 2H), 4.09-4.28 (m, 2H), 3.80 (ddd, J=19.3, 13.6, 1.8 Hz, 1H), 3.57 (ddd, J=35.6, 13.5, 3.5 Hz, 1H), 3.22 (t, J=8.2 Hz, 2H), 2.68-2.89 (m, 1H), 2.53-2.67 (m, 1H), 1.64-1.86 (m, 2H), 1.05 (t, J=7.4 Hz, 3H).

### Example 56

### (2S)—N-[(1S,2E)-4-(2,3-dihydro-1H-indol-1-yl)-1-ethyl-4-oxo-2-buten-1-yl]-2-piperidinecarboxamide trifluoroacetate

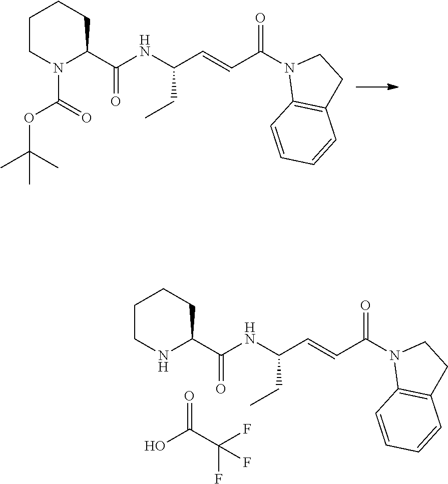

To a solution of 1,1-dimethylethyl (2S)-2-({[(1S,2E)-4-(2,3-dihydro-1H-indol-1-yl)-1-ethyl-4-oxo-2-buten-1-yl]amino}carbonyl)-1-piperidinecarboxylate (200 mg, 0.453 mmol) in CH₂Cl₂ (5.0 mL) was added TFA (0.279 mL, 3.62 mmol). The reaction mixture was stirred overnight at RT. The reaction mixture was then concentrated in vacuo and purified by reverse phase HPLC(YMC C18 S-15 μm/12 nm 75×30 mm preparatory column), eluting with a linear gradient running from 10% CH₃CN/H₂O (0.1% TFA) to 80% CH₃CN/H₂O (0.1% TFA) over 15 min. to afford the title compound (110 mg, 53%). LC-MS m/z 342 (M+H)⁺, 0.70 min (ret time). ¹H NMR (400 MHz, MeOD) δ ppm 8.48 (d, J=7.8 Hz, 1H), 8.17 (d, J=7.8 Hz, 1H), 7.26 (d, J=7.0 Hz, 1H), 7.20 (t, J=7.7 Hz, 1H), 7.04-7.11 (m, 1H), 6.82 (dd, J=15.1, 6.3 Hz, 1H), 6.48 (d, J=15.3 Hz, 1H), 4.54 (t, J=6.9 Hz, 1H), 4.19-4.27 (m, 2H), 3.84 (dd, J=11.7, 2.9 Hz, 1H), 3.43 (br. d, J=9.3 Hz, 1H), 3.21-3.28 (m, 2H), 3.02-3.10 (m, 1H), 2.23-2.31 (m, 1H), 1.88-2.02 (m, 2H), 1.63-1.82 (m, 5H), 1.03 (t, J=7.4 Hz, 3H).

### Example 57

### (2S)—N-[(1S,2E)-4-(2,3-dihydro-1H-indol-1-yl)-1-ethyl-4-oxo-2-buten-1-yl]-2-piperidinecarboxamide sulfate

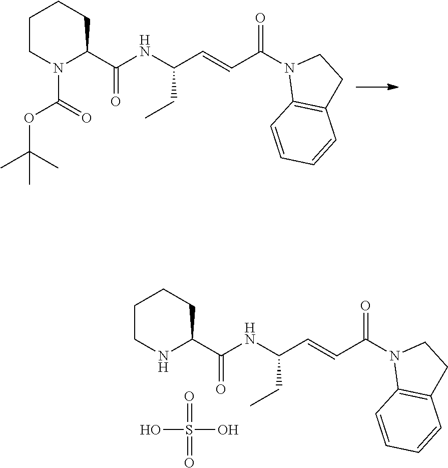

To a solution of 1,1-dimethylethyl (2S)-2-({[(1S,2E)-4-(2,3-dihydro-1H-indol-1-yl)-1-ethyl-4-oxo-2-buten-1-yl]amino}carbonyl)-1-piperidinecarboxylate (330 mg, 0.747 mmol) in 1,4-dioxane (7.0 mL) at RT was added 10% aq. H₂SO₄ (3.98 mL, 7.47 mmol). The reaction mixture was then heated to 50° C. overnight. The reaction mixture was then concentrated in vacuo and purified by reverse phase HPLC (10-40% CH₃CN/H₂O) to afford the title compound (212 mg, 65%) as a brown solid. LC-MS m/z 342 (M+H)⁺, 0.65 min (ret time). ¹H NMR (400 MHz, MeOD) δ ppm 8.17 (d, J=6.3 Hz, 1H), 7.26 (d, J=7.3 Hz, 1H), 7.19 (t, J=7.7 Hz, 1H), 7.07 (t, J=7.7 Hz, 1H), 6.84 (dd, J=15.1, 6.3 Hz, 1H), 6.50 (d, J=15.3 Hz, 1H), 4.49-4.55 (m, 1H), 4.15-4.32 (m, 2H), 3.86-3.98 (m, 1H), 3.39-3.44 (m, 1H), 3.19-3.30 (m, 2H), 3.05-3.19 (m, 1H), 2.24 (br d, J=12.8 Hz, 1H), 1.82-1.99 (m, 2H), 1.60-1.82 (m, 5H), 1.04 (t, J=7.4 Hz, 3H).

### Example 58

### (2S)—N-[(1S,2E)-4-(2,3-dihydro-1H-indol-1-yl)-1-(2-methylpropyl)-4-oxo-2-buten-1-yl]-2-piperidinecarboxamide trifluoroacetate

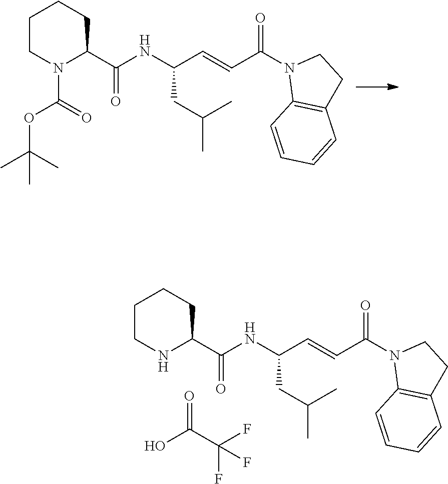

To a solution of 1,1-dimethylethyl (2S)-2-({[(1S,2E)-4-(2,3-dihydro-1H-indol-1-yl)-1-(2-methylpropyl)-4-oxo-2-buten-1-yl]amino}carbonyl)-1-piperidinecarboxylate (143 mg, 0.305 mmol) in CH₂Cl₂ (5.0 mL) was added TFA (0.188 mL, 2.436 mmol). The reaction mixture was stirred overnight at RT. The reaction mixture was then concentrated in vacuo and purified by reverse phase HPLC(YMC C18 S-15 μm/12 nm 75×30 mm preparatory column), eluting with a linear gradient running from 10% CH₃CN/H₂O (0.1% TFA) to 80% CH₃CN/H₂O (0.1% TFA) over 15 min. to afford the title compound (100 mg, 68%). LC-MS m/z 370 (M+H)⁺, 0.82 min (ret time). ¹H NMR (400 MHz, MeOD) δ ppm 8.47 (d, J=8.0 Hz, 1H), 8.17 (d, J=7.8 Hz, 1H), 7.26 (d, J=7.3 Hz, 1H), 7.20 (t, J=7.7 Hz, 1H), 7.07 (t, J=7.4 Hz, 1H), 6.81 (dd, J=15.1, 6.3 Hz, 1H), 6.47 (d, J=15.1 Hz, 1H), 4.72-4.76 (m, 1H), 4.19-4.26 (m, 2H), 3.81-3.86 (m, 1H), 3.39-3.46 (m, 1H), 3.24 (t, J=8.3 Hz, 2H), 3.03-3.10 (m, 1H), 2.24-2.31 (m, 1H), 1.87-2.02 (m, 2H), 1.65-1.80 (m, 4H), 1.49-1.63 (m, 2H), 1.00 (t, J=6.8 Hz, 6H).

### Example 59

### (2S)—N-[(1S,2E)-4-(2,3-dihydro-1H-indol-1-yl)-1-(2-methylpropyl)-4-oxo-2-buten-1-yl]-2-piperidinecarboxamide sulfate

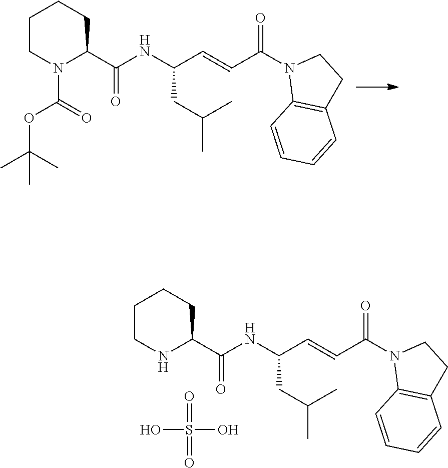

To a solution of 1,1-dimethylethyl (2S)-2-({[(1S,2E)-4-(2,3-dihydro-1H-indol-1-yl)-1-(2-methylpropyl)-4-oxo-2-buten-1-yl]amino}carbonyl)-1-piperidinecarboxylate (3.07 g, 6.54 mmol) in 1,4-dioxane (30.0 mL) was added concentrated H₂SO₄ (3.48 mL, 65.4 mmol). The reaction mixture was stirred for 1 h at RT. The reaction mixture was then concentrated in vacuo and purified by reverse phase HPLC (10-50% CH₃CN/H₂O) to afford the title compound (0.600 g, 20%) as a white solid. LC-MS m/z 370 (M+H)⁺, 0.81 min (ret time). ¹H NMR (400 MHz, MeOD) δ ppm 8.17 (d, J=8.0 Hz, 1H), 7.26 (d, J=7.4 Hz, 1H), 7.07 (t, J=7.4 Hz, 1H), 7.04-7.10 (m, 1H), 6.83 (dd, J=15.2, 6.1 Hz, 1H), 6.51 (d, J=15.2 Hz, 1H), 4.67-4.74 (m, 1H), 4.20-4.26 (m, 2H), 3.90-3.97 (m, 1H), 3.43 (d, J=13.1 Hz, 1H), 3.20-3.28 (m, 2H), 3.09-3.17 (m, 1H), 2.22-2.29 (m, 1H), 1.88-1.95 (m, 2H), 1.82-1.69 (m, 4H), 1.58-1.64 (m, 1H), 1.51-1.57 (m, 1H), 1.00 (t, J=6.9 Hz, 6H).

### Example 60

### (2S)—N-[(1S,2E)-4-(2,3-dihydro-1H-indol-1-yl)-1-(2-methylpropyl)-4-oxo-2-buten-1-yl]-2-piperidinecarboxamide hydrochloride

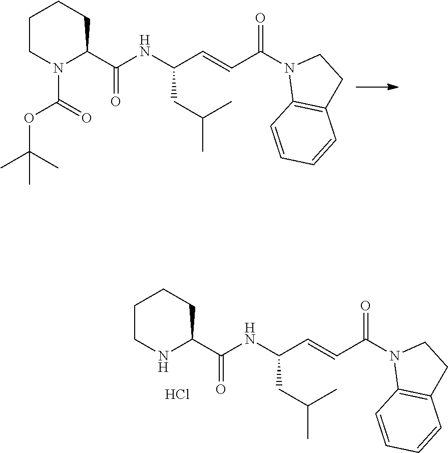

To a solution of 1,1-dimethylethyl (2S)-2-({[(1S,2E)-4-(2,3-dihydro-1H-indol-1-yl)-1-(2-methylpropyl)-4-oxo-2-buten-1-yl]amino}carbonyl)-1-piperidinecarboxylate (650 mg, 1.384 mmol) in THF (20.0 mL) was added 12 M aq. HCl (0.461 mL, 5.54 mmol). The reaction mixture was heated to 50° C. under nitrogen for 4 h and then allowed to cool to RT. The resulting solid was collected by filtration, washed with THF (5.0 mL), and dried in vacuo at 40° C. to afford the title compound (483 mg, 86%) as a white solid. LC-MS m/z 370 (M+H)⁺, 0.74 min (ret time). ¹H NMR (400 MHz, DMSO-d₆) δ ppm 8.75 (d, J=8.0 Hz, 1H), 8.60-9.00 (br s, 2H), 8.12 (d, J=7.0 Hz, 1H), 7.25 (d, J=7.3 Hz, 1H), 7.16 (t, J=7.7 Hz, 1H), 7.01 (t, J=7.7 Hz, 1H), 6.77 (dd, J=15.2, 5.6 Hz, 1H), 6.40 (d, J=15.3 Hz, 1H), 4.52-4.65 (m, 1H), 4.12-4.19 (m, 2H), 3.82 (d, J=11.5 Hz, 1H), 3.13-3.24 (m, 3H), 2.87-2.96 (m, 1H), 2.17-2.24 (m, 1H), 1.76-1.83 (m, 1H), 1.58-1.73 (m, 3H), 1.42-1.56 (m, 4H), 0.91 (t, J=7.0 Hz, 6H).

### Example 61

### (2S)—N-((1S,2E)-1-(2-methylpropyl)-4-oxo-4-{[5-(trifluoromethyl)-1,3,4-thiadiazol-2-yl]amino}-2-buten-1-yl)-2-azetidinecarboxamide trifluoroacetate

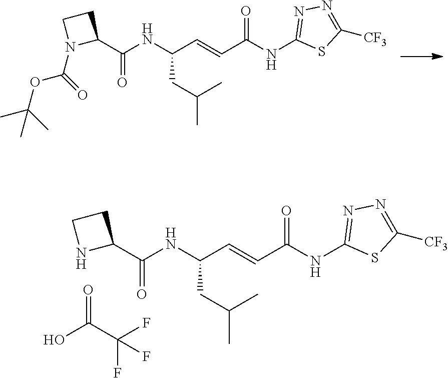

To a solution of 1,1-dimethylethyl (2S)-2-{[((1S,2E)-1-(2-methylpropyl)-4-oxo-4-{[5-(trifluoromethyl)-1,3,4-thiadiazol-2-yl]amino}-2-buten-1-yl)amino]carbonyl}-1-azetidinecarboxylate (656 mg, 1.335 mmol) in CH₂Cl₂ (4.0 mL) was added TFA (1.645 mL, 21.35 mmol). The reaction mixture was stirred overnight at RT. The reaction mixture was then concentrated in vacuo and purified by reverse phase HPLC(YMC C18 S-15 μm/12 nm 75×30 mm preparatory column), eluting with a linear gradient running from 10% CH₃CN/H₂O (0.1% TFA) to 80% CH₃CN/H₂O (0.1% TFA) over 15 min. to afford the title compound (309 mg, 46%). LC-MS m/z 392 (M+H)⁺, 0.83 min (ret time). ¹H NMR (400 MHz, MeOD) δ ppm 7.09 (dd, J=15.4, 5.9 Hz, 1H), 6.33 (dd, J=15.6, 1.5 Hz, 1H), 5.06 (dd, J=9.4, 7.7 Hz, 1H), 4.70-4.77 (m, 1H), 4.13-4.19 (m, 1H), 4.00 (td, J=10.0, 6.1 Hz, 1H), 2.87-2.96 (m, 1H), 2.60-2.66 (m, 1H), 1.67-1.75 (m, 1H), 1.51-1.59 (m, 2H), 0.96-1.06 (m, 1H), 0.99 (t, J=6.8 Hz, 6H).

### Example 62

### (2S)—N-((1S,2E)-1-(2-methylpropyl)-4-{methyl[5-(trifluoromethyl)-1,3,4-thiadiazol-2-yl]amino}-4-oxo-2-buten-1-yl)-2-azetidinecarboxamide trifluoroacetate

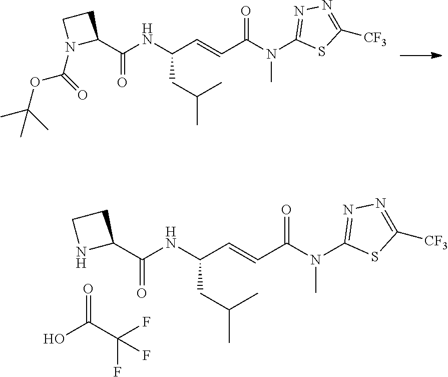

To a solution of 1,1-dimethylethyl (2S)-2-{[((1S,2E)-1-(2-methylpropyl)-4-{methyl[5-(trifluoromethyl)-1,3,4-thiadiazol-2-yl]amino}-4-oxo-2-buten-1-yl)amino]carbonyl}-1-azetidinecarboxylate (516 mg, 1.021 mmol) in CH₂Cl₂ (4.0 mL) was added TFA (1.258 mL, 16.33 mmol). The reaction mixture was stirred overnight at RT. The reaction mixture was then concentrated in vacuo and purified by reverse phase HPLC(YMC C18 S-15 μm/12 nm 75×30 mm preparatory column), eluting with a linear gradient running from 10% CH₃CN/H₂O (0.1% TFA) to 80% CH₃CN/H₂O (0.1% TFA) over 15 min. to afford the title compound (267 mg, 50%). LC-MS m/z 406 (M+H)⁺, 0.89 min (ret time). ¹H NMR (400 MHz, MeOD) δ ppm 7.07 (dd, J=15.2, 6.4 Hz, 1H), 6.82 (dd, J=15.1, 1.3 Hz, 1H), 5.06 (dd, J=9.5, 7.8 Hz, 1H), 4.75-4.81 (m, 1H), 4.12-4.18 (m, 1H), 3.97-4.02 (m, 1H), 3.94 (s, 3H), 2.86-2.92 (m, 1H), 2.55-2.61 (m, 1H), 1.78-1.68 (m, 1H), 1.53-1.64 (m, 2H), 1.00 (t, J=6.5 Hz, 6H).

### Example 63

### (4S)—N-((1S,2E)-1-ethyl-4-oxo-4-{[5-(trifluoromethyl)-1,3,4-thiadiazol-2-yl]amino}-2-buten-1-yl)-4-fluoro-L-prolinamide trifluoroacetate

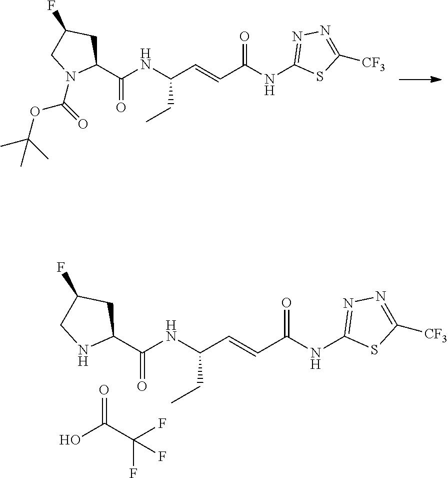

To a solution of 1,1-dimethylethyl (2S,4S)-2-{[((1S,2E)-1-ethyl-4-oxo-4-{[5-(trifluoromethyl)-1,3,4-thiadiazol-2-yl]amino}-2-buten-1-yl)amino]carbonyl}-4-fluoro-1-pyrrolidinecarboxylate (460 mg, 0.928 mmol) in CH₂Cl₂ (4.0 mL) was added TFA (1.144 mL, 14.85 mmol). The reaction mixture was stirred overnight at RT. The reaction mixture was then concentrated in vacuo and purified by reverse phase HPLC(YMC C18 S-15 μm/12 nm 75×30 mm preparatory column), eluting with a linear gradient running from 10% CH₃CN/H₂O (0.1% TFA) to 80% CH₃CN/H₂O (0.1% TFA) over 15 min. to afford the title compound (136 mg, 29%). LC-MS m/z 396 (M+H)⁺, 0.69 min (ret time). ¹H NMR (400 MHz, MeOD) δ ppm 7.11 (dd, J=15.4, 5.9 Hz, 1H), 6.33 (dd, J=15.6, 1.5 Hz, 1H), 5.48 (dt, J=50.1, 3.9 Hz, 1H), 4.58 (dt, J=6.2, 1.7 Hz, 1H), 4.54 (dd, J=10.5, 4.0 Hz, 1H), 3.80 (ddd, J=18.9, 13.4, 1.9 Hz, 1H), 3.55 (ddd, J=35.2, 13.5, 3.4 Hz, 1H), 2.71-2.92 (m, 1H), 2.50-2.68 (m, 1H), 1.64-1.87 (m, 2H), 1.04 (t, J=7.4 Hz, 3H).

### Example 64

### (2S)—N-((1S,2E)-1-(2-methylpropyl)-4-oxo-4-{[5-(trifluoromethyl)-1,3,4-thiadiazol-2-yl]amino}-2-buten-1-yl)-2-piperidinecarboxamide trifluoroacetate

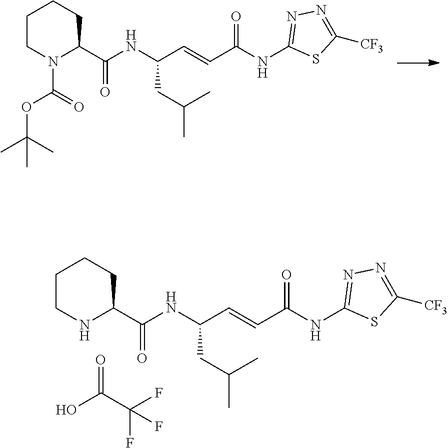

To a solution of 1,1-dimethylethyl (2S)-2-{[((1S,2E)-1-(2-methylpropyl)-4-oxo-4-{[5-(trifluoromethyl)-1,3,4-thiadiazol-2-yl]amino}-2-buten-1-yl)amino]carbonyl}-1-piperidinecarboxylate (60 mg, 0.115 mmol) in CH₂Cl₂ (6.0 mL) was added TFA (0.071 mL, 0.924 mmol). The reaction mixture was stirred overnight at RT. The reaction mixture was then concentrated in vacuo and purified by reverse phase HPLC(YMC C18 S-15 μm/12 nm 75×30 mm preparatory column), eluting with a linear gradient running from 10% CH₃CN/H₂O (0.1% TFA) to 80% CH₃CN/H₂O (0.1% TFA) over 15 min. to afford the title compound (48 mg, 73%). LC-MS m/z 420 (M+H)⁺, 0.88 min (ret time). ¹H NMR (400 MHz, MeOD) δ ppm 8.53 (d, J=8.0 Hz, 1H), 7.10 (dd, J=15.4, 5.6 Hz, 1H), 6.30 (dd, J=15.3, 1.5 Hz, 1H), 4.70-4.78 (m, 1H), 3.86 (dd, J=11.8, 3.0 Hz, 1H), 3.39-3.46 (m, 1H), 3.01-3.10 (m, 1H), 2.34 (br. d, J=13.8 Hz, 1H), 2.00-2.08 (m, 1H), 1.96-2.03 (m, 1H), 1.89-1.95 (m, 1H), 1.66-1.86 (m, 4H), 1.49-1.62 (m, 2H), 0.99 (t, J=6.8 Hz, 6H).

### Example 65

### (2S)—N-((1S,2E)-1-(2-methylpropyl)-4-oxo-4-{[5-(trifluoromethyl)-1,3,4-thiadiazol-2-yl]amino}-2-buten-1-yl)-2-piperidinecarboxamide sulfate

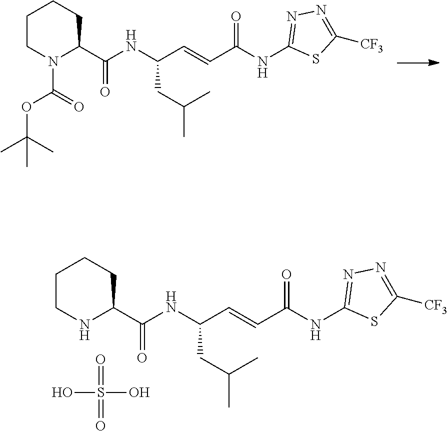

To a solution of 1,1-dimethylethyl (2S)-2-{[((1S,2E)-1-(2-methylpropyl)-4-oxo-4-{[5-(trifluoromethyl)-1,3,4-thiadiazol-2-yl]amino}-2-buten-1-yl)amino]carbonyl}-1-piperidinecarboxylate (1.20 g, 2.310 mmol) in 1,4-dioxane (20.0 mL) at RT was added 10% aq. H₂SO₄ (12.31 mL, 23.10 mmol). The reaction mixture was then heated to 50° C. overnight. The reaction mixture was then concentrated in vacuo and purified by reverse phase HPLC (10-50% CH₃CN/H₂O) to afford the title compound (294 mg, 25%) as a white solid. LC-MS m/z 420 (M+H)⁺, 0.76 min (ret time). ¹H NMR (400 MHz, DMSO-d₆) δ ppm 8.53 (d, J=7.8 Hz, 1H), 6.90 (dd, J=15.1, 4.5 Hz, 1H), 6.15 (d, J=15.6 Hz, 1H), 4.51-4.65 (m, 1H), 3.79 (dd, J=11.9, 2.1 Hz, 1H), 3.24 (br d, J=12.7 Hz, 1H), 2.94 (td, J=12.3, 2.3 Hz, 1H), 2.25 (d, J=11.3 Hz, 1H), 1.86 (d, J=11.8 Hz, 1H), 1.53-1.75 (m, 5H), 1.45 (t, J=7.2 Hz, 2H), 0.91 (d, J=6.5 Hz, 3H), 0.93 (d, J=6.8 Hz, 3H).

### Examples 66-187

### General Experimental for Table 3

The compounds in Table 3 were prepared from the indicated enoate intermediates (described above), commercially available Boc-protected α-amino acids, and amines (commercially available or prepared as described above) according to either Scheme P1 or Scheme P2 as indicated:

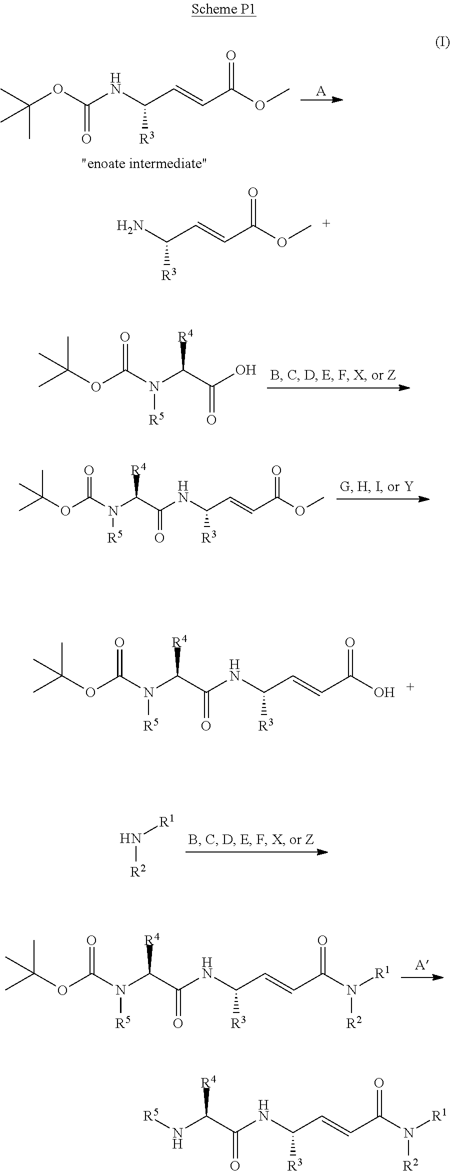

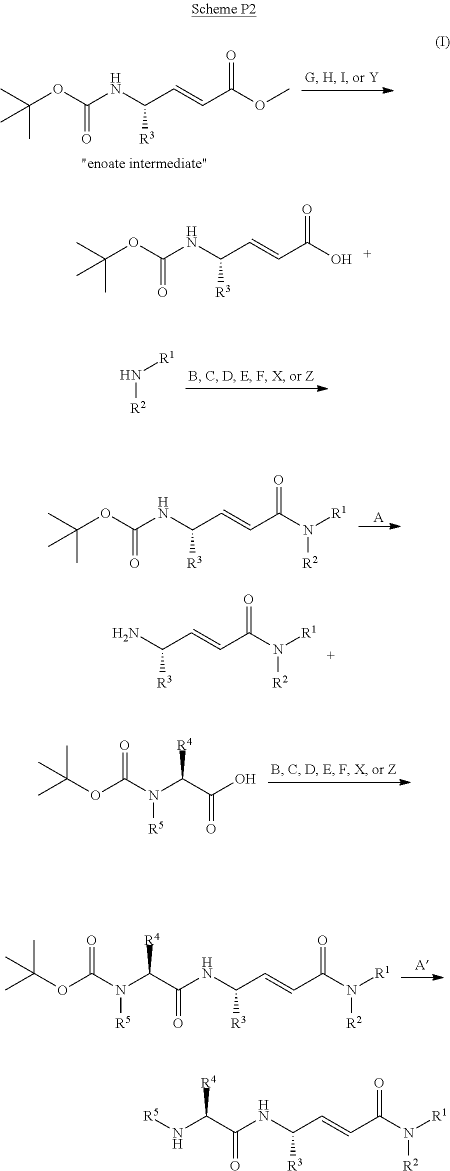

Representative examples of the reaction conditions A, A′, B, C, D, E, F, G, H, I, X, Y, and Z are described in the following experimental procedures (vide supra):

### A: Intermediate 34; B: Intermediate 37; C: Intermediate 53; D: Intermediate 50;

### E: Intermediate 27; F: Intermediate 48; G; Intermediate 45; H: Intermediate 61;

### I: Intermediate 72; Z: Intermediate 43; A′: Example 53.

Reaction condition X: A solution of the carboxylic acid (1.833 mmol), HATU (1.833 mmol), and DIPEA (6.42 mmol) in CH₂Cl₂ (10.0 mL) and DMF (5.0 mL) was stirred at RT for 30 min. A solution of the amine (1.833 mmol) in DMF (5.0 mL) was then added and stirring continued for approximately 1 h. Water was added (100 mL) with stirring. The reaction mixture was transferred to a separatory funnel and extracted with ethyl acetate (100 mL). The organic layer was washed with water (5×100 mL) followed by brine (100 mL), dried over Na₂SO₄, and concentrated in vacuo to afford the desired amide product. Reaction condition Y: The ester (11.27 mmol) was diluted in THF (60 mL) and water (12 mL). 4 M aq. LiOH (45.1 mmol) was added and the reaction mixture was stirred at RT for 15 h. An additional 1 equivalent of LiOH was added as a solution in water (5 mL), and stirring was continued at RT for an additional 5.5 h. The reaction mixture was acidified with 1M aq. HCl to pH ˜3 (pH paper) and then partitioned between water and EtOAc. The aqueous layer was extracted with EtOAc and the organic layer was washed with water followed by brine. The combined organic layers were filtered and concentrated in vacuo to afford the desired acid product.

TABLE 3

| | Scheme; | | | | |
|---|---|---|---|---|---|
| | Enoate | | | LC-MS | |
| | Intermediate; | | | m/z | ret time |
| Example | Procedures | Structure | Name | (M + H)⁺ | (min) |
| 66 | P1; 3; A-C-H-Z-A′ | 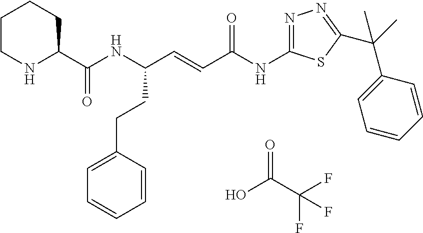 | (2S)-N-[(1S,2E)-4-{[5- (1-methyl-1- phenylethyl)-1,3,4- thiadiazol-2-yl]amino}- 4-oxo-1-(2-phenylethyl)- 2-buten-1-yl]-2- piperidinecarboxamide trifluoroacetate | 518 | 0.93 |
| 67 | P1; 3; A-F-I-F-A′ | 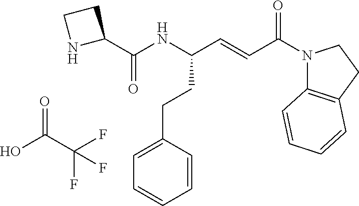 | (2S)-N-[(3S,4E)-6-(2,3- dihydro-1H-indol-1-yl)- 6-oxo-1-phenylhex-4-en- 3-yl]azetidine-2- carboxamide trifluoroacetate | 390 | 0.85 |
| 68 | P1; 3; A-F-I-F-A′ | 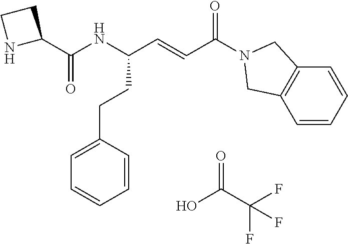 | (2S)-N-[(3S,4E)-6-(1,3- dihydro-2H-isoindol-2- yl)-6-oxo-1-phenylhex- 4-en-3-yl]azetidine-2- carboxamide trifluoroacetate | 390 | 0.8 |
| 69 | P2; 3; Y-Z-A-F-A′ | 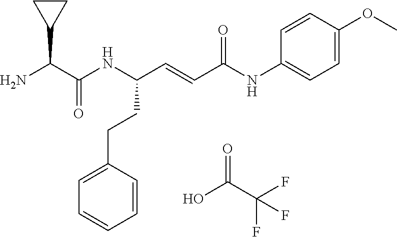 | (2E,4S)-4-{[(2S)-2- amino-2- cyclopropylacetyl]amino}- N-(4-methoxyphenyl)- 6-phenylhex-2-enamide trifluoroacetate | 408 | 0.79 |
| 70 | P1; 3; A-F-I-F-A′ | 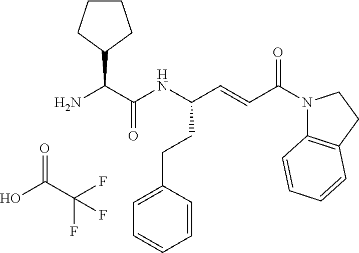 | (2S)-2-amino-2- cyclopentyl-N-[(3S,4E)- 6-(2,3-dihydro-1H-indol- 1-yl)-6-oxo-1- phenylhex-4-en-3- yl]ethanamide trifluoroacetate | 432 | 0.97 |
| 71 | P1; 3; A-F-I-F-A′ | 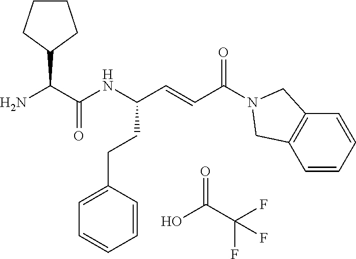 | (2S)-2-amino-2- cyclopentyl-N-[(3S,4E)- 6-(1,3-dehydro-2H- isoindol-2-yl)-6-oxo-1- phenylhex-4-en-3- yl]ethanamide trifluoroacetate | 432 | 0.93 |
| 72 | P1; 3; A-E-H-C-A′ | 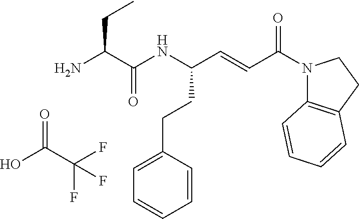 | (2S)-2-amino-N- [(3S,4E)-6-(2,3-dihydro- 1H-indol-1-yl)-6-oxo-1- phenylhex-4-en-3- yl]butanamide trifluoroacetate | 392 | 0.9 |
| 73 | P1; 3; A-E-G-C-A′ | 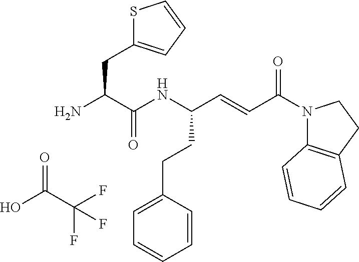 | N-[(3S,4E)-6-(2,3- dihydro-1H-indol-1-yl)- 6-oxo-1-phenylhex-4-en- 3-yl]-3-thiophen-2-yl-L- alaninamide trifluoroacetate | 460 | 0.97 |
| 74 | P2; 3; H-Z-A-E-A′ | 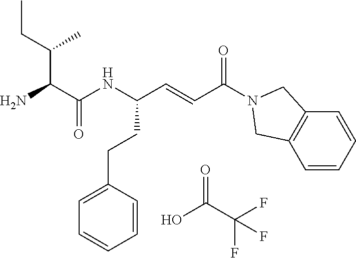 | N-[(3S,4E)-6-(1,3- dihydro-2H-isoindol-2- yl)-6-oxo-1-phenylhex- 4-en-3-yl]-L- isoleucinamide trifluoroacetate | 420 | 0.85 |
| 75 | P2; 3; H-Z-A-E-A′ | 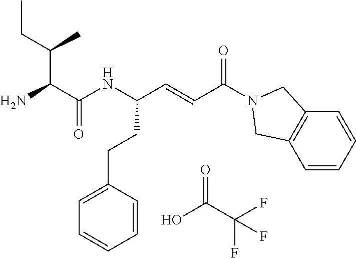 | N-[(3S,4E)-6-(1,3- dihydro-2H-isoindol-2- yl)-6-oxo-1-phenylhex- 4-en-3-yl]-L- alloisoleucinamide trifluoroacetate | 420 | 0.84 |
| 76 | P2; 3; H-Z-A-E-A′ | 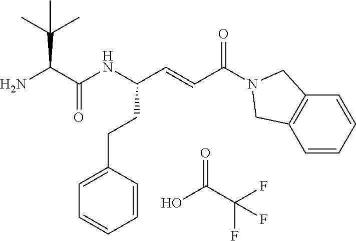 | N-[(3S,4E)-6-(1,3- dihydro-2H-isoindol-2- yl)-6-oxo-1-phenylhex- 4-en-3-yl]-3-methyl-L- valinamide trifluoroacetate | 420 | 0.84 |
| 77 | P2; 3; H-Z-A-E-A′ | 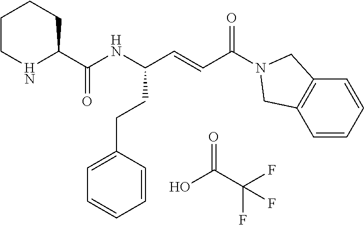 | (2S)-N-[(3S,4E)-6-(1,3- dihydro-2H-isoindol-2- yl)-6-oxo-1-phenylhex- 4-en-3-yl]piperidine-2- carboxamide trifluoroacetate | 418 | 0.81 |
| 78 | P2; 3; H-Z-A-E-A′ | 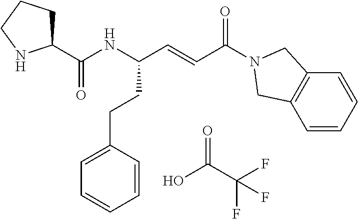 | N-[(3S,4E)-6-(1,3- dihydro-2H-isoindol-2- yl)-6-oxo-1-phenylhex- 4-en-3-yl]-L-prolinamide trifluoroacetate | 404 | 0.81 |
| 79 | P2; 3; H-Z-A-E-A′ | 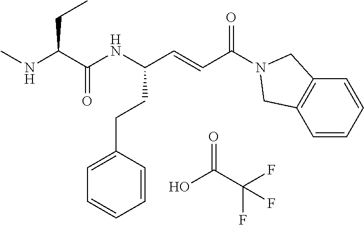 | (2S)-N-[(3S,4E)-6-(1,3- dihydro-2H-isoindol-2- yl)-6-oxo-1-phenylhex- 4-en-3-yl]-2- (methylamino)butanamide trifluoroacetate | 406 | 0.82 |
| 80 | P1; 3; A-C-H-Z-A′ | 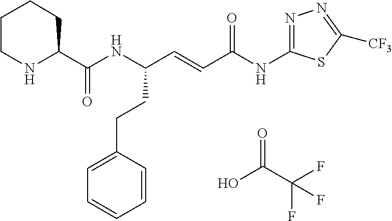 | (2S)-N-[(3S,4E)-6-oxo- 1-phenyl-6-{[5- (trifluoromethyl)-1,3,4- thiadiazol-2- yl]amino}hex-4-en-3- yl]piperidine-2- carboxamide trifluoroacetate | 468 | 0.89 |
| 81^{a} | P1; 3; A-C-H-Z-A′ | 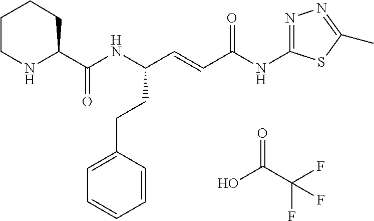 | (2S)-N-{(3S,4E)-6-[(5- methyl-1,3,4-thiadiazol- 2-yl)amino]-6-oxo-1- phenylhex-4-en-3- yl}piperidine-2- carboxamide trifluoroacetate | 414 | 0.67 |
| 82^{b} | P1; 3; A-C-H-Z-A′ | 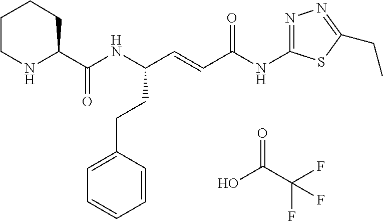 | (2S)-N-{(3S,4E)-6-[(5- ethyl-1,3,4-thiadiazol-2- yl)amino]-6-oxo-1- phenylhex-4-en-3- yl}piperidine-2- carboxamide trifluoroacetate | 428 | 0.72 |
| 83 | P1; 3; A-C-H-Z-A′ | 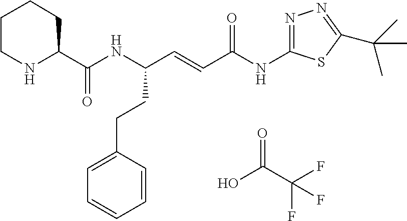 | (2S)-N-{(3S,4E)-6-[(5- tert-butyl-1,3,4- thiadiazol-2-yl)amino]- 6-oxo-1-phenylhex-4-en- 3-yl}piperidine-2- carboxamide trifluoroacetate | 456 | 0.85 |
| 84 | P1; 3; A-C-H-Z-A′ | 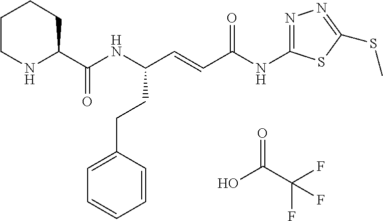 | (2S)-N-[(3S,4E)-6-{[5- (methylsulfanyl)-1,3,4- thiadiazol-2-yl]amino}- 6-oxo-1-phenylhex-4-en- 3-yl]piperidine-2- carboxamide trifluoroacetate | 446 | 0.77 |
| 85 | P1; 3; A-C-H-Z-A′ | 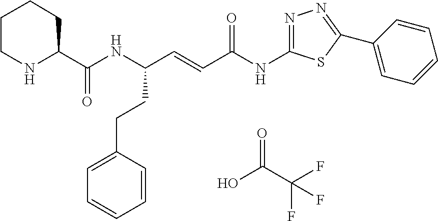 | (2S)-N-{(3S,4E)-6-oxo- 1-phenyl-6-[(5-phenyl- 1,3,4-thiadiazol-2- yl)amino]hex-4-en-3- yl}piperidine-2- carboxamide trifluoroacetate | 476 | 0.88 |
| 86 | P1; 3; A-C-H-Z-A′ | 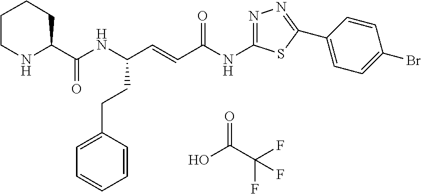 | (2S)-N-[(3S,4E)-6-{[5- (4-bromophenyl)-1,3,4- thiadiazol-2-yl]amino}- 6-oxo-1-phenylhex-4-en- 3-yl]piperidine-2- carboxamide trifluoroacetate | 554 | 0.98 |
| 87 | P1; 3; A-C-H-Z-A′ | 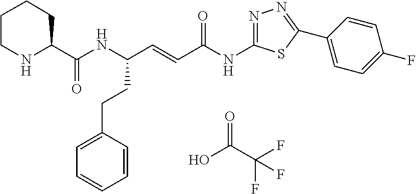 | (2S)-N-[(3S,4E)-6-1[5- (4-fluorophenyl)-1,3,4- thiadiazol-2-yl]amino}- 6-oxo-1-phenylhex-4-en- 3-yl]piperidine-2- trifluoroacetate | 494 | 0.9 |
| 88 | P1 3; A-C-H-Z-A′ | 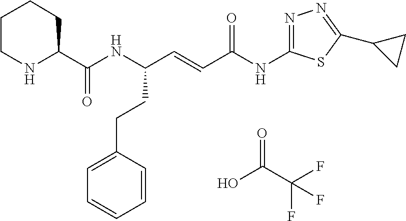 | (2S)-N-{(3S,4E)-6-[(5- cyclopropyl-1,3,4- thiadiazol-2-yl)amino]- 6-oxo-1-phenylhex-4-en- 3-yl}piperidine-2- carboxamide trifluoroacetate | 440 | 0.75 |
| 89 | P1; 3; A-C-H-Z-A′ | 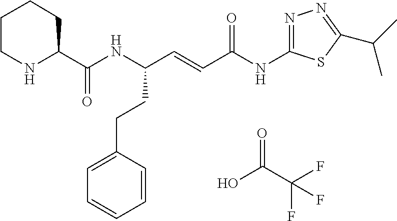 | (2S)-N-[(3S,4E)-6-oxo- 1-phenyl-6-{[5-(propan- 2-yl)-1,3,4-thiadiazol-2- yl]amino}hex-4-en-3- yl]piperidine-2- carboxamide trifluoroacetate | 442 | 0.79 |
| 90 | P1; 3; A-C-H-Z-A′ | 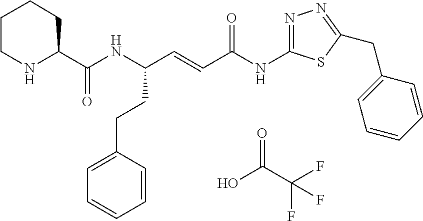 | (2S)-N-{(3S,4E)-6-[(5- benzyl-1,3,4-thiadiazol- 2-yl)amino]-6-oxo-1- phenylhex-4-en-3- yl}piperidine-2- carboxamide trifluoroacetate | 490 | 0.86 |
| 91 | P1; 3; A-C-H-Z-A′ | 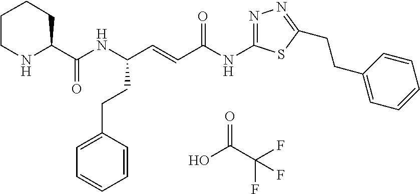 | (2S)-N-[(3S,4E)-6-oxo- 1-phenyl-6-{[5-(2 phenylethyl)-1,3,4- thiadiazol-2- yl]amino}hex-4-en-3- yl]piperidine-2- carboxamide trifluoroacetate | 504 | 0.9 |
| 92 | P1; 3; A-C-H-Z-A′ | 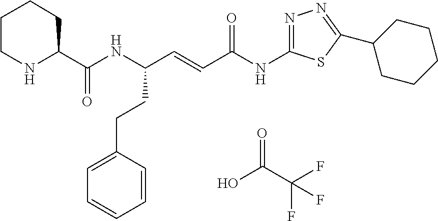 | (2S)-N-{(3S,4E)-6-[(5- cyclohexyl-1,3,4- thiadiazol-2-yl)amino]- 6-oxo-1-phenylhex-4-en- 3-yl}piperidine-2- carboxamide trifluoroacetate | 482 | 0.93 |
| 93 | P1; 3; A-F-I-F-A′ | 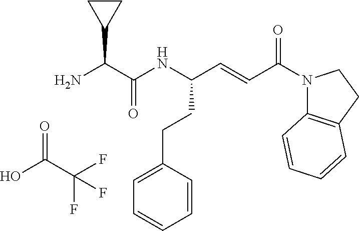 | (2S)-2-amino-2- cyclopropyl-N-[(3S,4E)- 6-(2,3-dihydro-1H-indol- 1-yl)-6-oxo-1- phenylhex-4-en-3 yl]ethanamide trifluoroacetate | 404 | 0.94 |
| 94 | P1; 3; A-F-I-F-A′ | 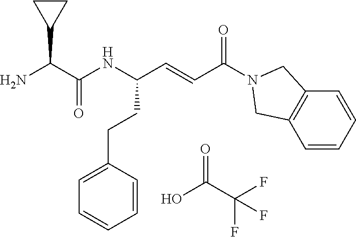 | (2S)-2-amino-2- cyclopropyl-N-[(3S,4E)- 6-(1,3-dehydro-2H- isoindol-2-yl)-6-oxo-1- phenylhex-4-en-3- yl]ethanamide trifluoroacetate | 404 | 0.9 |
| 95 | P1; 3; A-F-I-F-A′ | 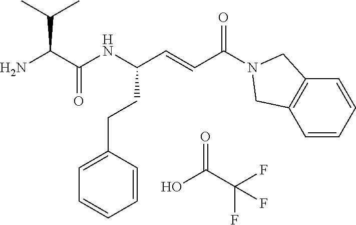 | N-[(3S,4E)-6-(1,3- dihydro-2H-isoindol-2- yl)-6-oxo-1-phenylhex- 4-en-3-yl]-L-valinamide trifluoroacetate | 406 | 0.88 |
| 96 | P2; 3; I-F-A-F-A | 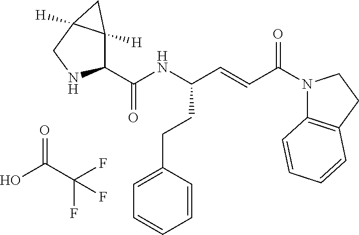 | (1R,2S,5S)-N-[(3S,4E)-6- (2,3-dihydro-1H-indol-1- yl)-6-oxo-1-phenylhex- 4-en-3-yl]-3- azabicyclo[3.1.0]hexane- 2-carboxamide trifluoroacetate | 416 | 0.87 |
| 97 | P2; 3; I-F-A-F-A | 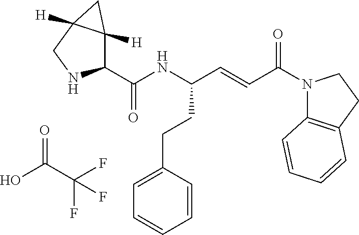 | (1S,2R,5R)-N-[(3S,4E)- 6-(2,3-dihydro-1H-indol- 1-yl)-6-oxo-1- phenylhex-4-en-3-yl]-3- azabicyclo[3.1.0]hexane- 2-carboxamide trifluoroacetate | 416 | 0.91 |
| 98 | P1; 3; A-X-H-C-A′ | 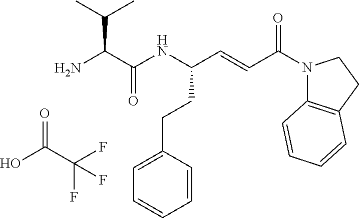 | N-[(3S,4E)-6-(2,3- dihydro-1H-indol-1-yl)- 6-oxo-1-phenylhex-4-en- 3-yl]-L-valinamide trifluoroacetate | 406 | 0.96 |
| 99 | P1; 3; A-X-H-C-A′ | 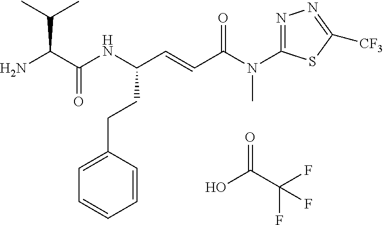 | (2E,4S)-N-methyl-6- phenyl-N-[5- (trifluoromethyl)-1,3,4- thiadiazol-2-yl]-4-(L- valylamino)hex-2- enamide trifluoroacetate | 470 | 1.04 |
| 100 | P1; 3; A-X-H-C-A′ | 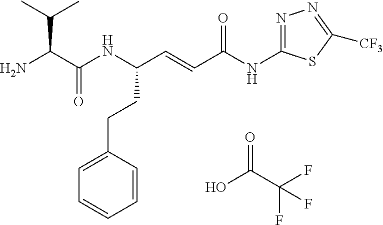 | (2E,4S)-6-phenyl-N-[5- (trifluoromethyl)-1,3,4- thiadiazol-2-yl]-4-(L- valylamino)hex-2- enamide trifluoroacetate | 456 | 1.04 |
| 101 | P1; 3; A-C-H-C-A′ | 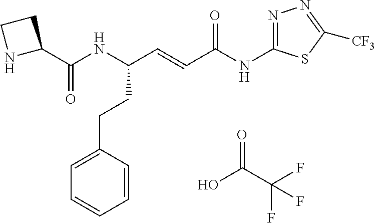 | (2S)-N-[(3S,4E)-6-oxo- 1-phenyl-6-{[5- (trifluoromethyl)-1,3,4- thiadiazol-2- yl]lamino}hex-4-en-3- yl]azetidine-2- carboxamide trifluoroacetate | 440 | 0.91 |
| 102 | P1; 3; A-C-H-C-A′ | 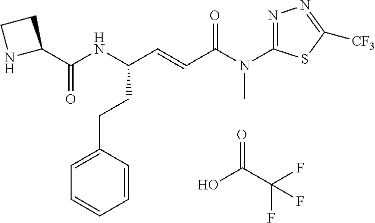 | (2S)-N-[(3S,4E)-6- {methyl[5- (trifluoromethyl)-1,3,4- thiadiazol-2-yl]amino}- 6-oxo-1-phenylhex-4-en- 3-yl]azetidine-2- carboxamide trifluoroacetate | 454 | 0.87 |
| 103 | P1; 3; A-C-H-C-A′ | 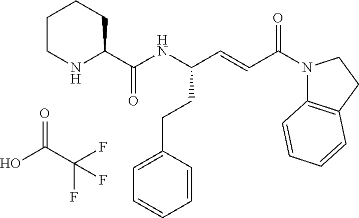 | (2S)-N-[(3S,4E)-6-(2,3- dihydro-1H-indol-1-yl)- 6-oxo-1-phenylhex-4-en- 3-yl]piperidine-2- carboxamide trifluoroacetate | 418 | 0.87 |
| 104 | P2; 3; I-F-A-F-A′ | 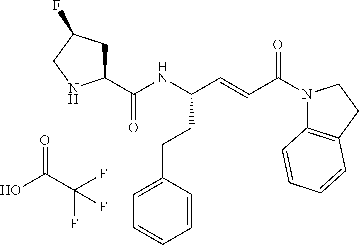 | (4S)-N-[(3S,4E)-6-(2,3- dihydro-1H-indol-1-yl)- 6-oxo-1-phenylhex-4-en- 3-yl]-4-fluoro-L- prolinamide trifluoroacetate | 422 | 0.86 |
| 105 | P1; 93; A-E-H-C-A′ | 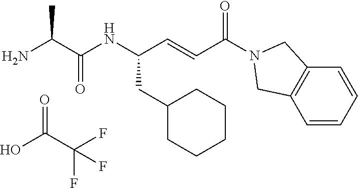 | N-[(2S,3E)-1- cyclohexyl-5-(1,3 dihydro-2H-isoindol-2- yl)-5-oxopent-3-en-2- yl]-L-alaninamide trifluoroacetate | 370 | 0.91 |
| 106 | P1; 93; A-E-H-C-A' | 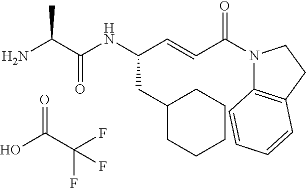 | N-[(2S,3E)-1- cyclohexyl-5-(2,3- dihydro-1H-indol-1-yl)- 5-oxopent-3-en-2-yl]-L- alaninamide trifluoroacetate | 370 | 0.97 |
| 107 | P1; 93; A-E-H-C-A′ | 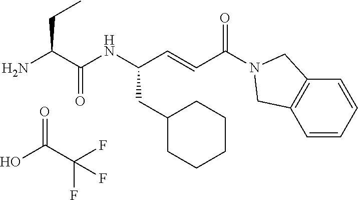 | (2S)-2-amino-N- [(2S,3E)-1-cyclohexyl-5- (1,3-dihydro-2H- isoindol-2-yl)-5- oxopent-3-en-2- yl]butanamide trifluoroacetate | 384 | 0.82 |
| 108 | P1; 93; A-E-H-C-A′ | 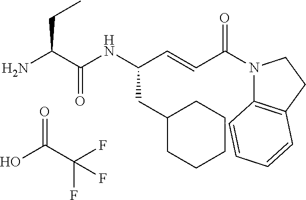 | (2S)-2-amino-N- [(2S,3E)-1-cyclohexyl-5- (2,3-dehydro-1H-indol-1- yl)-5-oxopent-3-en-2- yl]butanamide trifluoroacetate | 384 | 0.9 |
| 109 | P1; 93; A-E-H-C-A′ | 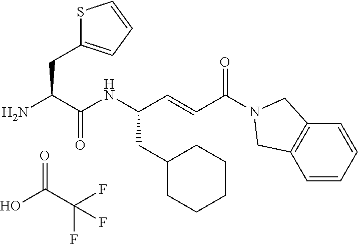 | N-[(2S,3E)-1- cyclohexyl-5-(1,3- dihydro-2H-isoindol-2- yl)-5-oxopent-3-en-2- yl]-3-thiophen-2-yl-L- alaninamide trifluoroacetate | 452 | 0.92 |
| 110 | P1; 93; A-E-H-C-A′ | 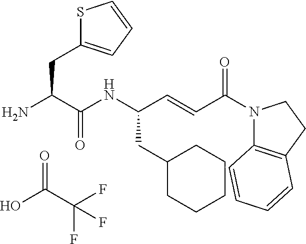 | N-[(2S,3E)-1- cyclohexyl-5-(2,3- dihydro-1H-indol-1-yl)- 5-oxopent-3-en-2-yl]-3- thiophen-2-yl-L- alaninamide trifluoroacetate | 452 | 0.96 |
| 111 | P1; 93; A-E-H-C-A′ | 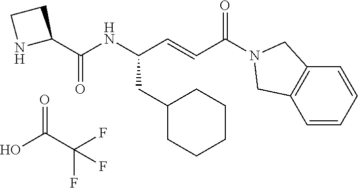 | (2S)-N-[(2S,3E)-1- cyclohexyl-5-(1,3- dihydro-2H-isoindol-2- yl)-5-oxopent-3-en-2- yl]azetidine-2- carboxamide trifluoroacetate | 382 | 0.86 |
| 112 | P1; 93; A-E-H-C-A′ | 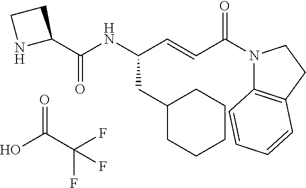 | (2S)-N-[(2S,3E)-1- cyclohexyl-5-(2,3- dihydro-1H-indol-1-yl)- 5-oxopent-3-en-2- yl]azetidine-2- carboxamide trifluoroacetate | 382 | 0.9 |
| 113 | P1; 93; A-E-H-C-A′ | 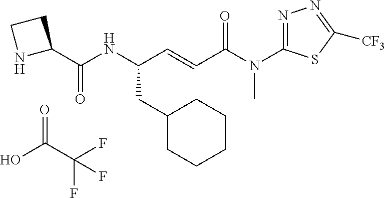 | (2S)-N-[(2S,3E)-1- cyclohexyl-5-{methyl[5- (trifluoromethyl)-1,3,4- thiadiazol-2-yl]amino}- 5-oxopent-3-en-2- yl]azetidine-2- carboxamide trifluoroacetate | 446 | 1 |
| 114 | P1; 93; A-E-H-C-A′ | 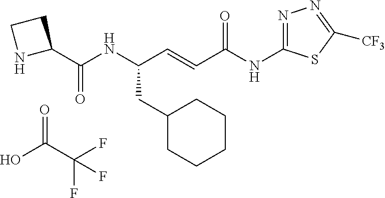 | (2S)-N-[(2S,3E)-1- cyclohexyl-5-oxo-5-{[5- (tnfluoromethyl)-1,3,4- thiadiazol-2- yl]amino}pent-3-en-2- yl]azetidine-2- carboxamide trifluoroacetate | 432 | 0.94 |
| 115 | P1; 93; A-E-H-C-A′ | 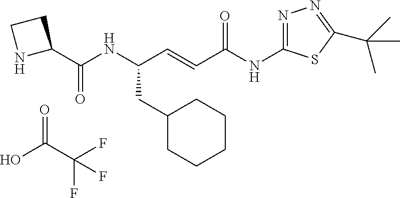 | (2S)-N-{(2S,3E)-5-[(5- tert-butyl-1,3,4- thiadiazol-2-yl)amino]- 1-cyclohexyl-5-oxopent- 3-en-2-yl}azetidine-2- carboxamide trifluoroacetate | 420 | 0.91 |
| 116 | P1; 88; A-C-H-C-A′ | 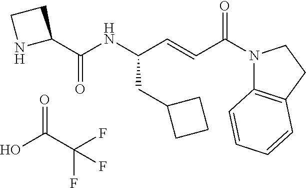 | (2S)-N-[(2S,3E)-1- cyclobutyl-5-(2,3- dihydro-1H-indol-1-yl)- 5-oxopent-3-en-2- yl]azetidine-2- carboxamide trifluoroacetate | 354 | 0.79 |
| 117^{c} | P1; 88; A-C-H-C-A′ | 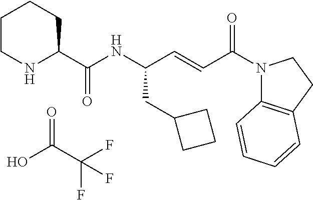 | (2S)-N-[(2S,3E)-1- cyclobutyl-5-(2,3 dihydro-1H-indol-1-yl)- 5-oxopent-3-en-2- yl]piperidine-2- carboxamide trifluoroacetate | 382 | 0.77 |
| 118^{d} | P1; 88; A-C-H-C-A′ | 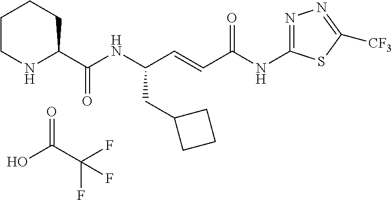 | (2S)-N-[(2S,3E)-1- cyclobutyl-5-oxo-5-{[5- (trifluoromethyl)-1,3,4- thiadiazol-2- yl]amino}pent-3-en-2- yl]piperidine-2- carboxamide trifluoroacetate | 432 | 0.87 |
| 119 | P1; 94; A-E-G-C-A′ | 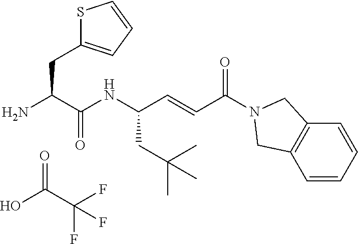 | N-[(2E,4S)-1-(1,3- dihydro-2H-isoindol-2- yl)-6,6-dimethyl-1- oxohept-2-en-4-yl]-3- thiophen-2-yl-L- alaninamide trifluoroacetate | 426 | 0.91 |
| 120 | P1; 94; A-E-G-C-A′ | 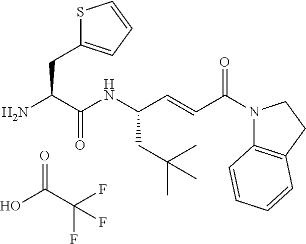 | N-[(2E,4S)-1-(2,3- dihydro-1H-indol-1-yl)- 6,6-dimethyl-1-oxohept- 2-en-4-yl]-3-thiophen-2- yl-L-alaninamide trifluoroacetate | 426 | 0.95 |
| 121 | P1; 94; A-E-G-C-A′ | 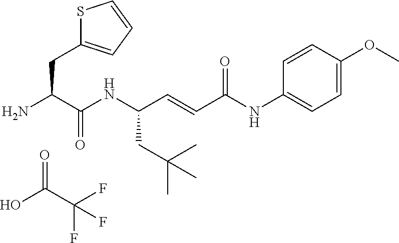 | (2E,4S)-N-(4- methoxyphenyl)-6,6- dimethyl-4-{[3- (thiophen-2-yl)-L- alanyl]amino}hept-2- enamide trifluoroacetate | 430 | 0.87 |
| 122 | P1; 94; A-E-G-C-A′ | 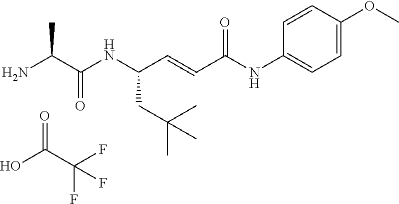 | (2E,4S)-4-(L- alanylamino)-N-(4- methoxyphenyl)-6,6- dimethylhept-2-enamide trifluoroacetate | 348 | 0.79 |
| 123 | P1; 94; A-E-G-C-A′ | 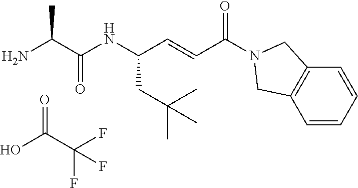 | N-[(2E,4S)-1-(1,3- dihydro-2H-isoindol-2- yl)-6,6-dimethyl-1- oxohept-2-en-4-yl]-L- alaninamide trifluoroacetate | 344 | 0.82 |
| 124 | P1; 94; A-E-G-C-A′ | 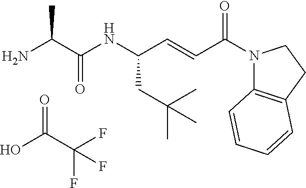 | N-[(2E,4S)-1-(2,3- dihydro-1H-indol-1-yl)- 6,6-dimethyl-1-oxohept- 2-en-4-yl]-L- alaninamide trifluoroacetate | 344 | 0.89 |
| 125 | P1; 94; A-E-G-C-A′ | 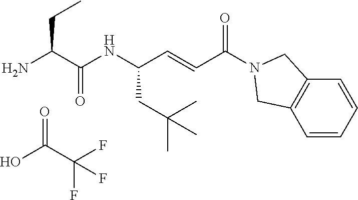 | (2S)-2-amino-N- [(2E,4S)-1-(1,3-dihydro- 2H-isoindol-2-yl)-6,6- dimethyl-1-oxohept-2- en-4-yl]butanamide trifluoroacetate | 358 | 0.85 |
| 126 | P1; 94; A-E-G-C-A′ | 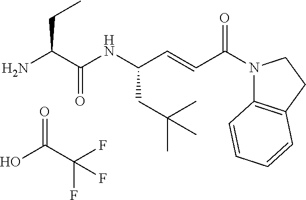 | (2S)-2-amino-N- [(2E,4S)-1-(2,3-dihydro- 1H-indol-1-yl)-6,6- dimethyl-1-oxohept-2- en-4-yl]butanamide trifluoroacetate | 358 | 0.9 |
| 127 | P1; 94; A-E-G-C-A′ | 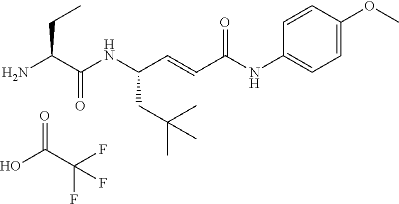 | (2E,4S)-4-{[(2S)-2- aminobutanoyl]amino}- N-(4-methoxyphenyl)- 6,6-dimethylhept-2- enamide trifluoroacetate | 362 | 0.83 |
| 128 | P1; 33; A-E-G-Z-A′ | 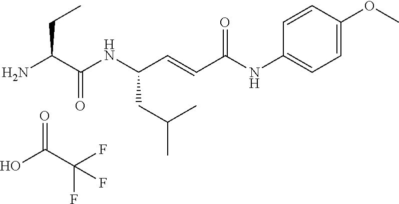 | (2E,4S)-4-{[(2S)-2- aminobutanoyl]amino}- N-(4-methoxyphenyl)-6- methylhept-2-enamide trifluoroacetate | 348 | 0.68 |
| 129 | P1; 33; A-E-G-Z-A′ | 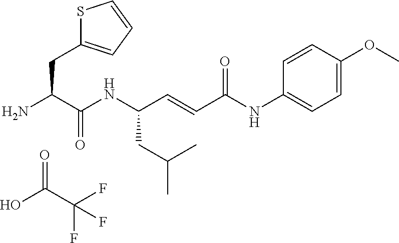 | (2E,4S)-N-(4- methoxyphenyl)-6- methyl-4-{[3-(thiophen- 2-yl)-L- alanyl]amino}hept-2- enamide trifluoroacetate | 416 | 0.77 |
| 130 | P1; 33; A-E-G-C-A′ | 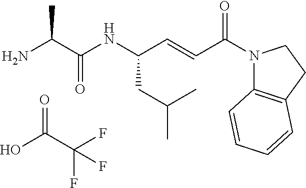 | N-[(2E,4S)-1-(2,3- dihydro-1H-indol-1-yl)- 6-methyl-1-oxohept-2- en-4-yl]-L-alaninamide trifluoroacetate | 330 | 0.81 |
| 131 | P1; 33; A-E-G-C-A′ | 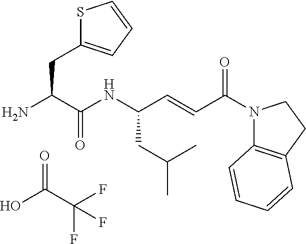 | N-[(2E,4S)-1-(2,3- dihydro-1H-indol-1-yl)- 6-methyl-1-oxohept-2- en-4-yl]-3-thiophen-2-yl- L-alaninamide trifluoroacetate | 412 | 0.86 |
| 132 | P1; 33; A-E-G-C-A′ | 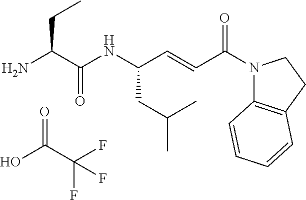 | (2S)-2-amino-N- [(2E,4S)-1-(2,3-dihydro- 1H-indol-1-yl)-6-methyl- 1-oxohept-2-en-4- yl]butanamide trifluoroacetate | 344 | 0.8 |
| 133 | P1; 33; A-E-G-C-A′ | 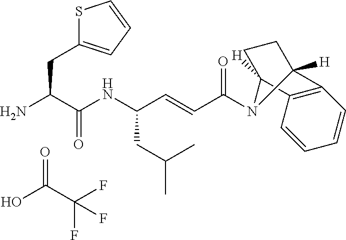 | N-{(2E,4S)-6-methyl-1- oxo-1-[(1R,4S)-1,2,3,4- tetrahydro-1,4- epiminonaphthalen-9- yl]hept-2-en-4-yl}-3- thiophen-2-yl-L- alaninamide trifluoroacetate | 438 | 0.86 |
| 134 | P1; 33; A-E-G-C-A′ | 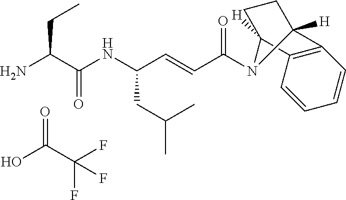 | (2S)-2-amino-N- {(2E,4S)-6-methyl-1- oxo-1-[(1R,4S)-1,2,3,4- tetrahydro-1,4- epiminonaphthalen-9- yl]hept-2-en-4- yl}butanamide trifluoroacetate | 370 | 0.79 |
| 135 | P1; 33; A-E-G-C-A′ | 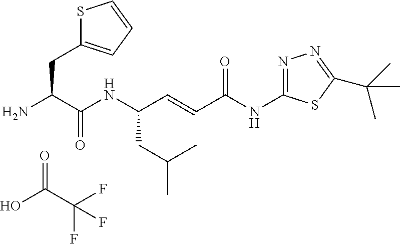 | (2E,4S)-N-(5-tert-butyl- 1,3,4-thiadiazol-2-yl)-6- methyl-4-{[3-(thiophen- 2-yl)-L- alanyl]amino}hept-2- enamide trifluoroacetate | 450 | 0.93 |
| 136 | P1; 25; A-E-G-C-A′ | 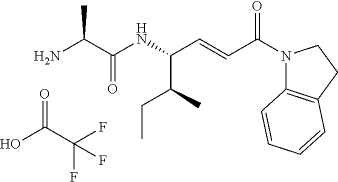 | N-[(2E,4S,5S)-1-(2,3- dihydro-1H-indol-1-yl)- 5-methyl-1-oxohept-2- en-4-yl]-L-alaninamide trifluoroacetate | 330 | 0.76 |
| 137 | P1; 33; A-E-G-C-A′ | 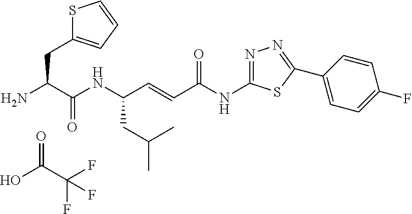 | (2E,4S)-N-[5(4- fluorophenyl)-1,3,4- thiadiazol-2-yl]-6- methyl-4-{[3-(thiophen- 2-yl)-L- alanyl]amino}hept-2- enamide trifluoroacetate | 488 | 0.99 |
| 138 | P1; 33; A-C-H-C-A′ | 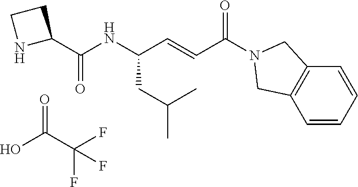 | (2S)-N-[(2E,4S)-1-(1,3- dihydro-2H-isoindol-2- yl)-6-methyl-l-oxohept- 2-en-4-yl]azetidine-2- carboxamide trifluoroacetate | 342 | 0.79 |
| 139 | P1; 33; A-C-H-C-A′ | 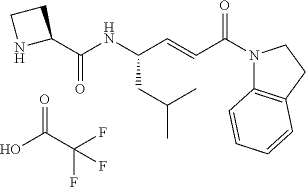 | (2S)-N-[(2E,4S)-1-(2,3- dihydro-1H-indol-1-yl)- 6-methyl-1-oxohept-2- en-4-yl]azetidine-2- carboxamide trifluoroacetate | 342 | 0.77 |
| 140 | P1; 33; A-C-H-C-A′ | 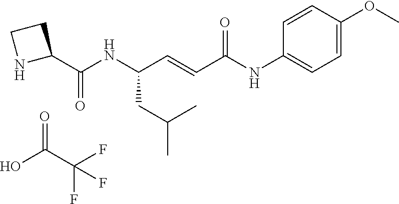 | (2S)-N-{(2E,4S)-1-[(4- methoxyphenyl)amino]- 6-methyl-l-oxohept-2- en-4-yl}azetidine-2- carboxamide trifluoroacetate | 346 | 0.73 |
| 141^{e} | P1; 33; A-C-H-C-A′ | 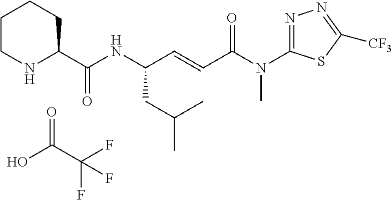 | (2S)-N-[(2E,4S)-6- methyl-1-{methyl[5- (trifluoromethyl)-1,3,4- thiadiazol-2-yl]amino}- 1-oxohept-2-en-4- yl]piperidine-2- carboxamide trifluoroacetate | 434 | 0.88 |
| 142 | P1; 58; A-C-H-C-A′ | 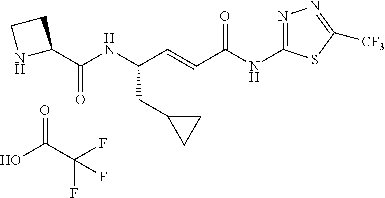 | (2S)-N-{(2S,3E)-5-[(5- tert-butyl-1,3,4- thiadiazol-2-yl)amino]- 1-cyclopropyl-5- oxopent-3-en-2- yl}azetidine-2- carboxamide trifluoroacetate | 378 | 0.66 |
| 143 | P1; 58; A-C-H-C-A′ | 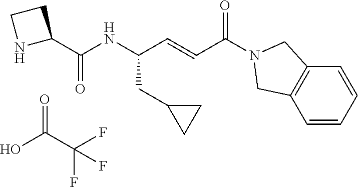 | (2S)-N-[(2S,3E)-1- cyclopropyl-5-(1,3- dihydro-2H-isoindol-2- yl)-5-oxopent-3-en-2- yl]azetidine-2- carboxamide trifluoroacetate | 340 | 0.71 |
| 144 | P1; 96; A-E-G-C-A′ | 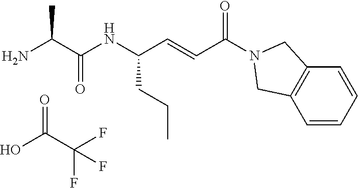 | N-[(2E,4S)-1-(1,3- dihydro-2H-isoindol-2- yl)-1-oxohept-2-en-4- yl]-L-alaninamide trifluoroacetate | 316 | 0.73 |
| 145 | P1; 96; A-E-G-C-A′ | 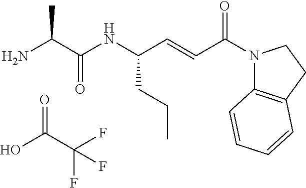 | N-[(2E,4S)-1-(2,3- dihydro-1H-indol-1-yl)- 1-oxohept-2-en-4-yl]-L- alaninamide trifluoroacetate | 316 | 0.78 |
| 146 | P1; 96; A-E-G-C-A′ | 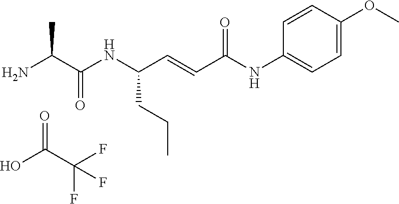 | (2E,4S)-4-(L- alanylamino)-N-(4- methoxyphenyl)hept-2- enamide trifluoroacetate | 320 | 0.74 |
| 147 | P1; 96; A-E-G-C-A′ | 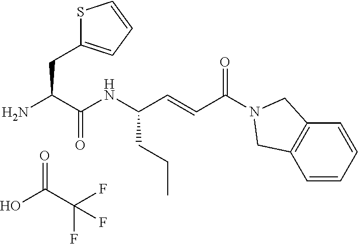 | N-(2E,4S)-1-(1,3- dihydro-2H-isoindol-2- yl)-1-oxohept-2-en-4- yl]-3-thiophen-2-yl-L- alaninamide trifluoroacetate | 398 | 0.89 |
| 148 | P1; 96; A-E-G-C-A′ | 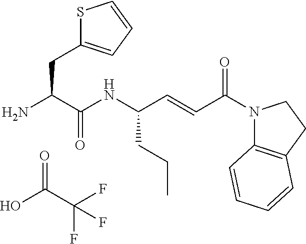 | N-[(2E,4,S)-1-(2,3- dihydro-1H-indol-1-yl)- 1-oxohept-2-en-4-yl]-3- thiophen-2-yl-L- alaninamide trifluoroacetate | 398 | 0.81 |
| 149 | P1; 96; A-E-G-C-A′ | 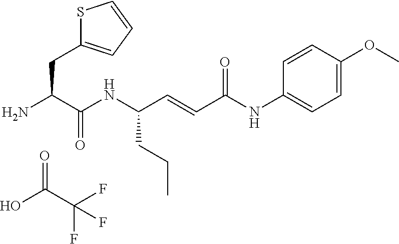 | (2E,4S)-N-(4- methoxyphenyl)-4-{[3 (thiophen-2-yl)-L- alanyl]amino}hept-2- enamide trifluoroacetate | 402 | 0.81 |
| 150 | P1; 96; A-E-G-C-A′ | 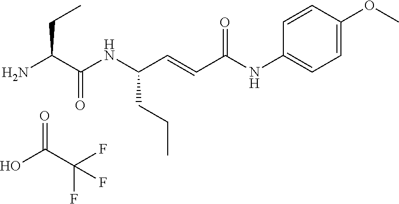 | (2E,4S)-4-{[(2S)-2- aminobutanoyl]amino}- N-(4- methoxyphenyl)hept-2- enamide trifluoroacetate | 334 | 0.74 |
| 151 | P1; 96; A-E-G-C-A′ | 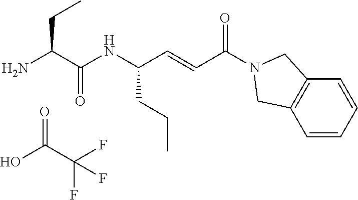 | (2S)-2-amino-N- [(2E,4S)-1-(1,3-dihydro- 2H-isoindol-2-yl)-1- oxohept-2-en-4- yl]butanamide trifluoroacetate | 330 | 0.74 |
| 152 | P1; 96; A-E-G-C-A′ | 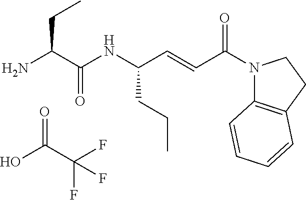 | (2S)-2-amino-N- [(2E,4S)-1-(2,3-dihydro- 1H-indol-1-yl)-1- oxohept-2-en-4- yl]butanamide trifluoroacetate | 330 | 0.8 |
| 153 | P1; 95; A-X-H-C-A′ | 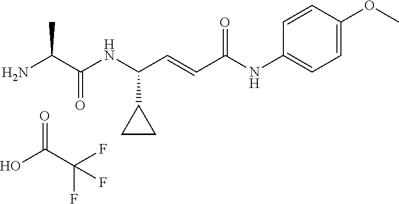 | (2E,4S)-4-(L- alanylamino)-4- cyclopropyl-N-(4- methoxyphenyl)but-2- enamide trifluoroacetate | 318 | 0.68 |
| 154 | P1; 95; A-X-H-C-A′ | 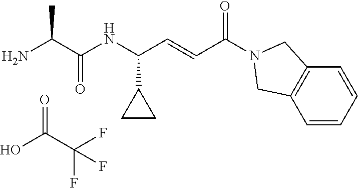 | N-[(1S,2E)-1- cyclopropyl-4-(1,3- dihydro-2H-isoindol-2- yl)-4-oxobut-2-en-1-yl]- L-alaninamide trifluoroacetate | 314 | 0.71 |
| 155 | P1; 95; A-X-H-C-A′ | 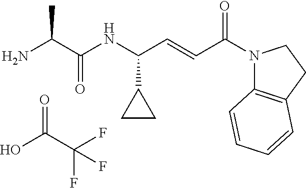 | N-[(1S,2E)-1- cyclopropyl-4-(2,3- dihydro-1H-indol-1-yl)- 4-oxobut-2-en-l-yl]-L- alaninamide trifluoroacetate | 314 | 0.78 |
| 156 | P1; 95; A-X-H-C-A′ | 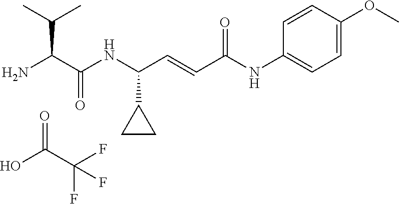 | N-{(1S,2E)-1- cyclopropyl-4-[(4- methoxyphenyl)amino]- 4-oxobut-2-en-l-yl}-L- valinamide trifluoroacetate | 346 | 0.74 |
| 157 | P1; 95; A-X-H-C-A′ | 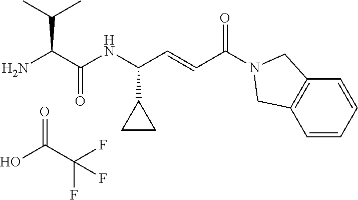 | N-[(1S,2E)-1- cyclopropyl-4-(1,3- dihydro-2H-isoindol-2- yl)-4-oxobut-2-en-1-yl]- L-valinamide trifluoroacetate | 342 | 0.74 |
| 158 | P1; 95; A-X-H-C-A′ | 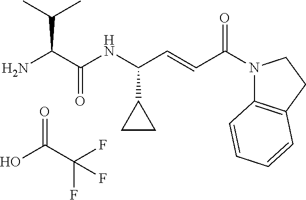 | N-[(1S,2E)-1- cyclopropyl-4-(2,3- dihydro-1H-indol-1-yl)- 4-oxobut-2-en-1-yl]-L valinamide trifluoroacetate | 342 | 0.82 |
| 159 | P1; 95; A-X-H-C-A′ | 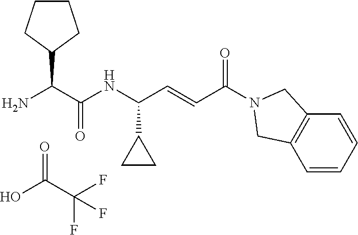 | (2S)-2-amino-2- cyclopentyl-N-[(1S,2E)- 1-cyclopropyl-4-(1,3- dihydro-2H-isoindol-2- yl)-4-oxobut-2-en-1- yl]ethanamide trifluoroacetate | 368 | 0.89 |
| 160 | P1; 95; A-X-H-C-A′ | 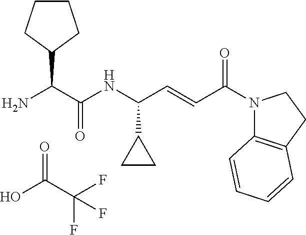 | (2S)-2-amino-2- cyclopentyl-N-[(1S,2E)- 1-cyclopropyl-4-(2,3- dihydro-1H-indol-1-yl)- 4-oxobut-2-en-1- yl]ethanamide trifluoroacetate | 368 | 0.96 |
| 161 | P1; 95; A-C-H-C-A′ | 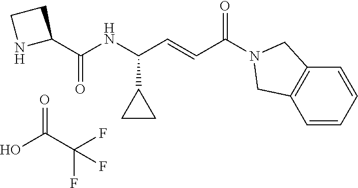 | (2S)-N-[(1S,2E)-1- cyclopropyl-4-(1,3- dihydro-2H-isoindol-2- yl)-4-oxobut-2-en-1- yl]azetidine-2- carboxamide trifluoroacetate | 326 | 0.65 |
| 162 | P1; 95; A-C-H-C-A′ | 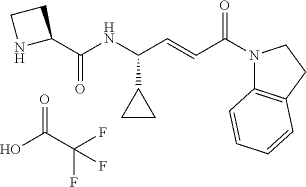 | (2S)-N-[(1S,2E)-1- cyclopropyl-4-(2,3- dihydro-1H-indol-1-yl)- 4-oxobut-2-en-1- yl]azetidine-2- carboxamide trifluoroacetate | 326 | 0.74 |
| 163 | P1; 95; A-X-H-C-A′ | 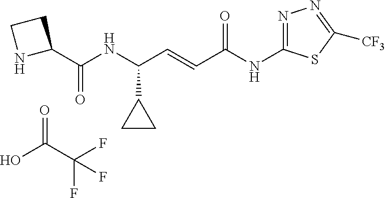 | (2S)-N-[(1S,2E)-1- cyclopropyl-4-oxo-4- {[5-(trifluoromethyl)- 1,3,4-thiadiazol-2- yl]amino}but-2-en-1- yl]azetidine-2- carboxamide trifluoroacetate | 376 | 0.77 |
| 164 | P1; 95; A-C-H-C-A′ | 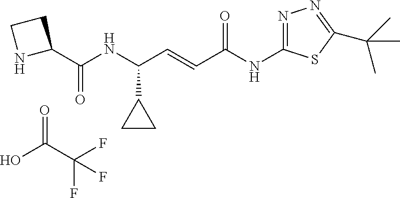 | (2S)-N-{(1S,2E)-4-[(5- tert-butyl-1,3,4- thiadiazol-2-yl)amino]- 1-cyclopropyl-4-oxobut- 2-en-1-yl}azetidine-2- carboxamide trifluoroacetate | 364 | 0.76 |
| 165 | P1; 18; A-E-G-Z-A′ | 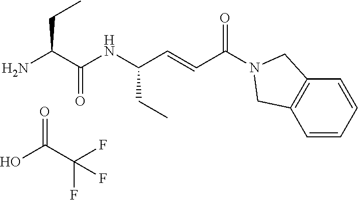 | (2S)-2-amino-N- [(3S,4E)-6-(1,3-dihydro- 2H-isoindol-2-yl)-6- oxohex-4-en-3- yl]butanamide trifluoroacetate | 316 | 0.65 |
| 166 | P1; 18; A-E-G-Z-A′ | 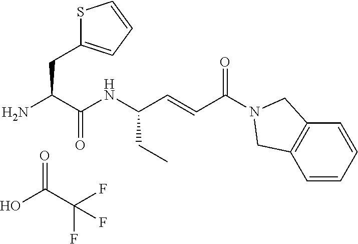 | N-[(3S,4E)-6-(1,3- dihydro-2H-isoindol-2- yl)-6-oxohex-4-en-3-yl]- 3-thiophen-2-yl-L- alaninamide trifluoroacetate | 384 | 0.71 |
| 167 | P1; 18; A-E-G-Z-A′ | 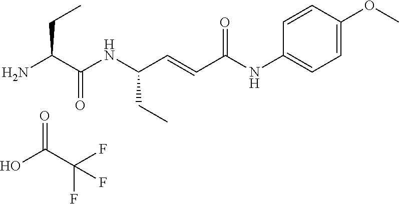 | (2E,4S)-4-{[(2S)-2- aminobutanoyl]amino}- N-(4- methoxyphenyl)hex-2- enamide trifluoroacetate | 320 | 0.66 |
| 168 | P1; 18; A-E-G-Z-A′ | 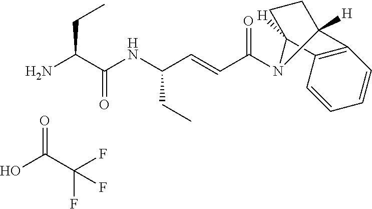 | (2S)-2-amino-N- {(3S,4E)-6-oxo-6- [(1R,4S)-1,2,3,4- tetrahydro-1,4- epiminonaphthalen-9- yl]hex-4-en-3- yl}butanamide trifluoroacetate | 342 | 0.7 |
| 169 | P1; 18; A-E-G-Z-A′ | 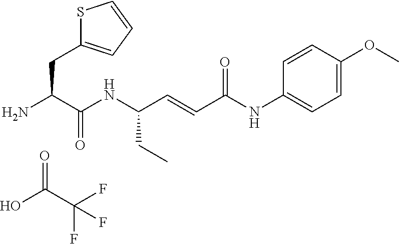 | (2E,4S)-N-(4- methoxyphenyl)-4-{[3- (thiophen-2-yl)-L- alanyl]amino}hex-2- enamide trifluoroacetate | 388 | 0.73 |
| 170 | P1; 18; A-E-G-Z-A′ | 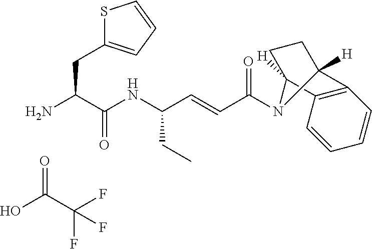 | N-[(3S,4E)-6-oxo-6- (1,2,3,4-tetrahydro-1,4- epiminonaphthalen-9- yl)hex-4-en-3-yl]-3- thiophen-2-yl-L- alaninamide trifluoroacetate | 410 | 0.77 |
| 171 | P1; 18; A-E-G-C-A′ | 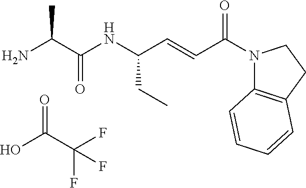 | N-[(3S,4E)-6-(2,3- dihydro-1H-indol-1-yl)- 6-oxohex-4-en-3-yl]-L- alaninamide trifluoroacetate | 302 | 0.7 |
| 172 | P1; 18; A-E-G-C-A′ | 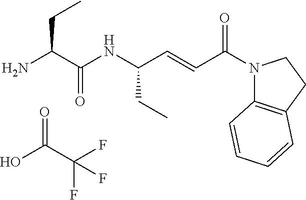 | (2S)-2-amino-N- [(3S,4E)-6-(2,3-dihydro- 1H-indol-1-yl)-6- oxohex-4-en-3- yl]butanamide trifluoroacetate | 316 | 0.72 |
| 173 | P1; 18; A-E-G-C-A′ | 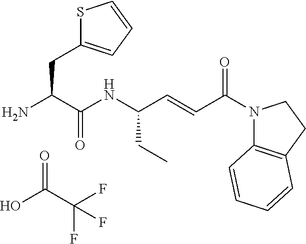 | N-[(3S,4E)-6-(2,3- dihydro-1H-indol-1-yl)- 6-oxohex-4-en-3-yl]-3- thiophen-2-yl-L- alaninamide trifluoroacetate | 384 | 0.79 |
| 174 | P1; 18; A-C-H-B-A′ | 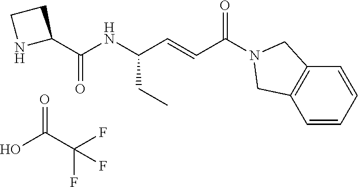 | (2S)-N-[(3S,4E)-6-(1,3- dihydro-2H-isoindol-2- yl)-6-oxohex-4-en-3- yl]azetidine-2- carboxamide trifluoroacetate | 314 | 0.68 |
| 175 | P1; 18; A-C-H-C-A′ | 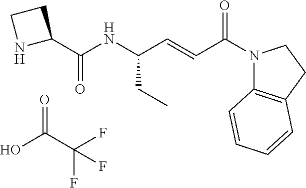 | (2S)-N-[(3S,4E)-6-(2,3- dihydro-1H-indol-1-yl)- 6-oxohex-4-en-3- yl]azetidine-2- carboxamide trifluoroacetate | 314 | 0.61 |
| 176 | P1; 18 A-C-H-C-A′ | 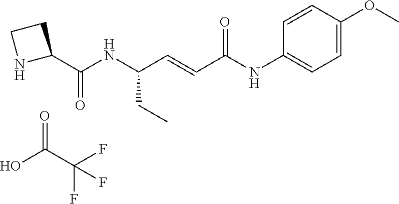 | (2S)-N-{(3S,4E)-6-[(4- methoxyphenyl)amino]- 6-oxohex-4-en-3- yl}azetidine-2- carboxamide trifluoroacetate | 318 | 0.66 |
| 177^{f} | P1; 18; A-C-H-C-A′ | 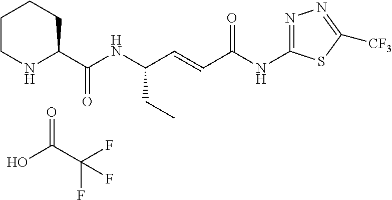 | (2S)-N-[(3S,4E)-6-oxo- 6-{[5-(trifluoromethyl)- 1,3,4-thiadiazol-2- yl]amino}hex-4-en-3- yl]piperidine-2- carboxamide trifluoroacetate | 392 | 0.79 |
| 178 | P1; 18; A-C-H-C-A′ | 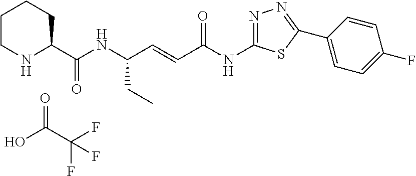 | (2S)-N-[(3S,4E)-6-{[5- (4-fluorophenyl)-1,3,4- thiadiazol-2-yl]amino}- 6-oxohex-4-en-3- yl]piperidine-2- carboxamide trifluoroacetate | 418 | 0.8 |
| 179^{g} | P1; 18; A-C-H-C-A′ | 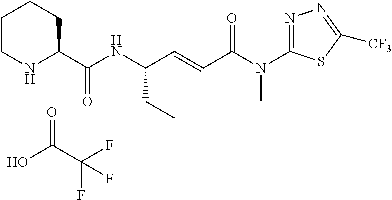 | (2S)-N-[(3S,4E)-6- {methyl[5- (trifluoromethyl)-1,3,4- thiadiazol-2-yl]amino}- 6-oxohex-4-en-3- yl]piperidine-2- carboxamide trifluoroacetate | 406 | 0.77 |
| 180 | P1; 94; A-E-H-C-A′ | 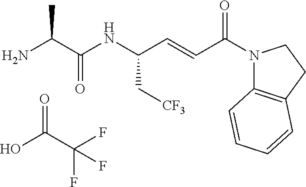 | N-[(4E)-6-(2,3-dihydro- 1H-indol-1-yl)-1,1,1- trifluoro-6-oxohex-4-en- 3-yl]-L-alaninamide trifluoroacetate | 356 | 0.77 |

^{a}Example 81 ¹H NMR (MeOD) δ ppm 7.13-7.35 (m, 5H), 7.03 (dd, J = 15.4, 5.9 Hz, 1H), 6.27 (dd, J = 15.3, 1.5 Hz, 1H), 4.60-4.66 (m, 1H), 3.85 (dd, J = 11.9, 3.1 Hz, 1H), 3.41-3.47 (m, 1H), 3.03-3.11 (m, 1H), 2.72-2.82 (m, 2H), 2.69 (s, 3H), 2.31-2.38 (m, 1H), 1.81-2.09 (m, 5H), 1.60-1.78 (m, 4H).
^{b}Example 82 ¹H NMR (MeOD) δ ppm 7.12-7.33 (m, 5H), 7.03 (dd, J = 15.4, 5.9 Hz, 1H), 6.27 (dd, J = 15.6, 1.5 Hz, 1H), 4.54-4.69 (m, 1H), 3.86 (dd, J = 11.9, 3.1 Hz, 1H), 3.38-3.54 (m, 1H), 3.07 (q, J = 7.5 Hz, 2H), 2.69-2.83 (m, 2H), 2.27-2.41 (m, 1H), 1.87-2.09 (m, 5H), 1.63-1.85 (m, 4H), 1.41 (t, J = 7.5 Hz, 3H).
^{c}Example 117 ¹H NMR (MeOD) δ ppm 8.44 (d, J = 8.0 Hz, 1H), 8.17 (d, J = 7.8 Hz, 1H), 7.26 (d, J = 7.0 Hz, 1H), 7.19 (t, J = 7.5 Hz, 1H), 7.07 (t, J = 7.4 Hz, 1H), 6.80 (dd, J = 15.2, 6.4 Hz, 1H), 6.44 (d, J = 15.1 Hz, 1H), 4.56 (t, J = 7.2 Hz, 1H), 4.08-4.29 (m, 2H), 3.71-3.91 (m, 1H), 3.42 (d, J=12.5 Hz, 1H), 3.24 (t, J = 7.9 Hz, 2H), 3.00-3.12 (m, 1H), 2.46 (q, J = 7.8 Hz, 1H), 2.27 (d, J = 11.0 Hz, 1H), 2.05-2.19 (m, 2H), 1.81-2.05 (m, 4H), 1.60-1.81 (m, 7H).
^{d}Example 118 ¹H NMR (MeOD) δ ppm 8.50 (d, J = 8.0 Hz, 1H), 7.08 (dd, J = 15.3, 5.8 Hz, 1H), 6.28 (dd, J = 15.6, 1.5 Hz, 1H), 4.49-4.68 (m, 1H), 3.76-3.92 (m, 1H), 3.35-3.52 (m, 1H), 2.93-3.16 (m, 1H), 2.46 (q, J = 7.8 Hz, 1H), 2.33 (d, J = 13.3 Hz, 1H), 2.04-2.19 (m, 2H), 1.81-2.04 (m, 5H), 1.59-1.81 (m, 7H).
^{e}Example 141 ¹H NMR (MeOD) δ ppm 8.53 (d, J = 8.0 Hz, 1H), 7.08 (dd, J = 15.2, 6.1 Hz, 1H), 6.76 (dd, J = 15.1, 1.3 Hz, 1H), 4.69-4.81 (m, 1H), 3.90-4.03 (m, 3H), 3.77-3.90 (m, 1H), 3.36-3.55 (m, 1H), 3.06 (td, J = 12.6, 3.1 Hz, 1H), 2.30 (d, J = 12.0 Hz, 1H), 1.83-2.07 (m, 2H), 1.68-1.79 (m, 3H), 1.53-1.67 (m, 3H), 1.01(d, J = 7.9 Hz, 3H), 1.00 (d, J = 7.9 Hz, 3H).
^{f}Example 177 ¹H NMR (MeOD) δ ppm 8.53 (d, J = 8.0 Hz, 1H), 7.11 (dd, J = 15.4, 5.6 Hz, 1H), 6.31 (dd, J = 15.3, 1.5 Hz, 1H), 4.49-4.65 (m, 1H), 3.87 (dd, J = 11.7, 3.1 Hz, 1H), 3.40-3.52 (m, 1H), 2.98-3.13 (m, 1H), 2.27-2.40 (m, 1H), 1.87-2.10 (m, 2H), 1.66-1.87 (m, 5H), 1.03 (t, J = 7.9 Hz, 3H).
^{g}Example 179 ¹H NMR (MeOD) δppm 8.56 (d, J = 8.0 Hz, 1H), 7.09 (dd, J = 15.2, 6.1 Hz, 1H), 6.78 (dd, J = 15.1, 1.5 Hz, 1H), 4.51-4.67 (m, 1H), 3.92 (s, 3H), 3.82-3.90 (m, 1H), 3.37-3.53 (m, 1H), 2.98-3.16 (m, 1H), 2.19-2.38 (m, 1H), 1.86-2.11 (m, 2H), 1.61-1.86 (m, 5H), 1.04 (t, J = 7.9 Hz, 3H).

### Example 181

### (4S)—N-[(1S,2E)-4-(2,3-dihydro-1H-indol-1-yl)-1-(2-methylpropyl)-4-oxo-2-buten-1-yl]-4-fluoro-L-prolinamide sulfate

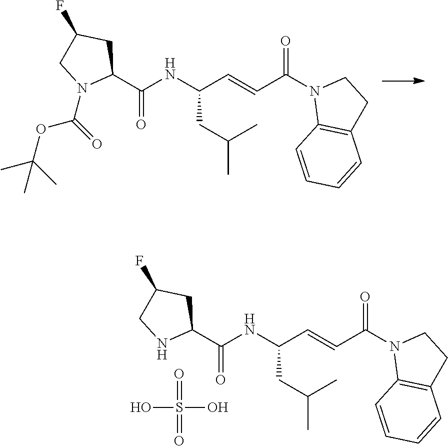

To a solution of 1,1-dimethylethyl (2S,4S)-2-({[(1S,2E)-4-(2,3-dihydro-1H-indol-1-yl)-1-(2-methylpropyl)-4-oxo-2-buten-1-yl]amino}carbonyl)-4-fluoro-1-pyrrolidinecarboxylate (52 mg, 0.110 mmol) in 1,4-dioxane (2.0 mL) was added concentrated H₂SO₄ (0.018 mL, 0.329 mmol). The reaction mixture was stirred overnight at RT. The reaction mixture was then concentrated in vacuo and purified by reverse phase HPLC (10-50% CH₃CN/H₂O) to afford the title compound (17 mg, 31%) as a white solid. LC-MS m/z 374 (M+H)⁺, 0.83 min (ret time). ¹H NMR (400 MHz, MeOD) δ ppm 8.17 (d, J=8.0 Hz, 1H), 7.26 (d, J=6.8 Hz, 1H), 7.13-7.22 (m, 1H), 6.97-7.09 (m, 1H), 6.84 (dd, J=15.1, 5.8 Hz, 1H), 6.46 (d, J=14.8 Hz, 1H), 5.46 (d, J=52.1 Hz, 1H), 4.70-4.76 (m, 1H), 4.59 (dd, J=10.2, 2.6 Hz, 1H), 4.07-4.29 (m, 2H), 3.71-3.91 (m, 1H), 3.52-3.69 (m, 1H), 3.23 (t, J=8.0 Hz, 2H), 2.71-2.90 (m, 1H), 2.52-2.65 (m, 1H), 1.69-1.86 (m, 1H), 1.47-1.68 (m, 2H), 1.01 (d, J=8.0 Hz, 3H), 0.99 (d, J=8.0 Hz, 3H).

### Biological Background:

### Biological Assay(s)

The compounds according to Formula (I) are cathepsin C inhibitors, which indirectly inhibit the activity of serine proteases that are activated by cathepsin C, such as NE. The compounds according to Formula (I), therefore, are useful in the treatment of COPD and other conditions involving cathepsin C and/or such serine proteases. The biological activity of the compounds according to Formula (I) can be determined using any suitable assay for determining the activity of a candidate compound as a cathepsin C inhibitor or for determining the ability of a candidate compound to prevent the cathepsin C mediated activation of certain serine proteases, as well as suitable tissue and in vivo models. All examples were found to be cathepsin C inhibitors.

### A. Transpeptidation of Leucine-Leucine-O-Methyl (LLOM) Cell-Based Luminescence Viability Assay

### Principle:

Cathepsin C has been shown to catalyze the transpeptidation of dipeptidyl methyl-(O)-esters within the lysosomes of cells from the monocytic lineage such as HL60, U937 or THP1 causing a membranolytic effect that results in cell death (DL. Thiele, P. Lipsky PNAS1990 Vol. 87, pp. 83-87). This mechanism was used to assess Cathepsin C in cells activity in the presence of the compounds of the invention.

Frozen HL-60 cells were resuspended at 1.25×10⁵ cells/mL in fresh prewarmed Iscove's modified Dulbeccos' medium (IMDM, contains 25 mM glutamine) with 20% FBS. This suspension was dispensed (8 μL) into white low volume 384 well plates. Plates were previously stamped with 100 mL of compound at a top concentration of 2.5 mM and serially diluted 1:3. Control and blank wells contained 100 mL of DMSO. Each well then received 2 μL of a fresh 1.25 mM solution of leucine-leucine-OMethyl (LLOM, Bachem) in IMDM plus 25 mM HEPES (final concentration LLOM 250 μM). The plates were covered and incubated for 4 h at 37° C. in a 5% CO₂ incubator, then removed and equilibrated to room temperature for 10 min. Cell viability was determined with a CellTiter-Glo luminescent assay (Promega) according to the manufacturer's instructions. Cell viability was compared to controls containing no LLOM (100%).

### B. Human Neutrophil Cathepsin C Assay

Neutrophils (PMN) were isolated from human peripheral blood using standard methods. In brief, 25 mL blood was layered over 15 mL Ficol-Paque Plus (Amersham Biosciences) and centrifuged at 400 g at room temperature for 30 min. The red blood cell pellets were resuspended in 35 mL phosphate-buffered saline without Ca²⁺ or Mg²⁺ (PBS). Dextran T-500 (Pharmacia, 6% solution in PBS) was added to each tube (12 mL), tubes were mixed by inversion, and allowed to stand at room temperature for 40 min. The layer above the red cells was collected, centrifuged at 800 g, and gently resuspended ˜3 mL. Red blood cells were lysed by addition of 18 mL sterile water for 30 sec, followed immediately by addition of 2 mL 10×PBS. Cells were recollected and resuspended to 2×10⁵ cells/mL in PBS with 0.1% gelatin.

Compounds were prepared in serial three-fold dilutions starting with a top concentration of 10 mM solution in DMSO. PMN were then were plated in wells in 96-well flat-bottom tissue culture plates in (20,000 cells in 100 μL). Compound was added (1 μL, each concentration) to the wells in triplicate, plates were mixed for 5 min on a plate shaker and then incubated for 30 min at 37° C., 5% CO₂. Substrate (H-Gly-Arg)₂ R110 was added (5 μL, of a 0.5 mM solution in PBS) and plates incubated for a further 3 h. The cleavage of substrate was measured at using an excitation wavelength of 485 nm and an emission wavelength of 530 nm. Compounds were compared to controls containing DMSO only and IC₅₀'s were determined using non-linear regression curve fit analysis (GraphPad Prism).

### C. Recombinant Cathepsin C In Vitro Assay:

The activity of recombinant human cathepsin C was measured by the cleavage of a fluorogenic substrate, H-Ser-Tyr-AMC. Briefly, 24 pM cathepsin C was incubated with test compound (e.g. inhibitor) in a buffer consisting of 50 mM sodium acetate, 30 mM sodium chloride, 1 mM CHAPS, 1 mM dithiothreitol, 1 mM EDTA, pH 5.5 at room temperature for one hour. After one hour of incubating test compound with cathepsin C, the activity assay was initiated by the addition of an equal volume of 0.010 mM H-Ser-Tyr-AMC in the same buffer. After one hour, the activity assay was stopped by the addition of ⅕ volume of 100 μM E-64. The reaction product was measured on a fluorescence reader set at an excitation wavelength of 360 nm and emission wavelength of 460 nm and equipped with a 400 nm dichroic mirror.

The following Example numbers represent preferred compounds of this invention: 1, 7, 10, 13, 14, 15, 17, 18, 19, 20, 21, 23, 26, 28, 29, 30, 31, 33, 43, 44, 50, 52, 53, 57, 59, 62, 65, 68, 76, 78, 79, 96, 97, 99, 107, 112, 114, 124, 125, 126, 128, 138, 139, 146, 157, 161, 162, 164, 167, 174, 175, and 179. The following Example numbers represent the more preferred compounds of this invention: 5, 6, 8, 9, 11, 12, 16, 38, 39, 40, 45, 46, 47, 48, 51, 54, 55, 56, 58, 60, 63, 64, 66, 67, 70, 71, 72, 73, 74, 75, 77, 80, 81, 82, 83, 84, 85, 86, 87, 88, 89, 90, 91, 92, 93, 94, 95, 98, 100, 103, 105, 106, 108, 109, 110, 116, 117, 118, 119, 120, 121, 129, 130, 131, 132, 133, 134, 135, 136, 137, 141, 142, 143, 144, 145, 147, 148, 149, 150, 151, 152, 154, 155, 158, 160, 165, 166, 168, 169, 170, 171, 172, 173, 177, 178, 180, and 181.

The compounds of the invention (Examples 1-181) exhibit 50% cathepsin C inhibition (as determined using the above method) at concentrations of from approximately 5,000 nM to approximately 0.01 nM. For instance, the compound of Example 3 exhibited 50% cathepsin C inhibition at a concentration of approximately 1,000 nM. Preferred compounds of the invention exhibit 50% inhibition at concentrations of from approximately 100 nM to approximately 0.01 nM. For instance, the compound of Example 1 exhibited 50% cathepsin C inhibition at a concentration of approximately 100 nM. More preferred compounds of the invention exhibit 50% inhibition at concentrations of from approximately 10 nM to approximately 0.01 nM.

The compounds of the invention are believed to be useful in therapy as defined above and to not have unacceptable or untoward effects when used in compliance with a permitted therapeutic regime.

The foregoing examples and assay have been set forth to illustrate the invention, not limit it. What is reserved to the inventors is to be determined by reference to the claims.

1-39. (canceled)

40. A compound according to Formula (I) or a pharmaceutically acceptable salt thereof: 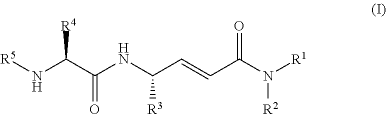
wherein:
R¹ and R² are each independently selected from the group consisting of hydrogen, (C₁-C₈)alkyl, (C₂-C₈)alkenyl, (C₂-C₈)alkynyl, (C₃-C₈)cycloalkyl, (C₅-C₈)cycloalkenyl, (C₆-C₁₀)bicycloalkyl, heterocycloalkyl, (C₃-C₈)cycloalkyl(C₁-C₆)alkyl, (C₅-C₈)cycloalkenyl(C₁-C₆)alkyl, heterocycloalkyl(C₁-C₆)alkyl, aryl, heteroaryl, aryl(C₁-C₆)alkyl, and heteroaryl(C₁-C₆)alkyl;
wherein any (C₁-C₈)alkyl, (C₂-C₈)alkenyl, or (C₂-C₈)alkynyl is optionally substituted one to three times, independently, by —CF₃, cyano, —CO₂(C₁-C₄)alkyl, —CONH(C₁-C₄)alkyl, —CON(C₁-C₄)alkyl(C₁-C₄)alkyl, —SO₂(C₁-C₄)alkyl, —SO₂NH(C₁-C₄)alkyl, —SO₂N(C₁-C₄)alkyl(C₁-C₄)alkyl, amino, (C₁-C₄)alkylamino, ((C₁-C₄)alkyl)((C₁-C₄)alkyl)amino, hydroxyl, or (C₁-C₄)alkoxy;
and wherein any cycloalkyl, cycloalkenyl, bicycloalkyl, or heterocycloalkyl group is optionally substituted one to three times, independently, by (C₁-C₄)alkyl, (C₁-C₄)haloalkyl, cyano, —CO₂(C₁-C₄)alkyl, —CONH(C₁-C₄)alkyl, —CON(C₁-C₄)alkyl(C₁-C₄)alkyl, —SO₂(C₁-C₄)alkyl, —SO₂NH(C₁-C₄)alkyl, —SO₂N(C₁-C₄)alkyl(C₁-C₄)alkyl, amino, (C₁-C₄)alkylamino, ((C₁-C₄)alkyl)((C₁-C₄)alkyl)amino, hydroxyl, (C₁-C₄)alkoxy, aryl, or aryl(C₁-C₄)alkyl, wherein the aryl moiety of said aryl or aryl(C₁-C₄)alkyl is optionally substituted one to three times, independently, by halogen, —CF₃, (C₁-C₄)alkyl, hydroxyl, or (C₁-C₄)alkoxy;
and wherein any aryl or heteroaryl group is optionally substituted one to three times, independently, by halogen, (C₁-C₆)alkyl, (C₃-C₆)cycloalkyl, (C₅-C₆)cycloalkenyl, (C₁-C₆)haloalkyl, cyano, —CO₂(C₁-C₁₄)alkyl, —CONH(C₁-C₄)alkyl, —CON(C₁-C₄)alkyl(C₁-C₄)alkyl, —SO₂(C₁-C₄)alkyl, —SO₂NH(C₁-C₄)alkyl, —SO₂N(C₁-C₄)alkyl(C₁-C₄)alkyl, amino, (C₁-C₄)alkylamino, ((C₁-C₄)alkyl)((C₁-C₄)alkyl)amino, hydroxyl, (C₁-C₄)alkoxy, (C₁-C₄)alkylthio-, aryl, heteroaryl, aryl(C₁-C₄)alkyl, or heteroaryl(C₁-C₄)alkyl;
wherein any aryl or heteroaryl moiety of said aryl, heteroaryl, aryl(C₁-C₄)alkyl, or heteroaryl(C₁-C₄)alkyl is optionally substituted one to three times, independently, by halogen, —CF₃, (C₁-C₄)alkyl, hydroxyl, or (C₁-C₄)alkoxy;
and wherein any (C₃-C₆)cycloalkyl is optionally substituted one to three times, independently, by (C₁-C₄)alkyl, aryl, or heteroaryl;
wherein said aryl or heteroaryl is optionally substituted one to three times, independently, by halogen, —CF₃, (C₁-C₄)alkyl, hydroxyl, or (C₁-C₄)alkoxy;
or R¹ and R² taken together with the nitrogen to which they are attached represent a 5- to 7-membered saturated or unsaturated ring optionally containing one other heteroatom which is oxygen, nitrogen, or sulfur, wherein said ring is optionally fused to a (C₃-C₈)cycloalkyl, heterocycloalkyl, aryl, or heteroaryl ring;
or R¹ and R² taken together with the nitrogen to which they are attached represent a 6- to 10-membered bridged bicyclic ring system optionally fused to a (C₃-C₈)cycloalkyl, heterocycloalkyl, aryl, or heteroaryl ring;
R³ is hydrogen, (C₁-C₈)alkyl, (C₁-C₈)haloalkyl, (C₂-C₈)alkenyl, (C₂-C₈)alkynyl, (C₃-C₆)cycloalkyl, (C₅-C₆)cycloalkenyl, (C₃-C₆)cycloalkyl(C₁-C₄)alkyl, (C₅-C₆)cycloalkenyl(C₁-C₄)alkyl, or aryl(C₁-C₄)alkyl, wherein the aryl moiety of the aryl(C₁-C₄)alkyl is optionally substituted one to three times, independently, by halogen, (C₁-C₄)alkyl, or —CF₃;
R⁴ is hydrogen, (C₁-C₄)alkyl, (C₂-C₅)alkenyl, (C₂-C₅)alkynyl, (C₃-C₅)cycloalkyl, (C₃-C₄)cycloalkyl(C₁-C₂)alkyl, cyano(C₁-C₂)alkyl, hydroxy(C₁-C₂)alkyl, methoxy(C₁-C₂)alkyl, aryl(C₁-C₂)alkyl, or heteroaryl(C₁-C₂)alkyl, wherein the heteroaryl moiety of said heteroaryl(C₁-C₂)alkyl is a 5-membered aromatic ring containing one heteroatom which is oxygen or sulfur and optionally containing one or two nitrogen atoms; and
R⁵ is hydrogen or methyl;
or R⁴ and R⁵ taken together with atoms through which they are connected form a 4- to 6-membered saturated ring optionally substituted one or two times, independently, by halogen, —CF₃, cyano, (C₁-C₄)alkyl, amino, (C₁-C₄)alkylamino, ((C₁-C₄)alkyl)((C₁-C₄)alkyl)amino, hydroxyl, (C₁-C₄)alkoxy, or (C₁-C₄)alkylthio-; wherein said ring is optionally fused to a (C₃-C₅)cycloalkyl ring.

41. The compound or salt according to claim 40, wherein R¹ is selected from the group consisting of (C₁-C₆)alkyl, (C₃-C₇)cycloalkyl, (C₇-C₉)bicycloalkyl, heterocycloalkyl, (C₃-C₇)cycloalkyl(C₁-C₂)alkyl, phenyl, heteroaryl, and phenyl(C₁-C₂)alkyl; wherein any cycloalkyl or heterocycloalkyl group is optionally substituted one to two times, independently, by (C₁-C₄)alkyl, —CF₃, hydroxyl, or (C₁-C₄)alkoxy, and wherein any phenyl or heteroaryl group is optionally substituted one to two times, independently, by halogen, (C₁-C₄)alkyl, —CF₃, cyano, —CO₂(C₁-C₄)alkyl, hydroxyl, (C₁-C₄)alkoxy, or (C₁-C₄)alkylthio-.

42. The compound or salt according to claim 41, wherein R² is hydrogen or methyl.

43. The compound or salt according to claim 40, wherein R¹ and R² taken together with the nitrogen to which they are attached represent a 5- to 7-membered saturated or unsaturated ring optionally containing one other heteroatom which is oxygen, nitrogen, or sulfur; wherein said ring is optionally fused to a (C₃-C₈)cycloalkyl, heterocycloalkyl, aryl, or heteroaryl ring.

44. The compound or salt according to claim 40, wherein R¹ and R² taken together with the nitrogen to which they are attached represent a 5- to 6-membered saturated or unsaturated ring optionally fused to a phenyl moiety.

45. The compound or salt according to claim 40, wherein R³ is hydrogen, (C₁-C₆)alkyl, (C₁-C₆)haloalkyl, (C₃-C₆)cycloalkyl, (C₃-C₆)cycloalkyl(C₁-C₄)alkyl, or phenyl(C₁-C₄)alkyl; wherein the phenyl moiety of the phenyl(C₁-C₄)alkyl is optionally substituted one to three times, independently, by halogen, (C₁-C₄)alkyl, or —CF₃.

46. The compound or salt according to claim 40, wherein R³ is (C₁-C₆)alkyl or (C₃-C₆)cycloalkyl(C₁-C₂)alkyl.

47. The compound or salt according to claim 40, wherein R⁴ is hydrogen, (C₁-C₄)alkyl, (C₃-C₅)cycloalkyl, or heteroaryl(C₁-C₂)alkyl; wherein the heteroaryl moiety of said heteroaryl(C₁-C₂)alkyl is a 5-membered aromatic ring containing one heteroatom which is oxygen or sulfur and optionally containing one or two nitrogen atoms.

48. The compound or salt according to claim 40, wherein R⁴ is (C₁-C₄)alkyl, (C₃-C₅)cycloalkyl, or thienyl(C₁-C₂)alkyl.

49. The compound or salt according to claim 40, wherein R⁴ is methyl, ethyl, isopropyl, cyclopentyl, or 2-thienylmethyl.

50. The compound or salt according to claim 40, wherein R⁵ is hydrogen.

51. The compound or salt according to claim 40, wherein R⁴ and R⁵ taken together with atoms through which they are connected form a 4- to 6-membered saturated ring optionally substituted by F, Cl, —CF₃, cyano, methyl, methoxy, or methylthio-.

52. The compound or salt according to claim 40, wherein R⁴ and R⁵ taken together with atoms through which they are connected form a 4- to 6-membered saturated ring optionally substituted by F.

53. The compound or salt according to claim 40, wherein:
R¹ and R² taken together with the nitrogen to which they are attached represent 2,3-dihydro-1H-indol-1-yl;
R³ is (C₁-C₆)alkyl or (C₃-C₆)cycloalkyl(C₁-C₂)alkyl; and
R⁴ and R⁵ taken together with atoms through which they are connected form a 4- to 6-membered saturated ring optionally substituted by F, Cl, —CF₃, cyano, methyl, methoxy, or methylthio-.

54. The compound or salt according to claim 40, wherein:
R¹ is thiadiazolyl optionally substituted by halogen, (C₁-C₄)alkyl, —CF₃, (C₃-C₆)cycloalkyl, phenyl, cyano, —CO₂(C₁-C₄)alkyl, or (C₁-C₄)alkoxy; wherein said (C₃-C₆)cycloalkyl is optionally substituted by (C₁-C₄)alkyl;
R² is hydrogen or methyl;
R³ is (C₁-C₆)alkyl or (C₃-C₆)cycloalkyl(C₁-C₂)alkyl; and
R⁴ and R⁵ taken together with atoms through which they are connected form a 4- to 6-membered saturated ring optionally substituted by F, Cl, —CF₃, cyano, methyl, methoxy, or methylthio-.

55. A compound which is:
(2E,4S)-4-(L-alanylamino)-6-phenyl-N-(phenylmethyl)-2-hexenamide;
(2E,4S)-4-(L-alanylamino)-N-methyl-6-phenyl-2-hexenamide;
(2E,4S)-4-(L-alanylamino)-N,N-dimethyl-6-phenyl-2-hexenamide;
N¹-[(1S,2E)-4-oxo-1-(2-phenylethyl)-4-(1-piperidinyl)-2-buten-1-yl]-L-alaninamide;
(2E,4S)-4-(L-alanylamino)-N-[4-(methyloxy)phenyl]-6-phenyl-2-hexenamide;
methyl 3-{[(2E,4S)-4-(L-alanylamino)-6-phenyl-2-hexenoyl]amino}benzoate;
(2E,4S)-4-(L-alanylamino)-N-[2-(methyloxy)phenyl]-6-phenyl-2-hexenamide;
(2E,4S)-4-(L-alanylamino)-6-phenyl-N-1,3-thiazol-2-yl-2-hexenamide;
(2E,4S)-4-(L-alanylamino)-6-phenyl-N-[3-(trifluoromethyl)phenyl]-2-hexenamide;
(2E,4S)-4-(L-alanylamino)-N-methyl-N,6-diphenyl-2-hexenamide;
(2E,4S)-4-(L-alanylamino)-6-phenyl-N-[4-(trifluoromethyl)phenyl]-2-hexenamide;
methyl 4-{[(2E,4S)-4-(L-alanylamino)-6-phenyl-2-hexenoyl]amino}benzoate;
(2E,4S)-4-(L-alanylamino)-N-cyclohexyl-N-methyl-6-phenyl-2-hexenamide;
(2E,4S)-4-(L-alanylamino)-N-[(1R,3S)-3-hydroxycyclopentyl]-6-phenyl-2-hexenamide;
(2E,4S)-4-(L-alanylamino)-N-[(1S,4R)-bicyclo[2.2.1]hept-2-yl]-6-phenyl-2-hexenamide;
N¹-[(1S,2E)-4-(1H-indol-1-yl)-4-oxo-1-(2-phenylethyl)-2-buten-1-yl]-L-alaninamide;
(2E,4S)-4-(L-alanylamino)-N-[3-(methyloxy)phenyl]-6-phenyl-2-hexenamide;
(2E,4S)-4-(L-alanylamino)-N-cyclohexyl-6-phenyl-2-hexenamide;
(2E,4S)-4-(L-alanylamino)-6-phenyl-N-3-pyridinyl-2-hexenamide;
(2E,4S)-4-(L-alanylamino)-6-phenyl-N-1H-pyrazol-4-yl-2-hexenamide;
(2E,4S)-4-(L-alanylamino)-6-phenyl-N-propyl-2-hexenamide;
(2E,4S)-4-(L-alanylamino)-N-(1,1-dimethylethyl)-6-phenyl-2-hexenamide;
(2E,4S)-4-(L-alanylamino)-N-cyclopentyl-6-phenyl-2-hexenamide;
(2E,4S)-4-(L-alanylamino)-N-(1-methyl-4-piperidinyl)-6-phenyl-2-hexenamide;
(2E,4S)-4-(L-alanylamino)-6-phenyl-N-(tetrahydro-2H-pyran-4-yl)-2-hexenamide;
(2E,4S)-4-(L-alanylamino)-N-(cyclohexylmethyl)-6-phenyl-2-hexenamide;
(2E,4S)-4-(L-alanylamino)-N-(1-methylethyl)-6-phenyl-2-hexenamide;
(2E,4S)-4-(L-alanylamino)-N-cycloheptyl-6-phenyl-2-hexenamide;
(2E,4S)-4-(L-alanylamino)-N-ethyl-6-phenyl-2-hexenamide;
(2E,4S)-4-(L-alanylamino)-N-[(1R,4S)-bicyclo[2.2.1]hept-2-yl]-6-phenyl-2-hexenamide;
(2E,4S)-4-(L-alanylamino)-N-(1-methylpropyl)-6-phenyl-2-hexenamide;
(2E,4S)-4-(L-alanylamino)-N-(2-methylcyclohexyl)-6-phenyl-2-hexenamide;
(2E,4S)-4-(L-alanylamino)-N-(4-hydroxycyclohexyl)-6-phenyl-2-hexenamide;
(2E,4S)-4-(L-alanylamino)-6-phenyl-N-(tetrahydro-3-furanyl)-2-hexenamide;
(2E,4S)-4-(L-alanylamino)-6-phenyl-N-(tetrahydro-2H-pyran-3-yl)-2-hexenamide;
(2E,4S)-4-(L-alanylamino)-N-(1-methyl-3-piperidinyl)-6-phenyl-2-hexenamide;
(2E,4S)-4-(L-alanylamino)-N-[(1S,3S)-3-hydroxycyclopentyl]-6-phenyl-2-hexenamide;
N¹-[(1S,2E)-4-[(1R,8S)-11-azatricyclo[6.2.1.0^{2,7}]undeca-2,4,6-trien-11-yl]-4-oxo-1-(2-phenylethyl)-2-buten-1-yl]-L-alaninamide;
(2S)-2-amino-N-[(1S,2E)-4-[(1R,8S)-11-azatricyclo[6.2.1.0^{2,7}]undeca-2,4,6-trien-11-yl]-4-oxo-1-(2-phenylethyl)-2-buten-1-yl]butanamide;
N¹-[(1S,2E)-4-(1,3-dihydro-2H-isoindol-2-yl)-1-ethyl-4-oxo-2-buten-1-yl]-L-alaninamide;
N¹-{(1S,2E)-4-[(1R,8S)-11-azatricyclo[6.2.1.0^{2,7}]undeca-2,4,6-trien-11-yl]-1-[(1S)-1-methylpropyl]-4-oxo-2-buten-1-yl}-L-alaninamide;
N¹-{(1S,2E)-4-(1,3-dihydro-2H-isoindol-2-yl)-1-[(1S)-1-methylpropyl]-4-oxo-2-buten-1-yl}-L-alaninamide;
(2E,4S)-4-(L-alanylamino)-6-methyl-N-[4-(methyloxy)phenyl]-2-heptenamide;
N¹-[(1S,2E)-4-(1,3-dihydro-2H-isoindol-2-yl)-1-(2-methylpropyl)-4-oxo-2-buten-1-yl]-1, alaninamide;
(2E,4S)—N-[4-(methyloxy)phenyl]-6-phenyl-4-{[3-(2-thienyl)-L-alanyl]amino}-2-hexenamide;
(2E,4S)-4-{[(2S)-2-aminobutanoyl]amino}-N-[4-(methyloxy)phenyl]-6-phenyl-2-hexenamide;
(2E,4S)-6-phenyl-N-propyl-4-{[3-(2-thienyl)-L-alanyl]amino}-2-hexenamide;
(2E,4S)-4-{[(2S)-2-aminobutanoyl]amino}-N-[5-(1-methylcyclobutyl)-1,3,4-thiadiazol-2-yl]-6-phenyl-2-hexenamide;
(2S)—N-[(1S,2E)-4-{[4-(methyloxy)phenyl]amino}-4-oxo-1-(2-phenylethyl)-2-buten-1-yl]-2-azetidinecarboxamide;
(2E,4S)-4-{[(2S)-2-amino-2-cyclopentylacetyl]amino}-N-[4-(methyloxy)phenyl]-6-phenyl-2-hexenamide;
(2E,4S)—N-[4-(methyloxy)phenyl]-6-phenyl-4-(L-valylamino)-2-hexenamide;
(2S)—N-[(1S,2E)-4-(2,3-dihydro-1H-indol-1-yl)-1-ethyl-4-oxo-2-buten-1-yl]-2-azetidinecarboxamide;
(2S)—N-[(1S,2E)-4-(2,3-dihydro-1H-indol-1-yl)-1-(2-methylpropyl)-4-oxo-2-buten-1-yl]-2-azetidinecarboxamide;
(2S)—N-[(1S,2E)-1-(cyclopropylmethyl)-4-(2,3-dihydro-1H-indol-1-yl)-4-oxo-2-buten-1-yl]-2-azetidinecarboxamide;
(4S)—N-[(1S,2E)-4-(2,3-dihydro-1H-indol-1-yl)-1-ethyl-4-oxo-2-buten-1-yl]-4-fluoro-L-prolinamide;
(2S)—N-[(1S,2E)-4-(2,3-dihydro-1H-indol-1-yl)-1-ethyl-4-oxo-2-buten-1-yl]-2-piperidinecarboxamide;
(2S)—N-[(1S,2E)-4-(2,3-dihydro-1H-indol-1-yl)-1-(2-methylpropyl)-4-oxo-2-buten-1-yl]-2-piperidinecarboxamide;
(2S)—N-((1S,2E)-1-(2-methylpropyl)-4-oxo-4-{[5-(trifluoromethyl)-1,3,4-thiadiazol-2-yl]amino}-2-buten-1-yl)-2-azetidinecarboxamide;
(2S)—N-((1S,2E)-1-(2-methylpropyl)-4-{methyl[5-(trifluoromethyl)-1,3,4-thiadiazol-2-yl]amino}-4-oxo-2-buten-1-yl)-2-azetidinecarboxamide;
(4S)—N-((1 S,2E)-1-ethyl-4-oxo-4-{[5-(trifluoromethyl)-1,3,4-thiadiazol-2-yl]amino}-2-buten-1-yl)-4-fluoro-L-prolinamide;
(2S)—N-((1S,2E)-1-(2-methylpropyl)-4-oxo-4-{[5-(trifluoromethyl)-1,3,4-thiadiazol-2-yl]amino}-2-buten-1-yl)-2-piperidinecarboxamide;
(2S)—N-[(1S,2E)-4-{[5-(1-methyl-1-phenylethyl)-1,3,4-thiadiazol-2-yl]amino}-4-oxo-1-(2-phenylethyl)-2-buten-1-yl]-2-piperidinecarboxamide;
(2S)—N-[(3S,4E)-6-(2,3-dihydro-1H-indol-1-yl)-6-oxo-1-phenylhex-4-en-3-yl]azetidine-2-carboxamide;
(2S)—N-[(3S,4E)-6-(1,3-dihydro-2H-isoindol-2-yl)-6-oxo-1-phenylhex-4-en-3-yl]azetidine-2-carboxamide;
(2E,4S)-4-{[(2S)-2-amino-2-cyclopropylacetyl]amino}-N-(4-methoxyphenyl)-6-phenylhex-2-enamide;
(2S)-2-amino-2-cyclopentyl-N-[(3S,4E)-6-(2,3-dihydro-1H-indol-1-yl)-6-oxo-1-phenylhex-4-en-3-yl]ethanamide;
(2S)-2-amino-2-cyclopentyl-N-[(3S,4E)-6-(1,3-dihydro-2H-isoindol-2-yl)-6-oxo-1-phenylhex-4-en-3-yl]ethanamide;
(2S)-2-amino-N-[(3S,4E)-6-(2,3-dihydro-1H-indol-1-yl)-6-oxo-1-phenylhex-4-en-3-yl]butanamide;
N-[(3S,4E)-6-(2,3-dihydro-1H-indol-1-yl)-6-oxo-1-phenylhex-4-en-3-yl]-3-thiophen-2-yl-L-alaninamide;
N-[(3S,4E)-6-(1,3-dihydro-2H-isoindol-2-yl)-6-oxo-1-phenylhex-4-en-3-yl]-L-isoleucinamide;
N-[(3S,4E)-6-(1,3-dihydro-2H-isoindol-2-yl)-6-oxo-1-phenylhex-4-en-3-yl]-L-alloisoleucinamide;
N-[(3S,4E)-6-(1,3-dihydro-2H-isoindol-2-yl)-6-oxo-1-phenylhex-4-en-3-yl]-3-methyl-L-valinamide;
(2S)—N-[(3S,4E)-6-(1,3-dihydro-2H-isoindol-2-yl)-6-oxo-1-phenylhex-4-en-3-yl]piperidine-2-carboxamide;
N-[(3S,4E)-6-(1,3-dihydro-2H-isoindol-2-yl)-6-oxo-1-phenylhex-4-en-3-yl]-L-prolinamide;
(2S)—N-[(3S,4E)-6-(1,3-dihydro-2H-isoindol-2-yl)-6-oxo-1-phenylhex-4-en-3-yl]-2-(methylamino)butanamide;
(2S)—N-[(3S,4E)-6-oxo-1-phenyl-6-{[5-(trifluoromethyl)-1,3,4-thiadiazol-2-yl]amino}hex-4-en-3-yl]piperidine-2-carboxamide;
(2S)—N-{(3S,4E)-6-[(5-methyl-1,3,4-thiadiazol-2-yl)amino]-6-oxo-1-phenylhex-4-en-3-yl}piperidine-2-carboxamide;
(2S)—N-{(3S,4E)-6-[(5-ethyl-1,3,4-thiadiazol-2-yl)amino]-6-oxo-1-phenylhex-4-en-3-yl}piperidine-2-carboxamide;
(2S)—N-{(3S,4E)-6-[(5-tert-butyl-1,3,4-thiadiazol-2-yl)amino]-6-oxo-1-phenylhex-4-en-3-yl}piperidine-2-carboxamide;
(2S)—N-[(3S,4E)-6-{[5-(methylsulfanyl)-1,3,4-thiadiazol-2-yl]amino}-6-oxo-1-phenylhex-4-en-3-yl]piperidine-2-carboxamide;
(2S)—N-{(3S,4E)-6-oxo-1-phenyl-6-[(5-phenyl-1,3,4-thiadiazol-2-yl)amino]hex-4-en-3-yl}piperidine-2-carboxamide;
(2S)—N-[(3S,4E)-6-{[5-(4-bromophenyl)-1,3,4-thiadiazol-2-yl]amino}-6-oxo-1-phenylhex-4-en-3-yl]piperidine-2-carboxamide;
(2S)—N-[(3S,4E)-6-{[5-(4-fluorophenyl)-1,3,4-thiadiazol-2-yl]amino}-6-oxo-1-phenylhex-4-en-3-yl]piperidine-2-carboxamide;
(2S)—N-{(3S,4E)-6-[(5-cyclopropyl-1,3,4-thiadiazol-2-yl)amino]-6-oxo-1-phenylhex-4-en-3-yl}piperidine-2-carboxamide;
(2S)—N-[(3S,4E)-6-oxo-1-phenyl-6-{[5-(propan-2-yl)-1,3,4-thiadiazol-2-yl]amino}hex-4-en-3-yl]piperidine-2-carboxamide;
(2S)—N-{(3S,4E)-6-[(5-benzyl-1,3,4-thiadiazol-2-yl)amino]-6-oxo-1-phenylhex-4-en-3-yl}piperidine-2-carboxamide;
(2S)—N-[(3S,4E)-6-oxo-1-phenyl-6-{[5-(2-phenylethyl)-1,3,4-thiadiazol-2-yl]amino}hex-4-en-3-yl]piperidine-2-carboxamide;
(2S)—N-{(3S,4E)-6-[(5-cyclohexyl-1,3,4-thiadiazol-2-yl)amino]-6-oxo-1-phenylhex-4-en-3-yl}piperidine-2-carboxamide;
(2S)-2-amino-2-cyclopropyl-N-[(3S,4E)-6-(2,3-dihydro-1H-indol-1-yl)-6-oxo-1-phenylhex-4-en-3-yl]ethanamide;
(2S)-2-amino-2-cyclopropyl-N-[(3S,4E)-6-(1,3-dihydro-2H-isoindol-2-yl)-6-oxo-1-phenylhex-4-en-3-yl]ethanamide;
N-[(3S,4E)-6-(1,3-dihydro-2H-isoindol-2-yl)-6-oxo-1-phenylhex-4-en-3-yl]-L-valinamide;
(1R,2S,5S)—N-[(3S,4E)-6-(2,3-dihydro-1H-indol-1-yl)-6-oxo-1-phenylhex-4-en-3-yl]-3-azabicyclo[3.1.0]hexane-2-carboxamide;
(1S,2R,5R)—N-[(3S,4E)-6-(2,3-dihydro-1H-indol-1-yl)-6-oxo-1-phenylhex-4-en-3-yl]-3-azabicyclo[3.1.0]hexane-2-carboxamide;
N-[(3S,4E)-6-(2,3-dihydro-1H-indol-1-yl)-6-oxo-1-phenylhex-4-en-3-yl]-L-valinamide;
(2E,4S)—N-methyl-6-phenyl-N-[5-(trifluoromethyl)-1,3,4-thiadiazol-2-yl]-4-(L-valylamino)hex-2-enamide;
(2E,4S)-6-phenyl-N-[5-(trifluoromethyl)-1,3,4-thiadiazol-2-yl]-4-(L-valylamino)hex-2-enamide;
(2S)—N-[(3S,4E)-6-oxo-1-phenyl-6-{[5-(trifluoromethyl)-1,3,4-thiadiazol-2-yl]amino}hex-4-en-3-yl]azetidine-2-carboxamide;
(2S)—N-[(3S,4E)-6-{methyl[5-(trifluoromethyl)-1,3,4-thiadiazol-2-yl]amino}-6-oxo-1-phenylhex-4-en-3-yl]azetidine-2-carboxamide;
(2S)—N-[(3S,4E)-6-(2,3-dihydro-1H-indol-1-yl)-6-oxo-1-phenylhex-4-en-3-yl]piperidine-2-carboxamide;
(4S)—N-[(3S,4E)-6-(2,3-dihydro-1H-indol-1-yl)-6-oxo-1-phenylhex-4-en-3-yl]-4-fluoro-L-prolinamide;
N-[(2S,3E)-1-cyclohexyl-5-(1,3-dihydro-2H-isoindol-2-yl)-5-oxopent-3-en-2-yl]-L-alaninamide;
N-[(2S,3E)-1-cyclohexyl-5-(2,3-dihydro-1H-indol-1-yl)-5-oxopent-3-en-2-yl]-L-alaninamide;
(2S)-2-amino-N-[(2S,3E)-1-cyclohexyl-5-(1,3-dihydro-2H-isoindol-2-yl)-5-oxopent-3-en-2-yl]butanamide;
(2S)-2-amino-N-[(2S,3E)-1-cyclohexyl-5-(2,3-dihydro-1H-indol-1-yl)-5-oxopent-3-en-2-yl]butanamide;
N-[(2S,3E)-1-cyclohexyl-5-(1,3-dihydro-2H-isoindol-2-yl)-5-oxopent-3-en-2-yl]-3-thiophen-2-yl-L-alaninamide;
N-[(2S,3E)-1-cyclohexyl-5-(2,3-dihydro-1H-indol-1-yl)-5-oxopent-3-en-2-yl]-3-thiophen-2-yl-L-alaninamide;
(2S)—N-[(2S,3E)-1-cyclohexyl-5-(1,3-dihydro-2H-isoindol-2-yl)-5-oxopent-3-en-2-yl]azetidine-2-carboxamide;
(2S)—N-[(2S,3E)-1-cyclohexyl-5-(2,3-dihydro-1H-indol-1-yl)-5-oxopent-3-en-2-yl]azetidine-2-carboxamide;
(2S)—N-[(2S,3E)-1-cyclohexyl-5-{methyl[5-(trifluoromethyl)-1,3,4-thiadiazol-2-yl]amino}-5-oxopent-3-en-2-yl]azetidine-2-carboxamide;
(2S)—N-[(2S,3E)-1-cyclohexyl-5-oxo-5-{[5-(trifluoromethyl)-1,3,4-thiadiazol-2-yl]amino}pent-3-en-2-yl]azetidine-2-carboxamide;
(2S)—N-{(2S,3E)-5-[(5-tert-butyl-1,3,4-thiadiazol-2-yl)amino]-1-cyclohexyl-5-oxopent-3-en-2-yl}azetidine-2-carboxamide;
(2S)—N-[(2S,3E)-1-cyclobutyl-5-(2,3-dihydro-1H-indol-1-yl)-5-oxopent-3-en-2-yl]azetidine-2-carboxamide;
(2S)—N-[(2S,3E)-1-cyclobutyl-5-(2,3-dihydro-1H-indol-1-yl)-5-oxopent-3-en-2-yl]piperidine-2-carboxamide;
(2S)—N-[(2S,3E)-1-cyclobutyl-5-oxo-5-{[5-(trifluoromethyl)-1,3,4-thiadiazol-2-yl]amino}pent-3-en-2-yl]piperidine-2-carboxamide;
N-[(2E,4S)-1-(1,3-dihydro-2H-isoindol-2-yl)-6,6-dimethyl-1-oxohept-2-en-4-yl]-3-thiophen-2-yl-L-alaninamide;
N-[(2E,4S)-1-(2,3-dihydro-1H-indol-1-yl)-6,6-dimethyl-1-oxohept-2-en-4-yl]-3-thiophen-2-yl-L-alaninamide;
(2E,4S)—N-(4-methoxyphenyl)-6,6-dimethyl-4-{[3-(thiophen-2-yl)-L-alanyl]amino}hept-2-enamide;
(2E,4S)-4-(L-alanylamino)-N-(4-methoxyphenyl)-6,6-dimethylhept-2-enamide;
N-[(2E,4S)-1-(1,3-dihydro-2H-isoindol-2-yl)-6,6-dimethyl-1-oxohept-2-en-4-yl]-1, alaninamide;
N-[(2E,4S)-1-(2,3-dihydro-1H-indol-1-yl)-6,6-dimethyl-1-oxohept-2-en-4-yl]-1, alaninamide;
(2S)-2-amino-N-[(2E,4S)-1-(1,3-dihydro-2H-isoindol-2-yl)-6,6-dimethyl-1-oxohept-2-en-4-yl]butanamide;
(2S)-2-amino-N-[(2E,4S)-1-(2,3-dihydro-1H-indol-1-yl)-6,6-dimethyl-1-oxohept-2-en-4-yl]butanamide;
(2E,4S)-4-{[(2S)-2-aminobutanoyl]amino}-N-(4-methoxyphenyl)-6,6-dimethylhept-2-enamide;
(2E,4S)-4-{[(2S)-2-aminobutanoyl]amino}-N-(4-methoxyphenyl)-6-methylhept-2-enamide;
(2E,4S)—N-(4-methoxyphenyl)-6-methyl-4-{[3-(thiophen-2-yl)-L-alanyl]amino}hept-2-enamide;
N-[(2E,4S)-1-(2,3-dihydro-1H-indol-1-yl)-6-methyl-1-oxohept-2-en-4-yl]-1, alaninamide;
N-[(2E,4S)-1-(2,3-dihydro-1H-indol-1-yl)-6-methyl-1-oxohept-2-en-4-yl]-3-thiophen-2-yl-L-alaninamide;
(2S)-2-amino-N-[(2E,4S)-1-(2,3-dihydro-1H-indol-1-yl)-6-methyl-1-oxohept-2-en-4-yl]butanamide;
N-{(2E,4S)-6-methyl-1-oxo-1-[(1R,4S)-1,2,3,4-tetrahydro-1,4-epiminonaphthalen-9-yl]hept-2-en-4-yl}-3-thiophen-2-yl-L-alaninamide;
(2S)-2-amino-N-{(2E,4S)-6-methyl-1-oxo-1-[(1R,4S)-1,2,3,4-tetrahydro-1,4-epiminonaphthalen-9-yl]hept-2-en-4-yl}butanamide;
(2E,4S)—N-(5-tert-butyl-1,3,4-thiadiazol-2-yl)-6-methyl-4-{[3-(thiophen-2-yl)-L-alanyl]amino}hept-2-enamide;
N-[(2E,4S,5S)-1-(2,3-dihydro-1H-indol-1-yl)-5-methyl-1-oxohept-2-en-4-yl]-1, alaninamide;
(2E,4S)—N-[5-(4-fluorophenyl)-1,3,4-thiadiazol-2-yl]-6-methyl-4-{[3-(thiophen-2-yl)-L-alanyl]amino}hept-2-enamide;
(2S)—N-[(2E,4S)-1-(1,3-dihydro-2H-isoindol-2-yl)-6-methyl-1-oxohept-2-en-4-yl]azetidine-2-carboxamide;
(2S)—N-[(2E,4S)-1-(2,3-dihydro-1H-indol-1-yl)-6-methyl-1-oxohept-2-en-4-yl]azetidine-2-carboxamide;
(2S)—N-{(2E,4S)-1-[(4-methoxyphenyl)amino]-6-methyl-1-oxohept-2-en-4-yl}azetidine-2-carboxamide;
(2S)—N-[(2E,4S)-6-methyl-1-{methyl[5-(trifluoromethyl)-1,3,4-thiadiazol-2-yl]amino}-1-oxohept-2-en-4-yl]piperidine-2-carboxamide;
(2S)—N-{(2S,3E)-5-[(5-tert-butyl-1,3,4-thiadiazol-2-yl)amino]-1-cyclopropyl-5-oxopent-3-en-2-yl}azetidine-2-carboxamide;
(2S)—N-[(2S,3E)-1-cyclopropyl-5-(1,3-dihydro-2H-isoindol-2-yl)-5-oxopent-3-en-2-yl]azetidine-2-carboxamide;
N-[(2E,4S)-1-(1,3-dihydro-2H-isoindol-2-yl)-1-oxohept-2-en-4-yl]-L-alaninamide;
N-[(2E,4S)-1-(2,3-dihydro-1H-indol-1-yl)-1-oxohept-2-en-4-yl]-L-alaninamide;
(2E,4S)-4-(L-alanylamino)-N-(4-methoxyphenyl)hept-2-enamide;
N-[(2E,4S)-1-(1,3-dihydro-2H-isoindol-2-yl)-1-oxohept-2-en-4-yl]-3-thiophen-2-yl-L-alaninamide;
N-[(2E,4S)-1-(2,3-dihydro-1H-indol-1-yl)-1-oxohept-2-en-4-yl]-3-thiophen-2-yl-L-alaninamide;
(2E,4S)—N-(4-methoxyphenyl)-4-{[3-(thiophen-2-yl)-L-alanyl]amino}hept-2-enamide;
(2E,4S)-4-{[(2S)-2-aminobutanoyl]amino}-N-(4-methoxyphenyl)hept-2-enamide;
(2S)-2-amino-N-[(2E,4S)-1-(1,3-dihydro-2H-isoindol-2-yl)-1-oxohept-2-en-4-yl]butanamide;
(2S)-2-amino-N-[(2E,4S)-1-(2,3-dihydro-1H-indol-1-yl)-1-oxohept-2-en-4-yl]butanamide;
(2E,4S)-4-(L-alanylamino)-4-cyclopropyl-N-(4-methoxyphenyl)but-2-enamide;
N-[(1S,2E)-1-cyclopropyl-4-(1,3-dihydro-2H-isoindol-2-yl)-4-oxobut-2-en-1-yl]-L-alaninamide;
N-{(1S,2E)-1-cyclopropyl-4-(2,3-dihydro-1H-indol-1-yl)-4-oxobut-2-en-1-yl}-1, alaninamide;
N-{(1S,2E)-1-cyclopropyl-4-[(4-methoxyphenyl)amino]-4-oxobut-2-en-1-yl}-L-valinamide;
N-[(1S,2E)-1-cyclopropyl-4-(1,3-dihydro-2H-isoindol-2-yl)-4-oxobut-2-en-1-yl]-L-valinamide;
N-[(1S,2E)-1-cyclopropyl-4-(2,3-dihydro-1H-indol-1-yl)-4-oxobut-2-en-1-yl]-1, valinamide;
(2S)-2-amino-2-cyclopentyl-N-[(1S,2E)-1-cyclopropyl-4-(1,3-dihydro-2H-isoindol-2-yl)-4-oxobut-2-en-1-yl]ethanamide;
(2S)-2-amino-2-cyclopentyl-N-[(1S,2E)-1-cyclopropyl-4-(2,3-dihydro-1H-indol-1-yl)-4-oxobut-2-en-1-yl]ethanamide;
(2S)—N-[(1S,2E)-1-cyclopropyl-4-(1,3-dihydro-2H-isoindol-2-yl)-4-oxobut-2-en-1-yl]azetidine-2-carboxamide;
(2S)—N-[(1S,2E)-1-cyclopropyl-4-(2,3-dihydro-1H-indol-1-yl)-4-oxobut-2-en-1-yl]azetidine-2-carboxamide;
(2S)—N-[(1S,2E)-1-cyclopropyl-4-oxo-4-{[5-(trifluoromethyl)-1,3,4-thiadiazol-2-yl]amino}but-2-en-1-yl]azetidine-2-carboxamide;
(2S)—N-{(1S,2E)-4-[(5-tert-butyl-1,3,4-thiadiazol-2-yl)amino]-1-cyclopropyl-4-oxobut-2-en-1-yl}azetidine-2-carboxamide;
(2S)-2-amino-N-[(3S,4E)-6-(1,3-dihydro-2H-isoindol-2-yl)-6-oxohex-4-en-3-yl]butanamide;
N-[(3S,4E)-6-(1,3-dihydro-2H-isoindol-2-yl)-6-oxohex-4-en-3-yl]-3-thiophen-2-yl-L-alaninamide;
(2E,4S)-4-{[(2S)-2-aminobutanoyl]amino}-N-(4-methoxyphenyl)hex-2-enamide;
(2S)-2-amino-N-{(3S,4E)-6-oxo-6-[(1R,4S)-1,2,3,4-tetrahydro-1,4-epiminonaphthalen-9-yl]hex-4-en-3-yl}butanamide;
(2E,4S)—N-(4-methoxyphenyl)-4-{[3-(thiophen-2-yl)-L-alanyl]amino}hex-2-enamide;
N-[(3S,4E)-6-oxo-6-(1,2,3,4-tetrahydro-1,4-epiminonaphthalen-9-yl)hex-4-en-3-yl]-3-thiophen-2-yl-L-alaninamide;
N-[(3S,4E)-6-(2,3-dihydro-1H-indol-1-yl)-6-oxohex-4-en-3-yl]-L-alaninamide;
(2S)-2-amino-N-[(3S,4E)-6-(2,3-dihydro-1H-indol-1-yl)-6-oxohex-4-en-3-yl]butanamide;
N-[(3S,4E)-6-(2,3-dihydro-1H-indol-1-yl)-6-oxohex-4-en-3-yl]-3-thiophen-2-yl-L-alaninamide;
(2S)—N-[(3S,4E)-6-(1,3-dihydro-2H-isoindol-2-yl)-6-oxohex-4-en-3-yl]azetidine-2-carboxamide;
(2S)—N-[(3S,4E)-6-(2,3-dihydro-1H-indol-1-yl)-6-oxohex-4-en-3-yl]azetidine-2-carboxamide;
(2S)—N-{(3S,4E)-6-[(4-methoxyphenyl)amino]-6-oxohex-4-en-3-yl}azetidine-2-carboxamide;
(2S)—N-[(3S,4E)-6-oxo-6-{[5-(trifluoromethyl)-1,3,4-thiadiazol-2-yl]amino}hex-4-en-3-yl]piperidine-2-carboxamide;
(2S)—N-[(3S,4E)-6-{[5-(4-fluorophenyl)-1,3,4-thiadiazol-2-yl]amino}-6-oxohex-4-en-3-yl]piperidine-2-carboxamide;
(2S)—N-[(3S,4E)-6-{methyl[5-(trifluoromethyl)-1,3,4-thiadiazol-2-yl]amino}-6-oxohex-4-en-3-yl]piperidine-2-carboxamide; or
N-[(4E)-6-(2,3-dihydro-1H-indol-1-yl)-1,1,1-trifluoro-6-oxohex-4-en-3-yl]-L-alaninamide;
or a pharmaceutically acceptable salt thereof.

56. A compound which is (4S)—N-[(1S,2E)-4-(2,3-dihydro-1H-indol-1-yl)-1-(2-methylpropyl)-4-oxo-2-buten-1-yl]-4-fluoro-L-prolinamide or a pharmaceutically acceptable salt thereof.

57. A pharmaceutical composition which comprises the compound or salt according to claim 40, and a pharmaceutically acceptable excipient.

58. A method for treating chronic obstructive pulmonary disease comprising administering to a patient in need thereof an effective amount of the compound or salt according to claim 40.

59. A method for treating chronic obstructive pulmonary disease comprising administering to a patient in need thereof the pharmaceutical composition according to claim 57.
